(19) United States
(12) Patent Application Publication
(10) Pub. No.: US 2012/0178682 A1
(43) Pub. Date: Jul. 12, 2012

(54) GB1 Peptidic Compounds and Methods for Making and Using the Same

(76) Inventors: SACHDEV S. SIDHU, Toronto (CA); Maruti Uppalapati, Toronto (CA)

(21) Appl. No.: 13/294,097

(22) Filed: Nov. 10, 2011

Related U.S. Application Data
(60) Provisional application No. 61/413,331, filed on Nov. 12, 2010.

(51) Int. Cl. A61K 38/16; A61P 27/02; C12N 5/071; A61P 35/00; A61P 35/04; C07K 14/00; A61P 25/00

(52) U.S. Cl. 514/8.1; 530/324; 435/375

(57) ABSTRACT

GB1 peptidic compounds that specifically bind to a target molecule are provided. Also provided are methods for making and using the compounds. These compounds and methods find use in a variety of applications in which specific binding to target molecules, e.g., target proteins, is desired.

### CROSS REFERENCE TO RELATED APPLICATIONS

Pursuant to 35 U.S.C. §119(e), this application claims priority to the filing date of U.S. provisional application Ser. No. 61/413,331, filed Nov. 12, 2010, the disclosure of which is herein incorporated by reference.

This application is related to copending U.S. application entitled “GB1 peptidic libraries and methods of screening the same” filed on Nov. 10, 2011 to Sidhu et al. (attorney reference number RFLX-001) and accorded Ser. No. ______, and U.S. provisional application Ser. No. 61/413,318 filed Nov. 12, 2010, which are entirely incorporated herein by reference.

This application is related to copending U.S. application entitled “Methods and compositions for identifying D-peptidic compounds that specifically bind target proteins” filed on Nov. 10, 2011 to Ault-Riché et al. (attorney reference number RFLX-002) and accorded Ser. No. ______, and U.S. provisional application Ser. No. 61/413,316 filed Nov. 12, 2010, which are entirely incorporated herein by reference.

### INTRODUCTION

Essentially all biological processes depend on molecular recognition mediated by proteins. The ability to manipulate the interactions of such proteins is of interest for both basic biological research and for the development of therapeutics and diagnostics.

Protein ligands can form large binding surfaces with multiple contacts to a target molecule that leads to binding events with high specificity and affinity. For example, antibodies are a class of protein that has yielded specific and tight binding ligands for various target antigens.

Because of the diversity of target molecules of interest and the binding properties of protein ligands, the preparation of binding proteins with useful functions is of interest.

For example, vascular endothelial growth factor (VEGF) has a key role in the regulation of normal and abnormal angiogenesis and in the development and differentiation of the vascular system (Ferrara et al. Endocr. Rev. 18:4 25 (1997)). Furthermore, VEGF has been shown to be a key mediator of neovascularization associated with tumors and intraocular disorders (Ferrara et al.). Therefore, compounds that inhibit VEGF action are of interest for the treatment of, e.g., solid tumors and various intraocular neovascular disorders.

### SUMMARY

GB 1 peptidic compounds that specifically bind to a target molecule are provided. Also provided are methods for making and using the compounds. These compounds and methods find use in a variety of applications in which specific binding to target molecules, e.g., target proteins, is desired.

### BRIEF DESCRIPTION OF THE DRAWINGS

FIG. 1 depicts a ribbon structure of a GB1 protein that illustrates a 4β-1α motif (Mayo et al., Nature Structural Biology, 5(6), 1998, p. 470-475).

FIGS. 2A and 2B depicts several groups of amino acids at various positions of a GB1 scaffold, both in a ribbon representation (top) and a space filling representation (bottom). Amino acids at several positions of the GB1 scaffold that are selected for mutation are highlighted in dark circles (top). The space filling representations of Libraries 1 to 6 (bottom) illustrate six different potential binding surfaces (shown in dark) on the GB1 scaffold.

FIG. 3 illustrates the underlying sequence of the GB1 scaffold domain of FIG. 2A-2B and the positions of the variant amino acids (shown in dark blocks) in Surfaces 1 to 6. The asterisks indicate positions (e.g., 1, 9, 19, 38, 47 and 55) at which mutations may include insertion of amino acids.

FIG. 4 illustrates the formulas of affinity maturation compounds prepared to optimize the specificity and binding affinity of VEGF-binding compounds. Each X is a variant amino acid. E01-AM1-1 to B03-AM-4 (SEQ ID NO:349 to SEQ ID NO:360)

FIG. 5 shows LC-MS data from the synthesis of D-VEGF. RP-HPLC chromatograms (top) illustrate each step in the synthesis of D-VEGF. Also shown (bottom) is ESI MS data for the product (Mass obs. 11,932 +/−1 Da, Mass calc. 11932.6 Da).

FIG. 6 shows LC chromatograms that illustrate the folding of synthetic D-VEGF (top), and LC-MS data of purified folded D-VEGF (bottom).

FIG. 7 shows an overlay of the X-ray crystal structures of the E01AM1 compound (dark strand) and native GB1 (light strand). The arrows indicate differences in the position of the W42 residue (left) and the position of the loop between α1 and β3 (top arrow).

FIG. 8 depicts front and back views of stability optimization library 1 (A) and library 2 (B) and the positions of mutations (dark residues).

FIGS. 9-20 illustrate the sequences of exemplary D-VEGF binding GB1 peptidic compounds.

FIGS. 21-24 illustrate the sequences of exemplary L-VEGF binding GB1 peptidic compounds.

FIG. 25 illustrates sequences of exemplary GB1 peptidic compounds that bind specifically to 3BP2-SH2, ABL-SH3, vSrc-SH3 or IL-13, respectively.

FIG. 26 illustrates sequences of exemplary GB1 peptidic compounds that bind specifically to IL-13, TNF-alpha, BAFF or RANKL, respectively.

### DEFINITIONS

As used herein, the term “peptidic” refers to a moiety that is composed of amino acid residues. The term “peptidic” includes compounds or libraries in which the conventional backbone has been replaced with non-naturally occurring or synthetic backbones, and peptides in which one or more naturally occurring amino acids have been replaced with one or more non-naturally occurring or synthetic amino acids, or a D-amino acid version thereof. Any of the depictions of sequences found herein (e.g., using one-letter or three-letter codes) may represent a L-amino acid or a D-amino acid version of the sequence. Unless noted otherwise, the capital and small letter codes for L- and D-amino acid residues, respectively, are not utilized.

As used herein, the terms “polypeptide” and “protein” are used interchangeably. The term “polypeptide” also includes post translational modified polypeptides or proteins. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. In some instances, polypeptides may be of any length, e.g., 2 or more amino acids, 4 or more amino acids, 10 or more amino acids, 20 or more amino acids, 30 or more amino acids, 40 or more amino acids, 50 or more amino acids, 60 or more amino acids, 100 or more amino acids, 300 or more amino acids, 500 or more or 1000 or more amino acids.

As used herein, the terms “naturally occurring amino acid” and “non-naturally occurring amino acid” may be used to refer to both L- and D-versions of these amino acids. For example, a D-peptidic compound may be described as including naturally occurring amino acids, e.g., D-enantiomers of amino acids such as A, C, D, E, F, H, I, K, L, M, N, P, Q, R, S, T, V, W and Y.

As used herein, the term “scaffold” or “scaffold domain” refers to a peptidic framework from which a compound arose, and against which the compound is able to be compared. When a compound arises from amino acid mutations at various positions within a scaffold, the amino acids at those positions are referred to as “variant amino acids.” Such variant amino acids may confer on the resulting peptidic compounds different functions, such as specific binding to a target protein.

As used herein, the term “mutation” refers to a deletion, insertion, or substitution of an amino acid(s) residue or nucleotide(s) residue relative to a reference sequence, such as a scaffold sequence.

As used herein, the term “domain” refers to a continuous or discontinuous sequence of amino acid residues. As used herein, the term “region” refers to a continuous sequence of amino acid residues.

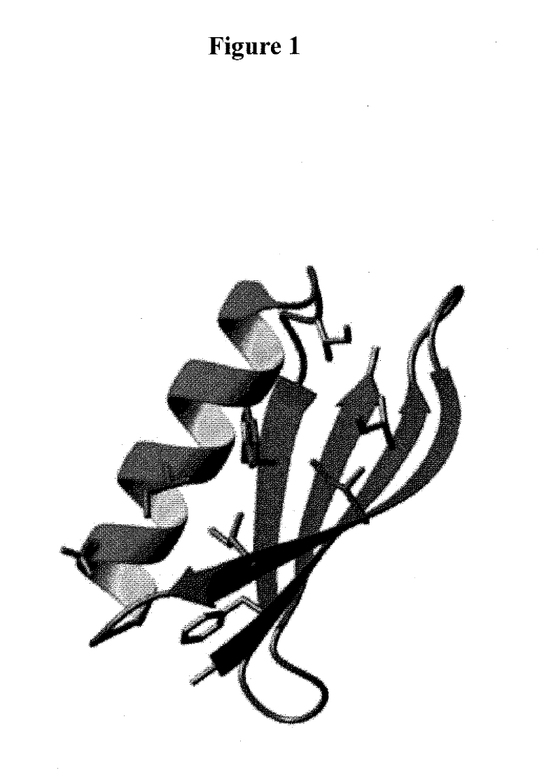

As used herein, the terms “GB1 scaffold domain” and “GB1 scaffold” refer to a scaffold that has a structural motif similar to the B1 domain of Protein G (GB1), where the structural motif is characterized by a motif including a four stranded β-sheet packed against a helix (also referred to as a 4β-1α motif). The arrangement of four β-strands and one α-helix may form a hairpin-helix-hairpin motif. An exemplary GB1 scaffold domain is depicted in FIG. 1. GB1 scaffold domains include members of the family of IgG binding B domains, e.g., Protein L B1 domain. Amino acid sequences of exemplary B domains that may be employed herein as GB1 scaffold domains are found in the Wellcome Trust Sanger Institute Pfam database (The Pfam protein families database: Finn et al., Nucleic Acids Research (2010) Database Issue 38:D211-222), see, e.g., Family: IgG_binding_B (PF01378) (pfam.sanger.ac.uk/family/PF01378.10#tabview=tab0) or in NCBI's protein database. Exemplary GB1 scaffold domain sequences include those described by SEQ ID NOs:314-348. A GB1 scaffold domain may be a native sequence of a member of the B domain protein family, a B domain sequence with pre-existing amino acid sequence modifications (such as additions, deletions and/or substitutions), or a fragment or analogue thereof. A GB1 scaffold domain may be L-peptidic, D-peptidic or a combination thereof. In some cases, a “GB1 scaffold domain” may also be referred to as a “parent amino acid sequence.”

As used herein, the term “GB1 peptidic compound” refers to a compound composed of peptidic residues that has a parent GB1 scaffold domain.

As used herein, the term “parent amino acid sequence” and “parent polypeptide” refer to a polypeptide comprising an amino acid sequence from which a variant GB1 peptidic compound arose and against which the variant GB1 peptidic compound is being compared. In some cases, the parent polypeptide lacks one or more of the modifications disclosed herein and differs in function compared to a variant GB1 peptidic compound as disclosed herein. The parent polypeptide may comprise a native GB1 sequence or GB1 scaffold sequence with pre-existing amino acid sequence modifications (such as additions, deletions and/or substitutions).

As used herein, the term “variable region” refers to a continuous sequence of residues that includes one or more variant amino acids. A variable region may include one or more conserved amino acids at fixed positions. As used herein, the term “fixed region” refers to a continuous sequence of conserved residues that does not include any mutations or variant amino acids, and is conserved across a library of compounds.

As used herein, the term “variable domain” refers to a domain that includes all of the variant amino acids of a GB1 scaffold. The variable domain may include one or more variable regions, and may encompass a continuous or a discontinuous sequence of residues. The variable domain may be part of the GB1 scaffold domain.

As used herein, the term “discontinuous sequence of residues” refers to a sequence of residues that is not continuous with respect to the primary sequence of a peptidic compound. A peptidic compound may fold to form a secondary or tertiary structure, e.g., a 4β-1α motif, where the amino acids of a discontinuous sequence of residues are adjacent to each other in space, i.e., contiguous. As used herein, the term “continuous sequence of residues” refers to a sequence of residues that is continuous in terms of the primary sequence of a peptidic compound.

As used herein, the term “non-core mutation” refers to an amino acid mutation of a GB1 peptidic compound that is located at a position in the 4β-1α structure that is not part of the hydrophobic core of the structure. Amino acid residues in the hydrophobic core of a GB1 peptidic compound are not significantly solvent exposed but rather tend to form intramolecular hydrophobic contacts. Unless explicitly defined otherwise, a hydrophobic core residue or core position, as described herein, of a GB1 scaffold domain that is described by SEQ ID NO:1 is defined by one of positions 2, 4, 6, 19, 25, 29, 33, 38, 42, 51 and 53 of the GB1 scaffold. The methodology used to specify hydrophobic core residues in GB1 is described by Dahiyat et al., (“Probing the role of packing specificity in protein design,” Proc. Natl. Acad. Sci. USA, 1997, 94, 10172-10177) where a PDB structure was used to calculate which side chains expose less than 10% of their surface area to solvent. Such methods can be modified for use with the GB1 scaffold domain.

As used herein, the term “surface mutation” refers to an amino acid mutation in a GB1 scaffold that is located at a position in the 4β-1α structure that is solvent exposed. Such variant amino acid residues at surface positions of a GB1 peptidic compound are capable of interacting directly with a target molecule, whether or not such an interaction occurs.

As used herein, the term “boundary mutation” refers to an amino acid mutation in a GB1 scaffold that is located at a position in the 4β-1α structure that is at the boundary between the hydrophobic core and the solvent exposed surface. Such variant amino acid residues at boundary positions of a GB1 peptidic compound may be in part contacting hydrophobic core residues and/or in part solvent exposed and capable of some interaction with a target molecule, whether or not such an interaction occurs. One criteria for describing core, surface and boundary residues of a GB1 peptidic structure is described by Mayo et al. Nature Structural Biology, 5(6), 1998, 470-475. Such methods and criteria can be modified for use with the GB1 scaffold domain.

As used herein, the term “linking sequence” refers to a continuous sequence of amino acid residues, or analogs thereof, that connect two peptidic motifs. In certain embodiments, a linking sequence is the loop connecting β-strands in a β-hairpin motif.

As used herein, the term “stable” refers to a compound that is able to maintain a folded state under physiological conditions at a certain temperature, such that it retains at least one of its normal functional activities, for example binding to a target protein. The stability of the compound can be determined using standard methods. For example, the “thermostability” of a compound can be determined by measuring the thermal melt (“Tm”) temperature. The Tm is the temperature in degrees Celsius at which half of the compounds become unfolded. In some instances, the higher the Tm, the more stable the compound.

As used herein, the term “linker” or “linkage” refers to a linking moiety that connects two groups and has a backbone of 20 atoms or less in length. A linker or linkage may be a covalent bond that connects two groups or a chain of between 1 and 40 atoms in length, for example of about 1, 2, 3, 4, 5, 6, 8, 10, 12, 14, 16, 18, 20, 30 or 40 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, oligo(ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

The compounds of the subject libraries may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids and polypeptides. The present invention is meant to include all such possible isomers, as well as, their racemic and optically pure forms. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the term “a target protein” refers to all members of the target family, and fragments and enantiomers thereof, and protein mimics thereof. The target proteins of interest that are described herein are intended to include all members of the target family, and fragments and enantiomers thereof, and protein mimics thereof, unless explicitly described otherwise. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially, as well as fusion proteins containing a target molecule, as well as synthetic L- or D-proteins.

As used herein, the term “protein mimic” refers to a peptidic compound that mimics a binding property of a protein of interest, e.g., a target protein. In general terms, the target protein mimic includes an essential part of the original target protein (e.g., an epitope or essential residues thereof) that is necessary for forming a potential binding surface, such that the target protein mimic and the original target protein are each capable of binding specifically to a binding moiety of interest, e.g., an antibody or a D-peptidic compound. In some embodiments, the part(s) of the original target protein that is essential for binding is displayed on a scaffold such that potential binding surface of the original target protein is mimicked. Any suitable scaffold for displaying the minimal essential part of the target protein may be used, including but not limited to antibody scaffolds, scFv, anticalins, non-antibody scaffolds, mimetics of protein secondary and tertiary structures. In some embodiments, a target protein mimic includes residues or fragments of the original target protein that are incorporated into a protein scaffold, where the scaffold mimics a structural motif of the target protein. For example, by incorporating residues of the target protein at desirable positions of a convenient scaffold, the protein mimic may present a potential binding surface that mimics that of the original target protein. In some embodiments, the native structure of the fragments of the original target protein are retained using methods of conformational constraint. Any convenient methods of conformationally constraining a peptidic compound may be used, such as but not limited to, bioconjugation, dimerization (e.g., via a linker), multimerization, or cyclization.

As used herein, the term “VEGF” or its non-abbreviated form “vascular endothelial growth factor”, as used herein, refers to the protein products encoded by the VEGF gene. The term VEGF includes all members of the VEGF family, such as, VEGF-A, VEGF-B, VEGF-C, VEGF-D, VEGF-E, and fragments and enantiomers thereof. The term VEGF is intended to include recombinant and synthetic VEGF molecules, which can be prepared using any convenient recombinant expression methods or using any convenient synthetic methods, or purchased commercially (e.g. R & D Systems, Catalog No. 210-TA, Minneapolis, Minn.), as well as fusion proteins containing a VEGF molecule, as well as synthetic L- or D-proteins. VEGF is involved in both vasculogenesis (the de novo formation of the embryonic circulatory system) and angiogenesis (the growth of blood vessels from pre-existing vasculature) and can also be involved in the growth of lymphatic vessels in a process known as lymphangiogenesis. Members of the VEGF family stimulate cellular responses by binding to tyrosine kinase receptors (the VEGFRs) on the cell surface, causing them to dimerize and become activated through transphosphorylation. The VEGF receptors have an extracellular portion containing 7 immunoglobulin-like domains, a single transmembrane spanning region and an intracellular portion containing a split tyrosine-kinase domain. VEGF-A binds to VEGFR-1 (Flt-1) and VEGFR-2 (KDR/Flk-1). VEGFR-2 appears to mediate several of the cellular responses to VEGF. VEGF, its biological activities, and its receptors are well studied and are described in Matsumoto et al. (VEGF receptor signal transduction Sci STKE. 2001:RE21 and Marti et al (Angiogenesis in ischemic disease. Thromb Haemost. 1999 Suppl 1:44-52). Amino acid sequences of exemplary VEGFs that may be employed herein are found in the NCBI's Genbank database and a full description of VEGF proteins and their roles in various diseases and conditions is found in NCBI's Online Mendelian Inheritance in Man database.

### DETAILED DESCRIPTION

GB1 peptidic compounds that specifically bind to target proteins are provided. In some embodiments, the subject compounds are D-peptidic and specifically bind a L-target protein. In other embodiments, the subject compounds are L-peptidic and specifically bind a L-target protein. The subject GB1 peptidic compounds have a GB1 scaffold domain of the same structural motif as the B1 domain of Protein G (GB1), where the structural motif of GB1 is characterized by a motif that includes an arrangement of four β-strands and one α-helix around a hydrophobic core (also referred to as a 4β-1α motif). The GB1 peptidic compounds of the invention include mutations at non-core positions, e.g., variant amino acids at positions within a GB1 scaffold domain that are not part of the hydrophobic core of the structure. A 4β-1α motif is depicted in FIG. 1.

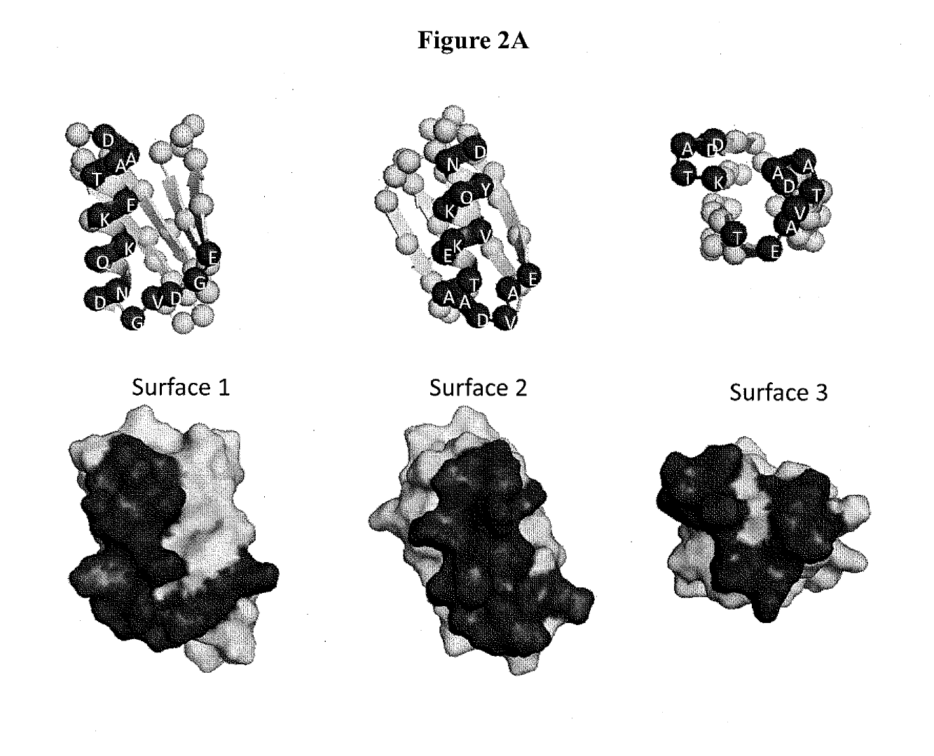

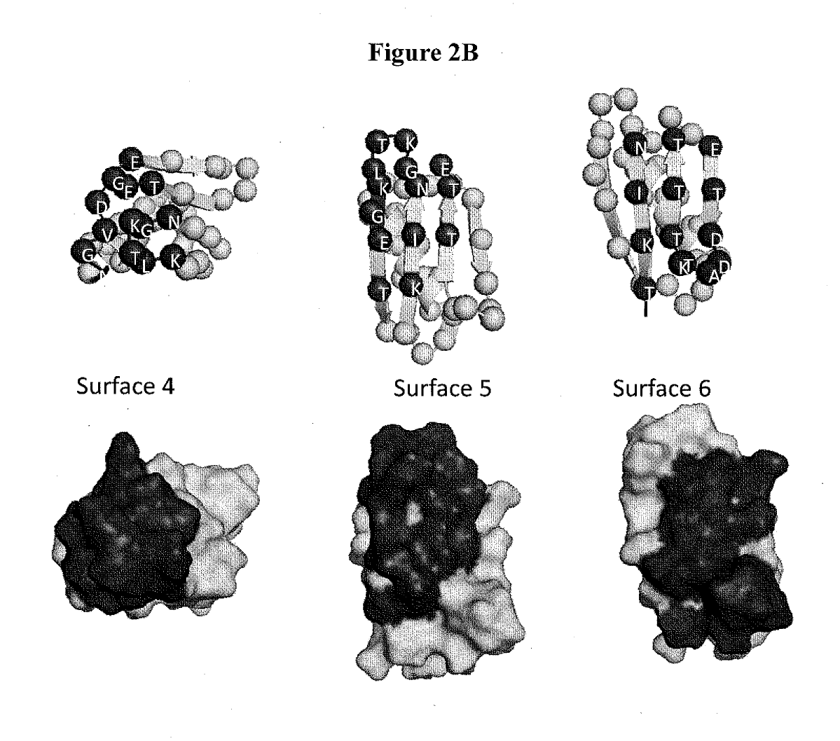

A number of GB1 peptidic compounds are provided. In some embodiments, the subject compounds are D-peptidic and specifically bind a L-target protein. In other embodiments, the subject compounds are L-peptidic and specifically bind a L-target protein. For the subject compounds, both the positions of the mutations and the nature of the mutation at each variable position of the scaffold may be varied. In some instances, the mutations are included at non-core positions, although mutations at core positions may also be included. The mutations may confer different functions on the resulting GB1 peptidic compounds, such as specific binding to a target molecule. The mutations may be selected at positions of a GB1 scaffold domain that are solvent exposed such that the variant amino acids at these positions can form part of a potential protein binding surface, although mutations at selected core and/or boundary positions may also be included. In the subject compounds, the mutations may be concentrated in a variable domain that defines a potential binding surface of the GB1 scaffold domain. GB1 peptidic compounds are provided that include distinct arrangements of mutations concentrated at various surfaces of the 4β-1α motif, for example, as depicted in FIGS. 2A-2B. The subject compounds may specifically bind to a target molecule via one of the several potential binding sites of the GB1 scaffold domain.

In the subject methods, a sample is contacted with a peptidic GB1 compound that specifically binds with high affinity to a target protein. A method including, administering to a subject a GB1 peptidic compound of the invention that specifically binds with high affinity to a target protein, is also provided. The subject methods and compounds find use in a variety of applications, including research and therapeutic applications.

Before certain embodiments are described in greater detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the various aspects of the invention, the structures and sequences of members of the various libraries are described first in greater detail, followed by a description of methods of screening and applications in which the libraries finds use.

### Compounds

As summarized above, aspects of the invention include GB1 peptidic compounds that have a GB1 scaffold domain of the same structural motif as the B1 domain of Protein G (GB1), where the structural motif of GB1 is characterized by a motif that includes an arrangement of four β-strands and one α-helix (also referred to as a 4β-1α motif) around a hydrophobic core. The subject GB1 peptidic compounds include mutations at various non-core positions of the 4β-1α motif, e.g., variant amino acids at non-core positions within a GB1 scaffold domain. In many embodiments, the four β-strands and one α-helix motifs of the structure are arranged in a hairpin-helix-hairpin motif, e.g., β1-β2-α1-β3-β4 where β1-β4 are β-strand motifs and α1 is a helix motif. A GB1 peptidic hairpin-helix-hairpin domain structure is depicted in FIG. 1.

The GB1 scaffold domain may be any polypeptide, or fragment thereof that includes the 4β-1α motif, whether naturally occurring or synthetic. The GB1 scaffold domain may be a native sequence of a member of the IgG binding B domain protein family, a IgG binding B domain sequence with pre-existing amino acid sequence modifications (such as additions, deletions and/or substitutions), or a fragment or analogue thereof. GB1 scaffold domains include those described in the following references Gronenborn et al., FEBS Letters 398 (1996), 312-316; Kotz et al., Eur. J. Biochem. 271, 1623-1629 (2004); Malakaukas et al., Nature Structural Biology, 5(6), 1998, p. 470-475; Minor Jr. et al., Nature, 367, 1994, 660-663; Nauli et al. Nature Structural Biology, 8(7), 2001, 602-605; Smith et al., Biochemistry, 1994, 33, 5510-5517; Wunderlich et al. J. Mol. Biol. (2006) 363, 545-557; Wellcome Trust Sanger Institute Pfam database (The Pfam protein families database: Finn et al., Nucleic Acids Research (2010) Database Issue 38:D211-222), see, e.g., Family: IgG_binding_B (PF01378) (pfam.sanger.ac.uk/family/PF01378.10#tabview=tab0); and analogs or fragments thereof; and those described in the definitions section above. In certain embodiments, a GB1 scaffold domain has an amino acid sequences as set forth in SEQ ID NOs: 1 and 314-348. In certain embodiments, a GB1 domain includes a sequence having 60% or more amino acid sequence identity, such as 70% or more, such as 80% or more, such as 90% or more, 95% or more or 98% or more amino acid sequence identity to an amino acid sequence set forth in SEQ ID NO:1 and 314-348. A GB1 scaffold domain sequence may include 1 or more, such as 2 or more, 3 or more, 4 or more, 5 or more, 10 or more, 15 or more, or even 20 or more additional peptidic residues compared to a native IgG binding B domain sequence. Alternatively, a GB1 scaffold domain sequence may include fewer peptidic residues compared a native IgG binding B domain sequence, such as 1, 2, 3, 4, 5, 6, 7, 8, 9 or 10, or even fewer residues.

Exemplary GB1 scaffold domain sequences from the Wellcome Trust Sanger Institute Pfam database are shown in the following sequence alignments:

B4U242_STREM/244-298
(SEQ ID NO: 314)
...S.YKLVIKGATFSGETATKAVDAAVAEQ.TFRDYANKNGVDGVWAYDAATKTFTVTE...
B4U242_STREM/316-370
(SEQ ID NO: 315)
....TYRLVIKGVTFSGETATKAVDAATAEQ.TFRQYANDNGITGEWAYDTATKTFTVTE...
C0MA37_STRE4/228-282
(SEQ ID NO: 316)
...S.YKLVIKGATFSGETATKAVDAAVAEQ.TFRDYANKNGVDGVWAYDAATKTFTVTE...
C0MA37_STRE4/300-354
(SEQ ID NO: 317)
....TYRLVIKGVTFSGETATKAVDAATAEQ.TFRQYANDNGVTGEWAYDAATKTFTVTE...
C0MCK9_STRS7/228-282
(SEQ ID NO: 318)
...S.YKLVIKGATFSGETATKAVDAAVAEQ.TFRDYANKNGVDGVWAYDAATKTFTVTE...
C0MCK9_STRS7/300-354
(SEQ ID NO: 319)
....TYRLVIKGVTFSGETSTKAVDAATAEQ.TFRQYANDNGVTGEWAYDAATKTFTVTE...
Q1JGB6_STRPD/117-137
(SEQ ID NO: 320)
ANIP........................AEK.AFRQYANDNGVDGV.................
Q53291_PEPMA/330-384
(SEQ ID NO: 321)
....TYKLILNGKTLKGETTTEAVDAATAEK.VFKQYANDNGVDGEWTYDDATKTFTVTE...
Q53291_PEPMA/400-454
(SEQ ID NO: 322)
....TYKLVINGKTLKGETTTKAVDAETAEK.AFKQYANDNGVDGVWTYDDATKTFTVTE...
Q53337_9STRE/3-57
(SEQ ID NO: 323)
....TYKLVINGKTLKGETTTKTVDAETAEK.AFKQYANDNGVDGVWTYDDATKTFTVTE...
Q53974_STRDY/258-312
(SEQ ID NO: 324)
....TYKLVINGKTLKGETTTKAVDAETAEK.AFKQYANENGVDGVWTYDDATKTFTVTE...
Q53975_STRDY/224-278
(SEQ ID NO: 325)
....TYKLVVKGNTFSGETTTKAIDTATAEK.EFKQYATANNVDGEWSYDDATKTFTVTE...
Q53975_STRDY/294-348
(SEQ ID NO: 326)
....TYKLIVKGNTFSGETTTKAVDAETAEK.AFKQYATANNVDGEWSYDDATKTFTVTE...
Q53975_STRDY/364-418
(SEQ ID NO: 327)
....TYKLIVKGNTFSGETTTKAIDAATAEK.EFKQYATANGVDGEWSYDDATKTFTVTE...
Q53975_STRDY/434-488
(SEQ ID NO: 328)
....TYKLIVKGNTFSGETTTKAVDAETAEK.AFKQYANENGVYGEWSYDDATKTFTVTE...
Q53975_STRDY/504-558
(SEQ ID NO: 329)
....TYKLVINGKTLKGETTTKAVDAETAEK.AFKQYANENGVDGVWTYDDATKTFTVTE...
Q54181_STRSG/1-45
(SEQ ID NO: 330)
..............MKGETTTEAVDAATAEK.VFKQYANDNGVDGEWTYDDATKTFTVTE...
Q54181_STRSG/131-185
(SEQ ID NO: 331)
....TYKLVINGKTLKGETTTKAVDAETAEK.AFKQYANDNGVDGVWTYDDATKTFTVTE...
Q54181_STRSG/61-115
(SEQ ID NO: 332)
....TYKLVINGKTLKGETTTEAVDAATAEK.VFKQYANDNGVDGEWTYDDATKTFTVTE...
Q56192_STAXY/238-290
(SEQ ID NO: 333)
....TYKLILNGKTLKGETTTEAVDAATARSFNFPILENSSSVPGDPLESTCMH......VEH
Q56193_STAXY/238-293
(SEQ ID NO: 334)
....TYKLILNGKTLKGETTTEAVDAATARSFNFPILENSSSVPGDPLESTCRHASFAQA...
Q56212_STRSZ/228-282
(SEQ ID NO: 335)
...S.YKLVIKGATFSGETATKAVDAAVAEQ.TFRDYANKNGVDGVWAYDAATKTFTVTE...
Q56212_STRSZ/300-354
(SEQ ID NO: 336)
....TYRLVIKGVTFSGETATKAVDAATAEQ.AFRQYANDNGVTGEWAYDAATKTFTVTE...
Q76K19_STRSZ/232-286
(SEQ ID NO: 337)
...S.YKLVIKGATFSGETATKAVDAAVAEQ.TFRDYANKNGVDGVWAYDAATKTFTVTE...
Q76K19_STRSZ/304-358
(SEQ ID NO: 338)
....TYRLVIKGVTFSGETATKAVDAATAEQ.TFRQYANDNGITGEWAYDTATKTFTVTE...
Q93EM8_STRDY/224-278
(SEQ ID NO: 339)
....TYKLVVKGNTFSGETTTKAIDTATAEK.EFKQYATANNVDGEWSYDDATKTFTVTE...
Q93EM8_STRDY/294-348
(SEQ ID NO: 340)
....TYKLIVKGNTFSGETTTKAIDAATAEK.EFKQYATANNVDGEWSYDYATKTFTVTE...
Q93EM8_STRDY/364-418
(SEQ ID NO: 341)
....TYKLIVKGNTFSGETTTKAIDAATAEK.EFKQYATANNVDGEWSYDDATKTFTVTE...
Q93EM8_STRDY/434-488
(SEQ ID NO: 342)
....TYKLIVKGNTFSGETTTKAVDAETAEK.AFKQYATANNVDGEWSYDDATKTFTVTE...
Q93EM8_STRDY/504-558
(SEQ ID NO: 343)
....TYKLVINGKTLKGETTTKAVDVETAEK.AFKQYANENGVDGVWTYDDATKTFTVTE...
SPG1_STRSG/228-282
(SEQ ID NO: 344)
....TYKLILNGKTLKGETTTEAVDAATAEK.VFKQYANDNGVDGEWTYDDATKTFTVTE...
SPG1_STRSG/298-352
(SEQ ID NO: 345)
....TYKLVINGKTLKGETTTKAVDAETAEK.AFKQYANDNGVDGVWTYDDATKTFTVTE...
SPG2_STRSG/303-357
(SEQ ID NO: 346)
....TYKLILNGKTLKGETTTEAVDAATAEK.VFKQYANDNGVDGEWTYDDATKTFTVTE...
SPG2_STRSG/373-427
(SEQ ID NO: 347)
....TYKLVINGKTLKGETTTEAVDAATAEK.VFKQYANDNGVDGEWTYDDATKTFTVTE...
SPG2_STRSG/443-497
(SEQ ID NO: 348)
....TYKLVINGKTLKGETTTKAVDAETAEK.AFKQYANDNGVDGVWTYDDATKTFTVTE...

In some embodiments, the GB1 scaffold domain is described by the following sequence: (T/S)Y(K/R)L(Z1)(Z1)(N/K)G(K/N/V/A)T(L/F)(K/S)GET(T/A/S)T(K/E)(A/T)(V/I)D(A/T/V)(A/E)(T/V)AE(K/Q)(A/E/T/V)F(K/R)(Q/D)YA(N/T)(A/D/E/K)N(G/N)(Z3)(D/T)G(E/V)W(A/T/S)YD(D/A/Y/T)ATKT(Z1)T(Z1)TE (SEQ ID NO:361), where each Z1 is independently a hydrophobic residue. In some embodiments, the GB1 scaffold domain is described by the following sequence: (T/S)Y(K/R)L(I/V)(L/I/V)(N/K)G(K/N/V/A)T(L/F)(K/S)GET(T/A/S)T(K/E)(A/T)(V/I)D(A/T/V)(A/E)(T/V)AE(K/Q)(A/E/T/V)F(K/R)(Q/D)YA(N/T)(A/D/E/K)N(G/N)(V/I)(D/T)G(E/V)W(A/T/S)YD(D/A/Y/T)ATKTFTVTE (SEQ ID NO:362). In certain embodiments, GB1 scaffold domain is described by the following sequence: TYKL(I/V)(L/I/V)(N/K)G(K/N)T(L/F)(K/S)GET(T/A)T(K/E)AVD(A/T/V)(A/E)TAE(K/Q)(A/E/T/V)F(K/R)QYA(N/T)(A/D/E/K)N(G/N)VDG(E/V)W(A/T/S)YD(D/A)ATKTFTVTE (SEQ ID NO:363). A mutation in a scaffold domain may include a deletion, insertion, or substitution of an amino acid residue at any convenient position to produce a sequence that is distinct from the reference scaffold domain sequence.

In some embodiments, the GB1 scaffold domain is described by the following sequence: T(Z2)K(Z1)(Z1)(Z1)(N/V)(G/L/I)(K/G)(Q/T/D)(L/A/R)(K/V)(G/E/V)(E/V)(A/T/R/I/P/V)(T/I)(R/W/L/K/V/T/I)E(A/L/I)VDA(A/G)(T/E)(A/V/F)EK(V/I/Y)(F/L/W/I/A)K(L/Q)(Z1)(Z3)N(A/D)(K/N)(T/G)(V/I)(E/D)G(V/E)(W/F)TY(D/K)D(E/A)(T/I)KT(Z1)T(Z1)TE (SEQ ID NO:364), where each Z1 is independently a hydrophobic residue, Z2 is an aromatic hydrophobic residue, and Z3 is a non-aromatic hydrophobic residue.

In some embodiments, the GB1 scaffold domain is described by the following sequence:

(SEQ ID NO: 365)
T(Y/F/W/A)K(L/V/I/M/F/Y/A)(L/V/I/F/M)(L/V/I/F/M/A/Y/S)(N/V)(G/L/I)(K/G)(Q/T/D)
(L/A/R)(K/V)(G/E/V)(E/V)(A/T/R/I/P/V)(T/I)(R/W/L/K/V/T/I)E(A/L/I)VDA(A/G)(T/E)
(A/V/F)EK(V/I/Y)(F/L/W/I/A)K(L/Q)(W/F/L/M/Y/I)(L/V/I/A)N(A/D)(K/N)(T/G)(V/I)
(E/D)G(V/E)(W/F)TY(D/K)D(E/A)(T/I)KT(L/V/I/F/M/W)T(L/V/I/F/M)TE.

The positions to be mutated are selected to minimize structural perturbations of the GB1 scaffold domain and to ensure that the subject GB1 peptidic compounds can maintain a folded state under physiological conditions. Another aspect of the subject compounds is the selection of amino acid positions to be mutated such that the amino acids can form a potential binding surface in the GB1 scaffold domain, whether or not the residues actually contact a target protein. One way of determining whether an amino acid position is part of a potential binding surface involves examining the three dimensional structure of the GB1 scaffold domain, using a computer program such as the UCSF Chimera program. Other ways include crystallographic and genetic mutational analysis. Any convenient method may be used to determine whether an amino acid position is part of a potential binding surface.

The mutations may be found at positions in the GB1 scaffold domain where the residue is at least in part solvent exposed. Solvent exposed positions can be determined using software suitable for protein modeling and three-dimensional structural information obtained from a crystal structure. For example, solvent exposed residues may be determined using the Protein Data Bank (PDB) structure 3 GB1 and estimating the solvent accessible surface area (SASA) for each residue using the GETarea tool (Fraczkiewicz & Braun, “Exact and efficient analytical calculation of the accessible surface areas and their gradients for macromolecules,” J. Comput. Chem. 1998, 19, 319-333). This tool calculates the ratio of SASA in structure compared to SASA in a random coil. A ratio of 0.4 was used in selecting the following solvent accessible residues (shown in bold):

(SEQ ID NO: 1)
TYKLILNGKTLKGETTTEAVDAATAEKVFKQYANDNGVDGEWTYDDATKT
FTVTE.

The mutations of the parent GB1 domain may be concentrated at one of several different potential binding surfaces of the scaffold domain. Several distinct arrangements of mutations of the GB1 scaffold domain at non-core positions of the hairpin-helix-hairpin scaffold domain are provided. The majority of the mutations are at non-core positions of the parent GB1 domain (e.g., solvent exposed or boundary positions) however in some cases one or more mutations (e.g., 1 or 2 mutations) may be located at hydrophobic core positions. In certain embodiments, mutations at hydrophobic core positions may be tolerated without significantly disrupting the GB1 scaffold structure, such as, when those core mutations are selected in a loop region. In certain embodiments, mutations at boundary positions may also be tolerated without significantly disrupting the GB1 scaffold structure. In such cases the loop region may form a structure or conformation that is different to that of the parent GB1 scaffold. Mutations at such positions may confer desirable properties upon the resulting GB1 compounds, such as stability, a certain structural property, or specific binding to a target molecule.

In some embodiments, the sequence of the GB1 scaffold domain is optimized for stability. In some embodiments, mutations at hydrophobic core and/or boundary positions are included to optimize the stability of a GB1 peptidic structure. In some cases, optimization of the stability is performed when the GB1 structure is disrupted by mutations at a target protein-binding surface. For example, one or more mutations in the variable domain of a GB1 peptidic compound that provide for specific binding to a target protein may lead to disruption of the hydrophobic core structure of the compound. In such cases, one or more further mutations may be introduced, or optimization of the underlying scaffold sequence may be performed, to optimize stability and minimize any disruptions to the structure and produce a stable GB1 peptidic compound that specifically binds to the target protein. In certain embodiments, the one or more stabilizing mutations may include mutations to hydrophobic core and/or boundary residues, and may be located in a fixed domain or a variable domain of the structure.

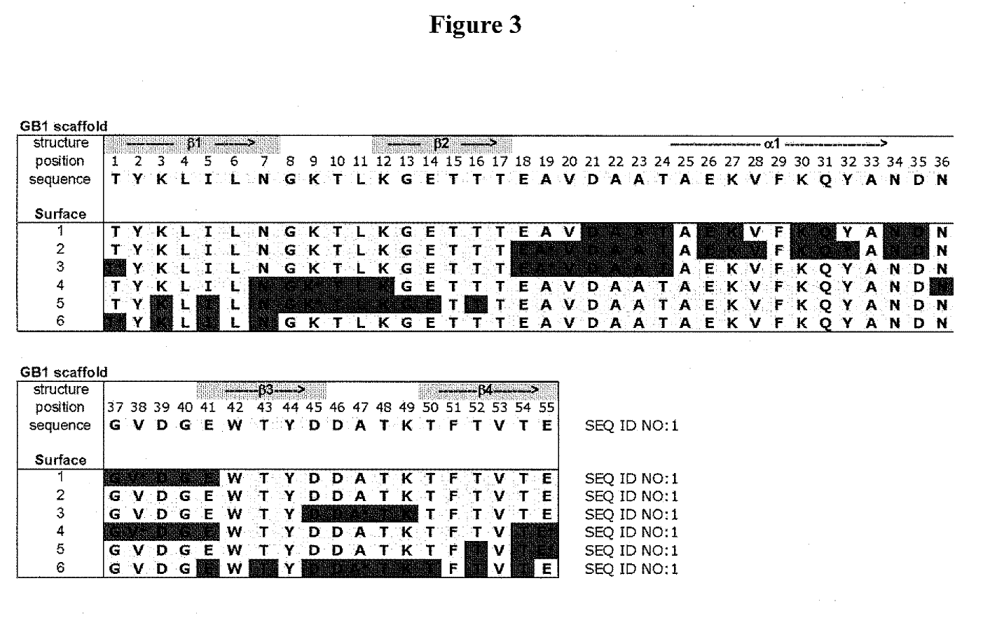

The positions of the mutations in the parent GB1 domain may be described herein either by reference to a structural motif or region, or by reference to a position number in the primary sequence of the domain. FIG. 3 illustrates the alignment of the position numbering scheme for a GB1 scaffold domain relative to its β1, β2, α1, β3 and β4 motifs, and relative to the positions of mutations found in certain subject compounds. Positions marked with an asterix indicate exemplary positions at which mutations that include the insertion of one or more amino acids may be included. Any GB1 peptidic domain sequence may be substituted for the GB1 scaffold sequence depicted in FIG. 3, and the positions of the mutations that define a subject compound may be transferred from one scaffold to another by any convenient method. For example, a sequence alignment method may be used to place any GB1 scaffold domain sequence within the framework of the position numbering scheme illustrated in FIG. 3. Alignment methods based on structural motifs such as β-strands and α-helices may also be used to place a GB1 scaffold domain sequence within the framework of the position numbering scheme illustrated in FIG. 3.

In some cases, a first GB1 scaffold domain sequence may be aligned with a second GB1 scaffold domain sequence that is one or more amino acids longer or shorter. For example, the second GB1 scaffold domain may have one or more additional amino acids at the N-terminal or C-terminal relative to the first GB1 scaffold, or may have one or more additional amino acids in one of the loop regions of the structure. In such cases, a numbering scheme such as is described below for insertion mutations may be used to relate two GB1 scaffold domain sequences.

A subject GB1 peptidic compound may include a hairpin-helix-hairpin domain described by formula (I):

P1-α1-P2 (I)

where P1 and P2 are independently beta-hairpin domains and α1 is a helix domain and P1, α1 and P2 are connected independently by linking sequences of between 1 and 10 residues in length. In some embodiments, in formula (I), P1 is β1-β2 and P2 is β3-β4 such that the compound is described by formula (II):

β1-β2-α1-β3-β4 (II)

where β1, β2, β3 and β4 are independently beta-strand domains and α1 is a helix domain, and β1, β2, α1, β3 and β4 are connected independently by linking sequences of between 1 and 10 residues in length, such as, between 2 and 8 residues, or between 3 and 6 residues in length. In certain embodiments, each linking sequence is independently of 3, 4, 5, 6, 7 or 8 residues in length. In certain embodiments, each linking sequence is independently of 3, 4, 5, 6, 7 or 8 residues in length, such as 4 or 5 residues in length.

In certain embodiments, the linking sequences may form a loop or a turn structure. For example, the two antiparallel β-strands of a hairpin motif may be connected via a loop. Mutations in a linking sequence that includes insertion or deletion of one or more amino acid residues may be tolerated without significantly disrupting the GB1 scaffold domain structure. In some embodiments, in formulas (I) and (II), the subject compound includes mutations in one or more linking sequences. In certain embodiments, 80% or more, 90% or more, 95% or more, or even 100% of the mutations are at positions within the regions of the linking sequences. In certain embodiments, in formulas (I) and (II), at least one of the linking sequences is one or more (e.g., such as 2 or more) residues longer in length than the corresponding linking sequence of the GB1 scaffold domain. In certain embodiments, in formulas (I) and (II), at least one of the linking sequences is one or more residues shorter in length than the corresponding linking sequence of the GB1 scaffold domain.

In some embodiments, one or more positions in the scaffold may be selected as positions at which to include insertion mutations, e.g., mutations that include the insertion of 1 or 2 additional amino acid residues in addition to the amino acid residue being substituted. In certain embodiments, the insertion mutations are selected for inclusion in one or more loop regions, or at the N-terminal or C-terminal of the scaffold. The positions of the variant amino acids that are inserted may be referred to using a letter designation with respect to the numbered position of the mutation, e.g., an insertion mutation of 2 amino acids at position 38 may be referred to as positions 38a and 38b.

In certain embodiments, the subject compound includes a mutation at position 38 that includes insertion of 0, 1 or 2 variant amino acids. In certain embodiments, the subject compound includes a mutation at position 19 that includes insertion of 0, 1 or 2 variant amino acids. In certain embodiments, the subject compound includes a mutation at position 1 that include insertion of 2 variant amino acids, and at positions 19 and 47 that each include insertion of 0, 1 or 2 variant amino acids. In certain embodiments, the subject compound includes mutations at positions 9 and 38 that each include insertion of 0, 1 or 2 variant amino acids, and at position 55 that includes insertion of 1 variant amino acid. In certain embodiments, the subject compound includes a mutation at position 9 that includes insertion of 0, 1 or 2 variant amino acids, and at position 55 that includes insertion of 1 variant amino acid. In certain embodiments, the subject compound includes a mutation at position 1 that includes insertion of 1 variant amino acid, and at position 47 that includes insertion of 0, 1 or 2 variant amino acids.

In some cases, when an insertion mutation (e.g., insertion of one or more additional variant amino acids) is made in a GB1 scaffold, the resulting GB1 compound variants may be aligned with the parent GB1 scaffold in different ways. For example, an insertion mutation including 2 additional variant amino acids at position 38 of the GB1 scaffold may lead to GB1 compound variants where the loop regions between the α1 and β3 regions can be aligned with the GB1 scaffold domain in two or more distinct ways. In other words, the resulting GB1 compounds may encompass various distinct loop sequences and/or structures that align differently with the parent GB1 scaffold domain. In some cases, the various distinct loop sequences are produced when the insertion mutation is in a variable loop region (e.g. where most of the loop region is being mutated).

In some embodiments, the subject compound includes 4 or more, such as, 5 or more, 6 or more, 7 or more, 8 or more, 9 or more, 10 or more, 11 or more, 12 or more, 13 or more, 14 or more, or 15 or more mutations at different positions of the hairpin-helix-hairpin domain. The mutations may involve the deletion, insertion, or substitution of the amino acid residue at the position being mutated. The mutations may include substitution with any naturally or non-naturally occurring amino acid, or an analog thereof.

In some embodiments, the subject compound includes 3 or more different non-core mutations, such as, 4 or more, 5 or more, 6 or more, 7 or more, 8 or more, 9 or more, 10 or more, 11 or more, or 12 or more different non-core mutations in a region outside of the β1-β2 region.

In some embodiments, the subject compound includes 3 or more different non-core mutations, such as, 4 or more, 5 or more, 6 or more, 7 or more, 8 or more, 9 or more, 10 or more or 11 or more different non-core mutations in the α1 region.

In some embodiments, the subject compound includes 3 or more different non-core mutations, such as 4 or more, 5 or more, 6 or more, 7 or more, 8 or more, 9 or more or 10 or more different non-core mutations in the β3-β4 region.

In some embodiments, the subject compound includes at least 5 or more different non-core mutations, such as 6 or more, 7 or more, 8 or more, 9 or more, 10 or more, 11 or more, or 12 or more different non-core mutations in the α1-β3 region.

In certain embodiments, the subject compound includes ten or more different mutations, where the ten or more different mutations are located at positions selected from the group consisting of positions 21-24, 26, 27, 30, 31, 34, 35, 37-41. In certain embodiments, one or more mutations (e.g., 1 or 2 mutations) are at core positions. In certain embodiments, one or more mutations (e.g., 1 or 2 mutations) are at boundary positions. In certain embodiments, ten or more mutations (e.g., 10, 11, 12, 13 or more mutations) are at solvent exposed positions.

In certain embodiments, the subject compound includes ten or more different mutations, where the ten or more different mutations are located at positions selected from the group consisting of positions 18-24, 26-28, 30-32, 34 and 35. In certain embodiments, one or more mutations (e.g., 1 mutation) are at core positions. In certain embodiments, ten or more mutations (e.g., 10, 11, 12, 13, 14 or more mutations) are at solvent exposed positions.

In certain embodiments, the subject compound includes ten or more different mutations, where the ten or more different mutations are located at positions selected from the group consisting of positions 1, 18-24 and 45-49. In certain embodiments, one or more mutations (e.g., 1 mutation) are at core positions. In certain embodiments, ten or more mutations (e.g., 10, 11, 12 or more mutations) are at solvent exposed positions.

In certain embodiments, the subject compound includes ten or more different mutations, where the ten or more different mutations are located at positions selected from the group consisting of positions 7-12, 36-41, 54 and 55. In certain embodiments, one or more mutations (e.g., 1 mutation) are at core positions. In certain embodiments, one or more mutations (e.g., 2, 3 or 4 or more mutations) are at boundary positions. In certain embodiments, five or more mutations (e.g., 6, 7, 8, 9 or more mutations) are at solvent exposed positions.

In certain embodiments, the subject compound includes ten or more different mutations, where the ten or more different mutations are located at positions selected from the group consisting of positions 3, 5, 7-14, 16, 52, 54 and 55. In certain embodiments, one or more mutations (e.g., 2, 3 or more mutations) are at boundary positions. In certain embodiments, nine or more mutations (e.g., 10, 11, 12 or more mutations) are at solvent exposed positions.

In certain embodiments, the subject compound includes ten or more different mutations, where the ten or more different mutations are located at positions selected from the group consisting of positions 1, 3, 5, 7, 41, 43, 45-50 52 and 54. In certain embodiments, one or more mutations (e.g., 2 or more mutations) are at boundary positions. In certain embodiments, nine or more mutations (e.g., 10, 11, 12 or more mutations) are at solvent exposed positions.

In certain embodiments, the subject compound includes five or more different mutations in the α1 region. In certain embodiments, five or more different mutations are located at positions selected from the group consisting of positions 22-24, 26, 27, 30, 31, 34 and 35.

In certain embodiments, the subject compound includes ten or more different mutations in the α1 region. In certain embodiments, the ten or more different mutations are located at positions selected from the group consisting of positions 22-24, 26, 27, 28, 30, 31, 32, 34 and 35.

In certain embodiments, the subject compound includes three or more different mutations in the β3-β4 region. In certain embodiments, the three or more different mutations are located at positions selected from the group consisting of positions 41, 54 and 55. In certain embodiments, the three or more different mutations are located at positions selected from the group consisting of positions 52, 54 and 55.

In certain embodiments, the subject compound includes five or more different mutations in the β3-β4 region. In certain embodiments, the five or more different mutations are located at positions selected from the group consisting of positions 45-49.

In certain embodiments, the subject compound includes nine or more different mutations in the β3-β4 region. In certain embodiments, the nine or more different mutations are located at positions selected from the group consisting of positions 41, 43, 45-50 52 and 54.

In certain embodiments, the subject compound includes two or more different mutations in the region between the α1 and β3 regions, e.g., mutations in the linking sequence between α1 and β3. In certain embodiments, the two or more different mutations are located at positions selected from the group consisting of positions 37-40.

In certain embodiments, the subject compound includes three or more, four or more, five or more, six or more, or ten or more different mutations in the β1-β2 region. In certain embodiments, the ten or more different mutations in the β1-β2 region are located at positions selected from the group consisting of positions 3, 5, 7-14 and 16.

### Intramolecularly Crosslinked Compounds

In some embodiments, the subject compound includes an intramolecular crosslink. Such crosslinks may provide a conformational constraint that stabilizes a structural motif of the compound. In certain embodiments, the subject compound includes an intramolecular crosslink that stabilizes the hydrophobic core, e.g., by constraining two secondary structural motifs of the GB1 structure. In certain embodiments, the subject compound includes an intramolecular crosslink between β2 and α1 (e.g., between positions 15 and 32). In certain embodiments, the subject compound includes an intramolecular crosslink between β3 and α1 (e.g., between positions 26 and 42). In certain embodiments, the subject compound includes an intramolecular crosslink between β4 and α1 (e.g., between positions 33 and 53). In certain embodiments, the subject compound includes an intramolecular crosslink between β1 and β4 (e.g., between positions 3 and 50). In certain embodiments, the subject compound includes an intramolecular crosslink between β1 and α1 (e.g., between positions 4 and 29).

An intramolecular crosslink may connect two moieties of the subject compound (e.g., two sidechain moieties, a backbone moiety and a sidechain moiety, or two backbone moieties) that are near to each other in space. The intramolecular crosslink may be formed directly via a covalent bond or via another linking moiety. In some embodiments, the crosslink connects two residues of the subject GB1 compound via their sidechain moieties. In certain embodiments, the crosslink includes a peptidic or polyethylene glycol moiety. In certain embodiments, the crosslink includes a carbon chain (e.g., a saturated C6 linker). Any two convenient residues of the GB1 compounds may be selected for intramolecular crosslinking. In some embodiments, the residues are naturally occurring residues. In certain embodiments, the two linked residues are independently selected from Cys, Lys, Ser, Thr, Tyr, Asp and Glu. In certain embodiments, one or both of the two linked residues are non-natural amino acids.

In certain embodiments, the intramolecular crosslink is a disulfide bond between two Cys residues of the GB1 compound. Any convenient ligation chemistries, linking moieties and functional groups may be used to produce an intramolecular crosslink.

As used herein, the terms “linker” and “crosslink” are used interchangeably and refer to a linking moiety that covalently connects two groups and has a backbone of 30 atoms or less (such as 20 atoms or less) in length. A linking moiety may be a covalent bond that connects two groups or a chain of between 1 and 30 atoms in length, for example of about 1, 2, 3, 4, 5, 6, 8, 10, 12, 14, 16, 18, 20 or 30 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, disulfide, amides, carbonates, carbamates, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

In some embodiments, the subject compound is described by a formula independently selected from the group consisting of:

F1-V1-F2 (III);

F3-V2-F4 (IV);

V3-F5-V4-F6-V5-F7 (V);

F8-V6-F9-V7-F10-V8 (VI);

V9-F11-V10 (VII); and

V11-F12-V12 (VIII)

where F1, F2, F3, F4, F5, F6, F7, F8, F9, F10, F11 and F12 are fixed regions and V1, V2, V3, V4, V5, V6, V7, V8, V9, V10, V11 and V12 are variable regions;

where the variable regions of any one formula include a combined total of three or more different non-core mutations in a region outside of the β1-β2 region.

In certain embodiments, the subject compound is described by formula (III), where:

F1 comprises a sequence having 60% or more (e.g., 70% or more, 80% or more, 90% or more, or 95% or more) amino acid sequence identity to the amino acid sequence TYKLILNGKTLKGETTTEA (SEQ ID NO:2);

F2 comprises a sequence having 60% or more (e.g., 70% or more, 80% or more, 90% or more, or 95% or more) amino acid sequence identity to an amino acid sequence TYDDATKTFTVTE (SEQ ID NO:3); and

V1 comprises a sequence that comprises 10 or more mutations (e.g., 11, 12, 13, 14 or 15 or more mutations) compared to the parent amino acid sequence VDAATAEKVFKQYANDNGVDGEW (SEQ ID NO:4).

In certain embodiments, in formula (III), V1 comprises a sequence of the following formula: VXXXXAXXVFXXYAXXNXXXXXW (SEQ ID NO:5), where each X is a variant amino acid.

In certain embodiments, in formula (III), F1 comprises the sequence TYKLILNGKTLKGETTTEA (SEQ ID NO:2), F2 comprises the sequence TYDDATKTFTVTE (SEQ ID NO:3), and V1 comprises a sequence of the following formula: VXXXXAXXVFXXYAXXNXXXXXW (SEQ ID NO:6) where each X is independently selected from the group consisting of A, D, F, S, V and Y.

In certain embodiments, in formula (III), the mutation at position 19 in V1 includes insertion of 0, 1 or 2 variant amino acids.

In certain embodiments, the subject compound is described by formula (IV), where:

F3 comprises a sequence having 60% or more (e.g., 70% or more, 80% or more, 90% or more, or 95% or more) amino acid sequence identity to the amino acid sequence TYKLILNGKTLKGETT (SEQ ID NO:7);

F4 comprises a sequence having 60% or more (e.g., 70% or more, 80% or more, 90% or more, or 95% or more) amino acid sequence identity to an amino acid sequence GVDGEWTYDDATKTFTVTE (SEQ ID NO:8); and

V2 comprises a sequence that comprises 10 or more mutations (e.g., 11, 12, 13, 14 or 15 or more mutations) compared to the parent amino acid sequence TEAVDAATAEKVFKQYANDN (SEQ ID NO:9).

In certain embodiments, in formula (IV), V2 comprises a sequence of the formula: TXXXXXXXAXXXFXXXAXXN (SEQ ID NO:10), where each X is a variant acid.

In certain embodiments, in formula (IV), F3 comprises the sequence TYKLILNGKTLKGETT (SEQ ID NO:7), F4 comprises the sequence GVDGEWTYDDATKTFTVTE (SEQ ID NO:8), and V2 comprises a sequence of the formula: TXXXXXXXAXXXFXXXAXXN (SEQ ID NO:11) where each X is independently selected from the group consisting of A, D, F, S, V and Y.

In certain embodiments, in formula (IV), the mutation at position 3 of V2 includes insertion of 0, 1 or 2 variant amino acids.

In certain embodiments, the subject compound is described by formula (V), where:

F5 comprises a sequence having 60% or more (e.g., 70% or more, 80% or more, 90% or more, or 95% or more) amino acid sequence identity to the amino acid sequence KLILNGKTLKGETT (SEQ ID NO:12);

F6 comprises a sequence having 60% or more (e.g., 70% or more, 80% or more, 90% or more, or 95% or more) amino acid sequence identity to an amino acid sequence EKVFKQYANDNGVDGEWT (SEQ ID NO:13);

F7 comprises a sequence having 60% or more (e.g., 70% or more, 80% or more, 90% or more, or 95% or more) amino acid sequence identity to an amino acid sequence FTVTE (SEQ ID NO:14);

V3 comprises a sequence that comprises one or more mutations (e.g., 2 or more mutations) compared to the parent amino acid sequence TY; and

V4 comprises a sequence that comprises 3 or more mutations (e.g., 4, 5, 6 or 7 or more mutations) compared to the parent amino acid sequence TEAVDAATA (SEQ ID NO:15); and

V5 comprises a sequence that comprises 3 or more mutations (e.g., 4 or 5 or more mutations) compared to the parent amino acid sequence YDDATKT (SEQ ID NO:16).

In certain embodiments, in formula (V), V3 comprises a sequence of the formula XY, V4 comprises a sequence of the formula TXXXXXXXA (SEQ ID NO:17), and V5 comprises a sequence of the formula YXXXXXT (SEQ ID NO:18) where each X is a variant amino acid.

In certain embodiments, in formula (V), F5 comprises the sequence KLILNGKTLKGETT (SEQ ID NO:12), F6 comprises the sequence EKVFKQYANDNGVDGEWT (SEQ ID NO:13), F7 comprises the sequence FTVTE (SEQ ID NO:14), V3 comprises a sequence of the formula XY, V4 comprises a sequence of the formula TXXXXXXXA (SEQ ID NO:19), and V5 comprises a sequence of the formula YXXXXXT (SEQ ID NO:20) where each X is independently selected from the group consisting of A, D, F, S, V and Y.

In certain embodiments, in formula (V), the mutation at position 1 of V3 includes insertion of +2 variant amino acids, and the mutations at positions 3 and 4 of V4 and V5, respectively, each include insertion of 0, 1 or 2 variant amino acids.

In certain embodiments, the subject compound is described by formula (VI), where:

F8 comprises a sequence having 60% or more (e.g., 70% or more, 80% or more, 90% or more, or 95% or more) amino acid sequence identity to the amino acid sequence TYKLI (SEQ ID NO:21);

F9 comprises a sequence having 60% or more (e.g., 70% or more, 80% or more, 90% or more, or 95% or more) amino acid sequence identity to the amino acid sequence ETTTEAVDAATAEKVFKQYAN (SEQ ID NO:22);

F10 comprises a sequence having 60% or more (e.g., 70% or more, 80% or more, 90% or more, or 95% or more) amino acid sequence identity to the amino acid sequence TYDDATKTFT (SEQ ID NO:23);

V6 comprises a sequence that comprises 3 or more mutations (e.g., 4, 5 or 6 or more mutations) compared to the parent amino acid sequence LNGKTLKG (SEQ ID NO:24);

V7 comprises a sequence that comprises 3 or more mutations (e.g., 4, 5 or 6 or more mutations) compared to the parent amino acid sequence DNGVDGEW (SEQ ID NO:25);

V8 comprises a sequence that comprises one or more mutations (e.g., 2 or more mutations) compared to the parent amino acid sequence VTE.

In certain embodiments, in formula (VI), V6 comprises a sequence of the formula LXXXXXXG (SEQ ID NO:26), V7 comprises a sequence of the formula DXXXXXXW (SEQ ID NO:27), and V8 comprises a sequence of the formula VXX where each X is a variant amino acid.

In certain embodiments, in formula (VI), F8 comprises the sequence TYKLI (SEQ ID NO:21), F9 comprises the sequence ETTTEAVDAATAEKVFKQYAN (SEQ ID NO:22), F10 comprises the sequence TYDDATKTFT (SEQ ID NO:23), V6 comprises a sequence of the formula LXXXXXXG (SEQ ID NO:28), V7 comprises a sequence of the formula DXXXXXXW (SEQ ID NO:29), and V8 comprises a sequence of the formula VXX where each X is independently selected from the group consisting of A, D, F, S, V and Y.

In certain embodiments, in formula (VI), the mutations at position 4 of V6 and V7 each include insertion of 0, 1 or 2 variant amino acids, and the mutation at position 3 of V8 includes insertion of 1 variant amino acid.

In certain embodiments, the subject compound is described by formula (VII), where:

F11 comprises a sequence having 60% or more (e.g., 70% or more, 80% or more, 90% or more, or 95% or more) amino acid sequence identity to an amino acid sequence EAVDAATAEKVFKQYANDNGVDGEWTYDDATKT (SEQ ID NO:30);

V9 comprises a sequence that comprises 6 or more mutations (e.g., 7, 8, 9, 10 or 11 or more mutations) compared to the parent amino acid sequence TYKLILNGKTLKGETTT (SEQ ID NO:31); and

V10 comprises a sequence that comprises 2 or more mutations (e.g., 3 or more mutations) compared to the parent amino acid sequence FTVTE (SEQ ID NO:32).

In certain embodiments, in formula (VII), V9 comprises a sequence of the formula TYXLXLXXXXXXXXTXT (SEQ ID NO:33), and V10 comprises a sequence of the formula FXVXX (SEQ ID NO:34), where each X is a variant amino acid.

In certain embodiments, in formula (VII), F11 comprises the sequence EAVDAATAEKVFKQYANDNGVDGEWTYDDATKT (SEQ ID NO:30); V9 comprises a sequence of the formula TYXLXLXXXXXXXXTXT (SEQ ID NO:35), and V10 comprises a sequence of the formula FXVXX (SEQ ID NO:36), where each X is independently selected from the group consisting of A, D, F, S, V and Y.

In certain embodiments, in formula (VII), the mutation at position 9 of V9 includes insertion of 0, 1 or 2 variant amino acids, and the mutation at position 5 of V10 includes insertion of 1 variant amino acid.

In certain embodiments, the subject compound is described by formula (VIII), where:

F12 comprises a sequence having 60% or more (e.g., 70% or more, 80% or more, 90% or more, or 95% or more) amino acid sequence identity to the amino acid sequence KTLKGETTTEAVDAATAEKVFKQYANDNGVD (SEQ ID NO:37);

V11 comprises a sequence that comprises 3 or more mutations (e.g., 4 or more mutations) compared to the parent amino acid sequence TYKLILNG (SEQ ID NO:38);

V12 comprises a sequence that comprises 5 or more mutations (e.g., 6, 7, 8, 9 or 10 or more mutations) compared to the parent amino acid sequence GEWTYDDATKTFTVTE (SEQ ID NO:39).

In certain embodiments, in formula (VIII), V11 comprises a sequence of the formula XYXLXLXG (SEQ ID NO:40), and V12 comprises a sequence of the formula GXWXYXXXXXXFXVXE (SEQ ID NO:41), where each X is a variant amino acid.

In certain embodiments, in formula (VIII), F12 comprises the sequence KTLKGETTTEAVDAATAEKVFKQYANDNGVD (SEQ ID NO:37), V11 comprises a sequence of the formula XYXLXLXG (SEQ ID NO:42), and V12 comprises a sequence of the formula GXWXYXXXXXXFXVXE (SEQ ID NO:43), where each X is independently selected from the group consisting of A, D, F, S, V and Y.

In certain embodiments, in formula (VIII), the mutation at position 8 of V12 includes insertion of 0, 1 or 2 variant amino acids, and the mutation at position 1 of V11 includes insertion of 2 variant amino acids.

In some embodiments, the subject compound includes a peptidic sequence of between 30 and 80 residues, such as between 40 and 70, between 45 and 60 residues, or between 50 and 56 residues. In certain embodiments, the subject compound includes a peptidic sequence of 50, 51, 52, 53, 54, 55, 56, 57, 58, 59 or 60 residues. In certain embodiments, the peptidic sequence is of 54, 55, 56 or 57 residues, such as 55 or 56 residues.

In some embodiments, the subject compound includes a GB1 scaffold domain and a variable domain. The variable domain may be a part of the GB1 scaffold domain and may be either a continuous or a discontinuous sequence of residues. A variable domain that is defined by a discontinuous sequence of residues may include contiguous variant amino acids at positions that are arranged close in space relative to each other in the structure of the compound. The variable domain may form a potential binding interface of the subject compound. The variable domain may define a binding surface area of a suitable size for forming protein-protein interactions of high affinity (e.g., 300 nM or less, such as 100 nM or less, 30 nM or less, 10 nM or less, 3 nM or less, or 1 nM or less) and specificity. The variable domain may include a surface area of between 600 and 1800 Å², such as between 800 and 1600 Å², between 1000 and 1400 Å², between 1100 and 1300 Å², or about 1200 Å².

The individual sequences of the subject compounds can be determined as follows. Any GB1 scaffold as defined herein may be selected as a scaffold for a subject compound. The positions of the mutations in the GB1 scaffold domain may be selected as described herein, e.g., as depicted in FIG. 3 for surfaces 1 to 6, where the GB1 scaffold domain may be aligned with the framework of FIG. 3 as described above. The nature of the mutation at each variant amino acid position may be selected, e.g., substitution with any naturally occurring or non naturally occurring amino acid and may confer on the compound variant a desirable property (e.g., increased solubility, stability or specific binding to a target molecule). Certain variant amino acid positions may be selected as positions where mutations can include the insertion or deletion of amino acids, e.g., the insertion of 1 or 2 amino acids where the variant amino acid position occurs in a loop or turn region of the scaffold. In certain embodiments, the mutations can include the insertion or amino acids at one or more positions selected from positions 1, 9, 19, 38, 47 and 55. After selection of the GB1 scaffold, selection of the positions of variant amino acids, and selection of the nature of the mutations at each position, the individual sequences of the subject compounds can be determined.

In some embodiments, a subject compound may include two distinct groups of mutations that define two distinct potential binding surfaces of the GB1 scaffold domain. The two distinct groups of mutations may be described by any two of formulas (III) to (VIII), as defined above, e.g., the subject compound includes two distinct arrangements of variable regions that define two distinct variable domains in the same compound. In some embodiments, the two distinct groups of mutations include a first group of mutations defined by one of formulas (III)-(V) and a second group of mutations defined by one of formulas (VI)-(VIII). In such cases, the subject compound may be bifunctional in the sense that the compound has specific binding properties for two target molecules. The compound may include two potential binding surfaces for the same target molecule, where the overall binding affinity of the compound may be modulated by an avidity effect. The compound may include a first potential binding surface for a first target molecule and a second binding surface that recruits a second target molecule.

Also provided are GB1 peptidic compounds that have been optimized for binding to a target molecule by affinity maturation, e.g., second generation GB1 peptidic compounds based on a parent GB1 peptidic compound that binds to a certain target molecule, where the second generation GB1 peptidic compounds are optimized for binding affinity and specificity. In certain embodiments, the subject peptidic compounds specifically bind to a target protein with high affinity, e.g., as determined by an SPR binding assay or an ELISA assay. The subject compounds may exhibit an affinity for a target protein of 1 uM or less, such as 300 nM or less, 100 nM or less, 30 nM or less, 10 nM or less, 5 nM or less, 2 nM or less, 1 nM or less, 300 pM or less, or even less. The subject peptidic compounds may exhibit a specificity for a target protein, e.g., as determined by comparing the affinity of the compound for the target protein with that for a reference protein (e.g., an albumin protein), that is 5:1 or more 10:1 or more, such as 30:1 or more, 100:1 or more, 300:1 or more, 1000:1 or more, or even more. In some cases, the GB1 peptidic compounds may be optimized for any desirable property, such as protein folding, protease stability, thermostability, compatibility with a pharmaceutical formulation, etc. Any convenient methods of affinity maturation may be used to select second generation GB1 peptidic compounds, e.g., phage display methods.

In some embodiments, the affinity maturation of a subject compound may include holding a fraction of the variant amino acid positions as fixed positions while the remaining variant amino acid positions are varied to select optimal amino acids at each position. A parent GB1 peptidic compound that specifically binds to a certain target molecule may be selected as a scaffold for an affinity maturation compound. In some cases, a number of affinity maturation compounds are prepared that include mutations at limited subsets of the variant amino acid positions of the parent (e.g., mutations at 4 of 15 variable positions), while the rest of the variant positions are held as fixed positions. The positions of the mutations may be tiled through the scaffold sequence to produce a series of compounds such that mutations at every variant position is represented and a diverse range of amino acids are substituted at every position (e.g., all 20 naturally occurring amino acids). Mutations that include deletion or insertion of one or more amino acids may also be included at variant positions of the affinity maturation compounds. An affinity maturation compound may be prepared and screened using any convenient method, e.g., phage display library screening, to identify second generation compounds having an improved property, e.g., increased binding affinity for a target molecule, protein folding, protease stability, thermostability, compatibility with a pharmaceutical formulation, etc.

In some embodiments, the affinity maturation of a subject compound may include holding most or all of the variant amino acid positions in the variable regions of the parent GB1 compound as fixed positions, and introducing contiguous mutations at positions adjacent to these variable regions. Such mutations may be introduced at positions in the parent GB1 compound that were previously considered fixed positions in the original GB1 scaffold domain. Such mutations may be used to optimize the GB1 compound variants for any desirable property, such as protein folding, protease stability, thermostability, compatibility with a pharmaceutical formulation, etc.

### VEGF Binding Compounds

In some embodiments, the subject compound specifically binds to a VEGF protein, such as VEGF-A, VEGF-B, VEGF-C, VEGF-D or VEGF-E, where the VEGF protein may be a synthetic D-protein or a L-protein.

In certain embodiments, the VEGF protein is a D-protein and the subject compound is L-peptidic. In certain embodiments, the VEGF protein is a L-protein and the subject compound is D-peptidic. In some embodiments, the VEGF protein is VEGF-A. In certain embodiments, the subject compound is described by formula (II) as defined above, and includes a GB1 scaffold domain and a variable domain, where the variable domain encompasses at least part of α1. In certain embodiments, the variable domain encompasses positions 21 to 41 of the scaffold domain.

In certain embodiments, the subject VEGF-binding compound is described by formula (III) as defined above, where the subject VEGF-binding compound includes a GB1 scaffold domain and a variable domain. In certain embodiments, the GB1 scaffold domain includes a first sequence having 80% or more (e.g., 90% or more) amino acid sequence identity to an amino acid sequence set forth in SEQ ID NO:2, and a second sequence having 80% or more (e.g., 90% or more) amino acid sequence identity to an amino acid sequence set forth in SEQ ID NO:3.

In certain embodiments, the variable domain comprises a sequence having 80% or more (e.g., 90% or more) amino acid sequence identity to an amino acid sequence set forth in one of SEQ ID NOs: 44-75. In certain embodiments, the variable domain comprises a sequence having 80% or more (e.g., 90% or more) amino acid sequence identity to an amino acid sequence set forth at positions 20-42 in one of SEQ ID NOs:76-162 and 529-809.

SEQ ID NO:
44 VDVFYAFDVFFDYASDNFADVADW
45 VDFYSAFDVFFYYASSNFYDVADW
46 VDVYSAVDVFYDYAAANFASFSDW
47 VDVYDAFDVFFVYAASNFKDFDDW
48 VDFFSAFDVFYAYADSNFDFYDDW
49 VDFYAAFSVFFDYAAFNFYDVDDW
50 VSYYDAFDVFFAYASSNFDFFDDW
51 VDFFAAFDVFYSYASFNFAFFDDW
52 VDVYDAFDVFYYYAAANYADFDDW
53 VDFFDAFDVFFDYAVANFADVDDW
54 VDVYSAFDVFYDYAVANFVDFSDW
55 VDFFAAFAVFDSYASSNFFSVADW
56 VDFYDAFDVFYDYASSNVSVFDDW
57 VDVFDAFDVFAVYAFDNFVYVDDW
58 VDFFDAFSVFDAYASSNYVVVDDW
59 VDVFDAFDVFFYYAAANFDVYDDW
60 VDFYSAFDVFFSYAASNFFVFDDW
61 VDFYDAFDVFFVYAVDNYFFDVDDW
62 VDLYDAFDVFFVYAASNFSDFDDW
63 VDVFDAFDVFFVYAASNFSDFDDW
64 VDVYGAFDVFFVYAASNFSDFDDW
65 VDVYDAFEVFFVYAASNFSDFDDW
66 VDVYDAFDVFFAYAASNFSDFDDW
67 VDVYDAFDVFFVYAASNFKDFDDW
68 VDVYDAFDVFFVYAARNFSDFDDW
69 VDVYDAFDVFFVYAAENFSDFDDW
70 VDVYDAFDVFFVYAAGNFSDFDDW
71 VDVYDAFDVFFVYAASNFKDFDDW
72 VDVYDAFDVFFVYAASNFGDFDDW
73 VDVYDAFDVFFVYAASNFSFFDDW
74 VDVYDAFDVFFVYAASNFSDIDDW
75 VDVYDAFDVFFVYAASNFKDFDDW

In certain embodiments, in formula (III), the mutation at position 38 in V1 includes insertion of 0, 1 or 2 variant amino acids.

In certain embodiments, the subject VEGF-binding compound comprises a sequence having 80% or more amino acid sequence identity (e.g., 85% or more, 90% or more, 95% or more, or 98% or more amino acid sequence identity) to an amino acid sequence set forth in one of SEQ ID NOs:76-162 and 529-809.

In some embodiments, the subject VEGF-binding compound comprises a sequence having 80% or more amino acid sequence identity (e.g., 85% or more, 90% or more, 95% or more, or 98% or more amino acid sequence identity) to that of clone E01 (SEQ ID NO:79). In certain embodiments, the VEGF-binding compound further includes one or more point mutations, selected from V22L, Y23F, D24G, D27E, V31A, A34S, S35R, S35E, S35G, S38K, S38G, D39F, F401 and D41V.

In certain embodiments, the subject VEGF-binding compound is described by formula (III) (F1-V1-F2), where F1 comprises a sequence having 60% or more (e.g., 70% or more, 80% or more, 90% or more, or 95% or more) amino acid sequence identity to the amino acid sequences TYKLILNGKTLKGETTTEA (SEQ ID NO:2).

In certain embodiments, the subject VEGF-binding compound is described by formula (III) (F1-V1-F2), where F1 comprises a sequence TYKX¹ILNGKTX²KX³EX⁴TX⁵EA (SEQ ID NO:528), where X¹ is selected from M and L, X² is selected from R, Q, M, L, C, S, Q, F, W, V, A, K, X³ is selected from F, R, D, S, W, D, G, A, E and C, X⁴ is selected from T, I, V, L, M, A, H, Y, C and F, and X⁵ is selected from T, I, A, L, V, M and V. In certain embodiments, X² is L. In certain embodiments, X³ is S or G. In certain embodiments, X⁴ is C or F. In certain embodiments, X⁵ is T or L. In certain embodiments, F1 comprises a sequence having 60% or more (e.g., 70% or more, 80% or more, 90% or more, or 95% or more) amino acid sequence identity to the amino acid sequence: TYKMILNGKTLKSECTTEA (SEQ ID NO:366). In certain embodiments, F1 comprises a sequence having 60% or more (e.g., 70% or more, 80% or more, 90% or more, or 95% or more) amino acid sequence identity to the amino acid sequence: TYKLILNGKTLKGEFTLEA (SEQ ID NO:367).

In certain embodiments, the subject VEGF-binding compound is described by formula (III) (F1-V1-F2), where F2 comprises a sequence having 60% or more (e.g., 70% or more, 80% or more, 90% or more, or 95% or more) amino acid sequence identity to the amino acid sequence: TYDDATKTFTVTE (SEQ ID NO:3). In certain embodiments, the subject VEGF-binding compound is described by formula (III) (F1-V1-F2), where F2 comprises a sequence having 60% or more (e.g., 70% or more, 80% or more, 90% or more, or 95% or more) amino acid sequence identity to the amino acid sequence: (T/R/I/V)YDDATKTFTVTE (SEQ ID NO:368).

In certain embodiments, the subject VEGF-binding compound is described by formula (III) (F1-V1-F2), where V1 comprises a sequence of the following formula: VDXXXAFXVLXXCAXXXFXXIXXW (SEQ ID NO:369), where each X is a variant amino acid. In some embodiments, the Cys residue at position 32 in V1 forms an intramolecular crosslink. In certain embodiments, in formula (III), the variant amino acid at position 22 in V1 is selected from L, F, I, M, R, V and W; the variant amino acid at position 23 in V1 is selected from F, I, N and Y; the variant amino acid at position 24 in V1 is selected from D, A, E, H, N, Q, T and V; the variant amino acid at position 27 in V1 is selected from D, A, E, G, H and Q; the variant amino acid at position 30 in V1 is selected from F, Y, L, S and W; the variant amino acid at position 31 in V1 is selected from V, A, D, E, G, I, L, M, P, S and T; the variant amino acid at position 34 in V1 is selected from A, D, E, I, S, T, V and Y; the variant amino acid at position 35 in V1 is selected from A, D, F, N, R, S, T and Y; the variant amino acid at position 36 in V1 is selected from N, E, H, I, K, Q, R, S, T, V and Y; the variant amino acid at position 38 in V1 is selected from S, A, D, E, F, G, L, N, P, Q, R, T and Y; the variant amino acid at position 39 in V1 is selected from F, D, E, H, I, L, N, V and Y; the variant amino acid at position 41 in V1 is selected from D, E, A, N, Q, S and V; and the variant amino acid at position 41a in V1 is selected from D, E, A, N and S.

In certain embodiments, in formula (III), the variant amino acid at position 22 in V1 is selected from L, F, I, M, R, V and W; the variant amino acid at position 23 in V1 is selected from F and Y; the variant amino acid at position 24 in V1 is selected from D and E; the variant amino acid at position 27 in V1 is selected from D and E; the variant amino acid at position 30 in V1 is selected from F and Y (e.g., F); the variant amino acid at position 31 in V1 is selected from V, A, D, E, G, I, L, M, P, S and T; the variant amino acid at position 34 in V1 is selected from A, E, S and T (e.g., A); the variant amino acid at position 35 in V1 is selected from A, N, R, S and Y (e.g., S); the variant amino acid at position 36 in V1 is selected from N, H, K, Q, S, V and Y (e.g., N); the variant amino acid at position 38 in V1 is selected from S, A, D, E, F, G, L, N, P, Q, R, T and Y; the variant amino acid at position 39 in V1 is selected from F, D, E, H, I, L, N, V and Y; the variant amino acid at position 41 in V1 is selected from D, E, A, N, Q, S and V; and the variant amino acid at position 41a in V1 is selected from D, E, A, N and S.

In certain embodiments, the subject VEGF-binding compound is described by formula (III) (F1-V1-F2), where:

F1 comprises a sequence having 60% or more (e.g., 70% or more, 80% or more, 90% or more, or 95% or more) amino acid sequence identity to the amino acid sequence:

(SEQ ID NO: 366)
TYKMILNGKTLKSECTTEA;

F2 comprises a sequence having 60% or more (e.g., 70% or more, 80% or more, 90% or more, or 95% or more) amino acid sequence identity to the amino acid sequence: TYDDATKTFTVTE (SEQ ID NO:3); and

V1 comprises a sequence of the following formula: VD(L/F/I/M/R/V/W)(F/Y)(D/E)AF(D/E)VL(F/Y)(V/A/D/E/G/I/L/M/P/S/T)CA(T/A/S/E)(S/A/N/R/Y)(N/H/K/Q/S/V/Y)F(S/P)(F/L/V/I/Y)I(D/E)DW (SEQ ID NO:370). In certain embodiments, V1 comprises a sequence of the following formula:

(SEQ ID NO: 371)
VD(L/F/I/M/R/V/W)FDAFDVLF(V/A/D/E/G/I/L/M/P/S/T)
CA(T/A/S/E)(S/A/N/R/Y)(N/H/K/Q/S/V/Y)FS(F/L/V/I/Y)
IDDW.

In certain embodiments, the subject VEGF-binding compound comprises a sequence having 80% or more amino acid sequence identity (e.g., 85% or more, 90% or more, 95% or more, or 98% or more amino acid sequence identity) to the amino acid sequence set forth in SEQ ID NO:372 (RFX-002 sequence) TYKMILNGKTLKSECTTEAVDLFDAFDVLFVCAASNFSFIDDWTYDDATKTFTVTE.

In certain embodiments, the subject VEGF-binding compound is described by formula (III) (F1-V1-F2), where V1 comprises a sequence of the following formula: VDXXXAFXVFXXYAXXXFXXXXDW (SEQ ID NO:373), where each X is a variant amino acid. In certain embodiments, in formula (III), the variant amino acid at position 22 in V1 is selected from L, F, M, Q and V; the variant amino acid at position 23 in V1 is selected from F, H and Y; the variant amino acid at position 24 in V1 is selected from D, E and H; the variant amino acid at position 27 in V1 is selected from D, E and G; the variant amino acid at position 30 in V1 is selected from F and Y; the variant amino acid at position 31 in V1 is selected from V, A, D, E, G and S; the variant amino acid at position 34 in V1 is selected from A, G, S and T; the variant amino acid at position 35 in V1 is selected from S, A, N and L; the variant amino acid at position 36 in V1 is selected from N, A, E, H, I, K, Q, R, S, T and Y; the variant amino acid at position 38 in V1 is selected from S, A, F, G, L, P, Q, T and Y; the variant amino acid at position 39 in V1 is selected from F, D, I, L, Q, S, T, V and Y; the variant amino acid at position 40 in V1 is selected from F and I; and the variant amino acid at position 41 in V1 is selected from D, E and S.

In certain embodiments, in formula (III), the variant amino acid at position 22 in V1 is selected from L and V; the variant amino acid at position 23 in V1 is F; the variant amino acid at position 24 in V1 is selected from D and E; the variant amino acid at position 27 in V1 is D; the variant amino acid at position 30 in V1 is F; the variant amino acid at position 31 in V1 is selected from V, A, D, E and G the variant amino acid at position 34 in V1 is selected from A, S and T; the variant amino acid at position 35 in V1 is selected from S, A and N; the variant amino acid at position 36 in V1 is selected from N, H, I, K, T and Y; the variant amino acid at position 38 in V1 is selected from S, A, L, P and Y; the variant amino acid at position 39 in V1 is selected from F, D, I, L, Q, S, T, V and Y; the variant amino acid at position 40 in V1 is F; and the variant amino acid at position 41 in V1 is D.

In certain embodiments, the subject VEGF-binding compound is described by formula (III) (F1-V1-F2), where:

F1 comprises a sequence having 60% or more (e.g., 70% or more, 80% or more, 90% or more, or 95% or more) amino acid sequence identity to the amino acid sequence:

(SEQ ID NO: 367)
TYKLILNGKTLKGEFTLEA;

F2 comprises a sequence having 60% or more (e.g., 70% or more, 80% or more, 90% or more, or 95% or more) amino acid sequence identity to the amino acid sequence: TYDDATKTFTVTE (SEQ ID NO:3); and

V1 comprises a sequence of the following formula: VD(L/V)F(D/E)AFDVFF(V/A/D/E/G/S)YA(A/S/T)(S/A/N)(N/H/I/K/T/Y)F(S/Y/A/P/L)(F/D/I/L/Q/S/T/V/Y)FDDW (SEQ ID NO:374). In certain embodiments, V1 comprises a sequence of the following formula:

(SEQ ID NO: 375)
VDLF(D/E)AFDVFF(V/G)YA(A/S/T)(S/A)(N/H)FS(F/D/V/Y)
FDDW.

In certain embodiments, the subject VEGF-binding compound comprises a sequence having 80% or more amino acid sequence identity (e.g., 85% or more, 90% or more, 95% or more, or 98% or more amino acid sequence identity) to the amino acid sequence set forth in SEQ ID NO:376 (RFX-025 sequence) TYKLILNGKTLKGEFTLEAVDLFDAFDVFFVYAASNFSFFDDWTYDDATKTFTVTE.

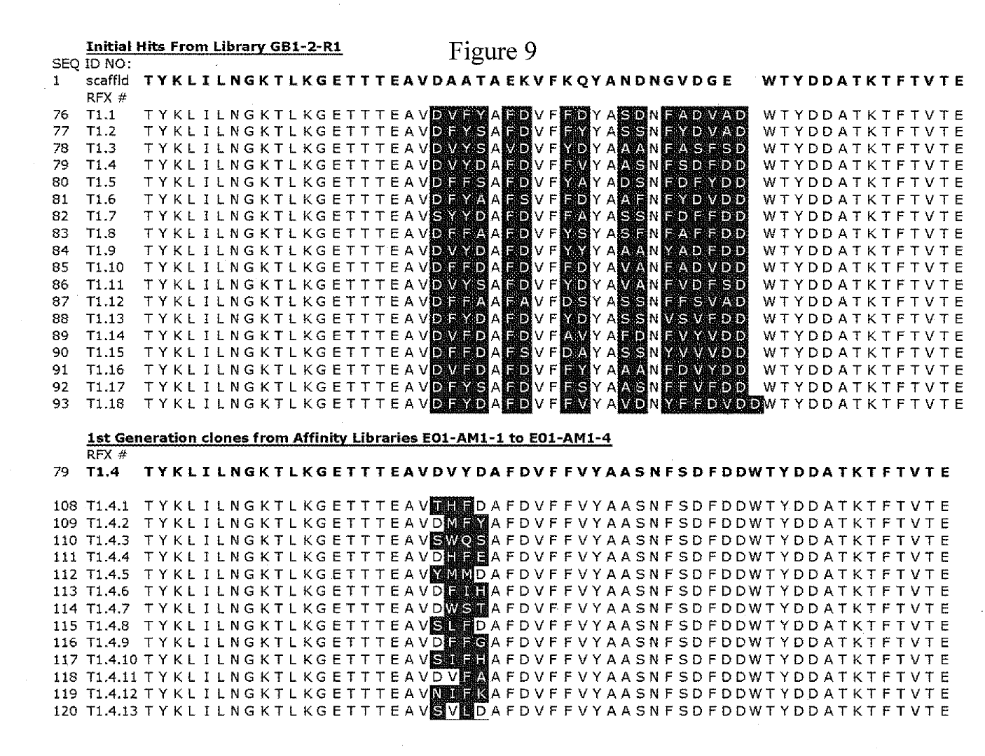

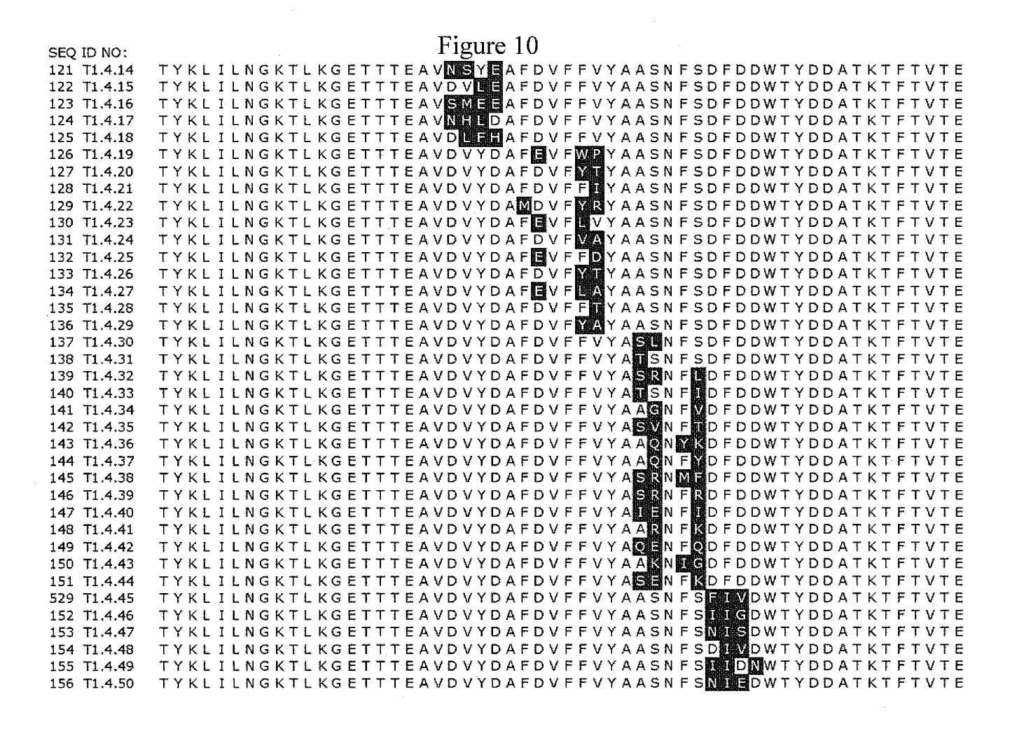

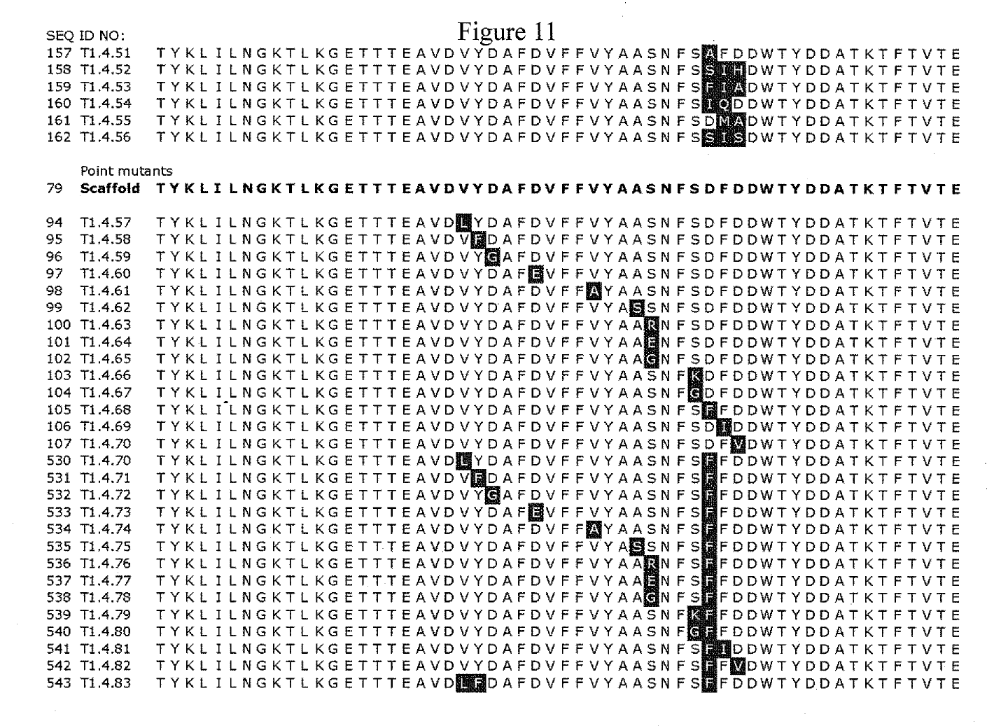

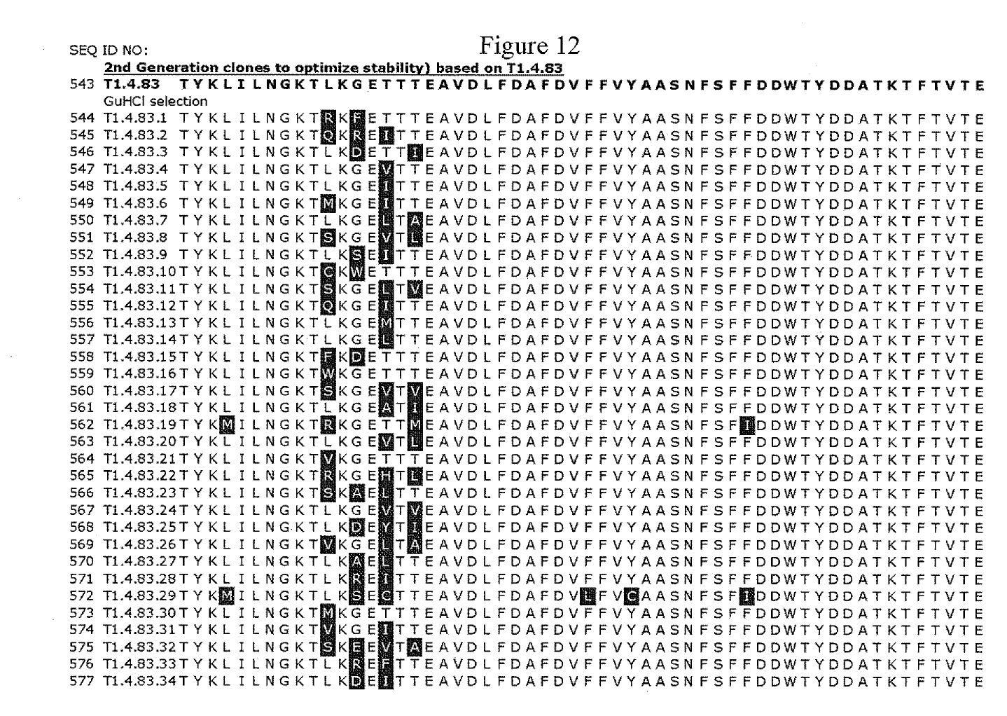

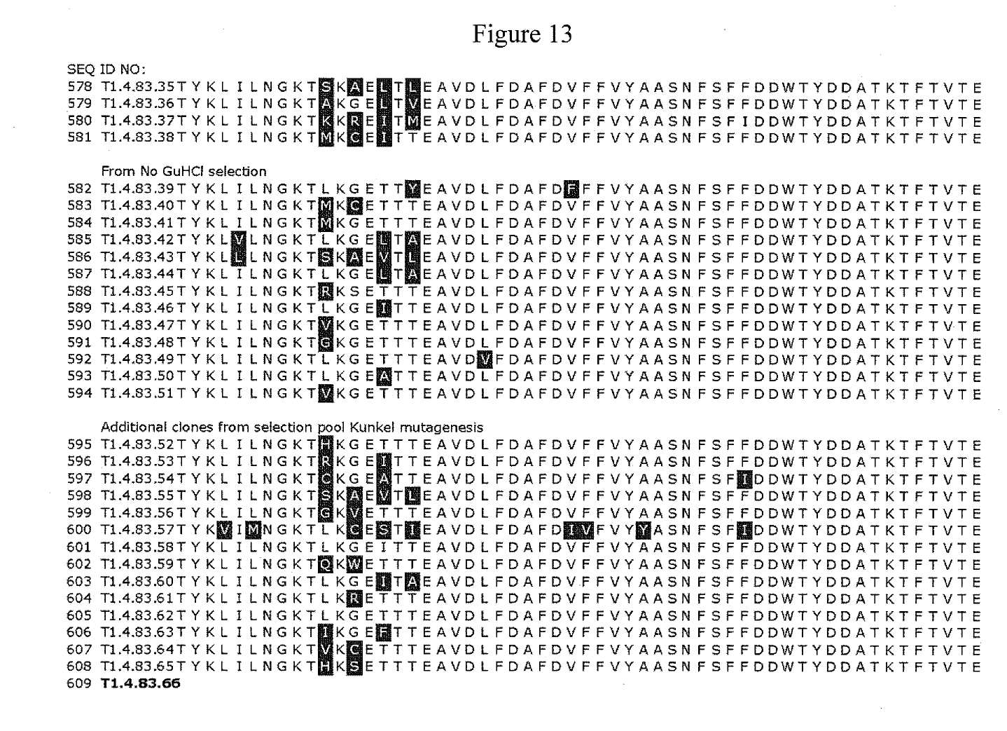

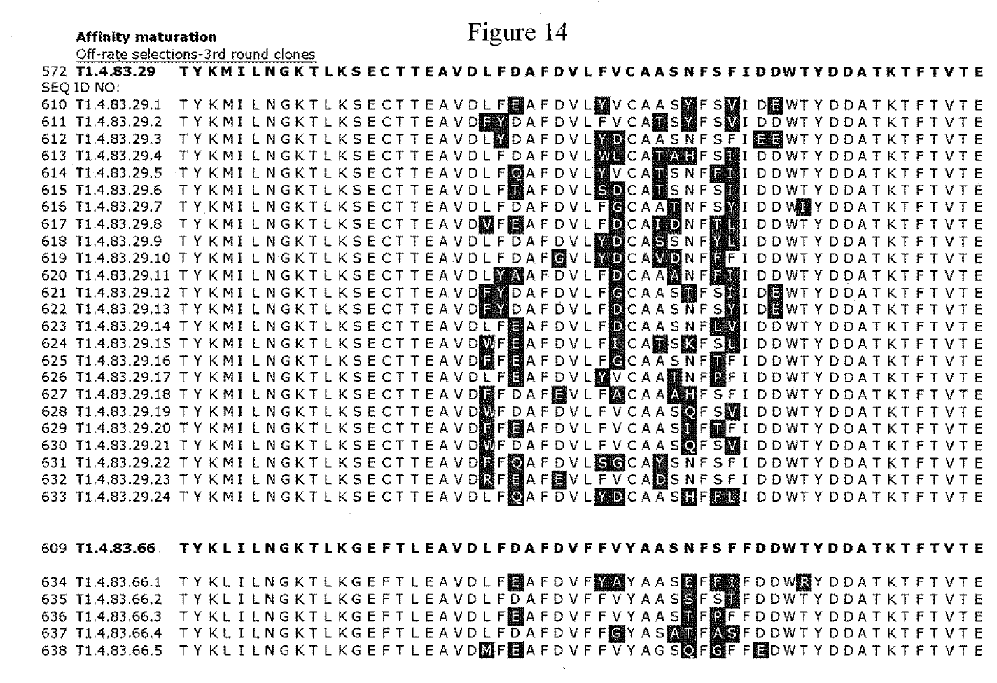

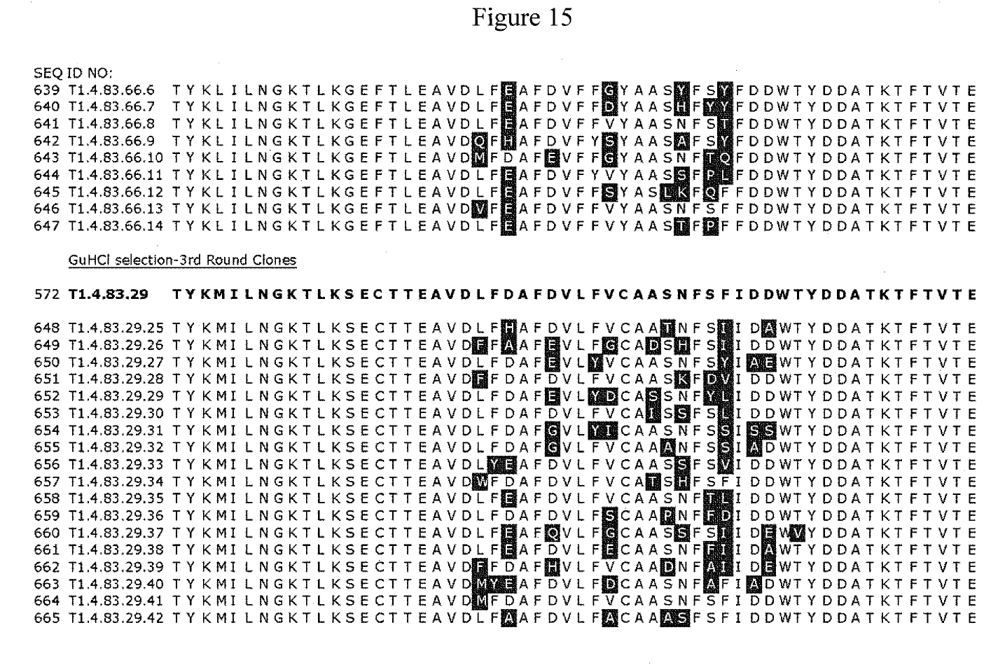

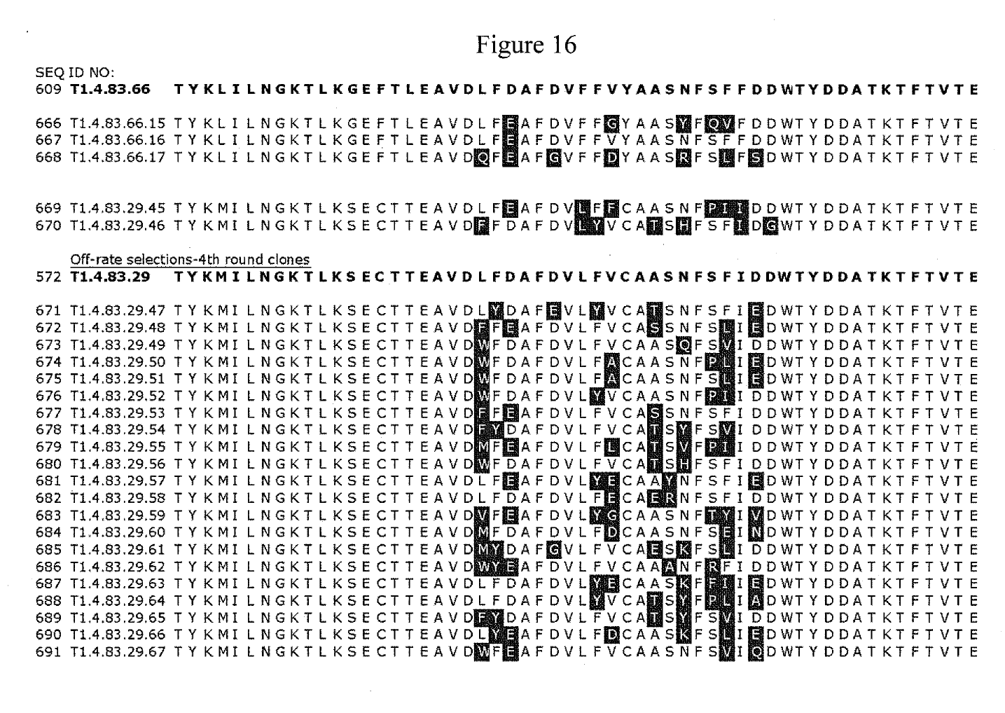

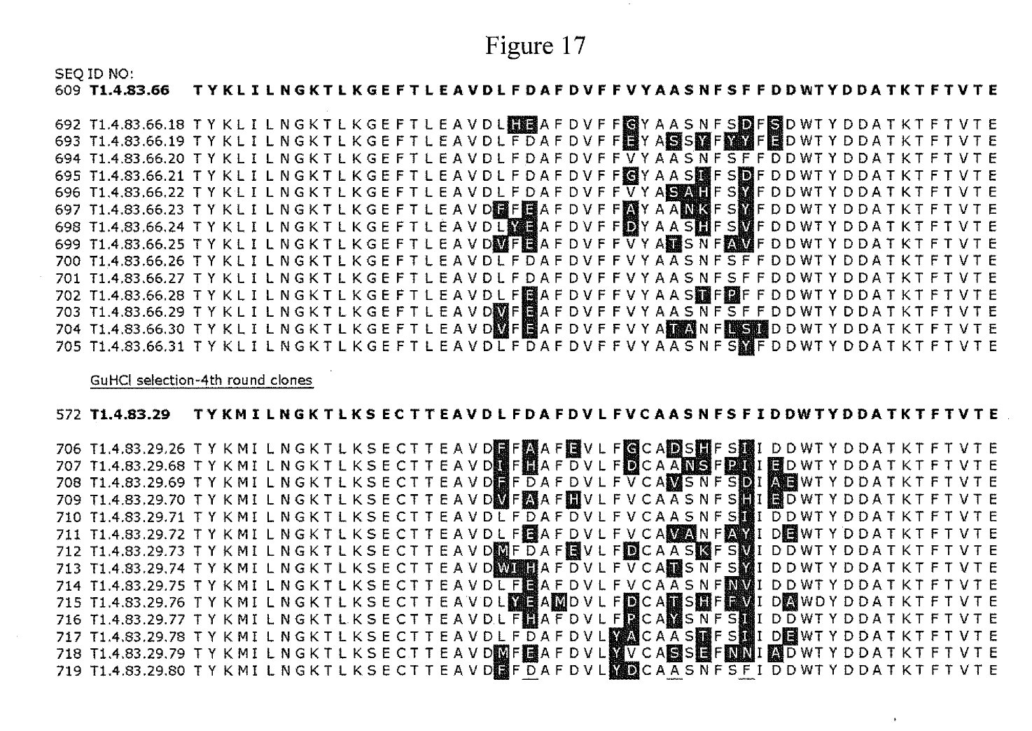

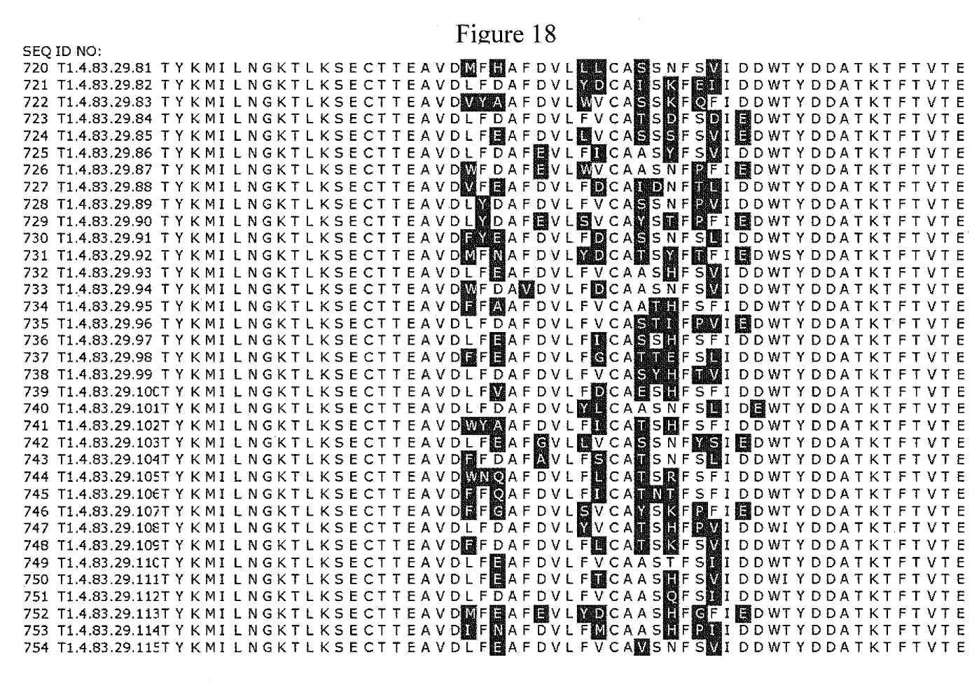

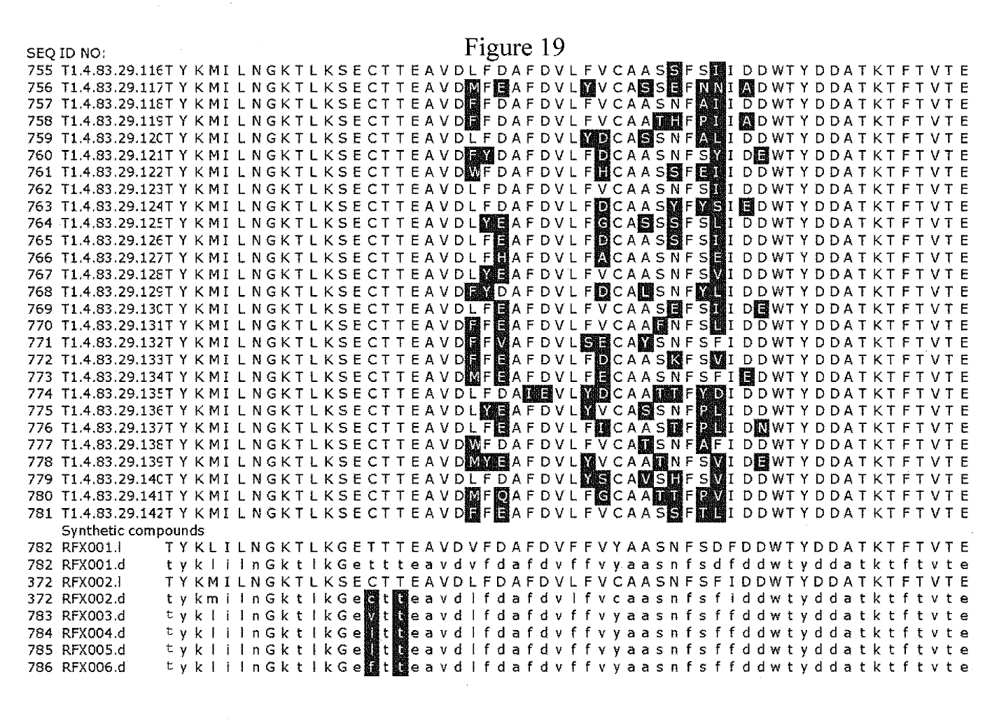

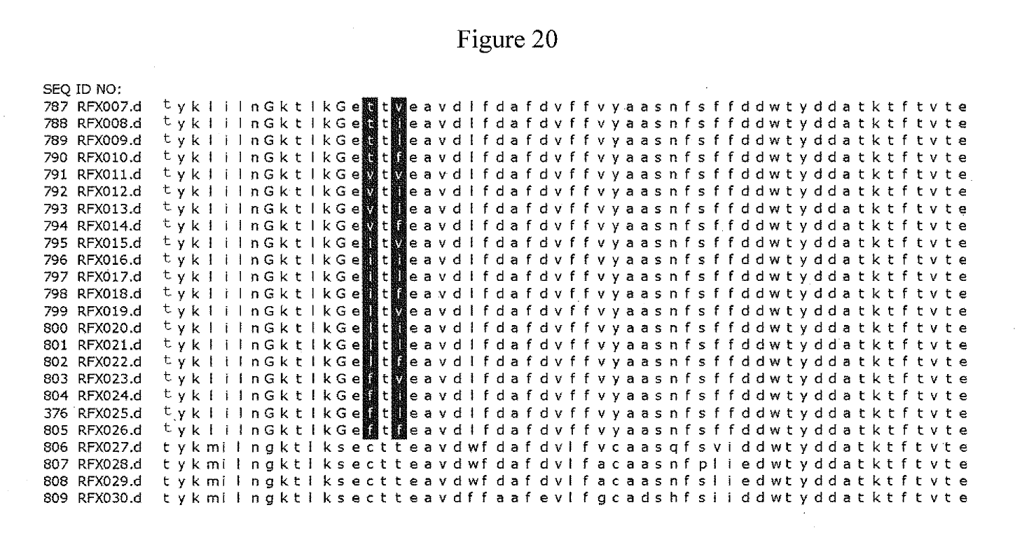

In certain embodiments, the subject VEGF-binding compound comprises a sequence having 80% or more amino acid sequence identity (e.g., 85% or more, 90% or more, 95% or more, or 98% or more amino acid sequence identity) to an amino acid sequence set forth in one of SEQ ID NOs:76-162 and 529-809 (see FIGS. 9-20). In certain embodiments, the VEGF protein is a D-protein and the subject compound is L-peptidic. In certain embodiments, the VEGF protein is a L-protein and the subject compound is D-peptidic.

In certain embodiments, the VEGF protein is a L-protein and the subject compound is L-peptidic. In some embodiments, the VEGF protein is VEGF-A. In certain embodiments, in formula (II), the variable domain of the subject VEGF-binding compound encompasses at least part of β1 and at least part of β4. In certain embodiments, the variable domain encompasses positions 3 to 16 and 52 to 55 of the GB1 scaffold domain.

In certain embodiments, the subject VEGF-binding compound is described by formula (VII) (V9-F11-V10) as defined above, where:

F11 comprises a sequence having 60% or more (e.g., 70% or more, 80% or more, 90% or more, or 95% or more) amino acid sequence identity to an amino acid sequence set forth in SEQ ID NO:30;

V9 comprises a sequence having 60% or more (e.g., 70% or more, 80% or more, 90% or more, or 95% or more) amino acid sequence identity to an amino acid sequence set forth in one of SEQ ID NOs:163-167 or to an amino acid sequence set forth at positions 1-17 of SEQ ID NOs:179-228 and 810-864; and

V10 comprises a sequence having 60% or more (e.g., 70% or more, 80% or more, 90% or more, or 95% or more) amino acid sequence identity to an amino acid sequence set forth in one of SEQ ID NOs:168-172 or to an amino acid sequence set forth at positions 51-55 of SEQ ID NOs:179-228 and 810-864.

SEQ ID NO:
163 T Y Y L F L F D S D S Y A D Y T V T
164 T Y S L F L F D Y Y A Y D V T S T
165 T Y Y L F L Y D A Y V S A D Y T D T
166 T Y Y L F L Y S V V D A D A Y F T V T
167 T Y S L F L F D F Y A F A V T A T
168 F F V D V D
169 F F V F F A
170 F F V V D Y
171 F F V D F S
172 F F V F F A

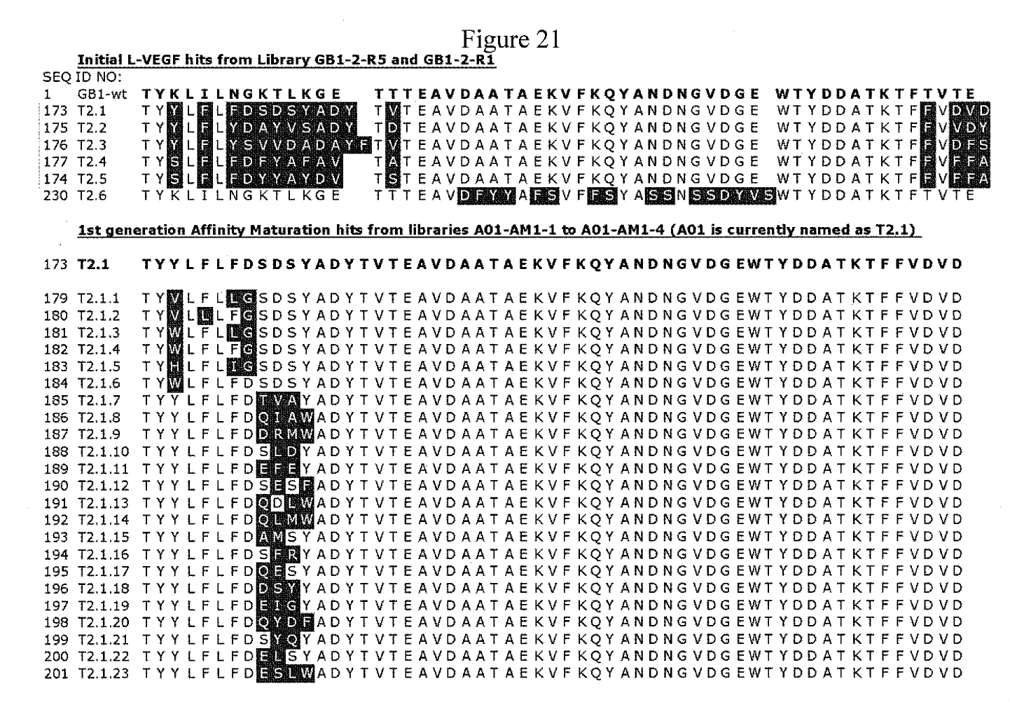

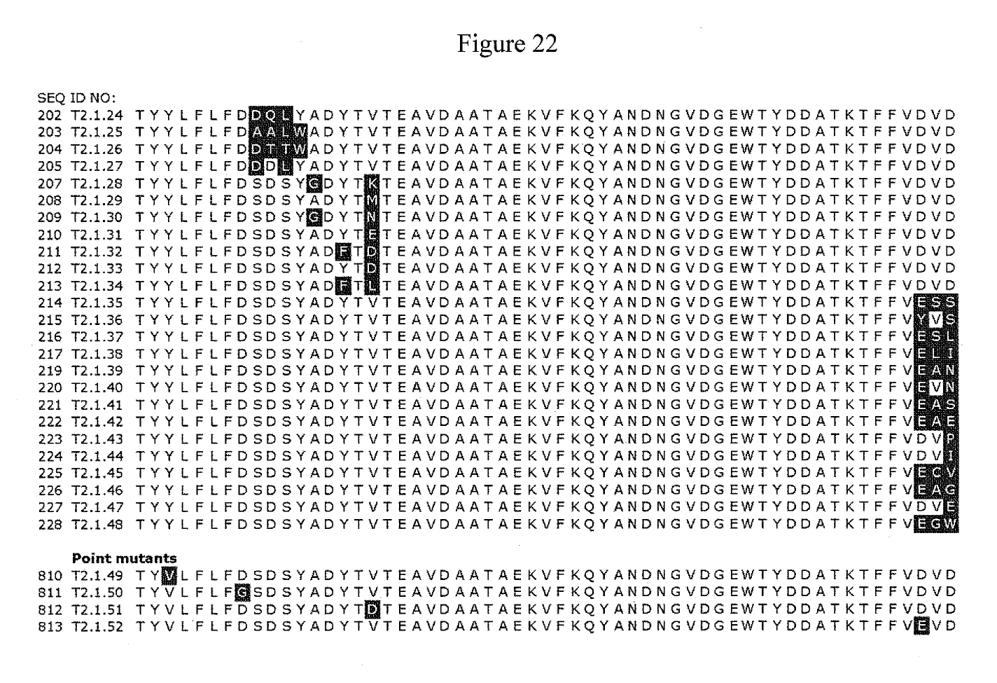

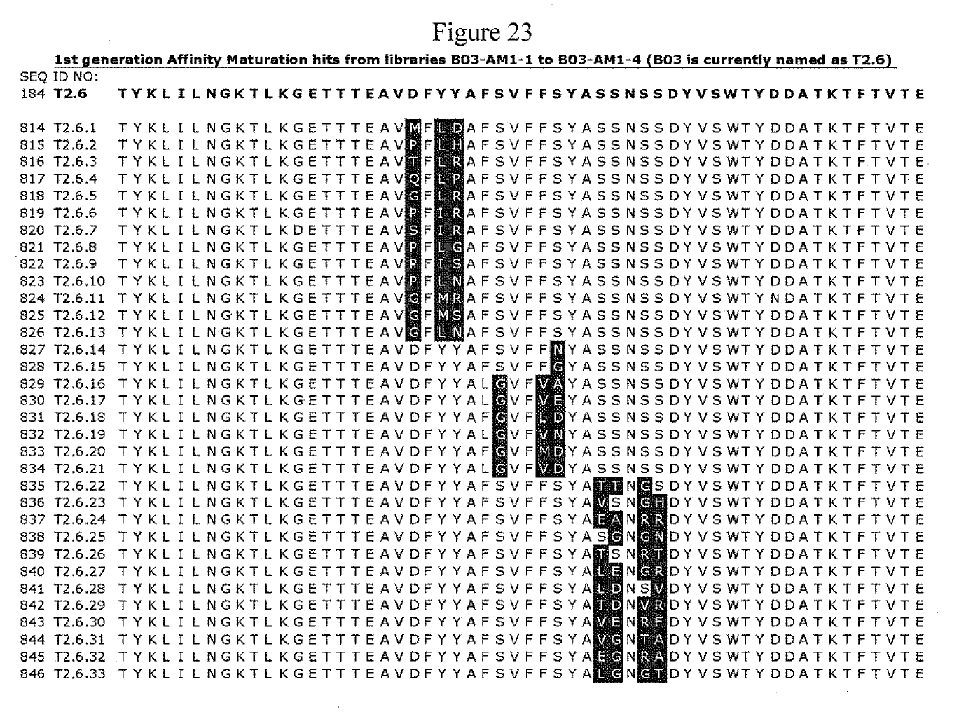

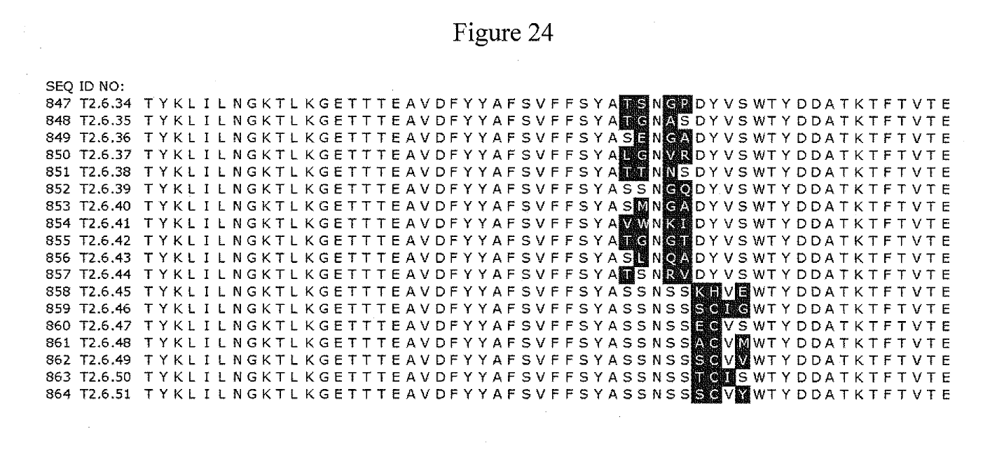

In certain embodiments, the subject VEGF-binding compound comprises a sequence having 80% or more amino acid sequence identity (e.g., 85% or more, 90% or more, 95% or more, or 98% or more amino acid sequence identity) to an amino acid sequence set forth in one of SEQ ID NOs: 173-228 and 810-864 (FIGS. 21-24).

In certain embodiments, the VEGF protein is a L-protein and the subject compound is L-peptidic. In some embodiments, the VEGF protein is VEGF-A. In certain embodiments, the subject compound is described by formula (II) as defined above, and includes a GB1 scaffold domain and a variable domain, where the variable domain encompasses at least part of α1. In certain embodiments, the variable domain encompasses positions 21 to 41 of the GB1 scaffold domain.

In certain embodiments, the subject VEGF-binding compound is described by formula (III) as defined above, where:

F1 comprises a sequence having 60% or more (e.g., 70% or more, 80% or more, 90% or more, or 95% or more) amino acid sequence identity to the amino acid sequence TYKLILNGKTLKGETTTEA (SEQ ID NO:2);

F2 comprises a sequence having 60% or more (e.g., 70% or more, 80% or more, 90% or more, or 95% or more) amino acid sequence identity to an amino acid sequence TYDDATKTFTVTE (SEQ ID NO:3); and

V1 comprises a sequence having 60% or more (e.g., 70% or more, 80% or more, 90% or more, or 95% or more) amino acid sequence identity to an amino acid sequence set forth in SEQ ID NO:229 (VDFYYAFSVFFSYASSNSSDYVSW).

In certain embodiments, the subject VEGF-binding compound comprises a sequence having 80% or more amino acid sequence identity (e.g., 85% or more, 90% or more, 95% or more, or 98% or more amino acid sequence identity) to an amino acid sequence set forth in one of SEQ ID NO:230 (TYKLILNGKTLKGETTTEAVDFYYAFSVFFSYASSNSSDYVSWTYDDATKTFTVTE). In certain embodiments, the VEGF protein is a L-protein and the subject compound is L-peptidic.

### SH2 Domain Containing Protein and SH3 Domain Containing Protein Binding Compounds

In some embodiments, the subject compound specifically binds to a SH2 domain containing protein (e.g., a 3BP2 protein) or a SH3 domain containing protein (e.g., an ABL or a Src protein).

In some embodiments, the subject compound specifically binds to a 3BP2 protein. In certain embodiments, in formula (II), the variable domain of the subject 3BP2-binding compound encompasses at least part of α1. In certain embodiments, the variable domain encompasses positions 18 to 35 of the GB1 scaffold domain.

In certain embodiments, the subject 3BP2-binding compound is described by formula (IV) (F3-V2-F4) as defined above, where:

F3 comprises a sequence having 60% or more (e.g., 70% or more, 80% or more, 90% or more, or 95% or more) amino acid sequence identity to the amino acid sequence TYKLILNGKTLKGETT (SEQ ID NO:7);

F4 comprises a sequence having 60% or more (e.g., 70% or more, 80% or more, 90% or more, or 95% or more) amino acid sequence identity to an amino acid sequence GVDGEWTYDDATKTFTVTE (SEQ ID NO:8); and

V2 comprises a sequence having 60% or more (e.g., 70% or more, 80% or more, 90% or more, or 95% or more) amino acid sequence identity to an amino acid sequence set forth in SEQ ID NO:231 (TVSSVFDDAYFYFVDYAFDN).

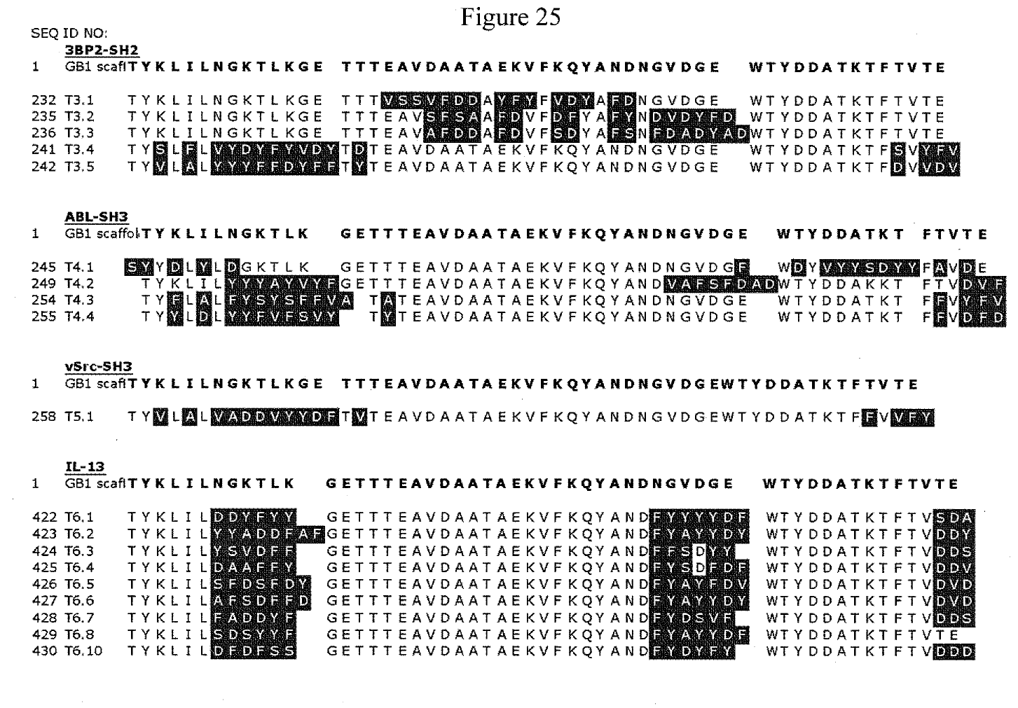

In certain embodiments, the subject 3BP2 binding compound comprises a sequence having 80% or more amino acid sequence identity (e.g., 85% or more, 90% or more, 95% or more, or 98% or more amino acid sequence identity) to an amino acid sequence set forth in SEQ ID NO:232 (TYKLILNGKTLKGETTTVSSVFDDAYFYFVDYAFDNGVDGEWTYDDATKTFTVTE) (FIG. 25). In certain embodiments, the 3BP2 protein is a L-protein and the subject compound is L-peptidic.

In certain embodiments, the subject 3BP2-binding compound is described by formula (II) as defined above, and includes a GB1 scaffold domain and a variable domain, where the variable domain encompasses at least part of α1. In certain embodiments, the variable domain encompasses positions 21 to 41 of the scaffold domain.

In certain embodiments, the subject 3BP2-binding compound is described by formula (III) (F1-V1-F2) as defined above, where:

F1 comprises a sequence having 60% or more (e.g., 70% or more, 80% or more, 90% or more, or 95% or more) amino acid sequence identity to the amino acid sequence TYKLILNGKTLKGETTTEA (SEQ ID NO:2);

F2 comprises a sequence having 60% or more (e.g., 70% or more, 80% or more, 90% or more, or 95% or more) amino acid sequence identity to an amino acid sequence TYDDATKTFTVTE (SEQ ID NO:3); and

V1 comprises a sequence having 60% or more (e.g., 70% or more, 80% or more, 90% or more, or 95% or more) amino acid sequence identity to an amino acid sequence set forth in one of SEQ ID NO: 233 (VSFSAAFDVFDFYAFYNDVDYFDW) and SEQ ID NO:234 (VAFDDAFDVFSDYAFSNFDADYADW).

In certain embodiments, the subject 3BP2 binding compound comprises a sequence having 80% or more amino acid sequence identity (e.g., 85% or more, 90% or more, 95% or more, or 98% or more amino acid sequence identity) to an amino acid sequence set forth in one of SEQ ID NOs: 235-236 (FIG. 25). In certain embodiments, the 3BP2 protein is a L-protein and the subject compound is L-peptidic.

In certain embodiments, the subject 3BP2-binding compound is described by formula (II) as defined above, and includes a GB1 scaffold domain and a variable domain, where the variable domain encompasses at least part of the loop between β1 and β2 and at least part of β4. In certain embodiments, the variable domain encompasses positions 3 to 16 and positions 52 to 55 of the scaffold domain.

In certain embodiments, the subject 3BP2-binding compound is described by formula (VII) as defined above, where:

F11 comprises a sequence having 60% or more (e.g., 70% or more, 80% or more, 90% or more, or 95% or more) amino acid sequence identity to an amino acid sequence set forth in SEQ ID NO:30;

V9 comprises a sequence having 60% or more (e.g., 70% or more, 80% or more, 90% or more, or 95% or more) amino acid sequence identity to an amino acid sequence set forth in one of SEQ ID NO:237 (TYSLFLVYDYFYVDYTDT) and SEQ ID NO:238 (TYVLALYYYFFDYFFTYT); and

V10 comprises a sequence having 60% or more (e.g., 70% or more, 80% or more, 90% or more, or 95% or more) amino acid sequence identity to an amino acid sequence set forth in one of SEQ ID NO:239 (FSVYFV) and SEQ ID NO:240 (FDVVDV).

In certain embodiments, the subject 3BP2-binding compound comprises a sequence having 80% or more amino acid sequence identity (e.g., 85% or more, 90% or more, 95% or more, or 98% or more amino acid sequence identity) to an amino acid sequence set forth in one of SEQ ID NOs: 232, 235-236 and 241-242 (FIG. 25). In certain embodiments, the 3BP2 protein is a L-protein and the subject compound is L-peptidic.

### ABL-SH3 Binding Compounds

In some embodiments, the subject compound specifically binds to an ABL protein. In certain embodiments, the subject ABL-binding compound is described by formula (II) as defined above, and includes a GB1 scaffold domain and a variable domain, where the variable domain encompasses at least part of β1 and at least part of the loop between β3 and β4. In certain embodiments, the variable domain encompasses positions 1 to 17 and positions 41 to 54 of the scaffold domain.

In certain embodiments, the subject ABL-binding compound is described by formula (VIII) (V11-F12-V12) as defined above, where:

F12 comprises a sequence having 60% or more (e.g., 70% or more, 80% or more, 90% or more, or 95% or more) amino acid sequence identity to an amino acid sequence set forth in SEQ ID NO:37;

V11 comprises a sequence having 60% or more (e.g., 70% or more, 80% or more, 90% or more, or 95% or more) amino acid sequence identity to an amino acid sequence set forth in SEQ ID NO:243 (SYYDLYLDG); and

V12 comprises a second sequence having 60% or more (e.g., 70% or more, 80% or more, 90% or more, or 95% or more) amino acid sequence identity to an amino acid sequence set forth in SEQ ID NO:244 (GFWDYVYYSDYYFAVDE).

In certain embodiments, the subject ABL-binding compound comprises a sequence having 80% or more amino acid sequence identity (e.g., 85% or more, 90% or more, 95% or more, or 98% or more amino acid sequence identity) to an amino acid sequence set forth in SEQ ID NO:245 (SYYDLYLDGKTLKGETTTEAVDAATAEKVFKQYANDNGVDGFWDYVYYSDYYFAVDE) (FIG. 25). In certain embodiments, the ABL protein is a L-protein and the subject compound is L-peptidic.

In certain embodiments, in formula (II), the variable domain of the subject ABL-binding compound encompasses at least part of the loop between β1 and β2, and at least part of the loop between α1 and β3. In certain embodiments, the variable domain encompasses positions 7 to 12 and positions 36 to 41 of the GB1 scaffold domain.

In certain embodiments, the subject ABL-binding compound is described by formula (VI) (F8-V6-F9-V7-F10-V8) as defined above, where:

F8 comprises a sequence having 60% or more (e.g., 70% or more, 80% or more, 90% or more, or 95% or more) amino acid sequence identity to an amino acid sequence set forth in SEQ ID NO:21;

F9 comprises a sequence having 60% or more (e.g., 70% or more, 80% or more, 90% or more, or 95% or more) amino acid sequence identity to an amino acid sequence set forth in SEQ ID NO:22;

F10 comprises a sequence having 60% or more (e.g., 70% or more, 80% or more, 90% or more, or 95% or more) amino acid sequence identity to an amino acid sequence set forth in SEQ ID NO:23;

V6 comprises a sequence having 60% or more (e.g., 70% or more, 80% or more, 90% or more, or 95% or more) amino acid sequence identity to an amino acid sequence set forth in SEQ ID NO:246 (LYYYAYVYFG);

V7 comprises a sequence having 60% or more (e.g., 70% or more, 80% or more, 90% or more, or 95% or more) amino acid sequence identity to an amino acid sequence set forth in SEQ ID NO:247 (DVAFSFDADW); and

V8 comprises a sequence having 60% or more (e.g., 70% or more, 80% or more, 90% or more, or 95% or more) amino acid sequence identity to an amino acid sequence set forth in SEQ ID NO:248 (VDVF).

In certain embodiments, the subject ABL-binding compound comprises a sequence having 80% or more amino acid sequence identity (e.g., 85% or more, 90% or more, 95% or more, or 98% or more amino acid sequence identity) to an amino acid sequence set forth in SEQ ID NO:249 (FIG. 25). In certain embodiments, the ABL protein is a L-protein and the subject compound is L-peptidic.

In certain embodiments, the subject compound is described by formula (II) as defined above, and includes a GB1 scaffold domain and a variable domain, where the variable domain encompasses at least part of the loop between β1 and β2 and at least part of β4. In certain embodiments, the variable domain encompasses positions 3 to 16 and positions 52 to 55 of the scaffold domain.

In certain embodiments, the subject ABL-binding compound is described by formula (VII) as defined above, where:

F11 comprises a sequence having 60% or more (e.g., 70% or more, 80% or more, 90% or more, or 95% or more) amino acid sequence identity to an amino acid sequence set forth in SEQ ID NO:30;

V9 comprises a sequence having 60% or more (e.g., 70% or more, 80% or more, 90% or more, or 95% or more) amino acid sequence identity to an amino acid sequence set forth in one of SEQ ID NO:250 (TYFLALFYSYSFFVATAT) and SEQ ID NO:251 (TYYLDLYYFVFSVYTYT); and

V10 comprises a sequence having 60% or more (e.g., 70% or more, 80% or more, 90% or more, or 95% or more) amino acid sequence identity to an amino acid sequence set forth in one of SEQ ID NO:252 (FFVYFV) and SEQ ID NO:253 (FFVDFD).

In certain embodiments, the subject ABL-binding compound comprises a sequence having 80% or more amino acid sequence identity (e.g., 85% or more, 90% or more, 95% or more, or 98% or more amino acid sequence identity) to an amino acid sequence set forth in one of SEQ ID NOs:245, 249 and 254-255 (FIG. 25). In certain embodiments, the ABL protein is a L-protein and the subject compound is L-peptidic.

### Src-SH3 Binding Compounds

In some embodiments, the subject compound specifically binds to a Src protein. In certain embodiments, the subject compound is described by formula (II) as defined above, and includes a GB1 scaffold domain and a variable domain, where the variable domain encompasses at least part of the loop between β1 and β2 and at least part of β4. In certain embodiments, the variable domain encompasses positions 3 to 16 and positions 52 to 55 of the scaffold domain.

In certain embodiments, the subject Src-binding compound is described by formula (VII) as defined above, where:

F11 comprises a sequence having 60% or more (e.g., 70% or more, 80% or more, 90% or more, or 95% or more) amino acid sequence identity to an amino acid sequence set forth in SEQ ID NO:30;

V9 comprises a sequence having 60% or more (e.g., 70% or more, 80% or more, 90% or more, or 95% or more) amino acid sequence identity to an amino acid sequence set forth in SEQ ID NO:256 (TYVLALVADDVYYDFTVT); and

V10 comprises a sequence having 60% or more (e.g., 70% or more, 80% or more, 90% or more, or 95% or more) amino acid sequence identity to an amino acid sequence set forth in SEQ ID NO:257 (FFVVFY).

In certain embodiments, the subject Src-binding compound comprises a sequence having 80% or more amino acid sequence identity (e.g., 85% or more, 90% or more, 95% or more, or 98% or more amino acid sequence identity) to an amino acid sequence set forth in SEQ ID NO:258 (FIG. 25). In certain embodiments, the Src protein is a L-protein and the subject compound is L-peptidic.

### Cytokine Binding Compounds

In some embodiments, the subject compound specifically binds to a cytokine, such as an interleukin protein (e.g., an IL-13 protein) or a tumor necrosis factor protein (e.g., a TNF-a protein, a B-cell activating factor (BAFF) protein, or a RANKL protein).

### IL-13 Binding Compounds

In certain embodiments, in formula (II), the variable domain of the subject IL-13 binding compound encompasses at least part of the loop between β1 and β2, and at least part of the loop between α1 and β3. In certain embodiments, the variable domain encompasses positions 7 to 12 and positions 36 to 41 of the GB1 scaffold domain.

In certain embodiments, the subject IL-13 binding compound is described by formula (VI) (F8-V6-F9-V7-F10-V8) as defined above, where:

F8 comprises a sequence having 60% or more (e.g., 70% or more, 80% or more, 90% or more, or 95% or more) amino acid sequence identity to an amino acid sequence set forth in SEQ ID NO:21;

F9 comprises a sequence having 60% or more (e.g., 70% or more, 80% or more, 90% or more, or 95% or more) amino acid sequence identity to an amino acid sequence set forth in SEQ ID NO:22;

F10 comprises a sequence having 60% or more (e.g., 70% or more, 80% or more, 90% or more, or 95% or more) amino acid sequence identity to an amino acid sequence set forth in SEQ ID NO:23;

V6 comprises a sequence having 60% or more (e.g., 70% or more, 80% or more, 90% or more, or 95% or more) amino acid sequence identity to an amino acid sequence set forth in one of SEQ ID NOs:377-391;

V7 comprises a sequence having 60% or more (e.g., 70% or more, 80% or more, 90% or more, or 95% or more) amino acid sequence identity to an amino acid sequence set forth in one of SEQ ID NOs:392-406; and

V8 comprises a sequence having 60% or more (e.g., 70% or more, 80% or more, 90% or more, or 95% or more) amino acid sequence identity to an amino acid sequence set forth in one of SEQ ID NOs:407-421.

| SEQ ID NO: | SEQ ID NO: |
|---|---|
| 377 L D D Y F Y Y G | 400 D F Y D Y F Y W |
| 378 L Y Y A D D F A F G | 401 D F Y Y Y Y D F W |
| 379 L Y S V D F F G | 402 D F Y S D F Y Y D W |
| 380 L D A A F F Y G | 403 D F Y A V F D Y W |
| 381 L S F D S F D Y G | 404 D F Y D A F Y F Y W |
| 382 L A F S D F F D G | 405 D F Y S D F D F W |
| 383 L F A D D Y F G | 406 D F Y S D F A V D W |
| 384 L S D S Y Y F G | 407 V S D A |
| 385 L D F D F S S G | 408 V D D Y |
| 386 L D D Y F Y Y G | 409 V D D S |
| 387 L S D Y A A F G | 410 V D D V |
| 388 L F D S Y S V G | 411 V D V D |
| 389 L D A S F A Y A G | 412 V D V D |
| 390 L D A A F F Y G | 413 V D D S |
| 391 L S D Y Y V Y G | 414 V T E |
| 392 D F Y Y Y Y D F W | 415 V D D D |
| 393 D F Y A Y Y D Y W | 416 V S D A |
| 394 D F F S D Y Y W | 417 V D V D |
| 395 D F Y S D F D F W | 418 V D S V |
| 396 D F Y A Y F D V W | 419 V D D S |
| 397 D F Y A Y Y D Y W | 420 V D D V |
| 398 D F Y D S V F W | 421 V D D V |
| 399 D F Y A Y Y D F W | |

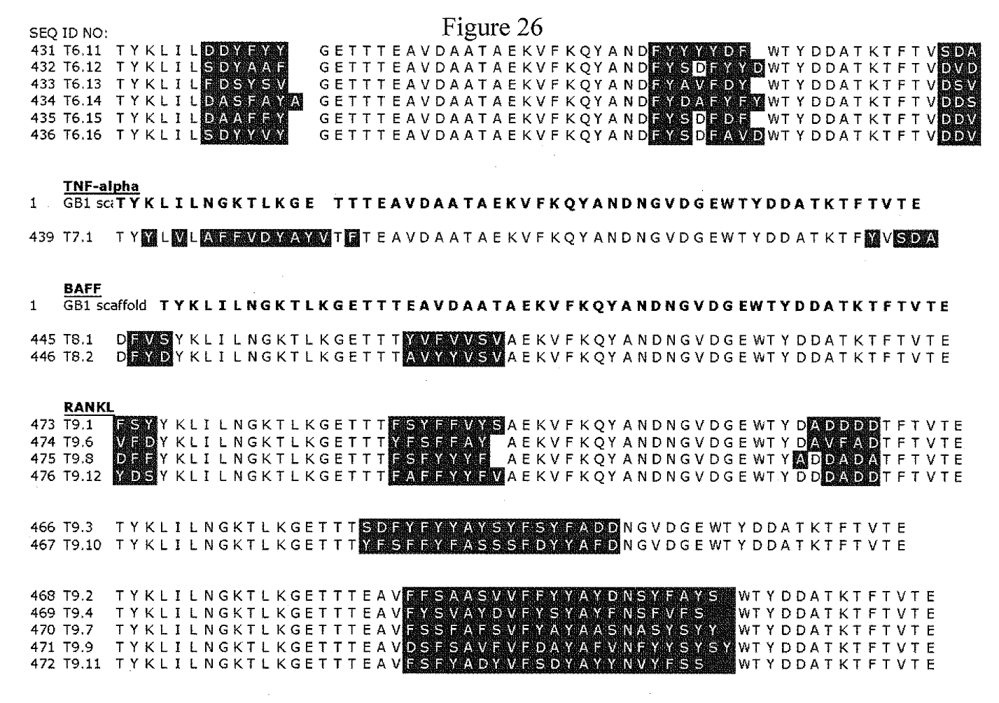

In certain embodiments, the subject IL-13 binding compound comprises a sequence having 80% or more amino acid sequence identity (e.g., 85% or more, 90% or more, 95% or more, or 98% or more amino acid sequence identity) to an amino acid sequence set forth in one of SEQ ID NOs:422-436 (FIGS. 25-26). In certain embodiments, the IL-13 protein is a L-protein and the subject compound is L-peptidic.

### TNF-α Binding Compounds

In some embodiments, the subject compound specifically binds to a TNF-α protein. In certain embodiments, the subject compound is described by formula (II) as defined above, and includes a GB1 scaffold domain and a variable domain, where the variable domain encompasses at least part of the loop between β1 and β2 and at least part of β4. In certain embodiments, the variable domain encompasses positions 3 to 16 and positions 52 to 55 of the scaffold domain.

In certain embodiments, the subject TNF-α binding compound is described by formula (VII) (V9-F11-V10) as defined above, where:

F11 comprises a sequence having 60% or more (e.g., 70% or more, 80% or more, 90% or more, or 95% or more) amino acid sequence identity to an amino acid sequence set forth in SEQ ID NO:30;

V9 comprises a sequence having 60% or more (e.g., 70% or more, 80% or more, 90% or more, or 95% or more) amino acid sequence identity to an amino acid sequence set forth in SEQ ID NO:437 (TYYLVLAFFVDYAYVTFT); and

V10 comprises a sequence having 60% or more (e.g., 70% or more, 80% or more, 90% or more, or 95% or more) amino acid sequence identity to an amino acid sequence set forth in SEQ ID NO:438 (FYVSDA).

In certain embodiments, the subject TNF-α binding compound comprises a sequence having 80% or more amino acid sequence identity (e.g., 85% or more, 90% or more, 95% or more, or 98% or more amino acid sequence identity) to an amino acid sequence set forth in SEQ ID NO:439 (FIG. 26). In certain embodiments, the TNF-α protein is a L-protein and the subject compound is L-peptidic.

### BAFF Binding Compounds

In some embodiments, the subject compound specifically binds to a BAFF protein. In certain embodiments, the subject compound is described by formula (V) (V3-F5-V4-F6-V5-F7) as defined above, where:

F5 comprises the sequence having 60% or more (e.g., 70% or more, 80% or more, 90% or more, or 95% or more) amino acid sequence identity to an amino acid sequence set forth in KLILNGKTLKGETT (SEQ ID NO:12);

F6 comprises the sequence having 60% or more (e.g., 70% or more, 80% or more, 90% or more, or 95% or more) amino acid sequence identity to an amino acid sequence set forth in EKVFKQYANDNGVDGEWT (SEQ ID NO:13);

F7 comprises the sequence having 60% or more (e.g., 70% or more, 80% or more, 90% or more, or 95% or more) amino acid sequence identity to an amino acid sequence set forth in FTVTE (SEQ ID NO:14);

V3 comprises a sequence having 60% or more (e.g., 70% or more, 80% or more, 90% or more, or 95% or more) amino acid sequence identity to an amino acid sequence set forth in one of SEQ ID NOs:440-441;

V4 comprises a sequence having 60% or more (e.g., 70% or more, 80% or more, 90% or more, or 95% or more) amino acid sequence identity to an amino acid sequence set forth in one of SEQ ID NOs:442-443; and

V5 comprises a sequence having 60% or more (e.g., 70% or more, 80% or more, 90% or more, or 95% or more) amino acid sequence identity to an amino acid sequence set forth in YDDATKT (SEQ ID NO:444).

SEQ ID NO:
440 F V S Y
441 F Y D Y
442 T Y V F V V S V A
443 T A V Y Y V S V A
444 Y D D A T K T

In certain embodiments, the subject BAFF binding compound comprises a sequence having 80% or more amino acid sequence identity (e.g., 85% or more, 90% or more, 95% or more, or 98% or more amino acid sequence identity) to an amino acid sequence set forth in one of SEQ ID NOs:445-446 (FIG. 26). In certain embodiments, the BAFF protein is a L-protein and the subject compound is L-peptidic.

### RANKL Binding Compounds

In certain embodiments, the RANKL protein is a L-protein and the subject compound is L-peptidic. In certain embodiments, the subject compound is described by formula (II) as defined above, and includes a GB1 scaffold domain and a variable domain, where the variable domain encompasses at least part of α1. In certain embodiments, the variable domain encompasses positions 21 to 41 of the GB1 scaffold domain.

In certain embodiments, the subject RANKL-binding compound is described by formula (III) as defined above, where:

F1 comprises a sequence having 60% or more (e.g., 70% or more, 80% or more, 90% or more, or 95% or more) amino acid sequence identity to the amino acid sequence TYKLILNGKTLKGETTTEA (SEQ ID NO:2);

F2 comprises a sequence having 60% or more (e.g., 70% or more, 80% or more, 90% or more, or 95% or more) amino acid sequence identity to an amino acid sequence TYDDATKTFTVTE (SEQ ID NO:3); and

V1 comprises a sequence having 60% or more (e.g., 70% or more, 80% or more, 90% or more, or 95% or more) amino acid sequence identity to an amino acid sequence set forth in one of SEQ ID NOs:447-448.

In certain embodiments, the subject RANKL-binding compound comprises a sequence having 80% or more amino acid sequence identity (e.g., 85% or more, 90% or more, 95% or more, or 98% or more amino acid sequence identity) to an amino acid sequence set forth in one of SEQ ID NOs:466-467 (FIG. 26).

SEQ ID NO:
447 T S D F Y F Y Y A Y S Y F S Y F A D D N
448 F F Y F A S F A S S S F D Y Y A F D N
449 V F F S A A S V V F F Y Y A Y D N S Y F A Y S W
450 V F Y S V A Y D V F Y S Y A Y F N S F V F S W
451 V F S S F A F S V F Y A Y A A S N A S Y S Y Y W
452 V D S F S A V F V F D A Y A F V N F Y Y S Y S Y W
453 V F S F Y A D Y V F S D Y A Y Y N V Y F S S W
454 F S Y Y
455 V F D Y
456 D F F Y
457 Y D S Y
458 T F S Y F F V Y S A
459 T Y F S F F A Y A
460 T F S F Y Y Y F A
461 T F A F F Y Y F V A
462 Y D A D D D D T
463 Y D A V F A D T
464 Y A D D A D A T
465 Y D D D A D D T

In some embodiments, the subject compound specifically binds to a RANKL protein. In certain embodiments, in formula (II), the variable domain of the subject RANKL-binding compound encompasses at least part of α1. In certain embodiments, the variable domain encompasses positions 18 to 35 of the GB1 scaffold domain.

In certain embodiments, the subject RANKL-binding compound is described by formula (IV) (F3-V2-F4) as defined above, where:

F3 comprises a sequence having 60% or more (e.g., 70% or more, 80% or more, 90% or more, or 95% or more) amino acid sequence identity to the amino acid sequence TYKLILNGKTLKGETT (SEQ ID NO:7);

F4 comprises a sequence having 60% or more (e.g., 70% or more, 80% or more, 90% or more, or 95% or more) amino acid sequence identity to an amino acid sequence GVDGEWTYDDATKTFTVTE (SEQ ID NO:8); and

V2 comprises a sequence having 60% or more (e.g., 70% or more, 80% or more, 90% or more, or 95% or more) amino acid sequence identity to an amino acid sequence set forth in one of SEQ ID NOs:449-453.

In certain embodiments, the subject RANKL-binding compound comprises a sequence having 80% or more amino acid sequence identity (e.g., 85% or more, 90% or more, 95% or more, or 98% or more amino acid sequence identity) to an amino acid sequence set forth in one of SEQ ID NOs: 468-472 (FIG. 26). In certain embodiments, the RANKL protein is a L-protein and the subject compound is L-peptidic.

In some embodiments, the subject compound specifically binds to a RANKL protein. In certain embodiments, the subject compound is described by formula (V) (V3-F5-V4-F6-V5-F7) as defined above, where:

F5 comprises the sequence having 60% or more (e.g., 70% or more, 80% or more, 90% or more, or 95% or more) amino acid sequence identity to an amino acid sequence set forth in KLILNGKTLKGETT (SEQ ID NO:12);

F6 comprises the sequence having 60% or more (e.g., 70% or more, 80% or more, 90% or more, or 95% or more) amino acid sequence identity to an amino acid sequence set forth in EKVFKQYANDNGVDGEWT (SEQ ID NO:13);

F7 comprises the sequence having 60% or more (e.g., 70% or more, 80% or more, 90% or more, or 95% or more) amino acid sequence identity to an amino acid sequence set forth in FTVTE (SEQ ID NO:14);

V3 comprises a sequence having 60% or more (e.g., 70% or more, 80% or more, 90% or more, or 95% or more) amino acid sequence identity to an amino acid sequence set forth in one of SEQ ID NOs:454-457;

V4 comprises a sequence having 60% or more (e.g., 70% or more, 80% or more, 90% or more, or 95% or more) amino acid sequence identity to an amino acid sequence set forth in one of SEQ ID NOs:458-461; and

V5 comprises a sequence having 60% or more (e.g., 70% or more, 80% or more, 90% or more, or 95% or more) amino acid sequence identity to an amino acid sequence set forth in one of (SEQ ID NO:462-465).

In certain embodiments, the subject RANKL binding compound comprises a sequence having 80% or more amino acid sequence identity (e.g., 85% or more, 90% or more, 95% or more, or 98% or more amino acid sequence identity) to an amino acid sequence set forth in one of SEQ ID NOs:473-476 (FIG. 26). In certain embodiments, the RANKL protein is a L-protein and the subject compound is L-peptidic.

### Modified Compounds

Also provided are modified GB1 peptidic compounds that include a GB1 peptidic domain that specifically binds to a target molecule, as described above, and one or more modifying domains. The modifying domain may be attached to the subject compound via covalent conjugation or crosslinking to a sidechain or terminal amino acid residue. The modifying domain may be attached to the GB1 peptidic domain via a single bond or a suitable linker, e.g., a PEG linker, a linker including one or more amino acids, or a saturated hydrocarbon linker. A variety of linkers find use in the subject modified compounds. Any convenient reagents and methods may be used to conjugate a modifying domain to a subject GB1 peptidic domain, for example, as described in G. T. Hermanson, “Bioconjugate Techniques” Academic Press, 2nd Ed., 2008. Functional groups that may be used in covalently bonding the domains together, via an optional linker, to produce the modified compound include: hydroxyl, sulfhydryl, amino, and the like. Certain moieties on the modifying domain and/or GB1 peptidic compound may be protected using convenient blocking groups, see, e.g. Green & Wuts, Protective Groups in Organic Synthesis (John Wiley & Sons) 3rd Ed. (1999). The particular portion(s) of the GB1 peptidic compound that is modified to provide for covalent linkage may be chosen so as not to substantially adversely interfere with the desired binding activity, e.g. for the target protein. A region that does not affect the target binding activity may be modified, such that a sufficient amount of the desired target binding activity is preserved.

While not wishing to be bound by any theory, the attachment of a modifying domain to a subject compound may confer on the resulting modified compound different properties and functions, e.g., an extended half-life in vivo, improved protease resistance, decreased elimination in vivo, a detectable property such as fluorescence, immobilization to a support, or affinity for a complementary binding moiety. The modifying domain may be peptidic or not peptidic, naturally occurring or synthetic. The modifying domain may be a specific binding moiety, a moiety that imparts desirable drug-like properties, a label or a support. A variety of modifying domains find use in the subject modified compounds.

In certain embodiments, the modified compounds are described by formula (IX):

X-L-Y (IX)

where X is a GB1 peptidic domain of the invention that specifically binds with high affinity to a target molecule, e.g., a target protein;

L is an optional linking group; and

Y is a modifying domain.

In certain embodiments, the modifying domain is a half-life extending moiety, a specific binding moiety, a label or a support.

The term “half-life extending moiety” refers to a pharmaceutically acceptable moiety, domain, or “vehicle” covalently linked or conjugated to the subject compound, that prevents or mitigates in vivo proteolytic degradation or other activity-diminishing chemical modification of the subject compound, increases half-life or other pharmacokinetic properties (e.g., rate of absorption), reduces toxicity, improves solubility, increases biological activity and/or target selectivity of the subject compound with respect to a target of interest, increases manufacturability, and/or reduces immunogenicity of the subject compound, compared to an unconjugated form of the subject compound.

In certain embodiments, the modifying domain is a half-life extending moiety (e.g., a polypeptide that binds serum proteins, such as an immunoglobulin (e.g., IgG) or a serum albumin (e.g., human serum albumin (HSA)). Polyethylene glycol is an example of a useful half-life extending moiety. Exemplary half-life extending moieties include a polyalkylene glycol moiety (e.g., PEG), a serum albumin or a fragment thereof, a transferrin receptor or a transferrin-binding portion thereof, and a moiety comprising a binding site for a polypeptide that enhances half-life in vivo, a copolymer of ethylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid (e.g., polylysine), a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polysialic acid, a polyacetal, a long chain fatty acid, a long chain hydrophobic aliphatic group, an immunoglobulin Fc domain (see, e.g., U.S. Pat. No. 6,660,843), an albumin (e.g., human serum albumin; see, e.g., U.S. Pat. No. 6,926,898 and US 2005/0054051; U.S. Pat. No. 6,887,470), a transthyretin (TTR; see, e.g., US 2003/0195154; 2003/0191056), or a thyroxine-binding globulin (TBG).

In certain embodiments, the half-life extending moiety is a fatty acid. Any convenient fatty acids may be used in the subject modified compounds. See e.g., Chae et al., “The fatty acid conjugated exendin-4 analogs for type 2 antidiabetic therapeutics”, J. Control Release. 2010 May 21; 144(1):10-6.

In certain embodiments, the modifying domain is a specific binding moiety. The specific binding moiety is capable of specifically binding to a second moiety that is complementary to it. Complementary binding moiety pairs of specific binding moieties include, but are not limited to, a ligand and a receptor, an antibody and an antigen, complementary polynucleotides, an aptamer and a small molecule, a polyhistidine tag and nickel, and a chemoselective reactive group (e.g., a thiol) and an electrophilic group (e.g., with which the reactive thiol group can undergo a Michael addition). The specific binding pairs may include analogs, derivatives and fragments of the original specific binding member. For example, an antibody directed to a protein antigen may also recognize peptide fragments, chemically synthesized, labeled protein, derivatized protein, etc. so long as an epitope is present.

In certain embodiments, the specific binding moiety is an affinity tag such as a biotin moiety. Exemplary biotin moieties include biotin, desthiobiotin, oxybiotin, 2′-iminobiotin, diaminobiotin, biotin sulfoxide, biocytin, etc. The biotin moiety is capable of specifically binding with high affinity to a chromatography support that contains immobilized avidin, neutravidin or streptavidin. In some cases, a monomeric avidin support may be used to specifically bind a biotin-modified compound with moderate affinity thereby allowing bound compounds to be later eluted competitively from the support (e.g., with a 2 mM biotin solution) after non-biotinylated polypeptides have been washed away.

In certain embodiments, the modifying domain is a support. Suitable supports may have a variety of forms and compositions and derive from naturally occurring materials, naturally occurring materials that have been synthetically modified, or synthetic materials. Examples of suitable support materials include, but are not limited to, silicon, plastic, glass, CPG, polymers, such as cellulose, nitrocellulose, agarose (e.g., Sepharose(r) from Pharmacia) and dextran (e.g., Sephadex(r) and Sephacyl(r), also from Pharmacia), polyacrylamides, polyvinyl alcohols, copolymers of hydroxyethyl methacrylate and methyl methacrylate, and the like, polyacrylamide, nylon, polystyrene, polyvinyl chloride, polypropylene, ceramic, photoresist, teflon or rubber support. In some cases, the silicon support is a silicon dioxide or a silicon nitride surface. The support may be in the form of planar solid supports, tubes, beads, discs, silicon chips, microplates, polyvinylidene difluoride (PVDF) membrane, nitrocellulose membrane, nylon membrane, other porous membrane, non-porous membrane, e.g., plastic, polymer, perspex, silicon, amongst others, a plurality of polymeric pins, or a plurality of microtitre wells, 96-well plate, array substrate, filter paper and the like. In some embodiments, the support is a planar solid support or a bead.

In certain embodiments, the modifying domain is a label. Examples of labels include labels that permit both the direct and indirect measurement of the presence of the modified compound. Examples of labels that permit direct measurement of the modified compound include radiolabels, fluorophores, dyes, beads, nanoparticles (e.g., quantum dots), chemiluminescers, colloidal particles, paramagnetic labels and the like. Radiolabels may include radioisotopes, such as ³⁵S, ¹⁴C, ¹²⁵I ³H, ⁶⁴Cu and ¹³¹I. The subject compounds can be labeled with the radioisotope using any convenient techniques, such as those described in Current Protocols in Immunology, Volumes 1 and 2, Coligen et al., Ed. Wiley-Interscience, New York, N.Y., Pubs. (1991), and radioactivity can be measured using scintillation counting or positron emmission. Examples of labels which permit indirect measurement of the presence of the modified compound include enzymes where a substrate may provide for a colored or fluorescent product. For example, the modified compound may include a covalently bound enzyme capable of providing a detectable product signal after addition of suitable substrate. Instead of covalently binding the enzyme to the modified compound, the modified compound may be modified to comprise a first member of specific binding pair which specifically binds with a second member of the specific binding pair that is conjugated to the enzyme, e.g. the modified compound may be covalently bound to biotin and the enzyme conjugate to streptavidin. Examples of suitable enzymes for use in conjugates include horseradish peroxidase, alkaline phosphatase, malate dehydrogenase and the like. Where not commercially available, such enzyme conjugates may be readily produced by any convenient techniques.

In certain embodiments, the label is a fluorophore. The term “fluorophore” refers to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE); cyanine dyes, e.g. Cy3, CY5, Cy5.5, QUASAR™ dyes etc.; dansyl derivatives; rhodamine dyes e.g. 6-carboxytetramethylrhodamine (TAMRA), CAL FLUOR dyes, tetrapropano-6-carboxyrhodamine (ROX). BODIPY fluorophores, ALEXA dyes, Oregon Green, pyrene, perylene, benzopyrene, squarine dyes, coumarin dyes, luminescent transition metal and lanthanide complexes and the like. The term fluorophore includes excimers and exciplexes of such dyes.

In some embodiments, the subject modified compound includes a modifying domain that is useful as a pharmaceutical (e.g., a protein, nucleic acid, organic small molecule, etc.). Exemplary pharmaceutical proteins include, e.g., cytokines, antibodies, chemokines, growth factors, interleukins, cell-surface proteins, extracellular domains, cell surface receptors, cytotoxins, etc. Exemplary small molecule pharmaceuticals include small molecule toxins or therapeutic agents.

The subject compounds may be modified in a way to form chimeric molecules comprising a GB1 peptidic compound fused to a heterologous polypeptide or amino acid sequence. In some cases, such a chimeric molecule comprises a fusion of a GB1 peptidic compound with a tag polypeptide which provides an epitope to which an anti-tag antibody can selectively bind. In some cases, the epitope tag is placed at the amino- or carboxyl-terminus of the subject GB1 peptidic compound, but could also be placed in any convenient region that does not significantly disrupt binding to the target, e.g. a loop region. The presence of such epitope-tagged forms of a GB1 peptidic compound can be detected using an antibody against the tag polypeptide. Also, provision of the epitope tag allows the GB1 peptidic compound to be readily isolated or purified by affinity purification using an anti-tag antibody or another type of affinity matrix that binds to the epitope tag. In certain embodiments, the chimeric molecule may include a fusion of a subject GB1 peptidic compound with an immunoglobulin or a particular region of an immunoglobulin. In certain embodiments, the chimeric molecule may include a cell penetrating peptide (e.g., tat). The cell penetrating peptide may facilitate cellular uptake of a chimeric molecule.

Any convenient tag polypeptides and their respective antibodies may be used. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

Also provided are arrays of probes. In some embodiments, a subject array includes two or more distinct probes, wherein one or more of the probes is a subject GB1 peptidic compound that specifically binds with high affinity to a target protein. An “array” refers to a solid support that provides a plurality of spatially addressable locations, also referred to as features, at which molecules may be bound. In some cases, only one kind of molecule (e.g. a GB1 peptidic compound) is bound at each feature. Similarly, “to array” a collection of molecules means to form an array of the molecules. In some embodiments, the subject array of probes specifically binds two or more distinct proteins of interest when contacted with a sample that includes the proteins. The array-bound proteins may be detected using any convenient method, e.g., using a protein specific fluorescent or colorimetric signal. The subject arrays of probes find use in the qualitative and quantitative determination of proteins of interest, if present, in a sample.

### Polynucleotides

Also provided are polynucleotides that encode GB1 peptidic compounds as described above. In some embodiments, the polynucleotide encodes a GB1 peptidic compound that includes at least 3 mutations at non-core positions in a region outside of the β1-β2 region.

In some embodiments, the polynucleotide encodes a GB1 peptidic compound that includes between 30 and 80 residues, such as between 40 and 70, between 45 and 60 residues, or between 50 and 56 residues. In certain embodiments, the polynucleotide encodes a GB1 peptidic compound that includes a peptidic sequence of 50, 51, 52, 53, 54, 55, 56, 57, 58, 59 or 60 residues. In certain embodiments, the peptidic sequence is of 54, 55, 56 or 57 residues, such as 55 or 56 residues.

In certain embodiments, the polynucleotide is a replicable expression vector that includes a nucleic acid sequence encoding a GB1 peptidic compound that may be expressed in a protein expression system. In certain embodiments, the polynucleotide is a replicable expression vector that includes a nucleic acid sequence encoding a gene fusion, where the gene fusion encodes a fusion protein including the GB1 peptidic compound fused to all or a portion of a viral coat protein.

In certain embodiments, the subject polynucleotides are capable of being expressed and displayed in a cell-based or cell-free display system. Any convenient display methods may be used to display GB1 peptidic compounds encoded by the subject polynucleotides, such as cell-based display techniques and cell-free display techniques. In certain embodiments, cell-based display techniques include phage display, bacterial display, yeast display and mammalian cell display. In certain embodiments, cell-free display techniques include mRNA display and ribosome display.

In some embodiments, a subject polynucleotide is expressed in a cell to produce a GB1 peptidic compound that specifically binds to a target within the cell, thereby modulating the activity of the target. In certain embodiments, the modulation of the activity of the target within the cell imparts one or more desirable traits on the cell, such as, tolerance to an environmental stimulus, pathogen or disease; increased size, or modified growth rate. In certain embodiments, the cell is a cell in a plant. In certain embodiments, the target is a transcription factor, a pathogen or a disease related target protein.

### Methods

Also provided are methods of making and using the compounds, e.g., as described above.

### Methods of Making

A variety of different methods may be employed to make compounds of the invention. Methods of making the subject compounds include, but are not limited to, protein expression methods, solid phase peptide synthesis methods, solution phase synthesis methods and native chemical ligation methods.

In some embodiments, the subject compounds are prepared in a protein expression system. The nucleic acids encoding a subject compound may be introduced directly into a host cell, and the cell incubated under conditions sufficient to induce expression of the encoded compound. Any convenient cell for expression may be used as a host cell. For example, yeast, insect, plant, etc., cells.

In some embodiments, the subject compounds are prepared using stepwise solid phase peptide synthesis methods, e.g., such as the stepwise addition of amino acids in a solid-phase Merrifield-type synthesis. Such methods may be used to prepare compounds of high purity that are free from undesirable side products. For the synthesis of a D-peptidic compound, D-amino acids or protected D-amino acids are utilized rather than the L-amino acids. A summary of some of the various methods available for synthesizing the subject compounds can be found in Steward et al., in “Solid Phase Peptide Synthesis”, W.H. Freeman Co., San Francisco, 1969; Bodanszky et al., in “Peptide Synthesis”, John Wiley & Sons, Second Edition, 1976 and Meienhofer, in “Hormonal Proteins and Peptides”, Vol. 2, p. 46, Academic Press (New York), 1983; and Kent, Ann. Rev. Biochem., 57, 957, 1988, for solid phase peptide synthesis, and Schroder et al., in “The Peptides”, Vol. 1, Academic Press (New York), 1965 for solution synthesis. Any convenient protecting group strategies may be used such as, but are not limited to, Fmoc solid-phase peptide synthesis and Boc solid-phase peptide synthesis strategies. In Boc solid-phase peptide synthesis a Boc-amino protecting group is used at the amino terminal and benzyl or benzyl-based protecting groups may be used for the protection of sidechain functional groups. In Fmoc solid-phase peptide synthesis a Fmoc-amino protecting group is used at the amino terminal and tert-butyl or benzyl-based protecting groups may be used for protection of sidechain functional groups. Convenient protecting groups that may be used in such synthetic methods are described in the above references and by McOmic in “Protective Groups in Organic Chemistry”, Plenum Press, New York, 1973; and Greene and Wuts, “Protective Groups in Organic Synthesis”, John Wiley & Sons, 4th Edition, 2006.

In some embodiments, the subject compounds are prepared using solution phase chemical synthesis methods, such as but not limited to, the hybrid method. In certain embodiments, the hybrid method includes the solid phase synthesis of protected peptide fragments, that may be synthesized and purified on a large scale. The protected fragments may then be assembled in solution using any convenient solution phase method, before final deprotection. For example, the hybrid method was used in the manufacture of the peptide drug Fuzeon.

In some embodiments, the subject compounds are prepared by the assembly of polypeptide building blocks using native chemical ligation methods. In this procedure, two or more polypeptide fragments are first synthesized, that contain termini adapted for native chemical ligation or for kinetically controlled ligation. After stepwise chemical synthesis and cleavage from their respective solid phase resins, and after purification e.g. by reverse phase HPLC, two of the two or more polypeptides are mixed and reacted to join the adapted termini and form a larger, linear polypeptide that includes the two polypeptides linked by a native amide bond. Further native chemical ligation and/or kinetically controlled ligation reactions can then be performed to add further polypeptide fragments in a convergent synthetic strategy. For a review of native chemical ligation and kinetically controlled ligation methods for the preparation of proteins see, e.g., “Total chemical synthesis of proteins,” Stephen B. H. Kent, Chem. Soc. Reviews, 38, 338-51 (2009).

Once a subject compound has been produced, it may be optionally purified or used without further purification. Purification may be performed using any convenient method, for example, by chromatography (e.g., RP-HPLC, ion exchange, affinity, and sizing column chromatography), centrifugation, differential solubility, or by any other convenient technique for the purification of proteins.

In some cases, the peptidic compound that is produced exists in a random coil or an unfolded state. The peptidic compound may then be folded using any convenient method, such that the compound folds from the random coil or unfolded state into a characteristic three-dimensional structure. In some cases, folding the compound includes dissolving the compound in an aqueous buffer under conditions that mimic physiological conditions (e.g., conditions of pH, ionic strength, temperature, and the like) and allowing the compound to fold into a characteristic three-dimensional structure in solution over a period of time (e.g., 2 days). The progress of folding of the compound may be followed using any convenient methods, such as HPLC, circular dichroism, etc.

### Methods of Use

The above-described compounds may be employed in a variety of methods. One such method includes contacting a subject compound with a target of the compound under conditions suitable for binding of the target to produce a complex. In some embodiments, a method of blocking binding of a ligand to its receptor is provided. In these embodiments, the method includes administering a GB1 peptidic compound to a subject, where the compound binds to either the receptor or the ligand in said subject and blocks binding thereof.

A subject compound may inhibit at least one activity of its target in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10⁻⁷ M or less (e.g., 1×10⁻⁷ M or less, 1×10⁻⁸ M or less, 1×10⁻⁹M or less, 1×10⁻¹⁰ M or less, or 1×10⁻¹¹ M or less). In assays in which a mouse is employed, a subject compound may have an ED₅₀ of less than 1 μg/mouse (e.g., 1 ng/mouse to about 1 μg/mouse).

The protocols that may be employed in these methods are numerous, and include but are not limited to cell-free assays, e.g., binding assays; cellular assays in which a cellular phenotype is measured, e.g., gene expression assays; and in vivo assays that involve a particular animal (which, in certain embodiments may be an animal model for a condition related to the target). In certain cases, the assay may be a vascularization assay.

In some embodiments, the subject method is an in vitro method that includes contacting a sample with a subject compound that specifically binds with high affinity to a target molecule. In certain embodiments, the sample is suspected of containing the target molecule and the subject method further comprises evaluating whether the compound specifically binds to the target molecule. In certain embodiments, the target molecule is a naturally occurring L-protein and the compound is D-peptidic.

In certain embodiments, the subject compound is a modified compound that includes a label, e.g., a fluorescent label, and the subject method further includes detecting the label, if present, in the sample, e.g., using optical detection.

In certain embodiments, the compound is modified with a support, such that any sample that does not bind to the compound may be removed (e.g., by washing). The specifically bound target protein, if present, may then be detected using any convenient means, such as, using the binding of a labeled target specific probe or using a fluorescent protein reactive reagent.

In another embodiment of the subject method, the sample is known to contain the target protein. In certain embodiments, the target protein is a synthetic D-protein and the compound is L-peptidic.

In some embodiments, the subject method includes administering to a subject a compound that specifically binds with high affinity to a target molecule. In certain embodiments, the subject is human. In certain embodiments, the compound is administered as a pharmaceutical preparation. In certain embodiments, the compound is D-peptidic.

In certain embodiments, the compound is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p 977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabelled and a secondary labeling agent is used for imaging. In certain embodiments, the subject methods include diagnosis of a disease condition in a subject by comparing the number, size, and/or intensity of labeled loci, to corresponding baseline values. The base line values can represent the mean levels in a population of undiseased subjects, or previous levels determined in the same subject.

The subject methods may be diagnostic methods for detecting the expression of a target protein in specific cells, tissues, or serum, in vitro or in vivo. In some cases, the subject method is a method for in vivo imaging of a target protein in a subject. The methods may include administering the compound to a subject presenting with symptoms of a disease condition related to a target protein. In some cases, the subject is asymptomatic. The subject methods may further include monitoring disease progression and/or response to treatment in subjects who have been previously diagnosed with the disease.

In certain embodiments, the subject methods include administering a compound, such as a VEGF-binding compound, and then detecting the compound after it has bound to its target protein, e.g., VEGF. In some methods, the same compound can serve as both a therapeutic and a diagnostic compound.

### Target Molecules

Once the subject compounds are prepared they can be used in subject methods for binding to one or more target molecules (e.g., a protein, nucleic acid, organic small molecule, etc.).

In some embodiments, the subject compounds are selected to bind to a disease-specific target protein. Disease-specific proteins are proteins that are expressed exclusively, or at a significantly higher level, in one or several diseased cells or tissues compared to other non-diseased cells or tissues in an animal. Examples of such diseases include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathycandidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and GerstmannStraussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss. Exemplary disease or conditions include, e.g., MS, SLE, ITP, IDDM, MG, CLL, CD, RA, Factor VIII Hemophilia, transplantation, arteriosclerosis, Sjogren's Syndrome, Kawasaki Disease, anti-phospholipid Ab, AHA, ulcerative colitis, multiple myeloma, Glomerulonephritis, seasonal allergies, and IgA Nephropathy.

In some embodiments, the subject compounds find use in the treatment of disease or conditions including: age-related macular degeneration (AMD), diabetic macular edema (DME), graft survival of transplanted corneas, osteoarthritis pain, chronic lower back pain, cancer-related pain, angiogenesis and metastasis in a variety of cancers, lymphangioleiomyomatosis (LAM), and ideopathic pulmonary fibrosis (IPF).

The target molecules may be any type of protein-binding or antigenic molecule, such as proteins, nucleic acids, carbohydrates or small molecules. In certain embodiments, the target molecule is a therapeutic target molecule or a diagnostic target molecule. In certain embodiments, the target molecule is a hormone, a growth factor, a receptor, an enzyme, a cytokine, an osteoinductive factor, a colony stimulating factor or an immunoglobulin.

In certain embodiments, the target molecule may be one or more of the following: growth hormone, bovine growth hormone, insulin like growth factors, human growth hormone including n-methionyl human growth hormone, parathyroid hormone, thyroxine, insulin, proinsulin, amylin, relaxin, prorelaxin, glycoprotein hormones such as follicle stimulating hormone (FSH), leutinizing hormone (LH), hemapoietic growth factor, Her-2, fibroblast growth factor, prolactin, placental lactogen, tumor necrosis factors, mullerian inhibiting substance, mouse gonadotropin-associated polypeptide, inhibin, activin, vascular endothelial growth factors, integrin, nerve growth factors such as NGF-beta, insulin-like growth factor-I and II, erythropoietin, osteoinductive factors, interferons, colony stimulating factors, interleukins (e.g., an IL-4 or an IL-8 protein), bone morphogenetic proteins, LIF, SCF, FLT-3 ligand, kit-ligand, SH3 domain, apoptosis protein, hepatocyte growth factor, hepatocyte growth factor receptor, neutravidin, maltose binding protein, angiostatin, aFGF, bFGF, TGF-alpha, TGF-beta, HGF, TNF-alpha, angiogenin, IL-8, thrombospondin, the 16-kilodalton N-terminal fragment of prolactin and endostatin.

In certain embodiments, the target molecule may be a therapeutic target protein for which structural information is known, such as, but not limited to: Raf kinase (a target for the treatment of melanoma), Rho kinase (a target in the prevention of pathogenesis of cardiovascular disease), nuclear factor kappaB (NF-.kappa.B, a target for the treatment of multiple myeloma), vascular endothelial growth factor (VEGF) receptor kinase (a target for action of anti-angiogenetic drugs), Janus kinase 3 (JAK-3, a target for the treatment of rheumatoid arthritis), cyclin dependent kinase (CDK) 2 (CDK2, a target for prevention of stroke), FMS-like tyrosine kinase (FLT) 3 (FLT-3; a target for the treatment of acute myelogenous leukemia (AML)), epidermal growth factor receptor (EGFR) kinase (a target for the treatment of cancer), protein kinase A (PKA, a therapeutic target in the prevention of cardiovascular disease), p21-activated kinase (a target for the treatment of breast cancer), mitogen-activated protein kinase (MAPK, a target for the treatment of cancer and arthritis), c-Jun NH.sub.2-terminal kinase (JNK, a target for treatment of diabetes), AMP-activated kinase (AMPK, a target for prevention and treatment of insulin resistance), lck kinase (a target for immuno-suppression), phosphodiesterase PDE4 (a target in treatment of inflammatory diseases such as rheumatoid arthritis and asthma), Abl kinase (a target in treatment of chronic myeloid leukemia (CML)), phosphodiesterase PDE5 (a target in treatment of erectile dysfunction), a disintegrin and metalloproteinase 33 (ADAM33, a target for the treatment of asthma), human immunodeficiency virus (HIV)-1 protease and HIV integrase (targets for the treatment of HIV infection), respiratory syncytial virus (RSV) integrase (a target for the treatment of infection with RSV), X-linked inhibitor of apoptosis (XIAP, a target for the treatment of neurodegenerative disease and ischemic injury), thrombin (a therapeutic target in the treatment and prevention of thromboembolic disorders), tissue type plasminogen activator (a target in prevention of neuronal death after injury of central nervous system), matrix metalloproteinases (targets of anti-cancer agents preventing angiogenesis), beta secretase (a target for the treatment of Alzheimer's disease), src kinase (a target for the treatment of cancer), fyn kinase, lyn kinase, zeta-chain associated protein 70 (ZAP-70) protein tyrosine kinase, extracellular signal-regulated kinase 1 (ERK-1), p38 MAPK, CDK4, CDK5, glycogen synthase kinase 3 (GSK-3), KIT tyrosine kinase, FLT-1, FLT-4, kinase insert domain-containing receptor (KDR) kinase, and cancer osaka thyroid (COT) kinase.

In certain embodiments, the target molecule is a target protein that is selected from the group consisting of a VEGF protein, a RANKL protein, a NGF protein, a TNF-alpha protein, a SH2 domain containing protein, a SH3 domain containing protein, an IgE protein, a BLyS protein (Oren et al., “Structural basis of BLyS receptor recognition”, Nature Structural Biology 9, 288-292, 2002), a PCSK9 protein (Ni et al., “A proprotein convertase subtilisin-like/kexin type 9 (PCSK9) C-terminal domain antibody antigen-binding fragment inhibits PCSK9 internalization and restores low density lipoprotein uptake”, J. Biol. Chem. 2010 Apr. 23; 285(17):12882-91), a DLL4 protein (Garber, “Targeting Vessel Abnormalization in Cancer”, JNCI Journal of the National Cancer Institute 2007 99(17):1284-1285), an Ang2 (Angiopoietin-2) protein, a Clostridium difficile Toxin A or B protein (e.g., Ho et al., “Crystal structure of receptor-binding C-terminal repeats from Clostridium difficile toxin A”, (2005) Proc. Natl. Acad. Sci. Usa 102: 18373-18378), a CTLA4 protein (Cytotoxic T-Lymphocyte Antigen 4), and fragments thereof. In certain embodiments, the target protein is a VEGF protein. In certain embodiments, the target protein is a SH2 domain containing protein (e.g., a 3BP2 protein), a SH3 domain containing protein (e.g., a ABL or a Src protein), or a cytokine (e.g., an IL-13 protein, a TNF-alpha protein, a BAFF protein, or a RANKL protein).

In some embodiments, the subject compounds are selected to bind to a tissue-specific target protein. Tissue-specific proteins are proteins that are expressed exclusively, or at a significantly higher level, in one or several particular tissue(s) compared to other tissues in an animal. Exemplary tissues where target proteins may be specifically expressed include, e.g., liver, pancreas, adrenal gland, thyroid, salivary gland, pituitary gland, brain, spinal cord, lung, heart, breast, skeletal muscle, bone marrow, thymus, spleen, lymph node, colorectal, stomach, ovarian, small intestine, uterus, placenta, prostate, testis, colon, colon, gastric, bladder, trachea, kidney, or adipose tissue.

In some embodiments, a subject compound that binds to the target protein is linked to the pharmaceutical protein or small molecule such that the resulting complex or fusion is targeted to the specific tissue or disease-related cell(s) where the target protein is expressed. Subject compounds for use in such complexes or fusions can be initially selected for binding to the target protein and may be subsequently selected by negative selection against other cells or tissue (e.g., to avoid targeting bone marrow or other tissues that set the lower limit of drug toxicity) where it is desired that binding be reduced or eliminated in other non-target cells or tissues.

Exemplary tissue-specific or disease-specific proteins can be found in, e.g., Tables I and II of U.S. Patent Publication No 2002/0107215.

### VEGF

In certain embodiments, the target protein is a VEGF protein. The VEGF-binding compounds of the invention are therapeutically useful for treating any disease or condition which is improved, ameliorated, inhibited or prevented by removal, inhibition, or reduction of a VEGF protein. In certain embodiments, the VEGF protein is VEGF-A, VEGF-B, VEGF-C, VEGF-D, VEGF-E, or a fragment thereof.

The VEGF-binding subject compounds are useful in the treatment of various neoplastic and non-neoplastic diseases and disorders. Neoplasms and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Non-neoplastic conditions that are amenable to treatment include rheumatoid arthritis, psoriasis, atherosclerosis, diabetic and other proliferative retinopathies including retinopathy of prematurity, retrolental fibroplasia, neovascular glaucoma, age-related macular degeneration, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the subject VEGF-binding compounds find use in reducing the severity of AMD.

Lymphangioleiomyomatosis is a rare, progressive, frequently fatal cystic lung disease that affects women almost exclusively. It occurs in up to 40% of women with the tuberous sclerosis complex, a tumor-suppressor syndrome associated with seizures, cognitive impairment, and hamartomas in multiple organs, and can also occur in a nonheritable sporadic form that involves only the lung, lymphatics, and kidney. VEGF is a major angiogenic growth factor produced by malignant cells. VEGF-D, a ligand for the lymphatic growth-factor receptor VEGFR-3/Flt-4, induces formation of lymphatics and promotes the spread of tumor cells to lymph nodes. In some cases, levels of VEGF-D, but not VEGF-A or VEGF-C, are elevated in patients with sporadic lymphangioleiomyomatosis as compared with healthy controls. Serum VEGF-D may be a clinically useful diagnostic test that can distinguish sporadic lymphangioleiomyomatosis from other cystic and chylous lung diseases, potentially decreasing the need for lung biopsy. See e.g., Young et al. “Diagnostic Potential of Serum VEGF-D for Lymphangioleiomyomatosis,” N. Engl. J. Med. 358, 2, 2008, 199-200.

In addition, LAM cells proliferate along lymphatic channels in the lung and in extrapulmonary sites, including the mediastinal, retroperitoneal, and pelvic lymphatics. In these locations, LAM cells are divided into fascicles or bundles by channels lined by lymphatic endothelial cells. LAM cells produce VEGF-C, and the degree to which it is produced by LAM cells correlated, in some cases, with the degree of lymphangiogenesis observed. The lymphatic channels recruited by LAM cells tend to divide the cells into clusters that are then shed from the lesion. This mechanism may account for the ability of LAM cells to metastasize to distant sites and facilitate the progressive invasion of the lung parenchyma by LAM cells. See e.g., Juvet et al., “Molecular Pathogenesis of Lymphangioleiomyomatosis,” Am. J. Respir. Cell Mol. Biol., Vol 36. pp 398-408, 2007. Thus, modulation of VEGF-C and VEGF-D is of interest in the treatment of LAM.

In certain embodiments, a subject compound may be contacted with a cell in the presence of VEGF, and a VEGF response phenotype of the cell monitored.

Exemplary VEGF assays include assays using isolated protein in a cell free systems, in vitro using cultured cells or in vivo assays. Exemplary VEGF assays include, but are not limited to a receptor tyrosine kinase inhibition assay (see, e.g., Cancer Research Jun. 15, 2006; 66:6025-6032), an in vitro HUVEC proliferation assay (FASEB Journal 2006; 20: 2027-2035), an in vivo solid tumor disease assay (U.S. Pat. No. 6,811,779) and an in vivo angiogenesis assay (FASEB Journal 2006; 20: 2027-2035). The descriptions of these assays are hereby incorporated by reference.

In some embodiments, the subject method is a method of modulating angiogenesis in a subject, the method comprising administering to the subject an effective amount of a subject compound that specifically binds with high affinity to a VEGF protein. In certain embodiments, the method further comprises diagnosing the presence of a disease condition in the subject. In certain embodiments, the disease condition is a condition that may be treated by enhancing angiogenesis. In certain embodiments, the disease condition is a condition that may be treated by decreasing angiogenesis. In certain embodiments, the subject method is a method of inhibiting angiogenesis, the target protein is a VEGF protein and the compound is a VEGF antagonist.

In some embodiments, the subject method is a method of treating a subject suffering from a cellular proliferative disease condition, the method including administering to the subject an effective amount of a subject compound that specifically binds with high affinity to a VEGF protein so that the subject is treated for the cellular proliferative disease condition.

In some embodiments, the subject method is a method of inhibiting tumor growth in a subject, the method comprising administering to a subject an effective amount of a subject compound that specifically binds with high affinity to the VEGF protein. In certain embodiments, the tumor is a solid tumor. In certain embodiments, the tumor is a non-solid tumor.

The subject VEGF-binding compounds may be used as affinity purification agents. In this process, the compounds are immobilized on a solid phase such a Sephadex resin or filter paper, using any convenient methods. The subject VEGF-binding compound is contacted with a sample containing the VEGF protein (or fragment thereof) to be purified, and thereafter the support is washed with a suitable solvent that will remove substantially all the material in the sample except the VEGF protein, which is bound to the immobilized compound. Finally, the support is washed with another suitable solvent, such as glycine buffer, pH 5.0, that will release the VEGF protein from the immobilized compound.

The subject VEGF-binding compounds may also be useful in diagnostic assays for VEGF protein, e.g., detecting its expression in specific cells, tissues, or serum. Such diagnostic methods may be useful in cancer diagnosis. For diagnostic applications, the subject compound may be modified as described above.

### SH2 Domain and SH3 Domain Containing Proteins

In certain embodiments, the target protein is a SH2 domain and SH3 domain containing protein. The subject compounds of the invention are therapeutically useful for treating any disease or condition which is improved, ameliorated, inhibited or prevented by modulation of the activity of a SH2 domain or SH3 domain containing protein.

### 3BP2

3BP2 (or SH3BP2 for Abl-SH3 Binding Protein-2) is an adapter protein composed of an amino-terminal pleckstrin homology (PH) domain, a central proline-rich (PR) region, and a carboxyl-terminal SH2 domain that was originally identified as a c-Abl SH3 binding protein in 1993. Functional studies have implicated a role for 3BP2 in immunoreceptor signaling through its interaction with a number of signaling molecules, including Src and Syk families of protein tyrosine kinases, LAT, Vav, PLCγ, and 14-3-3. Recently, the 3 bp2/sh3 bp2 locus was shown to be mutated in a rare human disease involved in cranial-facial development called cherubism, suggesting a role for 3BP2 in regulating osteoclast function. For a review of 3BP2, see, e.g., Deckert and Rottapel, “The Adapter 3BP2: How It Plugs into Leukocyte Signaling” in Lymphocyte Signal Transduction, Advances in Experimental Medicine and Biology, Ed. Tsoukas, C., 2006, 584, 107-114.

The Src homology 2 (SH2) domain is a sequence-specific phosphotyrosine-binding module present in many signaling molecules. In cytoplasmic tyrosine kinases, the SH2 domain is located N-terminally to the catalytic kinase domain (SH1) where it mediates cellular localization, substrate recruitment, and regulation of kinase activity. Initially, structural studies established a role of the SH2 domain stabilizing the inactive state of Src family members. However, biochemical characterization showed that the presence of the SH2 domain is frequently required for catalytic activity, suggesting a crucial function stabilizing the active state of many nonreceptor tyrosine kinases. Recently, the structure of the SH2-kinase domain of Fes revealed that the SH2 domain stabilizes the active kinase conformation by direct interactions with the regulatory helix αC. Stabilizing interactions between the SH2 and the kinase domains have also been observed in the structures of active Csk and Abl. Mutations in the SH2 domain found in human disease can be explained by SH2 domain destabilization or incorrect positioning of the SH2.

In some embodiments, the subject compounds find use in treating a SH2 domain-related disorder. In certain embodiments, the SH2 domain-related disorder is selected from lung cancer, X-linked agammaglobulinemia (XLA), therapy-resistant Epstein—Barr virus (EBV)-positive B cell proliferation. For a review of SH2 domains, see e.g., Filippakopoulos et al. “SH2 domains: modulators of nonreceptor tyrosine kinase activity,” Current Opinion in Structural Biology, 2009, 19: 643-649.

In some embodiments, the subject method is a method of treating an SH2 domain-mediated disorder that includes administering a therapeutically effective amount of a subject compound that specifically binds to the SH2 domain.

### ABL and Src

c-Src and Bcr-Abl are two non-receptor or cytoplasmic tyrosine kinases (TKs) that play important roles in the development of solid and hematological malignancies. Indeed, Src is overexpressed or hyperactivated in a variety of solid tumors, while Bcr-Abl is the causative agent of chronic myeloid leukemia (CML), where Src is also involved. The two enzymes share significant sequence homology and remarkable structural resemblance, including a SH3 domain.

Src is a non-receptor or cytoplasmic tyrosine kinase (TK), belonging to a family with nine currently identified members. Src affects cell proliferation, differentiation, migration, invasion, apoptosis and angiogenesis, by interaction with a diverse array of molecules, including growth factor receptors, cell-cell adhesion receptors, integrins and steroid hormone receptors. In normal cells it remains in an inactive state, being only transiently activated during the multiple cellular events in which it is involved. By contrast, Src is overexpressed and/or hyperactivated in a large variety of cancer cells and is probably a strong promoting factor for the development of metastatic cancer phenotypes. Moreover, Src plays an important role in osteoclast activation and bone resorption, which are often aberrantly activated in the setting of bone metastases. Given the role of Src in all these functions, its inhibition would be of interest for the treatment of patients with Src-dependent cancers.

Bcr-Abl, encoded by the aberrant gene Bcr-Abl on the chromosome Philadelphia, is another cytoplasmic TK and represents the causative agent of chronic myeloid leukemia (CML). Bcr-Abl, differently from its cellular counterpart, c-Abl, is constitutively activated and promotes multiple transduction cascades, leading to growth, proliferation and survival of hematopoietic cells; it also plays a role in defective DNA repair, alteration of cellular adhesion and inhibition of apoptosis. Since it has been demonstrated that the prototypic non-receptor TK c-Abl, normally present in cells, is not a vital enzyme, selective inhibition of all Abl activity is a potential treatment modality for CML. The Bcr-Abl inhibitor imatinib is (since 2001) an important drug for CML treatment, but relapse after an initial response has been observed in some patients, especially in the advanced phases of the disease.

For a review of Src/Abl see e.g., Schenone et al. “Novel dual Src/Abl inhibitors for hematologic and solid malignancies,” Expert Opin. Investig. Drugs (2010) 19(8):931-945.

In some embodiments, the subject compounds find use in treating a Src-mediated disorder. In certain embodiments, the Src-mediated disorder is CML or a lymphoma, e.g., diffuse large B-cell lymphoma (DLBCL). In certain embodiments, the Src-mediated disorder is selected from breast, prostate, lung, pancreatic, and head and neck, CNS (including glioblastoma), ovarian cancers, mesothelioma, sarcomas (including chondrosarcoma), neuroblastoma, and unspecified adult and childhood solid tumors.

In some embodiments, the subject method is a method of treating a Src-mediated disorder that includes administering a therapeutically effective amount of a subject compound that specifically binds to Src.

In certain embodiments, the subject method is a method of treating a Src- or Abl-mediated disorder that includes administering in combination therapeutically effective amounts of a subject compound that specifically binds to a Src protein and a subject compound that specifically binds to a Abl protein.

In some embodiments, the subject method is a method of treating a disease condition mediated by a SH3 domain containing protein. In some embodiments, the subject method is a method of treating an Abl-mediated disorder that includes administering a therapeutically effective amount of a subject compound that specifically binds to Src. In some embodiments, the Abl-mediated disorder is CML.

### TNF-α

The subject compounds may find use as agonists or antagonists to modulate the activity of tumor necrosis factor-α (TNF-α or TNF-alpha). TNF-α is a pleiotropic cytokine that is primarily produced by activated macrophages and lymphocytes; but is also expressed in endothelial cells and other cell types. TNF-alpha is a major mediator of inflammatory, immunological, and pathophysiological reactions. (Grell, M., et al., (1995) Cell, 83:793-802). Two distinct forms of TNF include a 26 kDa membrane expressed form and a soluble 17 kDa cytokine which is derived from proteolytic cleavage of the 26 kDa form. In some cases, the soluble biologically active TNF polypeptide is 157 amino acids long.

TNF-alpha exerts its biological effects through interaction with high-affinity cell surface receptors. Two distinct membrane TNF-alpha receptors that have been cloned and characterized include a 55 kDa species, designated p55 TNF-R and a 75 kDa species designated p75 TNF-R (Corcoran. A. E., et al., (1994) Eur. J. Biochem., 223:831-840). These two TNF receptors exhibit 28% similarity at the amino acid level. This is confined to the extracellular domain and consists of four repeating cysteine-rich motifs, each of approximately 40 amino acids. Each motif contains four to six cysteines in conserved positions. Dayhoff analysis shows the greatest intersubunit similarity among the first three repeats in each receptor. This characteristic structure is shared with a number of other receptors and cell surface molecules, which comprise the TNF-R/nerve growth factor receptor superfamily (Corcoran. A. E., et al., (1994) Eur. J. Biochem., 223:831-840).

In certain embodiments, the subject compounds specifically bind TNF-α protein. Exemplary TNF-α assays include in vitro assays using cell free systems or using cultured cells or in vivo assays. As such, TNF-α assays include in vitro human whole blood assay and cell mediated cytotoxicity assay (U.S. Pat. No. 6,090,382), in vitro tumor human killing assay (see, e.g., published U.S. patent application 20040185047), in vivo tumor regression assay (USP Application 20040002589). Additional TNF-α assays are described in a variety of publications, including 20040151722, 20050037008, 20040185047, 20040138427, 20030187231, 20030199679, and Balazovich (Blood 1996 88: 690-696).

The ability of the subject compounds to modulate the binding of a TNF-alpha protein to a TNF receptor protein such as p55 and p75 may be determined by any convenient method. Suitable assays include, but are not limited to, e.g., quantitative comparisons comparing kinetic and equilibrium binding constants. The kinetic association rate (Kon) and dissociation rate (Koff), and the equilibrium binding constants (Kd) may be determined using surface plasmon resonance on a BIAcore instrument following the standard procedure in the literature [Pearce et al., Biochemistry 38:81-89 (1999)].

In some embodiments, the subject methods are methods of treating a TNF-alpha mediated disease comprising the administration of the TNF-alpha binding molecule of the present invention to a subject (e.g., a human). In certain embodiments, the administration is under conditions such that the symptoms of the TNF-alpha mediated disease are reduced or eliminated. In some embodiments, the TNF-alpha mediated disease is selected from sepsis, an autoimmune disease, rheumatoid arthritis, allergy, multiple sclerosis, autoimmune uveitis, nephrotic syndrome, an infectious disease, a malignancy, transplant rejection, graft-versus-host disease, systemic lupus erythematosus, thyroidosis, scleroderma, diabetes mellitus, Graves' disease, a pulmonary disorder, a bone disorder, an intestinal disorder, a cardiac disorder, cachexia, circulatory collapse, shock resulting from acute or chronic bacterial infections, acute and chronic parasitic and/or infectious diseases, chronic inflammatory pathologies, vascular inflammatory pathologies, sarcoidosis, inflammatory bowel disease, ulcerative colitis, Crohn's disease, disseminated intravascular coagulation, atherosclerosis, Kawasaki's pathology, neurodegenerative diseases, demyelinating diseases, multiple sclerosis, acute transverse myelitis, extrapyramidal and cerebellar disorders, lesions of the corticospinal system, disorders of the basal ganglia, hyperkinetic movement disorders, Huntington's chorea, senile chorea, drug-induced movement disorders (e.g., those induced by drugs which block CNS dopamine receptors), hypokinetic movement disorders, Parkinson's disease, progressive supranuclear palsy, spinocerebellar disorders, structural lesions of the cerebellum, spinocerebellar degenerations, spinal ataxia, Friedreich's ataxia, cerebellar cortical degenerations, multiple systems degenerations, Shy-Drager syndrome, Mencel, Dejerine-Sottas, and Machado-Joseph diseases, systemic disorders, Refsum's disease, abetalipoprotemia, ataxia, telangiectasia, mitochondrial multi-system disorder, disorders of the motor unit, neurogenic muscular atrophies, anterior horn cell degeneration, amyotrophic lateral sclerosis, infantile spinal muscular atrophy, juvenile spinal muscular atrophy, Alzheimer's disease, Down's Syndrome, Diffuse Lewy body disease, senile dementia of Lewy body type, Wernicke-Korsakoff syndrome, chronic alcoholism, Creutzfeldt-Jakob disease, subacute sclerosing panencephalitis, Hallerrorden-Spatz disease, dementia pugilistica, malignant pathologies involving TNF-.alpha.-secreting tumors or other malignancies involving TNF, such as, leukemias (acute, chronic myelocytic, chronic lymphocytic and/or myelodyspastic syndrome), lymphomas (e.g., Hodgkin's, non-Hodgkin's and Burkitt's lymphomas), mycosis fungoides, alcohol-induced hepatitis, psoriasis, psoriatic arthritis, Wegener's granulomatosis, ankylosing spondylitis, heart failure, reperfusion injury, chronic obstructive pulmonary disease, pulmonary fibrosis, and hepatitis C infection. In preferred embodiments, the TNF-.alpha. mediated disease is selected from the group consisting of juvenile and adult rheumatoid arthritis, Crohn's disease, psoriasis, ulcerative colitis, psoriatic arhritis, ankylosing spondylitis, and other spondyloarthropathies, Wegener's granulomatosis, idiopathic pulmonary fibrosis, chronic obstructive pulmonary disease, asthma, oncology, and graft-versus-host disease.

### NGF

Nerve growth factor (NGF) was the first neurotrophin to be identified, and its role in the development and survival of both peripheral and central neurons has been well characterized. NGF has been shown to be a critical survival and maintenance factor in the development of peripheral sympathetic and embryonic sensory neurons and of basal forebrain cholinergic neurons (Smeyne et al., Nature 368:246-249 (1994); Crowley et al., Cell 76:1001-1011 (1994)). NGF upregulates expression of neuropeptides in sensory neurons (Lindsay and Harmer, Nature 337:362-364 (1989)) and its activity is mediated through two different membrane-bound receptors. The TrkA tyrosine kinase receptor mediates high affinity binding and the p75 receptor, which is structurally related to other members of the tumor necrosis factor receptor family, mediates low affinity binding (Chao et al., Science 232:518-521 (1988)).

In addition to its effects in the nervous system, NGF has been increasingly implicated in processes outside of the nervous system. For example, NGF has been shown to enhance vascular permeability (Otten et al., Eur. J. Pharmacol. 106:199-201 (1984)), enhance T- and B-cell immune responses (Otten et al., Proc. Natl. Acad. Sci. U.S.A. 86:10059-10063 (1989)), induce lymphocyte differentiation and mast cell proliferation and cause the release of soluble biological signals from mast cells (Matsuda et al., Proc. Natl. Acad. Sci. U.S.A. 85:6508-6512 (1988); Pearce et al., J. Physiol. 372:379-393 (1986); Bischoff et al., Blood 79:2662-2669 (1992); Horigoma et al., J. Biol. Chem. 268:14881-14887 (1993)).

NGF is produced by a number of cell types including mast cells (Leon et al., Proc. Natl. Acad. Sci. U.S.A. 91:3739-3743 (1994)), B-lymphocytes (Torcia et al., Cell 85:345-356 (1996), keratinocytes (Di Marco et al., J. Biol. Chem. 268:22838-22846)) and smooth muscle cells (Ueyama et al., J. Hypertens. 11:1061-1065 (1993)). NGF receptors have been found on a variety of cell types outside of the nervous system. For example, TrkA has been found on human monocytes, T- and B-lymphocytes and mast cells.

Consistent with a non-neuronal role for NGF, an association between increased NGF levels and a variety of inflammatory conditions has been observed in human patients as well as in several animal models. These include systemic lupus erythematosus (Bracci-Laudiero et al., Neuroreport 4:563-565 (1993)), multiple sclerosis (Bracci-Laudiero et al., Neurosci. Lett. 147:9-12 (1992)), psoriasis (Raychaudhuri et al., Acta Derm. Venereol. 78:84-86 (1998)), arthritis (Falcini et al., Ann. Rheum. Dis. 55:745-748 (1996)) and asthma (Braun et al., Eur. J. Immunol. 28:3240-3251 (1998)).

NGF plays a role in regulating innervation in the skin and also is known to upregulate neuropeptides, suggesting that increased NGF levels may be responsible for the upregulation of neuropeptides and the increased cutaneous innervation seen with psoriasis. In fact, increased expression of NGF has been observed in psoriatic keratinocytes (Raychaudhuri et al., Acta Derm. Venercol. 78:84-86 (1998)). It has been suggested that while NGF normally serves as a survival factor for keratinocytes, overexpression of NGF prevents normal cell death, leading to psoriasis (Pincelli et al., J. Derm. Sci. 22:71-79 (2000)).

NGF has been shown to affect mast cell degranulation (Bruni et al., FEBS Lett. 138:190-193 (1982)) and substance P release (Donnerer et al., Neurosci. 49:693-698 (1992)), implicating it in the pathogenesis of arthritis.

An elevated level of NGF in peripheral tissues is associated with both hyperalgesia and inflammation and has been observed in a number of forms of arthritis. The synovium of patients affected by rheumatoid arthritis expresses high levels of NGF while in non-inflamed synovium NGF has been reported to be undetectable (Aloe et al., Arch. Rheum. 35:351-355 (1992)). Similar results were seen in rats with experimentally induced rheumatoid arthritis (Aloe et al., Clin. Exp. Rheumatol. 10:203-204 (1992)). Elevated levels of NGF have bean reported in transgenic arthritic mice along with an increase in the number of mast cells. (Aloe et al., Int. J. Tissue Reactions-Exp. Clin. Aspects 15:139-143 (1993)). However, purified NGF injected into the joint synovium of normal rats does not induce knee joint inflammation, suggesting that NGF does not play a causative role in arthritis (Aloe et al., Growth Factors 9:149-155 (1993)).

High NGF levels have been associated with allergic inflammation and it has been suggested that this is related to mast cell degranulation (Bonini et al., Proc. Natl. Acad. Sci. U.S.A. 93:10955-10960 (1996)).

Elevated NGF levels are also observed in both allergic and non-allergic asthma (Bonini et al., supra). Mast cells, eosinophils and T-lymphocytes have all been proposed to play a role in this inflammatory disease and the correlation between NGF serum levels and total IgE antibody titers suggests that NGF contributes to the inflammatory immune response. Allergen induced airway inflammation has been associated with increased local production of NGF in both mice and humans (Braun et al., Int. Arch. Allergy Immunol. 118:163-165 (1999)).

NGF has been shown to regulate the development of increased airway hyperactive response, a hallmark of bronchial asthma (Braun et al., Eur. J. Immunol. 28:3240-3251 (1998)). Indeed, in one study, treatment of allergen-sensitized mice with anti-NGF antibody prevented the development of airway hyperresponsiveness following local allergen challenge (Braun et al., Int. Arch. Allergy Immunol. 118:163-165 (1999)).

In some embodiments, the subject compounds find use in modulating the activity of NGF. Thus, for example, the subject compounds can be identified by incubating the compound with NGF and monitoring binding and modulation of a biological activity of NGF. The binding assay may be performed with purified NGF polypeptide(s), or with cells naturally expressing, or transfected to express, NGF polypeptide(s). In certain embodiments, the binding assay is a competitive binding assay, where the ability of a subject compound to compete with a known anti-NGF antibody for NGF binding is evaluated. The assay may be performed in various formats, including the ELISA format.

The ability of a subject compound to modulate a biological activity of NGF can, for example, be carried out by monitoring the ability of the subject compound to inhibit NGF mediated survival in the embryonic rat dorsal root ganglia survival bioassay as described in Hongo et al. (Hybridoma 19:215-227 (2000)). A kinase-induced receptor activation (KIRA) assay may be used to measure the effect of the subject compounds on the NGF-dependent TrkA autophosphorylation in transfected cells in response to stimulation with a ligand, such as hNGF (Sadick et al., Exp. Cell Res. 234:354-361 (1997)).

In some embodiments, the subject compounds finds use in inhibiting the binding of hNGF to human TrkA (hTrkA) in vivo.

In some embodiments, the subject compounds find use in treating a NGF-related disorder. The inflammatory condition may be selected from the group consisting of asthma, arthritis, multiple sclerosis, lupus erythematosus and psoriasis. The NGF binding compounds may be used to prevent the onset of the active disease state, to treat symptoms that are currently being experienced and to treat the underlying disease itself.

In some embodiments, the subject compounds find use in treating other diseases that may be associated with increased levels of NGF including, for example, lupus erythematosus, shingles, postherpetic neuralgia, hyperalgesia, and chronic pain.

### IgE

One of the pathways by which inflammation is induced is through the immunoglobulin E (IgE). IgE plays a central role in allergies by virtue of their role as allergen receptors on the surface of mast cells and basophils. IgE antibodies are fixed to the surface of mast cells and basophils at the Fc portion of the molecule to a high affinity cell surface receptor, called Fc.epsilon.RI. The allergic reaction is initiated when the polyvalent allergen molecule binds to antibodies that are occupying these receptors. The result is a bridging of the Fc.epsilon.RI, which in turn signals intracellularly causing the release and activation of mediators of inflammation: histamine, leukotrienes, chemotactic factors, platelet-activating factor, and proteinases. These activated mediators act locally and cause increased vascular permeability, vasodilation, smooth muscle contraction and mucous gland secretion. Such events are termed clinically the immediate or early phase, and occur within the first 15-30 minutes following allergen exposure. Over the succeeding 12 hours there is progressive tissue infiltration of inflammatory cells, proceeding from neutrophils to eosinophils to mononuclear cells in response to other chemical mediators not quite fully understood. This period of time 6-12 hours after allergen exposure is designated the late phase and is characterized by clinical manifestations of cellular inflammation. Given that late phase reactions, especially in the lung, occur in the absence of early phase reactions, it is still not entirely understood if the late phase reaction is necessarily IgE mediated.

IgE exists in a membrane bound form and in a secreted form. These distinct forms appear to be splice variants. Previous approaches to achieve therapeutic effect by down regulating IgE targeting primarily the secreted form (e.g., XOLAIR® omalizumab), so as prevent or disarm further “arming” of the immune system. The secreted form of IgE is a shorter form, essentially the Fc region ends at the CH4 domain (FIG. 1), whereas the longer form includes additional C-terminal residues including the peptides encoded by the exons known as M1/M1′ and M2. While some have reported two distinct forms of membrane bound IgE, both with and without a 52 amino acid segment known as M1′ [Batista et al., J. Exp. Med. 184: 2197-2205 (1996)], Applicants were unable to verify that any membrane bound form lacks this M1′ segment. Conventional therapy with anti-IgE antibodies, which bind to the secreted form of IgE, results in reduction of free serum, but not total serum IgE. Casale et al., J. Allergy Clin. Immunol 100 (1): 110-121 (1997).

Targeting the M1′ segment of IgE with anti-IgE antibodies can result in inducing apoptosis of the B-cell. As the progeny of activated B-cells can result in plasma cells that make and secrete the secreted form of IgE, the depletion of the IgE-producing B-cell through apoptosis offers one therapeutic approach to the treatment of allergy.

In some embodiments, the subject compounds find use in treating a IgE-mediated disorder. In certain embodiments, the IgE-mediated disorder is selected from allergic rhinitis, asthma (e.g., allergic asthma and non-allergic asthma), atopic dermatitis, allergic gastroenteropathy, hypersensitivity (e.g., anaphylaxis, urticaria, food allergies etc.), allergic bronchopulmonary aspergillosis, parasitic diseases, interstitial cystitis, hyper-IgE syndrome, ataxia-telangiectasia, Wiskott-Aldrich syndrome, athymic lymphoplasia, IgE myeloma and graft-versus-host reaction. In certain embodiments, the IgE-mediated disorder is food allergy, anaphylaxis, contact dermatitis or allergic purpura.

In some embodiments, the subject method is a method of treating an IgE-mediated disorder including administering a therapeutically effective amount of a subject compound that specifically binds to the IgE and induces apoptosis in IgE-expressing B-cells. In certain embodiments, the subject compound specifically binds to the M1′ segment of IgE.

### RANKL

RANKL (receptor activator of NF-kB ligand, also referred to as OPGL, TRANCE or ODF) is a member of the tumour necrosis factor (TNF) family of cytokines that binds to its receptor RANK to control osteoclast differentiation, activation and survival. Osteoprotegerin (OPG) is a soluble decoy receptor for RANKL that blocks ligand binding to RANK, thereby preventing the signalling required for osteoclast differentiation and activation. RANK is also constitutively expressed in normal mammary gland epithelial cells, but RANKL expression is induced by sex hormones during pregnancy. Genetically, both RANKL and RANK are essential for the development of the lactating mammary gland during pregnancy and for lymph node organogenesis in mouse embryos.

Bone metastases are a frequent complication of many cancers that result in severe disease burden and pain. The microenvironment of the local host tissue actively participates in the propensity of certain cancers to metastasize to specific organs. In the case of breast cancers, the local chemokine milieu is implicated in the metastasis of these tumours to certain organs. However, as the inhibition of chemokine receptors in vivo only partially blocks metastatic behaviour, other factors exist that regulate the preferential metastasis of breast cancer cells. The cytokine RANKL (receptor activator of NF-kB ligand) triggers migration of human epithelial cancer cells and melanoma cells that express the receptor RANK. RANK is expressed on cancer cell lines and breast cancer cells in patients. Local differentiation factors such as RANKL have an important role in cell migration and the tissue-specific metastatic behaviour of cancer cells. See e.g., Jones et al., “Regulation of cancer cell migration and bone metastasis by RANKL”, Nature, 440, 2006, 692-6. Thus, inhibition of RANKL-RANK interactions is of interest in the treatment of tumour metastasis and progression in bones.

Osteoporosis is a systemic skeletal disorder characterized by low bone mass, microarchitectural deterioration of bone tissue, and compromised bone strength resulting in an increased risk of bone fractures. Osteoporosis can be further characterized as either primary or secondary. Primary osteoporosis can occur in both genders at all ages, but often follows menopause in women and occurs later in life in men. In contrast, secondary osteoporosis is a result of medications, other conditions, risk factors, or diseases. Examples include, but are not limited to, glucocorticoid-induced osteoporosis, hypogonadism, cancers, other endocrine disorders, celiac disease, genetic disorders, inflammatory diseases, malnutritive and/or malabsorption syndromes. An imbalance in bone formation relative to bone resorption results in the net bone loss that occurs in osteoporosis and inflammatory bone diseases. Treatments that normalize the balance of bone turnover by inhibiting bone resorption preserve bone mass and reduce fracture risk. RANKL is a pivotal regulator of osteoclast activity is a target for treatment of osteoporosis, e.g., denosumab, a highly specific anti-RANKL antibody, rapidly and substantially reduces bone resorption.

In some embodiments, the subject compounds find use in treating a RANKL-mediated disorder. In some embodiments, the subject method is a method of treating an RANKL-mediated disorder that includes administering a therapeutically effective amount of a subject compound that specifically binds to a RANKL protein.

### Utility

The compounds of the invention, e.g., as described above, find use in a variety of applications. Applications of interest include, but are not limited to: therapeutic applications, research applications, and screening applications. Each of these different applications are now reviewed in greater details below.

### Therapeutic Applications

The subject compounds find use in a variety of therapeutic applications. Therapeutic applications of interest include those applications in which the activity of the target is the cause or a compounding factor in disease progression. As such, the subject compounds find use in the treatment of a variety of different conditions in which the modulation of target activity in the host is desired.

The subject compounds are useful for treating a disorder relating to its target.

Examples of disease conditions which may be treated with compounds of the invention are described above.

In certain embodiments, the disease conditions include, but are not limited to: cancer, inhibition of angiogenesis and metastasis, osteoarthritis pain, chronic lower back pain, cancer-related pain, age-related macular degeneration (AMD), diabetic macular edema (DME), LAM: lymphangioleiomyomatosis, IPF: Ideopathic pulmonary fibrosis and graft survival of transplanted corneas.

In one embodiment, the invention provides a method of treating a subject for a VEGF-related condition. The method generally involves administering a subject compound to a subject having a VEGF-related disorder in an amount effective to treat at least one symptom of the VEGF-related disorder. VEGF-related conditions are generally characterized by excessive vascular endothelial cell proliferation, vascular permeability, edema or inflammation such as brain edema associated with injury, stroke or tumor; edema associated with inflammatory disorders such as psoriasis or arthritis, including rheumatoid arthritis; asthma; generalized edema associated with burns; ascites and pleural effusion associated with tumors, inflammation or trauma; chronic airway inflammation; capillary leak syndrome; sepsis; kidney disease associated with increased leakage of protein; and eye disorders such as age related macular degeneration and diabetic retinopathy. Such conditions include breast, lung, colorectal and renal cancer.

### Combination Therapies

In some embodiments, the subject compounds may be administered in combination with one or more additional compounds or therapies, including a second target-binding molecule, a chemotherapeutic agent, surgery, catheter devices, and radiation. Combination therapy includes administration of a single pharmaceutical dosage formulation which contains the subject compound and one or more additional agents; as well as administration of the subject compound and one or more additional agent(s) in its own separate pharmaceutical dosage formulation. For example, a subject compound and a cytotoxic agent, a chemotherapeutic agent or a growth inhibitory agent can be administered to the patient together in a single dosage composition such as a combined formulation, or each agent can be administered in a separate dosage formulation. Where separate dosage formulations are used, the subject compound and one or more additional agents can be administered concurrently, or at separately staggered times, e.g., sequentially.

### Research Applications

The subject compounds and methods find use in a variety of research applications. The subject compounds and methods may be used to analyze the roles of target proteins in modulating various biological processes, including but not limited to, angiogenesis, inflammation, cellular growth, metabolism, regulation of transcription and regulation of phosphorylation. Other target protein binding molecules such as antibodies have been similarly useful in similar areas of biological research. See e.g., Sidhu and Fellhouse, “Synthetic therapeutic antibodies,” Nature Chemical Biology, 2006, 2(12), 682-688. Such methods can be readily modified for use in a variety of research applications of the subject compounds and methods.

### Diagnostic Applications

The subject compounds and methods find use in a variety of diagnostic applications, including but not limited to, the development of clinical diagnostics, e.g., in vitro diagnostics or in vivo tumor imaging agents. Such applications are useful in diagnosing or confirming diagnosis of a disease condition, or susceptibility thereto. The methods are also useful for monitoring disease progression and/or response to treatment in patients who have been previously diagnosed with the disease.

Diagnostic applications of interest include diagnosis of disease conditions, such as those conditions described above, including but not limited to: cancer, inhibition of angiogenesis and metastasis, osteoarthritis pain, chronic lower back pain, cancer-related pain, age-related macular degeneration (AMD), diabetic macular edema (DME), LAM: lymphangioleiomyomatosis, IPF: Ideopathic pulmonary fibrosis and graft survival of transplanted corneas. In some cases, the disease condition is a target-mediated condition where the target is selected from the group consisting of a VEGF protein, a RANKL protein, a NGF protein, a TNF-alpha protein, a SH2 domain containing protein, a SH3 domain containing protein, a Src protein, an IL-4 protein, an IL-8 protein, an IgE protein and fragments thereof. In some methods, the same compound can serve as both a treatment and diagnostic reagent.

Other target protein binding molecules, such as aptamers and antibodies, have also found use in the development of clinical diagnostics. Such methods can be readily modified for use in a variety of diagnostics applications of the subject compounds and methods, see for example, Jayasena, “Aptamers: An Emerging Class of Molecules That Rival Antibodies in Diagnostics,” Clinical Chemistry. 1999; 45: 1628-1650.

### Pharmaceutical Preparations

Also provided are pharmaceutical preparations. Pharmaceutical preparations are compositions that include a compound (either alone or in the presence of one or more additional active agents) present in a pharmaceutically acceptable vehicle. The term “pharmaceutically acceptable” means approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the invention and pharmaceutically acceptable vehicles, excipients, or diluents may be sterile. In some instances, an aqueous medium is employed as a vehicle when the compound of the invention is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular compound, as well as by the particular method used to administer the composition. Accordingly, there is a wide variety of suitable formulations of the pharmaceutical composition of the present invention.

Administration of compounds of the invention may be systemic or local. In certain embodiments administration to a mammal will result in systemic release of a compound of the invention (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the compounds and compositions of the invention are administered orally. In certain embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

A compound of the invention may also be formulated for oral administration. For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers. A compound of the invention may also be incorporated into existing nutraceutical formulations, such as are available conventionally, which may also include an herbal extract.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Dose levels can vary as a function of the specific compound, the nature of the delivery vehicle, and the like. Desired dosages for a given compound are readily determinable by a variety of means.

The dose administered to an animal, particularly a human, in the context of the present invention should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the compounds may be administered in the form of a free base, their pharmaceutically acceptable salts, or they may also be used alone or in appropriate association, as well as in combination, with other pharmaceutically active compounds.

In some embodiments, a pharmaceutical composition includes a subject compound that specifically binds with high affinity to a target protein, and a pharmaceutically acceptable vehicle. In certain embodiments, the target protein is a VEGF protein and the subject compound is a VEGF antagonist.

### Systems and Kits

Also provided are kits that include compounds of the invention. Kits of the invention may include one or more dosages of the compound, and optionally one or more dosages of one or more additional active agents. Conveniently, the formulations may be provided in a unit dosage format. In such kits, in addition to the containers containing the formulation(s), e.g. unit doses, is an informational package insert describing the use of the subject formulations in the methods of the invention, e.g., instructions for using the subject unit doses to treat cellular proliferative disease conditions.

These instructions may be present in the subject systems and kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

In some embodiments, a kit includes a first dosage of a subject pharmaceutical composition and a second dosage of a subject pharmaceutical composition. In certain embodiments, the kit further includes a second angiogenesis modulatory agent.

The following examples are offered by way of illustration and not by way of limitation.

### Experimental

### 1.1 Synthesis and Folding of D-VEGF Target Protein

D-VEGF was synthesized utilizing standard solid phase peptide synthesis and native chemical ligation methods (see e.g., review by Ackrill et al., Biopolymers (Pept Sci) 94: 495-503, 2010; “Total chemical synthesis of proteins,” Stephen B. H. Kent, Chem. Soc. Reviews, 38, 338-51 (2009)). The following fragments of D-VEGF were prepared (may also be referred to as D-VEGF(8-109)):

(SEQ ID NO: 259)
1. GQNHHEVVKFMDVYQRSY-SR (1-18);
(SEQ ID NO: 260)
2. Thz-CHPIETLVDIFQEYPDEIEYIFKPSCVPLMR-SR (19-49)
(SEQ ID NO: 261)
3. CGGCCNDEGLECVPTEESNITMQIMRIKPHQGQHIGEMSFLQHNKCECRPKKD (50-102),

where SR refers to a C-terminal thioester and Thz is a protected form of Cys (Thz=1,3-thiazolidine-4-carboxo-).

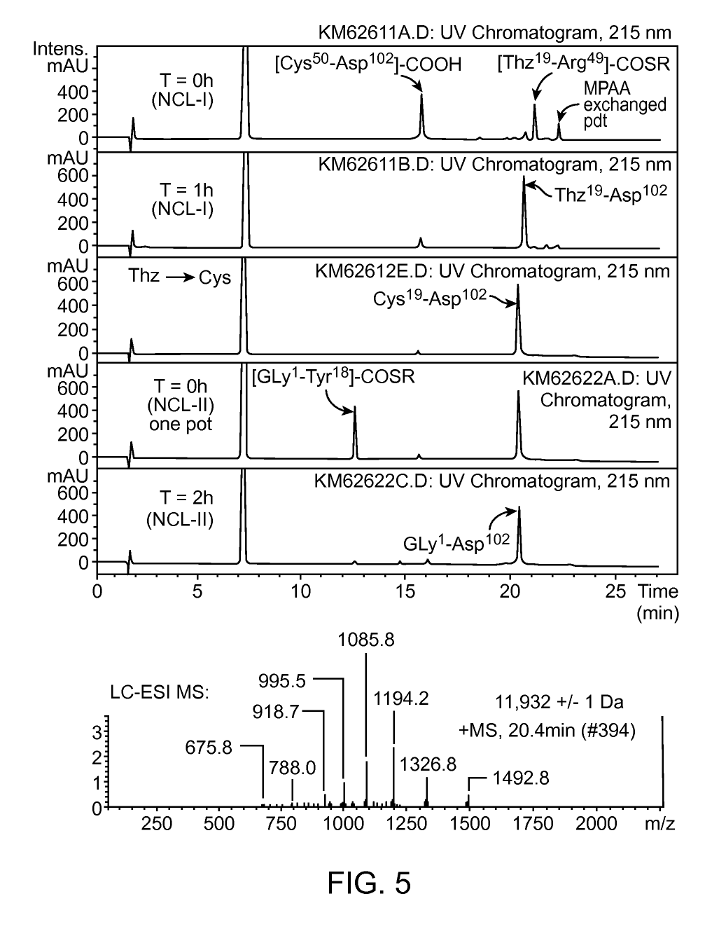

Native chemical ligation methods were used to assemble the fragments as follows: fragments 2 and 3 were ligated to generate fragment (19-102) (NCL-I); the N-terminal cysteine of fragment (19-102) was deprotected (i.e. Thz- was converted to Cys-); and fragment 1 and fragment (Cys19-102) (NCL-II) were ligated to generate the 102 amino acid residue polypeptide D-VEGF. FIG. 5 shows RP-HPLC chromatograms (top) that illustrate each step in the synthesis of the polypeptide D-VEGF (8-109). At bottom of FIG. 5 is shown ESI MS data for the product (11,932 +/−1 Da).

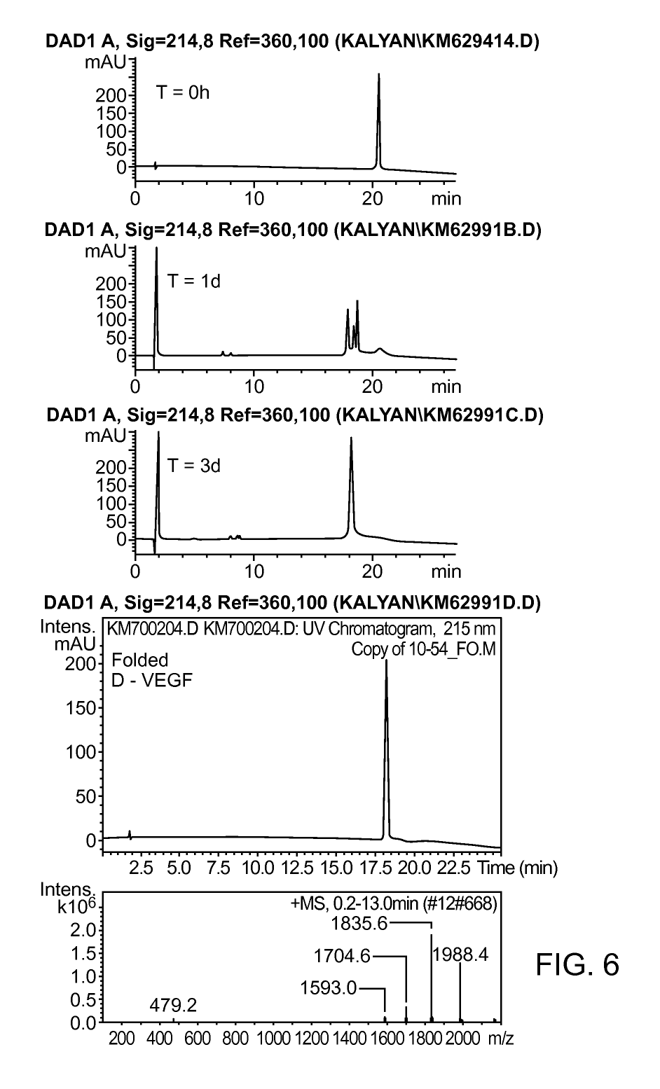

The synthetic D-VEGF was folded using similar conditions to those described below for L-VEGF by incubating the protein in an aqueous buffer for 3 days. FIG. 6 (top) illustrates LC chromatograms following the folding of the protein in solution after 0 hours, 1 day and 3 days. FIG. 6 (bottom) shows the LC chromatogram and direct infusion ESI MS data for purified, folded D-VEGF (observed mass: 23,849.2±0.5 Da, calculated mass: 23,849.1 Da (average isotopes)).

L-VEGF was prepared by the same method as described above. Synthetic L-VEGF-A (8-109) was folded under the following conditions: [VEGF]=0.5 mg/mL, [Glutathione]red=1.95 M, [Glutathione]ox=0.39 M, Tris=0.1 M, Gu.HCl=0.15 M, pH=8.4 incubation for 5 days. The folded L-VEGF was purified and analyzed by direct infusion ESI MS (observed mass: 23,849.3±0.5 Da, calculated mass: 23,849.1 Da (average isotopes)). L-VEGF was tested in a functional cell proliferation assay using human umbilical vein endothelial cells (HUVEC) and gave an ED₅₀ value of 4.6 ng/mL (0.2 nM).

### 1.2 Preparation of GB1 Peptidic Compounds

1.2.1 Mutagenesis of GB1 Point Mutants.

The following oligonucleotides were used to introduce a N-terminal 10×His tag and a stop codon at the C-terminus of GB1 mutants.

N-terminal His-Tag
(SEQ ID NO: 262)
GGACCATAGATTATGAAAATCGAAGAACACCACCATCATCACCACCATCATCACCACGGCGGTAGCACGTAC
(SEQ ID NO: 263)
M K I E E H H H H H H H H H H G G S T Y
C-terminal stop
(SEQ ID NO: 264)
GGCGGTTCTGACAAATAAGCGGCCGGCCCAGTG
(SEQ ID NO: 265)
G G S D K *

The N-terminal insertion deletes the StII secretion signal and introduces a KIEE (SEQ ID NO:266) leader sequence for intracellular expression. The C-terminal stop codon was inserted after the linker GGSDK (SEQ ID NO:267), but before the dimerization sequence. This was chosen such that this oligonucleotide can also be used with all GB1 variants including those with mutations close to C-terminus. Therefore there is a GGSDK (SEQ ID NO:267) linker at the C-terminus.

The pre-affinity matured parent clone E01 was mutated using Kunkel mutagenesis to insert the His-tag and stop codons. This sequence was used as a template for generating point mutants using the following oligonucleotides (Integrated DNA Technologies, Inc.) by Kunkel mutagenesis and confirmed by DNA sequencing.

```
E01-V22L
(SEQ ID NO: 268)
ACCGAAGCAGTGGATCTGTATGATGCATTTGAT
(SEQ ID NO: 269)
  T E A V D L Y D A F D
E01-Y23F
(SEQ ID NO: 270)
GAAGCAGTGGATGTTTTTGATGCATTTGATGTT
(SEQ ID NO: 271)
  E A V D V F D A F D V
E01-D24G
(SEQ ID NO: 272)
GCAGTGGATGTTTATGGTGCATTTGATGTTTTC
(SEQ ID NO: 273)
  A V D V Y G A F D V F
E01-D27E
(SEQ ID NO: 274)
GTTTATGATGCATTTGAAGTTTTCTTTGTTTAC
(SEQ ID NO: 275)
  V Y D A F E V F F V Y
E01-V31A
(SEQ ID NO: 276)
TTTGATGTTTTCTTTGCTTACGCCGCTTCTAAT
(SEQ ID NO: 277)
  F D V F F A Y A A S N
E01-A34S
(SEQ ID NO: 278)
TTCTTTGTTTACGCCTCTTCTAATTTTTCTGAT
(SEQ ID NO: 279)
  F F V Y A S S N F S D
E01-S35R
(SEQ ID NO: 280)
TTTGTTTACGCCGCTCGTAATTTTTCTGATTTT
(SEQ ID NO: 281)
  F V Y A A R N F S D F
E01-S35E
(SEQ ID NO: 282)
TTTGTTTACGCCGCTGAAAATTTTTCTGATTTT
(SEQ ID NO: 283)
  F V Y A A E N F S D F
E01-S35G
(SEQ ID NO: 284)
TTTGTTTACGCCGCTGGGAATTTTTCTGATTTT
(SEQ ID NO: 285)
  F V Y A A G N F S D F
E01-S38K
(SEQ ID NO: 286)
GCCGCTTCTAATTTTAAAGATTTTGATGATTGG
(SEQ ID NO: 287)
  A A S N F K D F D D W
E01-S38G
(SEQ ID NO: 288)
GCCGCTTCTAATTTTGGGGATTTTGATGATTGG
(SEQ ID NO: 289)
  A A S N F G D F D D W
E01-D39F
(SEQ ID NO: 290)
GCTTCTAATTTTTCTTTCTTTGATGATTGGACC
(SEQ ID NO: 291)
  A S N F S F F D D W T
E01-F40I
(SEQ ID NO: 292)
TCTAATTTTTCTGATATTGATGATTGGACCTAC
(SEQ ID NO: 293)
  S N F S D I D D W T Y
E01-D41V
(SEQ ID NO: 294)
AATTTTTCTGATTTTGTTGATTGGACCTACGAT
(SEQ ID NO: 295)
  N F S D F V D W T Y D
```

1.2.2 of GB1-E01 Double Mutants

GB1 compound E01-D39F was selected as a scaffold for construction of further point mutants, for affinity optimization. Compounds E01-D39F/V22L, E01-D39F/Y23F, E01-D39F/D24G, E01-D39F/D27E, and E01-D39F/V31A were prepared using the oligonucleotides and mutagenesis method described above. In addition, the following nucleotides were used to prepare GB1 compounds:

```
E01-D39F/A34S
(SEQ ID NO: 477)
TTCTTTGTTTACGCCTCTTCTAATTTTTCTTTC
(SEQ ID NO: 478)
  F F V Y A S S N F S F
E01-D39F/S35R
(SEQ ID NO: 479)
TTTGTTTACGCCGCTCGTAATTTTTCTTTCTTT
(SEQ ID NO: 480)
  F V Y A A R N F S F F
E01-D39F/S35E
(SEQ ID NO: 481)
TTTGTTTACGCCGCTGAAAATTTTTCTTTCTTT
(SEQ ID NO: 482)
  F V Y A A E N F S F F
E01-D39F/S35G
(SEQ ID NO: 483)
TTTGTTTACGCCGCTGGGAATTTTTCTTTCTTT
(SEQ ID NO: 484)
  F V Y A A G N F S F F
E01-D39F/S38K
(SEQ ID NO: 485)
GCCGCTTCTAATTTTAAATTCTTTGATGATTGG
(SEQ ID NO: 486)
  A A S N F K F F D D W
E01-D39F/S38G
(SEQ ID NO: 487)
GCCGCTTCTAATTTTGGGTTCTTTGATGATTGG
(SEQ ID NO: 488)
  A A S N F G F F D D W
E01-D39F/F401
(SEQ ID NO: 489)
TCTAATTTTTCTTTCATTGATGATTGGACCTAC
(SEQ ID NO: 490)
  S N F S F I D D W T Y
E01-D39F/D41V
(SEQ ID NO: 491)
AATTTTTCTTTCTTTGTTGATTGGACCTACGAT
(SEQ ID NO: 492)
  N F S F F V D W T Y D
E01-D39F/D41G
(SEQ ID NO: 493)
AATTTTTCTTTCTTTGGTGATTGGACCTACGAT
(SEQ ID NO: 494)
  N F S F F G D W T Y D
```

GB1 compounds based on the above sequences were cloned, expressed and assayed for binding affinity. The results are shown in Tables 2 and 3.

### 1.3 Expression and Purification of GB1 Compounds in E. Coli

The plasmids were chemically transformed into XL1Blue cells. Single colonies were grown in 2YT/carb media overnight in an incubator at 37° C. shaking at 200 rpm. This culture was used to inoculate 50 ml 2YT/carb at 1:200 dilution and grown to OD₆₀₀=0.8 at 37° C. with shaking at 200 rpm. The culture was cooled to 24° C. and 1 mM IPTG was added. The cells were then incubated at 24° C. with shaking at 200 rpm for 4 hrs 30 min.

The cells were pelleted, by centrifuging at 26000 g for 10 min, and the pellets were resuspended in 15 ml of Lysis Buffer (50 mM Sodium Phosphate, 40 mM Imidazole, 300 mM Sodium Chloride pH 7.5) supplemented with 1 mM PMSF. The cells were then sonicated using a SONICS vibra-cell sonicator (SONICS, Newton, Conn.) at 40% amplitude for 3 min (5s pulses with 5s breaks) on ice. This solution was centrifuged at 26000 g for 35 min and the supernatant was harvested to get the clarified lysate.

This clarified lysate was purified on gravity column using Ni-NTA SuperFlow (QIAGEN) resin. 250 μl of packed resin was equilibrated with 20 ml of Lysis buffer. The clarified lysate was then applied to the column. The column was then washed with 20 ml of Wash Buffer (50 mM Sodium Phosphate, 60 mM Imidazole, 300 mM Sodium Chloride pH 7.5). The column was washed again with 750 μl Wash Buffer 2 (50 mM Sodium Phosphate, 100 mM Imidazole, 300 mM Sodium Chloride pH 8.0). The column was then eluted with Elution Buffer (50 mM Sodium Phosphate, 400 mM Imidazole, 300 mM Sodium Chloride pH 8.0) in 250 μl fractions an stored at 4° C.

The purified proteins were run on a 4-15% Mini-Protein TGX precast SDS-PAGE gel (BioRad) to check purity. While there were few impurities the proteins are nearly ˜90-95% pure. The concentrations of the proteins were estimated using Bradford Assay using BSA as a standard. The results of SPR analysis of binding for selected clones is shown in Tables 1 and 2.

### 1.4 Synthesis of GB1 Peptidic Compounds

GB1 peptidic compounds are synthesized utilizing standard solid phase peptide synthesis methods similar to those described for the synthesis of VEGF target protein above. For example, the L-enantiomer of the Cys-containing GB1 peptidic compound, RFX002, was prepared from two peptide fragments and assembled using native chemical ligation methods. The following fragments of RFX002 were prepared:

(SEQ ID NO: 495)
1. TYKMILNGKTLKSECTTEAVDLFDAFDVLFV-COSR;
and
(SEQ ID NO: 496)
2. CAASNFSFIDDWTYDDATKTFTVTE.

where -COSR refers to a C-terminal thioester. After native chemical ligation of the two fragments the full length polypeptide was purified by RP-HPLC and subsequently folded with concomitant formation of disulfide bond under air oxidation conditions using ammonium bicarbonate buffer at pH 8.5. The D-enantiomer of RFX002 linear polypeptide was prepared using stepwise solid phase peptide synthesis. The folding of the D-RFX002 was performed following the same air oxidation condition used for the L-RFX002 mentioned above.

The following compounds are prepared using a similar method similar to that described above where the ligation site is shown in bold:

RFX.T1.4.83.29.49 RFX027.d
(SEQ ID NO: 497)
tykmilnGkt lksectteav dwfdafdvlf vcaasqfsvi ddwtyddatk tftvte
RFX.T1.4.83.29.50 RFX028.d
(SEQ ID NO: 498)
tykmilnGkt lksectteav dwfdafdvlf acaasnfpli edwtyddatk tftvte
RFX.T1.4.83.29.51 RFX029.d
(SEQ ID NO: 499)
tykmilnGkt lksectteav dwfdafdvlf acaasnfsli edwtyddatk tftvte
RFX.T1.4.83.29.26 RFX030.d
(SEQ ID NO: 500)
tykmilnGkt lksectteav dffaafevlf Gcadshfsii ddwtyddatk tftvte

### 1.5 X-Ray Crystallography

Synthetic D-VEGF-A was crystallized, and X-ray diffraction data was acquired to a resolution of 1.86 Å. The structure was solved by molecular replacement, using inverted coordinates of the previously reported VEGF-A structure (3QTK) as a search model. Within experimental uncertainty, chemically synthesized D-VEGF-A had a structure that was the mirror image of VEGF-A.

Protein crystals form more readily from a racemic mixture resulting in the formation of centrosymmetric crystals. Some of the potential advantages of racemic protein crystallography are i) racemic crystals tend to be well ordered; ii) diffract to a higher resolution; and iii) phases are quantized (0 or π), which can facilitate structure solution. Racemic protein crystallography was used to determine the crystal structure of a diastereochiral (meaning interacting proteins of opposite chirality that are not mirror images of one another) VEGF-ligand complex that consists of: {L-VEGF-A+2 copies of a D-protein ligand} plus {D-VEGF-A+2 copies of an L-protein ligand}.

Structure of a 70 kDa Racemic Complex of Six Synthetic Proteins. The racemic VEGF-D-protein ligand complex was crystallized in the space group P21/n and synchrotron X-ray diffraction data was collected to 1.6 Å resolution (see PDB XXXX). The structure was solved in both P21 and P21/n by molecular replacement using identical and inverted coordinates of the previously reported crystal structure of VEGF (PDB 3QTK) and GB1 (PDB 2QMT). The P21 asymmetric unit contains a total of six synthetic protein molecules: two D-_{(EO1AM1)} binder molecules bound to the two opposite poles of one L-VEGF; and, two L-_{(EO1AM1)} binder molecules bound to the two opposite poles of one D-VEGF molecule.

The structure of the VEGF protein molecule in VEGF-binder complex is the same as in previously reported structures. A comparison of the VEGF-binder interface with the VEGF-VEGFR1 binding interface indicates that binding of the D-E01-AM1 blocks binding of VEGF to its receptor as an antagonist. For example, the compound binds to at least part of the receptor binding site of VEGF-A (e.g., that part of the VEGF-A molecule which is responsible for VEGF-A binding to its receptor).

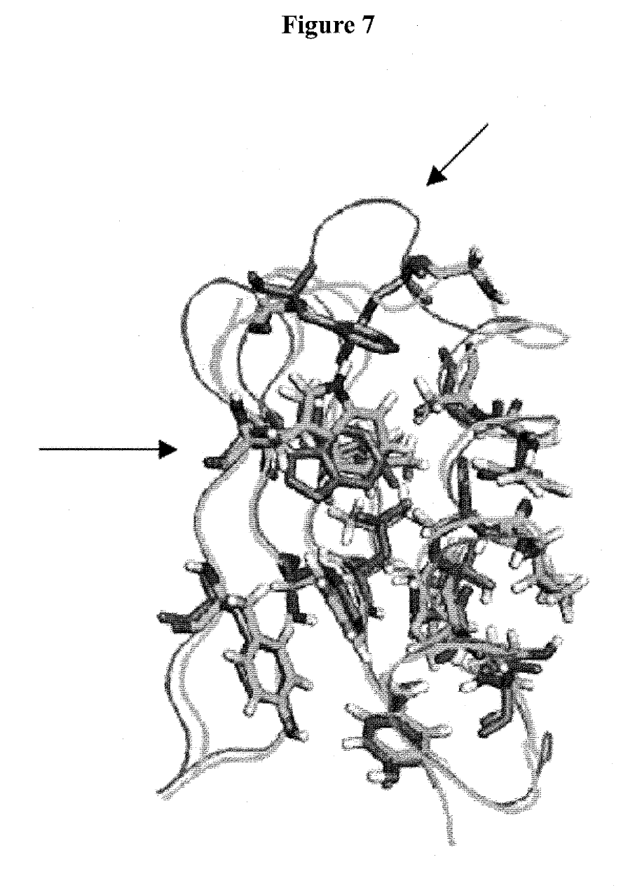

The structure of the E01AM1 binder (FIG. 7, darker strand) in the complex was compared to that of GB1 (FIG. 7, lighter strand). In the structure of E01AM1 residues W42 and F51 are shifted relative to GB1 (see left arrow). In addition, in the E01AM1 binder, there is an insertion in the loop between α1 and β3, which includes one additional D residue at position 41a and is shifted significantly (see top arrow) relative to the GB1 structure. In order to accommodate an additional Asp residue at position 41a, the loop (residues 37 to 41a) between α1 and β3, in the E01AM1 binder, adopts an extended loop conformation compared to the GB1 loop structure (see top arrow) and participates in a number of crystal contacts with the VEGF. Several residues in this loop region (residue 37 to 41a) makes direct polar contact with VEGF at the binding interface. The maximum deviation from GB1 structure is observed at residue Ser38 which makes direct backbone-backbone H-bond contact (3.1 Å) with the Gln 82 of VEGF. Residue 40, in E01AM1, makes two additional direct H-bonds (3.0 Å and 2.9 Å) with residue 82 and 84 of VEGF involving the main chain amide bonds. In addition to the direct H-bonding network, the protein-ligand interaction includes a salt bridge between the side chain of Asp39 of E01AM1 and the side chain of the His83 of VEGF at the binding interface. The additional residue Asp41a is positioned in the loop region and stabilized by a water mediated interaction with the VEGF.

### 2. Activity of GB1 Peptidic Compounds

### 2.1 Direct Binding of Single-Clones by ELISA

The following protocol is an adapted version of previous protocols (Fellouse & Sidhu 2007; Tonikian et al., “Identifying specificity profiles for peptide recognition modules from phage-displayed peptide libraries,” Nat. Protoc., 2007, 2, 1368-86). Although, the protocol is described for L-VEGF, the protocol can be adapted for use with any suitable target.

1. Inoculate 450 μl aliquots of 2YT/carb/KO7 medium in 96-well microtubes with single colonies harboring phagemids and grow for 21 hrs at 37° C. with shaking at 200 rpm.

2. Centrifuge at 4,000 rpm for 10 min and transfer phage supernatants to fresh tubes.

3. Coat 3 wells of a 384 well NUNC maxisorb plate per clone, with 2 μg/ml of L-VEGF, Neutravidn, Erbin-GST respectively and leave one well uncoated. Incubate for 2 hrs at room temperature and block the plates (all 4 well).

4. Wash the plate four times with PT buffer.

5. Transfer 30 μl of phage supernatant to each well and incubate for 2 hrs at room temperature with gentle shaking.

6. Wash four times with PT buffer.

7. Add 30 μl of anti-M13-HRP conjugate (diluted 1:5000 in PBT buffer). Incubate 30 min with gentle shaking.

8. Wash four times with PT buffer 9. Add 30 μl of freshly prepared TMB substrate. Allow color to develop for 5-10 min.

10. Stop the reaction with 100 μl of 1.0 M H₃PO₄ and read absorbance at 450 nm in a microtiter plate reader.

The results of direct binding assays of various clones with L-VEGF and D-VEGF performed using a method as described in above demonstrated specific binding to the target.

### 2.2 VEGF Binding Affinity Using SPR

VEGF binding compounds were expressed. Binding affinities were measured using the Biacore SPR system. SPR analysis was performed on a ProteOn XPR36 Protein Interaction Array System (BioRad). Chemically synthesized L-VEGF, D-VEGF and VEGF165 (Peprotech) were immobilized in 50 mM Sodium Acetate (pH 5.5) to a non-dilute EDAC/sulfo-NHS activated GLC surface on separate channels using a flow rate of 30 ml/min for 5 minutes in the vertical direction. Immobilization levels were monitored to ensure immobilization of approximately 500 response units of each protein. The domains were then stabilized with PBS for 30 seconds and 0.85% H3PO4 for 18 seconds each at 100 ml/min. GB1 variants were diluted in PBS plus 0.05% Tween 20 at a starting concentration of 200 nM. The binders were further diluted with PBST 2-fold in series to produce 5 concentrations of variants. A PBST blank was also included. GB1 variant injection parameters were: 100 ml/min, 60 seconds contact time, and 600 seconds dissociation time, in the horizontal direction. VEGF proteins were regenerated with an injection of 0.85% H3PO4 at a flow rate of 100 mL/min followed by a PBST wash of 30 seconds at 100 mL/min flow rate.

L-E01AM1 produced by recombinant means specifically bound D-VEGF as determined by the Biacore SPR method described above with no significant binding observed to L-VEGF (or L-VEGF165).

TABLE 1
SPR affinity measurements of exemplary GB1 peptidic compounds

| Clone | Target | Kₒₙ (M⁻¹s⁻¹) | K_{off} (s⁻¹) | Affinity (nM) |
|---|---|---|---|---|
| E01 | D-VEGF | 2.60E+05 | 3.47E−03 | 13.4 |
| E01-V22L | D-VEGF | 4.11E+05 | 2.39E−03 | 5.8 |
| E01-Y23F (AM1) | D-VEGF | 3.98E+05 | 2.91E−03 | 7.3 |
| E01-D24G | D-VEGF | 1.66E+05 | 2.47E−03 | 14.9 |
| E01-D27E | D-VEGF | 3.47E+05 | 3.33E−03 | 9.6 |
| E01-V31A | D-VEGF | 2.50E+05 | 1.94E−03 | 7.8 |
| E01-A34S | D-VEGF | 3.56E+05 | 2.78E−03 | 7.8 |
| E01-S35R | D-VEGF | 2.79E+05 | 2.58E−03 | 9.3 |
| E01-S35E | D-VEGF | 4.07E+05 | 3.04E−03 | 7.47 |
| E01-S35G | D-VEGF | 3.96E+05 | 3.48E−03 | 8.79 |
| E01-S38K | D-VEGF | 2.04E+05 | 2.80E−03 | 13.7 |
| E01-S38G | D-VEGF | 4.28E+05 | 3.87E−03 | 9 |
| E01-D39F | D-VEGF | 5.04E+05 | 2.13E−03 | 4.23 |
| E01-F40I | D-VEGF | 3.98E+05 | 4.41E−03 | 11.1 |
| E01-D41V | D-VEGF | 4.93E+05 | 3.33E−02 | 67.5 |

TABLE 2
SPR affinity measurements for GB1-E01 double mutants

| Clone | Target | Kₒₙ (M⁻¹s⁻¹) | K_{off} (s⁻¹) | Affinity (M) |
|---|---|---|---|---|
| E01 | D-VEGF | 3.38E+05 | 1.17E−03 | 3.46E−09 |
| E01-D39F | D-VEGF | 3.80E+05 | 1.32E−03 | 3.48E−09 |
| E01-D39F/V22L | D-VEGF | 7.19E+05 | 8.20E−04 | 1.14E−09 |
| E01-D39F/D24G | D-VEGF | ^{x} | ^{x} | ^{x} |
| E01-D39F/D27E | D-VEGF | 5.11E+05 | 1.58E−03 | 3.10E−09 |
| E01-D39F/V31A | D-VEGF | 5.58E+05 | 1.67E−03 | 2.99E−09 |
| E01-D39F/A34S | D-VEGF | 3.87E+05 | 1.74E−03 | 4.49E−09 |
| E01-D39F/S35R | D-VEGF | 3.41E+05 | 1.83E−03 | 5.35E−09 |
| E01-D39F/S35E | D-VEGF | 4.54E+05 | 2.05E−03 | 4.52E−09 |
| E01-D39F/S35G | D-VEGF | 3.58E+05 | 1.93E−03 | 5.40E−09 |
| E01-D39F/S38K | D-VEGF | ^{x} | ^{x} | ^{x} |
| E01-D39F/S38G | D-VEGF | 4.63E+05 | 1.31E−03 | 2.83E−09 |
| E01-D39F/F40I | D-VEGF | 6.02E+05 | 1.38E−03 | 2.29E−09 |
| E01-D39F/D41V | D-VEGF | ^{x} | ^{x} | ^{x} |

^{x}affinity values not accurately determined

### 2.3 Functional Assay for Blocking VEGF-VEGFR1 Binding

To test whether the D-VEGF and L-VEGF binders have the potential to block VEGF-VEGFR1 interaction, a competition assay was performed using VEGF binding peptides v114 and v128a described by Fairbrother et al. (“Novel peptides selected to bind vascular endothelial growth factor target the receptor-binding site,” Biochemistry, 1998, 37, 17754-64). These peptides block the binding of VEGF to VEGFR1 and inhibit angiogenesis.

The peptides v114 (VEPNCDIHVMWEWECFERL-NH₂; SEQ ID NO:296) and v128a (RGWVEICESDVWGRCL-NH₂; SEQ ID NO:297) were prepared in both D- and L-forms (CSBio, Inc.). The following protocol was used in the competitive binding assays:
1. 5 wells of NUNC 96 well maxisorb plate were coated with D-VEGF (5 μg/ml in PBS) for testing clone E01. Similarly 5 wells were coated with L-VEGF (5 μg/ml in PBS) for each of E01 and B03. The plate was incubated at room temperature with gentle shaking for 2 hours;
2. The plates were blocked with PBS+0.2% BSA (5 wells+1 extra well for control) for 1 hr at room temperature with gentle shaking;
3. In the meantime, peptides were diluted in PBT buffer to a concentration of 150 μM;
4. The plate was washed four times with PT buffer;
5. 66 μl of each diluted peptide was added to 4 different wells coated with L or D-VEGF (depending on the clone tested). 66 μl of PBT was added to control wells (1 VEGF coated well+1 control well). Incubate for 30 min at room temperature with shaking;
6. Add 33 μl of fresh phage supernatant from overnight culture to each of the six wells containing peptide solution. Incubate for 15 min at room temperature with shaking.
7. Wash plate 6 times with PT buffer;
8. Add 66 μl of anti-M13-HRP antibody (1:3000 dilution in PBT) to each well and incubate for 30 min at room temperature with shaking;
9. Wash plate 4 times with PT buffer. Develop and read plates at 450 nm.

Phage binding to L and D-VEGF was tested in the presence and absence of 100 μM of each peptide (e.g., at 100 times affinity to saturate VEGF) using anti-M13-HRP to detect the bound phage. Binding of E01 to D-VEGF is completely blocked by D-v114 and partially blocked by D-v128a. L-peptides do not inhibit E01 binding to D-VEGF and D-peptides do not inhibit A01 and B03 binding to L-VEGF.

### 2.6 Solution Phase Measure of VEGF Antagonism by Octet Assay

Solutions were prepared in diluent containing 1×PBS w/0.1% BSA, 0.5% Proclin. Inhibitor solutions were prepared by incubating 0-2000 nM D-E01 analogue, KM7246-F6, with or without 5 nM hVEGF165 (Peprotech) for 1 hour at ambient temperature. A VEGR1 sensor was prepared by incubating ForteBio Octet anti-hIgGFc sensors for 700 seconds with 25 nM rhVEGFR1/hIgGFc (R & D Systems) followed by a 400 second incubation with 2000 nM human IgG (Jackson Immunoresearch). The VEGFR1 sensors were then incubated with Diluent for 120 seconds to establish a baseline wavelength. Then, VEGFR1 sensors were incubated with the inhibitor solutions for 600 seconds at 30° C. with mixing, and the wavelength shift was measured. Wavelength shifts for VEGF165 binding to the VEGFR1 sensor at each inhibitor concentration were normalized by subtracting the wavelength shift of the inhibitor alone at each respective concentration. Percent inhibition was calculated using the wavelength shift for VEGF165 binding in the absence of inhibitor and the wavelength shift for VEGF165 binding with a non-reactive sensor as 0% and 100% inhibition, respectively. The calculated % inhibition at 600 seconds for each concentration of D-E01 analogue F6 was plotted and the ED50 was calculated using 4-parameter logistics analysis.

D-E01AM1 binds L-VEGF165 with a Kd of 4 nM as determined by the Octet assay described above and no significant binding was observed to D-VEGF.

### 3. Affinity Maturation

3.1 Affinity Maturation of Clones E01, A01 and B03

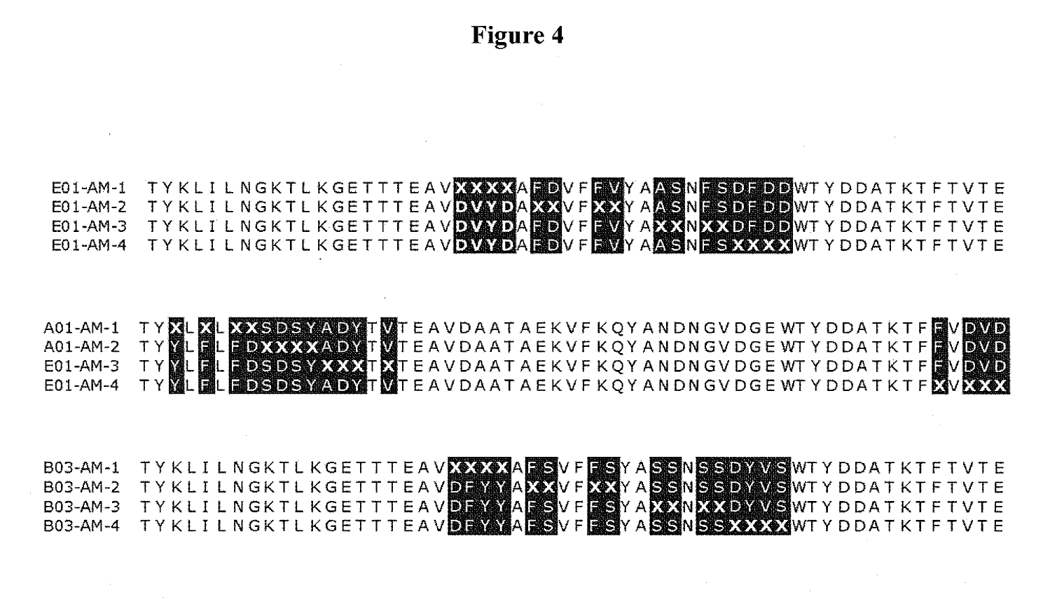

Clones E01, A01 and B03 have 16 variant amino acid residues. Affinity maturation libraries were prepared in order to optimize residues at each variant amino acid position. The following design was utilized. Four libraries were made for each clone, randomizing 4 variant amino acid positions (X) at a time with NNK codons encoding all 20 amino acids at each position as shown in FIG. 4 (E01-AM-1 to 4, A01-AM-1 to 4 and B03-AM-1 to 4).

The following oligonucleotides were prepared to mutate the GB1 template with Fab dimerization sequence:

E01-AM-1
(SEQ ID NO: 298)
5′-ACGACCGAAGCAGTG NNK NNK NNK NNK GCA TTT GAT GTT TTC TTT GTT
TAC GCC GCT TCT AAT TTT TCT GAT TTT GAT GAT TGGACCTACGATGAT-3′;
E01-AM-2
(SEQ ID NO: 299)
5′-ACGACCGAAGCAGTG GAT GTT TAT GAT GCA NNK NNK GTT TTC NNK NNK
TAC GCC GCT TCT AAT TTT TCT GAT TTT GAT GAT TGGACCTACGATGAT-3′;
E01-AM-3
(SEQ ID NO: 300)
5′-ACGACCGAAGCAGTG GAT GTT TAT GAT GCA TTT GAT GTT TTC TTT GTT
TAC GCC NNK NNK AAT NNK NNK GAT TTT GAT GAT TGGACCTACGATGAT-3′;
E01-AM-4
(SEQ ID NO: 301)
5′-ACGACCGAAGCAGTG GAT GTT TAT GAT GCA TTT GAT GTT TTC TTT GTT
TAC GCC GCT TCT AAT TTT TCT NNK NNK NNK NNK TGGACCTACGATGAT-3′;
A01-AM-1
(SEQ ID NO: 302)
5′-GGCGGTAGCACGTAC NNK CTG NNK CTG NNK NNK TCT GAT TCT TAT GCT
GAT TAT ACC GTT ACCGAAGCAGTGGAT-3′;
(SEQ ID NO: 303)
5′-GCGACGAAAACCTTC TTT GTT GAT GTT GAT GGCGGTTCTGACAAA-3′;
A01-AM-2
(SEQ ID NO: 304)
5′-GGCGGTAGCACGTAC TAT CTG TTT CTG TTT GAT NNK NNK NNK NNK GCT
GAT TAT ACC GTT ACCGAAGCAGTGGAT-3′;
(SEQ ID NO: 305)
5′-GCGACGAAAACCTTC TTT GTT GAT GTT GAT GGCGGTTCTGACAAA-3′;
A01-AM-3
(SEQ ID NO: 306)
5′-GGCGGTAGCACGTAC TAT CTG TTT CTG TTT GAT TCT GAT TCT TAT NNK
NNK NNK ACC NNK ACCGAAGCAGTGGAT-3′;
(SEQ ID NO: 307)
5′-GCGACGAAAACCTTC TTT GTT GAT GTT GAT GGCGGTTCTGACAAA-3′;
A01-AM-4
(SEQ ID NO: 308)
5′-GGCGGTAGCACGTAC TAT CTG TTT CTG TTT GAT TCT GAT TCT TAT GCT
GAT TAT ACC GTT ACCGAAGCAGTGGAT-3′
(SEQ ID NO: 309)
5′-GCGACGAAAACCTTC NNK GTT NNK NNK NNK GGCGGTTCTGACAAA-3′;
B03-AM-1
(SEQ ID NO: 310)
5′-ACGACCGAAGCAGTG NNK NNK NNK NNK GCA TTT TCT GTT TTC TTT TCT
TAC GCC TCT TCT AAT TCT TCT GAT TAT GTT TCT TGGACCTACGATGAT-3′;
B03-AM-2
(SEQ ID NO: 311)
5′-ACGACCGAAGCAGTG GAT TTT TAT TAT GCA NNK NNK GTT TTC NNK NNK
TAC GCC TCT TCT AAT TCT TCT GAT TAT GTT TCT TGGACCTACGATGAT-3′;
B03-AM-3
(SEQ ID NO: 312)
5′-ACGACCGAAGCAGTG GAT TTT TAT TAT GCA TTT TCT GTT TTC TTT TCT
TAC GCC NNK NNK AAT NNK NNK GAT TAT GTT TCT TGGACCTACGATGAT-3′;
B03-AM-4
(SEQ ID NO: 313)
5′-ACGACCGAAGCAGTG GAT TTT TAT TAT GCA TTT TCT GTT TTC TTT TCT
TAC GCC TCT TCT AAT TCT TCT NNK NNK NNK NNK TGGACCTACGATGAT-3′.

Phage display libraries were synthesized (see e.g., Fellouse & Sidhu, “Making antibodies in bacteria. Making and using antibodies” Howard & Kaser, Eds., CRC Press, Boca Raton, Fla., 2007) at a 10 μg ssDNA template scale, as the theoretical diversity is small (1.6×10⁵). The library diversity was ˜1×10⁹ transformants for all the libraries, e.g. 10000 times oversampling the sequence space.

The following general selection protocol was performed except that only three rounds of selections were done. The preincubation of library with GST tagged protein was on the plate instead of solution for Round 3.

3.2 Affinity Maturation Selections Against Target Protein and Negative Selection with BSA

The selection procedure is essentially the same as described in previous protocols (Fellouse & Sidhu, 2007) with some minor changes. Although the protocol below is described for L-VEGF, the protocol can be adapted to screen for binding to any target. The media and buffer recipes are the same as in the described protocol.

1. Coat NUNC Maxisorb plate wells with 100 μl of L-VEGF (5 μg/ml in PBS) for 2 h at room temperature. Coat 5 wells for selection and 1 well for phage pool ELISA.
2. Remove the coating solution and block for 1 h with 200 μl of PBS, 0.2% BSA. At the same time, block an uncoated well as a negative control for pool ELISA. Also block 7 wells for pre-incubation of library on a separate plate.
3. Remove the block solution from the pre-incubation plate and wash four times with PT buffer.
4. Add 100 μl of library phage solution (precipitated and resuspended in PBT buffer) to each blocked wells. Incubate at room temperature for 1 h with gentle shaking.
5. Remove the block solution from selection plate and wash four times with PT buffer.
6. Transfer library phage solution from pre-incubation plate to selection plate (5 selection wells+2 controls for pool ELISA)
7. Remove the phage solution and wash 8-10 times with PT buffer (increased based pool ELISA signal from previous round).
8. To elute bound phage from selection wells, add 100 μl of 100 mM HCl. Incubate 5 min at room temperature. Transfer the HCl solution to a 1.5-ml microfuge tube. Adjust to neutral pH with 11 μl of 1.0 M Tris-HCl, pH 11.0.
9. In the meantime add 100 μl of anti-M13 HRP conjugate (1:5000 dilution in PBT buffer) to the control wells and incubate for 30 min.
10. Wash control wells four times with PT buffer. Add 100 μl of freshly prepared TMB substrate. Allow color to develop for 5-10 min.
11. Stop the reaction with 100 μl of 1.0 M H₃PO₄ and read absorbance at 450 nm in a microtiter plate reader. The enrichment ratio can be calculated as the ratio of signal from coated vs uncoated well.
12. Add 250 μl eluted phage solution to 2.5 ml of actively growing E. coli XL1-Blue (OD₆₀₀<0.8) in 2YT/tet medium. Incubate for 20 min at 37° C. with shaking at 200 rpm.
13. Add M13KO7 helper phage to a final concentration of 10¹⁰ phage/ml. Incubate for 45 min at 37° C. with shaking at 200 rpm.
14. Transfer the culture from the antigen-coated wells to 25 volumes of 2YT/carb/kan medium and incubate overnight at 37° C. with shaking at 200 rpm.
15. Isolate phage by precipitation with PEG/NaCl solution, resuspend in 1.0 ml of PBT buffer
16. Repeat the selection cycle for 3 rounds.

3.3. Negative Selection with GST Tagged Protein

A more stringent negative selection procedure is as follows. The selection process is essentially the same as described above except that:

i) For Rounds 1 and 2 the libraries were pre-incubated on GST coated (10 μg/ml in PBS) and blocked wells.
ii) For Rounds 3 and 4, the libraries were pre-incubated with 0.2 mg/ml GST in solution for 1 hr before transfer to selection wells
iii) The control wells for pool ELISA were coated with GST (5 μg/ml in PBS)

### 4. Optimization of GB1 Compounds for Stability

While the following methods are described for an exemplary VEGF-binding GB1 compound, the stabilization methods can be readily adapted to optimize any GB1 compounds for stability.

The VEGF binder E01-AM1 (E01-Y23F) was chemically synthesized in both L- and D-forms. The melting temperature of the synthetic version of E01-AM1 was measured to be 33.4° C.

The X-ray crystal structure of racemic synthetic RFX001 (E01-AM1) bound to racemic VEGF-A(8-109) was solved and revealed some perturbation of the native hydrophobic core structure in RFX001 by comparison to the structure of GB1. A library of GB1 compounds based on E01-D39F/V22L that includes core mutations and fixed region mutations was designed and screened for D-VEGF binding affinity and stability.

The residues of GB1 compound E01-D39F/V22L selected for mutation are shown in bold and underlined:

(SEQ ID NO: 501)
TYKLILNGKTLKGETTTEAVDLFDAFDVFFVYAASNFSFFDDWTYDDAT
KTFTVTE

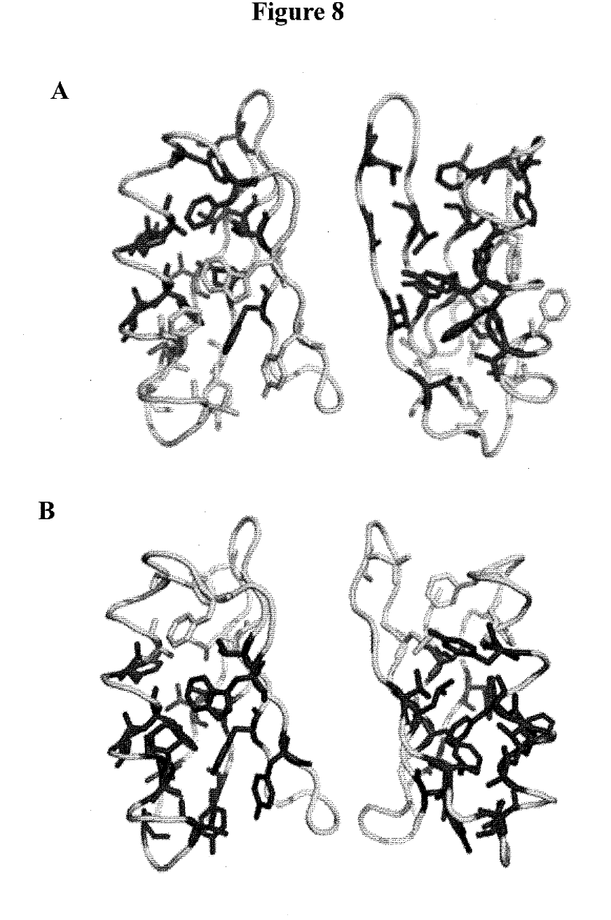

The residues selected were divided between two libraries as shown below, although any convenient combinations of residues may be selected in stability optimization libraries. FIGS. 8A and 8B depict the spatial arrangement of residues mutated in Libraries 1 and 2, respectively.

### Library 1:

(SEQ ID NO: 501)
TYKLILNGKTLKGETTTEAVDLFDAFDVFFVYAASNFSFFDDWTYDDAT
KTFTVTE

### Library 2:

(SEQ ID NO: 501)
TYKLILNGKTLKGETTTEAVDLFDAFDVFFVYAASNFSFFDDWTYDDAT
KTFTVTE

Two randomization strategies were adopted at the mutation positions shown for libraries 1 and 2: 1) a soft randomization strategy (e.g., where there is a 0.5 probability of maintaining the original residue at the mutation position, and a 0.5 probability of incorporating any of the 20 amino acids); and 2) a homolog scan strategy (e.g., where at each mutation position the original residue is replaced with one of the residues as noted in Table 3). Table 3 shows a homolog scan strategy for use in core optimization where the mutations selected at each position are shown, plus the codon used for introducing the mutations into a phage display library for screening. Note that the numbering of the residues in Table 3 is based on the E01-AM1 scaffold sequence, and differs from FIG. 3 with respect to residues W43, Y45, F52 and V54, because E01-AM1 has one additional residue in the α1-β3 region.

TABLE 3
Homolog scan strategy

| Residue | Codon | Amino Acid mutations |
|---|---|---|
| Y2 | TWT | Y/F |
| L4 | KYG | L/V/A/S |
| L6 | KYG | L/V/A/S |
| L11 | KYG | L/V/A/S |
| G13 | GST | G/A |
| T15 | KYG | L/V/A/S |
| T17 | KYG | L/V/A/S |
| A25 | KYG | L/V/A/S |
| V28 | KYG | L/V/A/S |
| F29 | TWT | Y/F |
| Y32 | TWT | Y/F |
| A33 | KYG | L/V/A/S |
| F37 | TWT | Y/F |
| F40 | BTT | F/L/V |
| W43 | TDK | F/Y/W/L/C |
| Y45 | TWT | Y/F |
| F52 | TWT | Y/F |
| V54 | KYG | L/V/A/S |

GB1 compound E01-D39F/V22L was mutated to include Y23F mutation using the following oligonucleotide:

(SEQ ID NO: 502)
ACCGAAGCAGTGGATCTGTTTGATGCATTTGATGTT
(SEQ ID NO: 503)
T E A V D L F D A F D V

This clone was then used as the template for making the core optimization libraries where the following oligonucleotides were used from Kunkel mutagenesis, where N1 denoted a mix of (70% A, 10% C, 10% G, 10% T); N2 denoted a mix of (10% A, 70% C, 10% G, 10% T); N3 denoted a mix of (10% A, 10% C, 70% G, 10% T); and N4 denoted a mix of (10% A, 10% C, 10% G, 70% T):

### Library 1: Soft Randomization:

(SEQ ID NO: 504)
GGTAGCACGTACAAA (N2)(N4)(N3)ATT(N2)(N4)(N3)AAC GGC AAA ACC
(N2)(N4)(N3)AAA(N3)(N3)(N4)GAA(N1)(N2)(N2)ACG(N1)(N2)(N2)GAAGCAGTGGAT
CTGTTTGATGCATTTGAT (N3)(N4)(N4)(N4)(N4)(N2) TTT GTT (N4)(N1)(N2)
(N3)(N2)(N2)GCTTCTAAT(N4)(N4)(N4)TCTTTC(N4)(N4)(N4)GATGATTGGACCTACG
ATGCGACGAAAACC (N4)(N4)(N2) ACG (N3)(N4)(N4) ACCGAAGGCGGTTCT
(SEQ ID NO: 505)
GSTYKLILNGKTLKGETTTEAVDLFDAFDVFFVYAASNFSFFDDWTYDATKTFTVT
EGGS

### Library 2: Soft Randomization:

(SEQ ID NO: 506)
AAAGGCGGTAGCACG(N4)(N1)(N2)AAA(N2)(N4)(N3)ATTCTGAACGGCAAAACCCT
GAAAGGTGAA(N1)(N2)(N2)ACG(N1)(N2)(N2)GAA(N3)(N2)(N1)GTGGAT(N2)(N4)(N3)
TTTGAT(N3)(N2)(N1)(N4)(N4)(N4)GAT(N3)(N4)(N4)(N4)(N4)(N2)TTTGTT(N4)(N1)
(N2)GCCGCTTCTAATTTTTCTTTCTTTGATGAT(N4)(N3)(N3)ACC(N4)(N1)(N2)GATG
ATGCGACGAAAACC(N4)(N4)(N2)ACG(N3)(N4)(N4)ACCGAAGGCGGTTCT
(SEQ ID NO: 507)
KGGSTYKLILNGKTLKGETTTEAVDLFDAFDVFFVYAASNFSFFDDWTYDDATKTF
TVTEGGS

### Library 1: Homolog Scan

```
(SEQ ID NO: 508)
GGTAGCACGTACAAA KYG KYG KYG AAC GGC AAA ACC KYG AAA GST GAA KYG ACG KYG GAAGCAGTGGATCTG
  G S T Y K L I L N G K T L K G E T T T E A V D L
TTTGATGCATTTGAT KYG TWC TTT GTT TWC KYG GCT TCT AAT TWT TCT TTC BTT GATGATTGGACCTAC
  F D A F D V F F V Y A A S N F S F F D D W T Y
GATGCGACGAAAACC TWC ACG KYG ACCGAAGGCGGTTCT
(SEQ ID NO: 509)
  D A T K T F T V T E G G S
```

### Library 2: Homolog Scan

```
(SEQ ID NO: 510)
AAAGGCGGTAGCACG TWC AAA KYG ATTCTGAACGGCAAAACCCTGAAAGGTGAA
(SEQ ID NO: 511)
  K G G S T Y K L I L N G K T L K G E
KYG ACG KYG GAA KYG GTG GAT KYG TTT GAT KYG TWT GAT KYG TWC TTT GTT TWC GCCGCTTCTAATTTT
T T T E A V D L F D A F D V F F V Y A A S N F
TCTTTCTTTGATGAT TDK ACC TWC GAT GAT GCG ACG AAA ACC TWC ACG KYG ACCGAAGGCGGTTCT
  S F F D D W T Y D D A T K T F T V T E G G S
```

The libraries were prepared as described above and the molecular diversity was determined as follows: Library 1 (soft)—5.1×10⁹, Library 1 (homolog)—3.9×10⁹, Library 2 (soft)—8.4×10⁹ and Library 2 (homolog)—8.7×10⁹.

4.1 Screening for Stability

Compounds were screened for specific binding to target protein under conditions that select for stable clones. During screening for stability, selections and washes were performed at 37° C. In another variation, the selections were performed in the presence of various concentrations of guanidine hydrochloride (GuHCl). The phage display screening was performed (see Table 4) using a similar procedure to that described above. The incubation of phage with negative selection plate and D-VEGF coated plate was performed at 37° C. and washes were performed with PT buffer preincubated at 37° C. Where applicable GuHCl was added to phage re-suspended in PBT buffer prior to adding phage to negative selection plates. The GuHCl concentrations were chosen to be below the unfolding transition of BSA (least stable component) that occurs at 1.5M GuHCl.

TABLE 4
Selection conditions

| | Coating | # | # | | |
|---|---|---|---|---|---|
| | Conc. | wells | Washes | Preselection (45 min) | [GuHCl] |
| Round 1 | 2 ug/ml | 10 | 8 | Neutravidin (5 ug/ml) | 0.8M |
| Round 2 | 2 ug/ml | 10 | 8 | Streptavidin (5 ug/ml) | 0.8M |
| Round 3 | 2 ug/ml | 10 | 8 | Neutravidin | 0.9M |
| | | | | (5 ug/ml) + | |
| | | | | Streptavidin (5 ug/ml) | |
| Round 4 | 2 ug/ml | 6 | 8 | Neutravidin | 1.0M |
| | | | | (5 ug/ml) + | |
| | | | | Streptavidin (5 ug/ml) | |
| Round 5 | 2 ug/ml | 6 | 8 | Neutravidin | 1.0M |
| | | | | (5 ug/ml) + | |
| | | | | Streptavidin (5 ug/ml) | |

4.2 Sequence Analysis and Combinatorial Synthesis of Stable Mutants Several clones were sequenced from the fourth round selection pool. A panel of compounds (RFX002-RFX026) was chemically synthesized that included I, L, F, V or T point mutations at positions 15 and 17, and assessed for stability.

4.3 Determination of Melting Temperature (Tm) by Circular Dichroism (CD)

RFX002 and RFX025 showed a Tm of 66.9 and 66.6° C., respectively, compared to RFX001 which had a Tm of 33.4° C., as measured using a standard CD spectroscopy method.

### 5. Affinity Maturation of D-VEGF Binders, RFX002 and RFX025

Preliminary estimates of the VEGF binding affinity of RFX002 as compared to that of RFX001 showed a 5 fold increase by Octet-SPR or a 2-fold increase by standard SPR. Further optimization of binding affinity may be performed by utilizing the crystal structure of the E01-AM1/VEGF complex to select particular residues for optimization (e.g., residues involved in VEGF binding contacts). The VEGF contacting residues of the compound were identified by examining the change in solvent exposed surface area (SASA) upon binding and the distance of the residue from the VEGF chain. One rationale for selecting residues is to include surface exposed residues that show a reduction in SASA upon binding, and also to include residues that are in close vicinity of the D-VEGF chain. In general, partially buried residues were not selected for optimization. Residues in bold (Table 5) were selected for optimization.

TABLE 5
Analysis of the crystal structure of E01-AM1/VEGF complex to identify residues for optimization

| | Solvent Accesible Surface Area (SASA) | | | | | PYMOL Analysis | | | |
|---|---|---|---|---|---|---|---|---|---|
| | | | | Maximum (In | | Within | Within | Conserved | |
| | | | | Random Coil | | 4Ang of | 6Ang of | in hard | Other |
| | Uncomplexed | Complex | Difference | (G-X-G motif)) | Core residues | VEGF | VEGF | randomization | data |
| Thr 1 | 99.5 | 82.1 | 17.4 | 106.2 | No | No | No | N/A | |
| Tyr 2 | 22.5 | 10.5 | 12 | 193.1 | Yes | No | No | N/A | |
| Lys 3 | 67.2 | 67.2 | 0 | 164.5 | No | No | No | N/A | |
| Leu 4 | 0 | 0 | 0 | 146.2 | Yes | No | No | N/A | |
| Ile 5 | 53.6 | 53.5 | 0.1 | 147.3 | No | No | No | N/A | |
| Leu 6 | 2 | 2 | 0 | 146.2 | Yes | No | No | N/A | |
| Asn 7 | 75.3 | 75.3 | 0 | 114.3 | No | No | No | N/A | |
| Gly 8 | 5.7 | 5.7 | 0 | 87.2 | Buried | No | No | N/A | |
| Lys 9 | 158.6 | 158.6 | 0 | 164.5 | No | No | No | N/A | |
| Thr 10 | 138.3 | 138.3 | 0 | 106.2 | No | No | No | N/A | |
| Leu 11 | 61.7 | 61.7 | 0 | 146.2 | Partially buried | No | No | N/A | |
| Lys 12 | 145.4 | 145.4 | 0 | 164.5 | No | No | No | N/A | |
| Gly 13 | 19.2 | 19.2 | 0 | 87.2 | Partially buried | No | No | N/A | |
| Glu 14 | 126 | 126 | 0 | 141.2 | No | No | No | N/A | |
| Thr 15 | 32.9 | 32.9 | 0 | 106.2 | Partially buried | No | No | N/A | |
| Thr 16 | 92 | 92 | 0 | 106.2 | No | No | No | N/A | |
| Thr 17 | 15.4 | 15.4 | 0 | 106.2 | Buried | No | No | N/A | |
| Glu 18 | 162.3 | 156.3 | 6 | 141.2 | No | No | No | N/A | |
| Ala 19 | 20.1 | 16 | 4.1 | 64.9 | Partially buried | No | No | N/A | |
| Val 20 | 122.3 | 104.4 | 17.9 | 122.3 | No | No | No | N/A | |
| Asp 21 | 58.3 | 58.3 | 0 | 113 | No | No | No | N/A | |
| Val 22 | 53 | 21.9 | 31.1 | 122.3 | Partially buried | Yes | Yes | Hydrophobic | ² |
| Phe 23 | 149.7 | 36.6 | 113.1 | 180.1 | No | Yes | Yes | F/Hydrophobic | |
| Asp 24 | 69.9 | 65.7 | 4.2 | 113 | No | Yes | Yes | No | |
| Ala 25 | 0 | 0 | 0 | 64.9 | Yes | No | Yes | N/A | |
| Phe 26 | 80 | 0.5 | 79.5 | 180.1 | Partially buried | Yes | Yes | Strongly F | |
| Asp 27 | 89.8 | 52.7 | 37.1 | 113 | No | Yes | Yes | Strongly D/E | |
| Val 28 | 72.4 | 72.4 | 0 | 122.3 | Partially buried | No | Yes | N/A | |
| Phe 29 | 1 | 1 | 0 | 180.1 | Yes | No | No | N/A | |
| Phe 30 | 95.1 | 28.3 | 66.8 | 180.1 | Partially buried | Yes | Yes | Hydrophobic | |
| Val 31 | 92.5 | 92.5 | 0 | 122.3 | No | No | Yes | No | |
| Tyr 32 | 99.2 | 99.2 | 0 | 193.1 | Partially buried | No | Yes | N/A | |
| Ala 33 | 0 | 0 | 0 | 64.9 | Yes | No | Yes | N/A | |
| Ala 34 | 49.2 | 25.9 | 23.3 | 64.9 | Partially buried | Yes | Yes | No | |
| Ser 35 | 101.4 | 88 | 13.4 | 77.4 | No | Yes | Yes | No | |
| Asn 36 | 60.8 | 41.7 | 19.1 | 114.3 | No | No | Yes | N/A | |
| Phe 37 | 5.7 | 2 | 3.7 | 180.1 | Yes | Yes | Yes | F/Hydrophobic | |
| Ser 38 | 126.2 | 41.5 | 84.7 | 77.4 | No | Yes | Yes | No | |
| Asp 39 | 96 | 63.6 | 32.4 | 113 | No | Yes | Yes | No | ³ |
| Phe 40 | 62 | 0 | 62 | 180.1 | Partially buried | Yes | Yes | I/Hydrophobic | |
| Asp 41 | 116.6 | 88.4 | 28.2 | 113 | No | Yes | Yes | No | |
| Asp 42 | 78.4 | 42.6 | 35.8 | 113 | No | Yes | Yes | Strongly D | |
| Trp 43 | 59.6 | 0 | 59.6 | 224.6 | Partially buried | Yes | Yes | N/A | |
| Thr 44 | 63.6 | 42.4 | 21.2 | 106.2 | No | Yes | Yes | N/A | |
| Tyr 45 | 73.2 | 39.6 | 33.6 | 193.1 | Partially buried | Yes | Yes | N/A | |
| Asp 46 | 72.4 | 66.8 | 5.6 | 113 | No | No | Yes | N/A | |
| Asp 47 | 132.2 | 121 | 11.2 | 113 | No | No | Yes | N/A | |
| Ala 48 | 94.8 | 94.8 | 0 | 64.9 | No | No | No | N/A | |
| Thr 49 | 80.3 | 78.6 | 1.7 | 106.2 | No | No | No | N/A | |
| Lys 50 | 86.7 | 65 | 21.7 | 164.5 | No | No | No | N/A | |
| Thr 51 | 17.7 | 12.3 | 5.4 | 106.2 | No | No | No | N/A | |
| Phe 52 | 5.3 | 0 | 5.3 | 180.1 | Yes | Yes | Yes | N/A | |
| Thr 53 | 32 | 20.2 | 11.8 | 106.2 | No | No | Yes | N/A | |
| Val 54 | 0 | 0 | 0 | 122.3 | Yes | No | Yes | N/A | |
| Thr 55 | 37.5 | 32.6 | 4.9 | 106.2 | No | No | Yes | N/A | |
| Glu 56 | 63 | 54.3 | 8.7 | 141.2 | No | No | Yes | N/A | |

Notes:
¹ N/A not applicable;
² V22L point mutant increased affinity;
³ D39F point mutant increased affinity

5.1 Affinity Maturation Library Construction

In constructing the library, no N-terminal FLAG tag was used and at the C-terminal sequence was used a short GGS linker between the GB1 compounds and phage protein p3. The display construct for RFX025 was made from the clone E01-D39F/V22L/Y23F that was used in the stabilization selections. The following oligonucleotides were used to construct a tagless RFX0025.

### (Removal of FLAG for RFX025)

(SEQ ID NO: 512)
ACAAATGCCTATGCA ACGTACAAACTGATT
(SEQ ID NO: 513)
T N A Y A T Y K L I

(creation of RFX025 from E01-D39F/V22L/Y23F)

(SEQ ID NO: 514)
ACCCTGAAAGGTGAA TTC ACG CTC GAAGCAGTGGATCTG
(SEQ ID NO: 515)
T L K G E F T L E A V D L

(creation of stop template for RFX025)

(SEQ ID NO: 516)
ACCCTGAAAGGTGAA TTC ACG CTC GAA GCA GTG GAT TAA TAA TAA TACGATGATGCGACG
(SEQ ID NO: 517)
T L K G E F T L E A V D X X X V D D A T

(Removal of dimerization sequence)

(SEQ ID NO: 518)
ACCGAAGGCGGTTCT GATTTTGATTATGAA
(SEQ ID NO: 519)
T E G G S D F D Y E

The tagless template for RFX002 was constructed using the original clone as template and the following nucleotides:

(Removal of FLAG from disulfide mutant)

(SEQ ID NO: 520)
ACAAATGCCTATGCA ACGTACAAAATGATT
(SEQ ID NO: 521)
T N A Y A T Y K M I

(Stop template disulfide mutant)

(SEQ ID NO: 522)
ACCGAAGCAGTGGAT TAA TAA TAA TACGATGATGCGACG
(SEQ ID NO: 523)
T E A V D X X X Y D D A T

(Removal of dimerization sequence)

(SEQ ID NO: 524)
ACCGAAGGCGGTTCT GATTTTGATTATGAA
(SEQ ID NO: 525)
T E G G S D F D Y E

Using a stop template prevented a bias towards the parent clone in the library. The stop template was made for RFX002. The libraries were made with a stop template for RFX002 and regular tagless version for RFX025. The libraries were constructed as described using the methods described herein with molecular diversities of 8.1×10⁹ and 9×10⁹. The following oligonucleotides were used to make the library:

RFX002 Library

(SEQ ID NO: 526)
ACCGAAGCAGTGGAT (N2)(N4)(N3) (N4)(N4)(N4) (N3)(N1)(N4) GCA (N4)(N4)(N4)
(N3)(N1)(N4) GTT TTA (N4)(N4)(N4) (N3)(N4)(N4) TGC GCC (N3)(N2)(N4)
(N4)(N2)(N4) (N1)(N1)(N4) TTT (N4)(N2)(N4)(N4) (N4)(N2) ATT (N3)(N1)(N4)
(N3)(N1)(N4) TGG (N1)(N2)(N2) TACGATGATGCGACG

RFX025 Library

(SEQ ID NO: 527)
CTCGAAGCAGTGGAT (N2)(N4)(N3) (N4)(N4)(N4) (N3)(N1)(N4) GCA
(N4)(N4)(N4) (N3)(N1)(N4) GTT TTC (N4)(N4)(N4) (N3)(N4)(N4) TAC GCC
(N3)(N2)(N4) (N4)(N2)(N4) (N1)(N1)(N4) TTT (N4)(N2)(N4) (N4)(N4)(N2)
TTT (N3)(N1)(N4) (N3)(N1)(N4) TGG (N1)(N2)(N2) TACGATGATGCGACG

5.2 Off-Rate Selections

A selection process was performed using methods similar to those described above. The details of the selection are shown in Table 6. Prior to elution with 100 mM HCl, the bound clones in each round were incubated with 1 μM bacterially expressed L-RFX002 competitor.

TABLE 6
Selection process

| | | | Off Rate |
|---|---|---|---|
| | D-VEGF | | Incubation |
| | coating conc. | Washes | Time |
| Round 1 | 5 ug/ml | 8 | 1 h |
| Round 2 | 5 ug/ml | 8 | 2.5 h |
| Round 3 | 5 ug/ml | 8 | 5 h |
| Round 4 | 5 ug/ml | 8 | 15 h |

5.3 GuHCl Selections

During screening for specific binding to target, the presence of a chaotropic agent/denaturant can help select clones with improved stability and affinity. Phage were incubated overnight in the presence of various concentrations of GuHCl and urea and assayed using an ELISA method. Compounds RFX002 and RFX025 were shown to bind specifically to D-VEGF at various concentrations of urea or GuHCl. Various concentrations GuHCl were added to selection wells prior to elution with 100 mM HCl. The details of the selections are shown in Table 7. The selected clones were identified by competitive ELISA and analyzed for IC₅₀ using similar protocols to those described above.

TABLE 7
GuHCl selections

| | | | | |
|---|---|---|---|---|
| | | | GuHCl | Elution |
| D-VEGF | | GuHCl | Incubation | Titers |
| coating conc. | Washes | conc. | Time | (cfu/ml) |

RFX002 Library:

| | | | | | |
|---|---|---|---|---|---|
| Round 1 | 5 ug/ml | 8 | 2.5M | 2 h | 6 × 10\hat{ }4 |
| Round 2 | 5 ug/ml | 8 | 3M | 2.5 h | 4 × 10\hat{ }6 |
| Round 3 | 5 ug/ml | 8 | 3.5M | 5 h | 1.3 × 10\hat{ }6 |
| Round 4 | 5 ug/ml | 8 | 4M | 15 h | 1.5 × 10\hat{ }7 |

RFX025 Library:

| | | | | | |
|---|---|---|---|---|---|
| Round 1 | 5 ug/ml | 8 | 1.5M | 2 h | 5.8 × 10\hat{ }5 |
| Round 2 | 5 ug/ml | 8 | 2.5M | 2.5 h | 2.2 × 10\hat{ }6 |
| Round 3 | 5 ug/ml | 8 | 3.0M | 5 h | 1.2 × 10\hat{ }6 |
| Round 4 | 5 ug/ml | 8 | 3.5M | 15 h | 1.2 × 10\hat{ }7 |

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A GB1 peptidic compound that specifically binds with high affinity to a target protein, wherein the compound comprises a β1-β2 region and has three or more different non-core mutations in a region outside of the β1-β2 region.

2. (canceled)

3. The compound according to claim 1, wherein the target protein is a VEGF protein.

4-13. (canceled)

14. The compound according to claim 3, wherein the VEGF protein is a L-protein.

15. The compound according to claim 3, wherein the VEGF protein is a synthetic D-protein.

16-37. (canceled)

38. The compound according to claim 1, wherein the compound is an L-peptidic compound.

39. The compound according to claim 1, wherein the compound is a D-peptidic compound.

40-49. (canceled)

50. The compound according to claim 3, wherein the compound comprises at least six different non-core mutations in a region outside of the β1-β2 region.

51. The compound according to claim 50, wherein the compound comprises ten or more different mutations that are located at positions selected from the group consisting of positions 21-24, 26, 27, 30, 31, 34, 35, 37-41.

52-56. (canceled)

57. The compound according to claim 50, wherein the compound comprises five or more different mutations in the α1 region.

58-67. (canceled)

68. The compound according to claim 57, wherein the compound comprises two or more different mutations in the loop region between the α1 and β3 regions.

69-71. (canceled)

72. The compound according to claim 3, wherein the compound has a structure described by formula (I):
P1-α1-P2 (I)
wherein P1 and P2 are independently beta-hairpin domains and α1 is a helix domain; and
P1, α1 and P2 are connected independently by linking sequences of between 1 and 10 residues in length.

73. The compound according to claim 72, wherein P1 is β1-β2 and P2 is β3-β4 such that the compound is described by formula (II):
β1-β2-α1-β3-β4 (II)
wherein β1, β2, β3 and β4 are independently beta-strand domains; and
β1, β2, α1, β3 and β4 are connected independently by linking sequences of between 1 and 10 residues in length.

74. The compound according to claim 73, wherein the compound is described by a formula independently selected from the group consisting of:
F1-V1-F2 (III);
F3-V2-F4 (IV);
V3-F5-V4-F6-V5-F7 (V);
F8-V6-F9-V7-F10-V8 (VI);
V9-F11-V10 (VII); and
V11-F12-V12 (VIII)
wherein F1, F2, F3, F4, F5, F6, F7, F8, F9, F10, F11 and F12 are fixed regions and V1, V2, V3, V4, V5, V6, V7, V8, V9, V10, V11 and V12 are variable regions;
wherein the variable regions of the formula comprise the three or more different non-core mutations.

75. The compound according to claim 74, wherein the compound is described by formula (III), wherein:
F1 comprises a sequence having 75% or more amino acid sequence identity to the amino acid sequence set forth in TYKLILNGKTLKGETTTEA (SEQ ID NO:2);
F2 comprises a sequence having 75% or more amino acid sequence identity to an amino acid sequence set forth in TYDDATKTFTVTE (SEQ ID NO:3); and
V1 comprises a sequence that comprises at least 10 mutations compared to a parent amino acid sequence set forth in VDAATAEKVFKQYANDNGVDGEW (SEQ ID NO:4).

76. The compound according to claim 75, wherein V1 comprises a sequence of the formula:
(SEQ ID NO: 5)
VXXXXAXXVFXXYAXXNXXXXXW
wherein each X is independently a mutation that comprises substitution with a variant amino acid, wherein the mutation at position 19 of V1 comprises insertion of 0, 1 or 2 additional variant amino acids.

77. The compound according to claim 76, wherein:
F1 comprises the sequence set forth in TYKLILNGKTLKGETTTEA (SEQ ID NO:2);
F2 comprises the sequence set forth in TYDDATKTFTVTE (SEQ ID NO:3); and
each variant amino acid is independently selected from the group consisting of A, D, F, S, V and Y.

78-93. (canceled)

94. The compound according to claim 75, wherein the variable domain comprises a sequence having 80% or more amino acid sequence identity to an amino acid sequence set forth in one of SEQ ID NOs: 44-75 or set forth at positions 20-42 of one of SEQ ID NOs:76-162 and 529-809.

95-98. (canceled)

99. The compound according to claim 75, wherein the compound comprises a sequence having 80% or more amino acid sequence identity to an amino acid sequence set forth in one of SEQ ID NOs: 76-162 and 529-809.

100-103. (canceled)

104. The compound according to claim 99, wherein the VEGF protein is a D-protein and the compound is L-peptidic.

105. The compound according to claim 99, wherein the VEGF protein is a L-protein and the compound is D-peptidic.

106-140. (canceled)

141. An in vitro method comprising contacting a sample with a GB1 peptidic compound that specifically binds with high affinity to a target protein.

142. The method according to claim 141, wherein the sample is suspected of containing the target protein and the method further comprises evaluating whether the compound specifically binds to the target protein.

143. The method according to claim 142, wherein the target protein is a naturally occurring L-protein and the compound is D-peptidic.

144. (canceled)

145. The method according to claim 141, wherein the sample is known to contain the target protein.

146. The method according to claim 145, wherein the target protein is a synthetic D-protein and the compound is L-peptidic.

147. (canceled)

148. A method comprising administering to a subject a GB1 peptidic compound that specifically binds with high affinity to a target protein.

149. (canceled)

150. The method according to claim 148, wherein the target protein is a VEGF protein

151. (canceled)

152. The method according to claim 148, wherein the subject is human.

153. The method according to claim 152, wherein the compound is administered as a pharmaceutical preparation.

154. The method according to claim 153, wherein the compound is D-peptidic.

155-169. (canceled)


SEQUENCE LISTING (864 sequences)

SEQ ID NO: 1 - protein, 55 aa, Artificial Sequence
  Synthetic polypeptide
TYKLILNGKTLKGETTTEAVDAATAEKVFKQYANDNGVDGEWTYDDATKTFTVTE

SEQ ID NO: 2 - protein, 19 aa, Artificial Sequence
  Synthetic polypeptide
TYKLILNGKTLKGETTTEA

SEQ ID NO: 3 - protein, 13 aa, Artificial Sequence
  Synthetic polypeptide
TYDDATKTFTVTE

SEQ ID NO: 4 - protein, 23 aa, Artificial Sequence
  Synthetic polypeptide
VDAATAEKVFKQYANDNGVDGEW

SEQ ID NO: 5 - protein, 23 aa, Artificial Sequence
  Synthetic polypeptide
VXXXXAXXVFXXYAXXNXXXXXW

SEQ ID NO: 6 - protein, 23 aa, Artificial Sequence
  Synthetic polypeptide
VXXXXAXXVFXXYAXXNXXXXXW

SEQ ID NO: 7 - protein, 16 aa, Artificial Sequence
  Synthetic polypeptide
TYKLILNGKTLKGETT

SEQ ID NO: 8 - protein, 19 aa, Artificial Sequence
  Synthetic polypeptide
GVDGEWTYDDATKTFTVTE

SEQ ID NO: 9 - protein, 20 aa, Artificial Sequence
  Synthetic polypeptide
TEAVDAATAEKVFKQYANDN

SEQ ID NO: 10 - protein, 20 aa, Artificial Sequence
  Synthetic polypeptide
TXXXXXXXAXXXFXXXAXXN

SEQ ID NO: 11 - protein, 20 aa, Artificial Sequence
  Synthetic polypeptide
TXXXXXXXAXXXFXXXAXXN

SEQ ID NO: 12 - protein, 14 aa, Artificial Sequence
  Synthetic polypeptide
KLILNGKTLKGETT

SEQ ID NO: 13 - protein, 18 aa, Artificial Sequence
  Synthetic polypeptide
EKVFKQYANDNGVDGEWT

SEQ ID NO: 14 - protein, 5 aa, Artificial Sequence
  Synthetic polypeptide
FTVTE

SEQ ID NO: 15 - protein, 9 aa, Artificial Sequence
  Synthetic polypeptide
TEAVDAATA

SEQ ID NO: 16 - protein, 7 aa, Artificial Sequence
  Synthetic polypeptide
YDDATKT

SEQ ID NO: 17 - protein, 9 aa, Artificial Sequence
  Synthetic polypeptide
TXXXXXXXA

SEQ ID NO: 18 - protein, 7 aa, Artificial Sequence
  Synthetic polypeptide
YXXXXXT

SEQ ID NO: 19 - protein, 9 aa, Artificial Sequence
  Synthetic polypeptide
TXXXXXXXA

SEQ ID NO: 20 - protein, 7 aa, Artificial Sequence
  Synthetic polypeptide
YXXXXXT

SEQ ID NO: 21 - protein, 5 aa, Artificial Sequence
  Synthetic polypeptide
TYKLI

SEQ ID NO: 22 - protein, 21 aa, Artificial Sequence
  Synthetic polypeptide
ETTTEAVDAATAEKVFKQYAN

SEQ ID NO: 23 - protein, 10 aa, Artificial Sequence
  Synthetic polypeptide
TYDDATKTFT

SEQ ID NO: 24 - protein, 8 aa, Artificial Sequence
  Synthetic polypeptide
LNGKTLKG

SEQ ID NO: 25 - protein, 8 aa, Artificial Sequence
  Synthetic polypeptide
DNGVDGEW

SEQ ID NO: 26 - protein, 8 aa, Artificial Sequence
  Synthetic polypeptide
LXXXXXXG

SEQ ID NO: 27 - protein, 8 aa, Artificial Sequence
  Synthetic polypeptide
DXXXXXXW

SEQ ID NO: 28 - protein, 8 aa, Artificial Sequence
  Synthetic polypeptide
LXXXXXXG

SEQ ID NO: 29 - protein, 8 aa, Artificial Sequence
  Synthetic polypeptide
DXXXXXXW

SEQ ID NO: 30 - protein, 33 aa, Artificial Sequence
  Synthetic polypeptide
EAVDAATAEKVFKQYANDNGVDGEWTYDDATKT

SEQ ID NO: 31 - protein, 17 aa, Artificial Sequence
  Synthetic polypeptide
TYKLILNGKTLKGETTT

SEQ ID NO: 32 - protein, 5 aa, Artificial Sequence
  Synthetic polypeptide
FTVTE

SEQ ID NO: 33 - protein, 17 aa, Artificial Sequence
  Synthetic polypeptide
TYXLXLXXXXXXXXTXT

SEQ ID NO: 34 - protein, 5 aa, Artificial Sequence
  Synthetic polypeptide
FXVXX

SEQ ID NO: 35 - protein, 17 aa, Artificial Sequence
  Synthetic polypeptide
TYXLXLXXXXXXXXTXT

SEQ ID NO: 36 - protein, 5 aa, Artificial Sequence
  Synthetic polypeptide
FXVXX

SEQ ID NO: 37 - protein, 31 aa, Artificial Sequence
  Synthetic polypeptide
KTLKGETTTEAVDAATAEKVFKQYANDNGVD

SEQ ID NO: 38 - protein, 8 aa, Artificial Sequence
  Synthetic polypeptide
TYKLILNG

SEQ ID NO: 39 - protein, 16 aa, Artificial Sequence
  Synthetic polypeptide
GEWTYDDATKTFTVTE

SEQ ID NO: 40 - protein, 8 aa, Artificial Sequence
  Synthetic polypeptide
XYXLXLXG

SEQ ID NO: 41 - protein, 16 aa, Artificial Sequence
  Synthetic polypeptide
GXWXYXXXXXXFXVXE

SEQ ID NO: 42 - protein, 8 aa, Artificial Sequence
  Synthetic polypeptide
XYXLXLXG

SEQ ID NO: 43 - protein, 16 aa, Artificial Sequence
  Synthetic polypeptide
GXWXYXXXXXXFXVXE

SEQ ID NO: 44 - protein, 24 aa, Artificial Sequence
  Synthetic polypeptide
VDVFYAFDVFFDYASDNFADVADW

SEQ ID NO: 45 - protein, 24 aa, Artificial Sequence
  Synthetic polypeptide
VDFYSAFDVFFYYASSNFYDVADW

SEQ ID NO: 46 - protein, 24 aa, Artificial Sequence
  Synthetic polypeptide
VDVYSAVDVFYDYAAANFASFSDW

SEQ ID NO: 47 - protein, 24 aa, Artificial Sequence
  Synthetic polypeptide
VDVYDAFDVFFVYAASNFSDFDDW

SEQ ID NO: 48 - protein, 24 aa, Artificial Sequence
  Synthetic polypeptide
VDFFSAFDVFYAYADSNFDFYDDW

SEQ ID NO: 49 - protein, 24 aa, Artificial Sequence
  Synthetic polypeptide
VDFYAAFSVFFDYAAFNFYDVDDW

SEQ ID NO: 50 - protein, 24 aa, Artificial Sequence
  Synthetic polypeptide
VSYYDAFDVFFAYASSNFDFFDDW

SEQ ID NO: 51 - protein, 24 aa, Artificial Sequence
  Synthetic polypeptide
VDFFAAFDVFYSYASFNFAFFDDW

SEQ ID NO: 52 - protein, 24 aa, Artificial Sequence
  Synthetic polypeptide
VDVYDAFDVFYYYAAANYADFDDW

SEQ ID NO: 53 - protein, 24 aa, Artificial Sequence
  Synthetic polypeptide
VDFFDAFDVFFDYAVANFADVDDW

SEQ ID NO: 54 - protein, 24 aa, Artificial Sequence
  Synthetic polypeptide
VDVYSAFDVFYDYAVANFVDFSDW

SEQ ID NO: 55 - protein, 24 aa, Artificial Sequence
  Synthetic polypeptide
VDFFAAFAVFDSYASSNFFSVADW

SEQ ID NO: 56 - protein, 24 aa, Artificial Sequence
  Synthetic polypeptide
VDFYDAFDVFYDYASSNVSVFDDW

SEQ ID NO: 57 - protein, 24 aa, Artificial Sequence
  Synthetic polypeptide
VDVFDAFDVFAVYAFDNFVYVDDW

SEQ ID NO: 58 - protein, 24 aa, Artificial Sequence
  Synthetic polypeptide
VDFFDAFSVFDAYASSNYVVVDDW

SEQ ID NO: 59 - protein, 24 aa, Artificial Sequence
  Synthetic polypeptide
VDVFDAFDVFFYYAAANFDVYDDW

SEQ ID NO: 60 - protein, 24 aa, Artificial Sequence
  Synthetic polypeptide
VDFYSAFDVFFSYAASNFFVFDDW

SEQ ID NO: 61 - protein, 25 aa, Artificial Sequence
  Synthetic polypeptide
VDFYDAFDVFFVYAVDNYFFDVDDW

SEQ ID NO: 62 - protein, 24 aa, Artificial Sequence
  Synthetic polypeptide
VDLYDAFDVFFVYAASNFSDFDDW

SEQ ID NO: 63 - protein, 24 aa, Artificial Sequence
  Synthetic polypeptide
VDVFDAFDVFFVYAASNFSDFDDW

SEQ ID NO: 64 - protein, 24 aa, Artificial Sequence
  Synthetic polypeptide
VDVYGAFDVFFVYAASNFSDFDDW

SEQ ID NO: 65 - protein, 24 aa, Artificial Sequence
  Synthetic polypeptide
VDVYDAFEVFFVYAASNFSDFDDW

SEQ ID NO: 66 - protein, 24 aa, Artificial Sequence
  Synthetic polypeptide
VDVYDAFDVFFAYAASNFSDFDDW

SEQ ID NO: 67 - protein, 24 aa, Artificial Sequence
  Synthetic polypeptide
VDVYDAFDVFFVYASSNFSDFDDW

SEQ ID NO: 68 - protein, 24 aa, Artificial Sequence
  Synthetic polypeptide
VDVYDAFDVFFVYAARNFSDFDDW

SEQ ID NO: 69 - protein, 24 aa, Artificial Sequence
  Synthetic polypeptide
VDVYDAFDVFFVYAAENFSDFDDW

SEQ ID NO: 70 - protein, 24 aa, Artificial Sequence
  Synthetic polypeptide
VDVYDAFDVFFVYAAGNFSDFDDW

SEQ ID NO: 71 - protein, 24 aa, Artificial Sequence
  Synthetic polypeptide
VDVYDAFDVFFVYAASNFKDFDDW

SEQ ID NO: 72 - protein, 24 aa, Artificial Sequence
  Synthetic polypeptide
VDVYDAFDVFFVYAASNFGDFDDW

SEQ ID NO: 73 - protein, 24 aa, Artificial Sequence
  Synthetic polypeptide
VDVYDAFDVFFVYAASNFSFFDDW

SEQ ID NO: 74 - protein, 24 aa, Artificial Sequence
  Synthetic polypeptide
VDVYDAFDVFFVYAASNFSDIDDW

SEQ ID NO: 75 - protein, 24 aa, Artificial Sequence
  Synthetic polypeptide
VDVYDAFDVFFVYAASNFSDFVDW

SEQ ID NO: 76 - protein, 56 aa, Artificial Sequence
  Synthetic polypeptide
TYKLILNGKTLKGETTTEAVDVFYAFDVFFDYASDNFADVADWTYDDATKTFTVTE

SEQ ID NO: 77 - protein, 56 aa, Artificial Sequence
  Synthetic polypeptide
TYKLILNGKTLKGETTTEAVDFYSAFDVFFYYASSNFYDVADWTYDDATKTFTVTE

SEQ ID NO: 78 - protein, 56 aa, Artificial Sequence
  Synthetic polypeptide
TYKLILNGKTLKGETTTEAVDVYSAVDVFYDYAAANFASFSDWTYDDATKTFTVTE

SEQ ID NO: 79 - protein, 56 aa, Artificial Sequence
  Synthetic polypeptide
TYKLILNGKTLKGETTTEAVDVYDAFDVFFVYAASNFSDFDDWTYDDATKTFTVTE

SEQ ID NO: 80 - protein, 56 aa, Artificial Sequence
  Synthetic polypeptide
TYKLILNGKTLKGETTTEAVDFFSAFDVFYAYADSNFDFYDDWTYDDATKTFTVTE

SEQ ID NO: 81 - protein, 56 aa, Artificial Sequence
  Synthetic polypeptide
TYKLILNGKTLKGETTTEAVDFYAAFSVFFDYAAFNFYDVDDWTYDDATKTFTVTE

SEQ ID NO: 82 - protein, 56 aa, Artificial Sequence
  Synthetic polypeptide
TYKLILNGKTLKGETTTEAVSYYDAFDVFFAYASSNFDFFDDWTYDDATKTFTVTE

SEQ ID NO: 83 - protein, 56 aa, Artificial Sequence
  Synthetic polypeptide
TYKLILNGKTLKGETTTEAVDFFAAFDVFYSYASFNFAFFDDWTYDDATKTFTVTE

SEQ ID NO: 84 - protein, 56 aa, Artificial Sequence
  Synthetic polypeptide
TYKLILNGKTLKGETTTEAVDVYDAFDVFYYYAAANYADFDDWTYDDATKTFTVTE

SEQ ID NO: 85 - protein, 56 aa, Artificial Sequence
  Synthetic polypeptide
TYKLILNGKTLKGETTTEAVDFFDAFDVFFDYAVANFADVDDWTYDDATKTFTVTE

SEQ ID NO: 86 - protein, 56 aa, Artificial Sequence
  Synthetic polypeptide
TYKLILNGKTLKGETTTEAVDVYSAFDVFYDYAVANFVDFSDWTYDDATKTFTVTE

SEQ ID NO: 87 - protein, 56 aa, Artificial Sequence
  Synthetic polypeptide
TYKLILNGKTLKGETTTEAVDFFAAFAVFDSYASSNFFSVADWTYDDATKTFTVTE

SEQ ID NO: 88 - protein, 56 aa, Artificial Sequence
  Synthetic polypeptide
TYKLILNGKTLKGETTTEAVDFYDAFDVFYDYASSNVSVFDDWTYDDATKTFTVTE

SEQ ID NO: 89 - protein, 56 aa, Artificial Sequence
  Synthetic polypeptide
TYKLILNGKTLKGETTTEAVDVFDAFDVFAVYAFDNFVYVDDWTYDDATKTFTVTE

SEQ ID NO: 90 - protein, 56 aa, Artificial Sequence
  Synthetic polypeptide
TYKLILNGKTLKGETTTEAVDFFDAFSVFDAYASSNYVVVDDWTYDDATKTFTVTE

SEQ ID NO: 91 - protein, 56 aa, Artificial Sequence
  Synthetic polypeptide
TYKLILNGKTLKGETTTEAVDVFDAFDVFFYYAAANFDVYDDWTYDDATKTFTVTE

SEQ ID NO: 92 - protein, 56 aa, Artificial Sequence
  Synthetic polypeptide
TYKLILNGKTLKGETTTEAVDFYSAFDVFFSYAASNFFVFDDWTYDDATKTFTVTE

SEQ ID NO: 93 - protein, 57 aa, Artificial Sequence
  Synthetic polypeptide
TYKLILNGKTLKGETTTEAVDFYDAFDVFFVYAVDNYFFDVDDWTYDDATKTFTVTE

SEQ ID NO: 94 - protein, 56 aa, Artificial Sequence
  Synthetic polypeptide
TYKLILNGKTLKGETTTEAVDLYDAFDVFFVYAASNFSDFDDWTYDDATKTFTVTE

SEQ ID NO: 95 - protein, 56 aa, Artificial Sequence
  Synthetic polypeptide
TYKLILNGKTLKGETTTEAVDVFDAFDVFFVYAASNFSDFDDWTYDDATKTFTVTE

SEQ ID NO: 96 - protein, 56 aa, Artificial Sequence
  Synthetic polypeptide
TYKLILNGKTLKGETTTEAVDVYGAFDVFFVYAASNFSDFDDWTYDDATKTFTVTE

SEQ ID NO: 97 - protein, 56 aa, Artificial Sequence
  Synthetic polypeptide
TYKLILNGKTLKGETTTEAVDVYDAFEVFFVYAASNFSDFDDWTYDDATKTFTVTE

SEQ ID NO: 98 - protein, 56 aa, Artificial Sequence
  Synthetic polypeptide
TYKLILNGKTLKGETTTEAVDVYDAFDVFFAYAASNFSDFDDWTYDDATKTFTVTE

SEQ ID NO: 99 - protein, 56 aa, Artificial Sequence
  Synthetic polypeptide
TYKLILNGKTLKGETTTEAVDVYDAFDVFFVYASSNFSDFDDWTYDDATKTFTVTE

SEQ ID NO: 100 - protein, 56 aa, Artificial Sequence
  Synthetic polypeptide
TYKLILNGKTLKGETTTEAVDVYDAFDVFFVYAARNFSDFDDWTYDDATKTFTVTE

SEQ ID NO: 101 - protein, 56 aa, Artificial Sequence
  Synthetic polypeptide
TYKLILNGKTLKGETTTEAVDVYDAFDVFFVYAAENFSDFDDWTYDDATKTFTVTE

SEQ ID NO: 102 - protein, 56 aa, Artificial Sequence
  Synthetic polypeptide
TYKLILNGKTLKGETTTEAVDVYDAFDVFFVYAAGNFSDFDDWTYDDATKTFTVTE

SEQ ID NO: 103 - protein, 56 aa, Artificial Sequence
  Synthetic polypeptide
TYKLILNGKTLKGETTTEAVDVYDAFDVFFVYAASNFKDFDDWTYDDATKTFTVTE

SEQ ID NO: 104 - protein, 56 aa, Artificial Sequence
  Synthetic polypeptide
TYKLILNGKTLKGETTTEAVDVYDAFDVFFVYAASNFGDFDDWTYDDATKTFTVTE

SEQ ID NO: 105 - protein, 56 aa, Artificial Sequence
  Synthetic polypeptide
TYKLILNGKTLKGETTTEAVDVYDAFDVFFVYAASNFSFFDDWTYDDATKTFTVTE

SEQ ID NO: 106 - protein, 56 aa, Artificial Sequence
  Synthetic polypeptide
TYKLILNGKTLKGETTTEAVDVYDAFDVFFVYAASNFSDIDDWTYDDATKTFTVTE

SEQ ID NO: 107 - protein, 56 aa, Artificial Sequence
  Synthetic polypeptide
TYKLILNGKTLKGETTTEAVDVYDAFDVFFVYAASNFSDFVDWTYDDATKTFTVTE

SEQ ID NO: 108 - protein, 56 aa, Artificial Sequence
  Synthetic polypeptide
TYKLILNGKTLKGETTTEAVTHFDAFDVFFVYAASNFSDFDDWTYDDATKTFTVTE

SEQ ID NO: 109 - protein, 56 aa, Artificial Sequence
  Synthetic polypeptide
TYKLILNGKTLKGETTTEAVDMFYAFDVFFVYAASNFSDFDDWTYDDATKTFTVTE

SEQ ID NO: 110 - protein, 56 aa, Artificial Sequence
  Synthetic polypeptide
TYKLILNGKTLKGETTTEAVSWQSAFDVFFVYAASNFSDFDDWTYDDATKTFTVTE

SEQ ID NO: 111 - protein, 56 aa, Artificial Sequence
  Synthetic polypeptide
TYKLILNGKTLKGETTTEAVDHFEAFDVFFVYAASNFSDFDDWTYDDATKTFTVTE

SEQ ID NO: 112 - protein, 56 aa, Artificial Sequence
  Synthetic polypeptide
TYKLILNGKTLKGETTTEAVYMMDAFDVFFVYAASNFSDFDDWTYDDATKTFTVTE

SEQ ID NO: 113 - protein, 56 aa, Artificial Sequence
  Synthetic polypeptide
TYKLILNGKTLKGETTTEAVDFIHAFDVFFVYAASNFSDFDDWTYDDATKTFTVTE

SEQ ID NO: 114 - protein, 56 aa, Artificial Sequence
  Synthetic polypeptide
TYKLILNGKTLKGETTTEAVDWSTAFDVFFVYAASNFSDFDDWTYDDATKTFTVTE

SEQ ID NO: 115 - protein, 56 aa, Artificial Sequence
  Synthetic polypeptide
TYKLILNGKTLKGETTTEAVSLFDAFDVFFVYAASNFSDFDDWTYDDATKTFTVTE

SEQ ID NO: 116 - protein, 56 aa, Artificial Sequence
  Synthetic polypeptide
TYKLILNGKTLKGETTTEAVDFFGAFDVFFVYAASNFSDFDDWTYDDATKTFTVTE

SEQ ID NO: 117 - protein, 56 aa, Artificial Sequence
  Synthetic polypeptide
TYKLILNGKTLKGETTTEAVSIFHAFDVFFVYAASNFSDFDDWTYDDATKTFTVTE

SEQ ID NO: 118 - protein, 56 aa, Artificial Sequence
  Synthetic polypeptide
TYKLILNGKTLKGETTTEAVDVFAAFDVFFVYAASNFSDFDDWTYDDATKTFTVTE

SEQ ID NO: 119 - protein, 56 aa, Artificial Sequence
  Synthetic polypeptide
TYKLILNGKTLKGETTTEAVNIFKAFDVFFVYAASNFSDFDDWTYDDATKTFTVTE

SEQ ID NO: 120 - protein, 56 aa, Artificial Sequence
  Synthetic polypeptide
TYKLILNGKTLKGETTTEAVSVLDAFDVFFVYAASNFSDFDDWTYDDATKTFTVTE

SEQ ID NO: 121 - protein, 56 aa, Artificial Sequence
  Synthetic polypeptide
TYKLILNGKTLKGETTTEAVNSYEAFDVFFVYAASNFSDFDDWTYDDATKTFTVTE

SEQ ID NO: 122 - protein, 56 aa, Artificial Sequence
  Synthetic polypeptide
TYKLILNGKTLKGETTTEAVDVLEAFDVFFVYAASNFSDFDDWTYDDATKTFTVTE

SEQ ID NO: 123 - protein, 56 aa, Artificial Sequence
  Synthetic polypeptide
TYKLILNGKTLKGETTTEAVSMEEAFDVFFVYAASNFSDFDDWTYDDATKTFTVTE

SEQ ID NO: 124 - protein, 56 aa, Artificial Sequence
  Synthetic polypeptide
TYKLILNGKTLKGETTTEAVNHLDAFDVFFVYAASNFSDFDDWTYDDATKTFTVTE

SEQ ID NO: 125 - protein, 56 aa, Artificial Sequence
  Synthetic polypeptide
TYKLILNGKTLKGETTTEAVDLFHAFDVFFVYAASNFSDFDDWTYDDATKTFTVTE

SEQ ID NO: 126 - protein, 56 aa, Artificial Sequence
  Synthetic polypeptide
TYKLILNGKTLKGETTTEAVDVYDAFEVFWPYAASNFSDFDDWTYDDATKTFTVTE

SEQ ID NO: 127 - protein, 56 aa, Artificial Sequence
  Synthetic polypeptide
TYKLILNGKTLKGETTTEAVDVYDAFDVFYTYAASNFSDFDDWTYDDATKTFTVTE

SEQ ID NO: 128 - protein, 56 aa, Artificial Sequence
  Synthetic polypeptide
TYKLILNGKTLKGETTTEAVDVYDAFDVFFIYAASNFSDFDDWTYDDATKTFTVTE

SEQ ID NO: 129 - protein, 56 aa, Artificial Sequence
  Synthetic polypeptide
TYKLILNGKTLKGETTTEAVDVYDAMDVFYRYAASNFSDFDDWTYDDATKTFTVTE

SEQ ID NO: 130 - protein, 56 aa, Artificial Sequence
  Synthetic polypeptide
TYKLILNGKTLKGETTTEAVDVYDAFEVFLVYAASNFSDFDDWTYDDATKTFTVTE

SEQ ID NO: 131 - protein, 56 aa, Artificial Sequence
  Synthetic polypeptide
TYKLILNGKTLKGETTTEAVDVYDAFDVFVAYAASNFSDFDDWTYDDATKTFTVTE

SEQ ID NO: 132 - protein, 56 aa, Artificial Sequence
  Synthetic polypeptide
TYKLILNGKTLKGETTTEAVDVYDAFEVFFDYAASNFSDFDDWTYDDATKTFTVTE

SEQ ID NO: 133 - protein, 56 aa, Artificial Sequence; identical to SEQ ID NO: 127
  Synthetic polypeptide

SEQ ID NO: 134 - protein, 56 aa, Artificial Sequence
  Synthetic polypeptide
TYKLILNGKTLKGETTTEAVDVYDAFEVFLAYAASNFSDFDDWTYDDATKTFTVTE

SEQ ID NO: 135 - protein, 56 aa, Artificial Sequence
  Synthetic polypeptide
TYKLILNGKTLKGETTTEAVDVYDAFDVFFTYAASNFSDFDDWTYDDATKTFTVTE

SEQ ID NO: 136 - protein, 56 aa, Artificial Sequence
  Synthetic polypeptide
TYKLILNGKTLKGETTTEAVDVYDAFDVFYAYAASNFSDFDDWTYDDATKTFTVTE

SEQ ID NO: 137 - protein, 56 aa, Artificial Sequence
  Synthetic polypeptide
TYKLILNGKTLKGETTTEAVDVYDAFDVFFVYASLNFSDFDDWTYDDATKTFTVTE

SEQ ID NO: 138 - protein, 56 aa, Artificial Sequence
  Synthetic polypeptide
TYKLILNGKTLKGETTTEAVDVYDAFDVFFVYATSNFSDFDDWTYDDATKTFTVTE

SEQ ID NO: 139 - protein, 56 aa, Artificial Sequence
  Synthetic polypeptide
TYKLILNGKTLKGETTTEAVDVYDAFDVFFVYASRNFLDFDDWTYDDATKTFTVTE

SEQ ID NO: 140 - protein, 56 aa, Artificial Sequence
  Synthetic polypeptide
TYKLILNGKTLKGETTTEAVDVYDAFDVFFVYATSNFIDFDDWTYDDATKTFTVTE

SEQ ID NO: 141 - protein, 56 aa, Artificial Sequence
  Synthetic polypeptide
TYKLILNGKTLKGETTTEAVDVYDAFDVFFVYAAGNFVDFDDWTYDDATKTFTVTE

SEQ ID NO: 142 - protein, 56 aa, Artificial Sequence
  Synthetic polypeptide
TYKLILNGKTLKGETTTEAVDVYDAFDVFFVYASVNFTDFDDWTYDDATKTFTVTE

SEQ ID NO: 143 - protein, 56 aa, Artificial Sequence
  Synthetic polypeptide
TYKLILNGKTLKGETTTEAVDVYDAFDVFFVYAAQNYKDFDDWTYDDATKTFTVTE

SEQ ID NO: 144 - protein, 56 aa, Artificial Sequence
  Synthetic polypeptide
TYKLILNGKTLKGETTTEAVDVYDAFDVFFVYAAQNFYDFDDWTYDDATKTFTVTE

SEQ ID NO: 145 - protein, 56 aa, Artificial Sequence
  Synthetic polypeptide
TYKLILNGKTLKGETTTEAVDVYDAFDVFFVYASRNMFDFDDWTYDDATKTFTVTE

SEQ ID NO: 146 - protein, 56 aa, Artificial Sequence
  Synthetic polypeptide
TYKLILNGKTLKGETTTEAVDVYDAFDVFFVYASRNFRDFDDWTYDDATKTFTVTE

SEQ ID NO: 147 - protein, 56 aa, Artificial Sequence
  Synthetic polypeptide
TYKLILNGKTLKGETTTEAVDVYDAFDVFFVYAIENFIDFDDWTYDDATKTFTVTE

SEQ ID NO: 148 - protein, 56 aa, Artificial Sequence
  Synthetic polypeptide
TYKLILNGKTLKGETTTEAVDVYDAFDVFFVYAARNFKDFDDWTYDDATKTFTVTE

SEQ ID NO: 149 - protein, 56 aa, Artificial Sequence
  Synthetic polypeptide
TYKLILNGKTLKGETTTEAVDVYDAFDVFFVYAQENFQDFDDWTYDDATKTFTVTE

SEQ ID NO: 150 - protein, 56 aa, Artificial Sequence
  Synthetic polypeptide
TYKLILNGKTLKGETTTEAVDVYDAFDVFFVYAAKNIGDFDDWTYDDATKTFTVTE

SEQ ID NO: 151 - protein, 56 aa, Artificial Sequence
  Synthetic polypeptide
TYKLILNGKTLKGETTTEAVDVYDAFDVFFVYASENFKDFDDWTYDDATKTFTVTE

SEQ ID NO: 152 - protein, 56 aa, Artificial Sequence
  Synthetic polypeptide
TYKLILNGKTLKGETTTEAVDVYDAFDVFFVYAASNFSIIGDWTYDDATKTFTVTE

SEQ ID NO: 153 - protein, 56 aa, Artificial Sequence
  Synthetic polypeptide
TYKLILNGKTLKGETTTEAVDVYDAFDVFFVYAASNFSNISDWTYDDATKTFTVTE

SEQ ID NO: 154 - protein, 56 aa, Artificial Sequence
  Synthetic polypeptide
TYKLILNGKTLKGETTTEAVDVYDAFDVFFVYAASNFSDIVDWTYDDATKTFTVTE

SEQ ID NO: 155 - protein, 56 aa, Artificial Sequence
  Synthetic polypeptide
TYKLILNGKTLKGETTTEAVDVYDAFDVFFVYAASNFSIIDNWTYDDATKTFTVTE

SEQ ID NO: 156 - protein, 56 aa, Artificial Sequence
  Synthetic polypeptide
TYKLILNGKTLKGETTTEAVDVYDAFDVFFVYAASNFSNIEDWTYDDATKTFTVTE

SEQ ID NO: 157 - protein, 56 aa, Artificial Sequence
  Synthetic polypeptide
TYKLILNGKTLKGETTTEAVDVYDAFDVFFVYAASNFSAFDDWTYDDATKTFTVTE

SEQ ID NO: 158 - protein, 56 aa, Artificial Sequence
  Synthetic polypeptide
TYKLILNGKTLKGETTTEAVDVYDAFDVFFVYAASNFSSIHDWTYDDATKTFTVTE

SEQ ID NO: 159 - protein, 56 aa, Artificial Sequence
  Synthetic polypeptide
TYKLILNGKTLKGETTTEAVDVYDAFDVFFVYAASNFSFIADWTYDDATKTFTVTE

SEQ ID NO: 160 - protein, 56 aa, Artificial Sequence
  Synthetic polypeptide
TYKLILNGKTLKGETTTEAVDVYDAFDVFFVYAASNFSIQDDWTYDDATKTFTVTE

SEQ ID NO: 161 - protein, 56 aa, Artificial Sequence
  Synthetic polypeptide
TYKLILNGKTLKGETTTEAVDVYDAFDVFFVYAASNFSDMADWTYDDATKTFTVTE

SEQ ID NO: 162 - protein, 56 aa, Artificial Sequence
  Synthetic polypeptide
TYKLILNGKTLKGETTTEAVDVYDAFDVFFVYAASNFSSISDWTYDDATKTFTVTE

SEQ ID NO: 163 - protein, 18 aa, Artificial Sequence
  Synthetic polypeptide
TYYLFLFDSDSYADYTVT

SEQ ID NO: 164 - protein, 17 aa, Artificial Sequence
  Synthetic polypeptide
TYSLFLFDYYAYDVTST

SEQ ID NO: 165 - protein, 18 aa, Artificial Sequence
  Synthetic polypeptide
TYYLFLYDAYVSADYTDT

SEQ ID NO: 166 - protein, 19 aa, Artificial Sequence
  Synthetic polypeptide
TYYLFLYSVVDADAYFTVT

SEQ ID NO: 167 - protein, 17 aa, Artificial Sequence
  Synthetic polypeptide
TYSLFLFDFYAFAVTAT

SEQ ID NO: 168 - protein, 6 aa, Artificial Sequence
  Synthetic polypeptide
FFVDVD

SEQ ID NO: 169 - protein, 6 aa, Artificial Sequence
  Synthetic polypeptide
FFVFFA

SEQ ID NO: 170 - protein, 6 aa, Artificial Sequence
  Synthetic polypeptide
FFVVDY

SEQ ID NO: 171 - protein, 6 aa, Artificial Sequence
  Synthetic polypeptide
FFVDFS

SEQ ID NO: 172 - protein, 6 aa, Artificial Sequence
  Synthetic polypeptide
FFVFFA

SEQ ID NO: 173 - protein, 57 aa, Artificial Sequence
  Synthetic polypeptide
TYYLFLFDSDSYADYTVTEAVDAATAEKVFKQYANDNGVDGEWTYDDATKTFFVDVD

SEQ ID NO: 174 - protein, 56 aa, Artificial Sequence
  Synthetic polypeptide
TYSLFLFDYYAYDVTSTEAVDAATAEKVFKQYANDNGVDGEWTYDDATKTFFVFFA

SEQ ID NO: 175 - protein, 57 aa, Artificial Sequence
  Synthetic polypeptide
TYYLFLYDAYVSADYTDTEAVDAATAEKVFKQYANDNGVDGEWTYDDATKTFFVVDY

SEQ ID NO: 176 - protein, 58 aa, Artificial Sequence
  Synthetic polypeptide
TYYLFLYSVVDADAYFTVTEAVDAATAEKVFKQYANDNGVDGEWTYDDATKTFFVDFS

SEQ ID NO: 177 - protein, 56 aa, Artificial Sequence
  Synthetic polypeptide
TYSLFLFDFYAFAVTATEAVDAATAEKVFKQYANDNGVDGEWTYDDATKTFFVFFA

SEQ ID NO: 178 - protein, 57 aa, Artificial Sequence; identical to SEQ ID NO: 173
  Synthetic polypeptide

SEQ ID NO: 179 - protein, 57 aa, Artificial Sequence
  Synthetic polypeptide
TYVLFLLGSDSYADYTVTEAVDAATAEKVFKQYANDNGVDGEWTYDDATKTFFVDVD

SEQ ID NO: 180 - protein, 57 aa, Artificial Sequence
  Synthetic polypeptide
TYVLLLFGSDSYADYTVTEAVDAATAEKVFKQYANDNGVDGEWTYDDATKTFFVDVD

SEQ ID NO: 181 - protein, 57 aa, Artificial Sequence
  Synthetic polypeptide
TYWLFLLGSDSYADYTVTEAVDAATAEKVFKQYANDNGVDGEWTYDDATKTFFVDVD

SEQ ID NO: 182 - protein, 57 aa, Artificial Sequence
  Synthetic polypeptide
TYWLFLFGSDSYADYTVTEAVDAATAEKVFKQYANDNGVDGEWTYDDATKTFFVDVD

SEQ ID NO: 183 - protein, 57 aa, Artificial Sequence
  Synthetic polypeptide
TYHLFLIGSDSYADYTVTEAVDAATAEKVFKQYANDNGVDGEWTYDDATKTFFVDVD

SEQ ID NO: 184 - protein, 57 aa, Artificial Sequence
  Synthetic polypeptide
TYWLFLFDSDSYADYTVTEAVDAATAEKVFKQYANDNGVDGEWTYDDATKTFFVDVD

SEQ ID NO: 185 - protein, 57 aa, Artificial Sequence
  Synthetic polypeptide
TYYLFLFDTVAYADYTVTEAVDAATAEKVFKQYANDNGVDGEWTYDDATKTFFVDVD

SEQ ID NO: 186 - protein, 57 aa, Artificial Sequence
  Synthetic polypeptide
TYYLFLFDQIAWADYTVTEAVDAATAEKVFKQYANDNGVDGEWTYDDATKTFFVDVD

SEQ ID NO: 187 - protein, 57 aa, Artificial Sequence
  Synthetic polypeptide
TYYLFLFDDRMWADYTVTEAVDAATAEKVFKQYANDNGVDGEWTYDDATKTFFVDVD

SEQ ID NO: 188 - protein, 57 aa, Artificial Sequence
  Synthetic polypeptide
TYYLFLFDSLDYADYTVTEAVDAATAEKVFKQYANDNGVDGEWTYDDATKTFFVDVD

SEQ ID NO: 189 - protein, 57 aa, Artificial Sequence
  Synthetic polypeptide
TYYLFLFDEFEYADYTVTEAVDAATAEKVFKQYANDNGVDGEWTYDDATKTFFVDVD

SEQ ID NO: 190 - protein, 57 aa, Artificial Sequence
  Synthetic polypeptide
TYYLFLFDSESFADYTVTEAVDAATAEKVFKQYANDNGVDGEWTYDDATKTFFVDVD

SEQ ID NO: 191 - protein, 57 aa, Artificial Sequence
  Synthetic polypeptide
TYYLFLFDQDLWADYTVTEAVDAATAEKVFKQYANDNGVDGEWTYDDATKTFFVDVD

SEQ ID NO: 192 - protein, 57 aa, Artificial Sequence
  Synthetic polypeptide
TYYLFLFDQLMWADYTVTEAVDAATAEKVFKQYANDNGVDGEWTYDDATKTFFVDVD

SEQ ID NO: 193 - protein, 57 aa, Artificial Sequence
  Synthetic polypeptide
TYYLFLFDAMSYADYTVTEAVDAATAEKVFKQYANDNGVDGEWTYDDATKTFFVDVD

SEQ ID NO: 194 - protein, 57 aa, Artificial Sequence
  Synthetic polypeptide
TYYLFLFDSFRYADYTVTEAVDAATAEKVFKQYANDNGVDGEWTYDDATKTFFVDVD

SEQ ID NO: 195 - protein, 57 aa, Artificial Sequence
  Synthetic polypeptide
TYYLFLFDQESYADYTVTEAVDAATAEKVFKQYANDNGVDGEWTYDDATKTFFVDVD

SEQ ID NO: 196 - protein, 57 aa, Artificial Sequence
  Synthetic polypeptide
TYYLFLFDDSYYADYTVTEAVDAATAEKVFKQYANDNGVDGEWTYDDATKTFFVDVD

SEQ ID NO: 197 - protein, 57 aa, Artificial Sequence
  Synthetic polypeptide
TYYLFLFDEIGYADYTVTEAVDAATAEKVFKQYANDNGVDGEWTYDDATKTFFVDVD

SEQ ID NO: 198 - protein, 57 aa, Artificial Sequence
  Synthetic polypeptide
TYYLFLFDQYDFADYTVTEAVDAATAEKVFKQYANDNGVDGEWTYDDATKTFFVDVD

SEQ ID NO: 199 - protein, 57 aa, Artificial Sequence
  Synthetic polypeptide
TYYLFLFDSYQYADYTVTEAVDAATAEKVFKQYANDNGVDGEWTYDDATKTFFVDVD

SEQ ID NO: 200 - protein, 57 aa, Artificial Sequence
  Synthetic polypeptide
TYYLFLFDELSYADYTVTEAVDAATAEKVFKQYANDNGVDGEWTYDDATKTFFVDVD

SEQ ID NO: 201 - protein, 57 aa, Artificial Sequence
  Synthetic polypeptide
TYYLFLFDESLWADYTVTEAVDAATAEKVFKQYANDNGVDGEWTYDDATKTFFVDVD

SEQ ID NO: 202 - protein, 57 aa, Artificial Sequence
  Synthetic polypeptide
TYYLFLFDDQLYADYTVTEAVDAATAEKVFKQYANDNGVDGEWTYDDATKTFFVDVD

SEQ ID NO: 203 - protein, 57 aa, Artificial Sequence
  Synthetic polypeptide
TYYLFLFDAALWADYTVTEAVDAATAEKVFKQYANDNGVDGEWTYDDATKTFFVDVD

SEQ ID NO: 204 - protein, 57 aa, Artificial Sequence
  Synthetic polypeptide
TYYLFLFDDTTWADYTVTEAVDAATAEKVFKQYANDNGVDGEWTYDDATKTFFVDVD

SEQ ID NO: 205 - protein, 57 aa, Artificial Sequence
  Synthetic polypeptide
TYYLFLFDDDLYADYTVTEAVDAATAEKVFKQYANDNGVDGEWTYDDATKTFFVDVD

SEQ ID NO: 206 - protein, 57 aa, Artificial Sequence; identical to SEQ ID NO: 173
  Synthetic polypeptide

SEQ ID NO: 207 - protein, 57 aa, Artificial Sequence
  Synthetic polypeptide
TYYLFLFDSDSYGDYTKTEAVDAATAEKVFKQYANDNGVDGEWTYDDATKTFFVDVD

SEQ ID NO: 208 - protein, 57 aa, Artificial Sequence
  Synthetic polypeptide
TYYLFLFDSDSYADYTMTEAVDAATAEKVFKQYANDNGVDGEWTYDDATKTFFVDVD

SEQ ID NO: 209 - protein, 57 aa, Artificial Sequence
  Synthetic polypeptide
TYYLFLFDSDSYGDYTNTEAVDAATAEKVFKQYANDNGVDGEWTYDDATKTFFVDVD

SEQ ID NO: 210 - protein, 57 aa, Artificial Sequence
  Synthetic polypeptide
TYYLFLFDSDSYADYTETEAVDAATAEKVFKQYANDNGVDGEWTYDDATKTFFVDVD

SEQ ID NO: 211 - protein, 57 aa, Artificial Sequence
  Synthetic polypeptide
TYYLFLFDSDSYADFTDTEAVDAATAEKVFKQYANDNGVDGEWTYDDATKTFFVDVD

SEQ ID NO: 212 - protein, 57 aa, Artificial Sequence
  Synthetic polypeptide
TYYLFLFDSDSYADYTDTEAVDAATAEKVFKQYANDNGVDGEWTYDDATKTFFVDVD

SEQ ID NO: 213 - protein, 57 aa, Artificial Sequence
  Synthetic polypeptide
TYYLFLFDSDSYADFTLTEAVDAATAEKVFKQYANDNGVDGEWTYDDATKTFFVDVD

SEQ ID NO: 214 - protein, 57 aa, Artificial Sequence
  Synthetic polypeptide
TYYLFLFDSDSYADYTVTEAVDAATAEKVFKQYANDNGVDGEWTYDDATKTFFVESS

SEQ ID NO: 215 - protein, 57 aa, Artificial Sequence
  Synthetic polypeptide
TYYLFLFDSDSYADYTVTEAVDAATAEKVFKQYANDNGVDGEWTYDDATKTFFVYVS

SEQ ID NO: 216 - protein, 57 aa, Artificial Sequence
  Synthetic polypeptide
TYYLFLFDSDSYADYTVTEAVDAATAEKVFKQYANDNGVDGEWTYDDATKTFFVESL

SEQ ID NO: 217 - protein, 57 aa, Artificial Sequence
  Synthetic polypeptide
TYYLFLFDSDSYADYTVTEAVDAATAEKVFKQYANDNGVDGEWTYDDATKTFFVELI

SEQ ID NO: 218 - protein, 57 aa, Artificial Sequence; identical to SEQ ID NO: 173
  Synthetic polypeptide

SEQ ID NO: 219 - protein, 57 aa, Artificial Sequence
  Synthetic polypeptide
TYYLFLFDSDSYADYTVTEAVDAATAEKVFKQYANDNGVDGEWTYDDATKTFFVEAN

SEQ ID NO: 220 - protein, 57 aa, Artificial Sequence
  Synthetic polypeptide
TYYLFLFDSDSYADYTVTEAVDAATAEKVFKQYANDNGVDGEWTYDDATKTFFVEVN

SEQ ID NO: 221 - protein, 57 aa, Artificial Sequence
  Synthetic polypeptide
TYYLFLFDSDSYADYTVTEAVDAATAEKVFKQYANDNGVDGEWTYDDATKTFFVEAS

SEQ ID NO: 222 - protein, 57 aa, Artificial Sequence
  Synthetic polypeptide
TYYLFLFDSDSYADYTVTEAVDAATAEKVFKQYANDNGVDGEWTYDDATKTFFVEAE

SEQ ID NO: 223 - protein, 57 aa, Artificial Sequence
  Synthetic polypeptide
TYYLFLFDSDSYADYTVTEAVDAATAEKVFKQYANDNGVDGEWTYDDATKTFFVDVP

SEQ ID NO: 224 - protein, 57 aa, Artificial Sequence
  Synthetic polypeptide
TYYLFLFDSDSYADYTVTEAVDAATAEKVFKQYANDNGVDGEWTYDDATKTFFVDVI

SEQ ID NO: 225 - protein, 57 aa, Artificial Sequence
  Synthetic polypeptide
TYYLFLFDSDSYADYTVTEAVDAATAEKVFKQYANDNGVDGEWTYDDATKTFFVECV

SEQ ID NO: 226 - protein, 57 aa, Artificial Sequence
  Synthetic polypeptide
TYYLFLFDSDSYADYTVTEAVDAATAEKVFKQYANDNGVDGEWTYDDATKTFFVEAG

SEQ ID NO: 227 - protein, 57 aa, Artificial Sequence
  Synthetic polypeptide
TYYLFLFDSDSYADYTVTEAVDAATAEKVFKQYANDNGVDGEWTYDDATKTFFVDVE

SEQ ID NO: 228 - protein, 57 aa, Artificial Sequence
  Synthetic polypeptide
TYYLFLFDSDSYADYTVTEAVDAATAEKVFKQYANDNGVDGEWTYDDATKTFFVEGW

SEQ ID NO: 229 - protein, 24 aa, Artificial Sequence
  Synthetic polypeptide
VDFYYAFSVFFSYASSNSSDYVSW

SEQ ID NO: 230 - protein, 56 aa, Artificial Sequence
  Synthetic polypeptide
TYKLILNGKTLKGETTTEAVDFYYAFSVFFSYASSNSSDYVSWTYDDATKTFTVTE

SEQ ID NO: 231 - protein, 20 aa, Artificial Sequence
  Synthetic polypeptide
TVSSVFDDAYFYFVDYAFDN

SEQ ID NO: 232 - protein, 55 aa, Artificial Sequence
  Synthetic polypeptide
TYKLILNGKTLKGETTTVSSVFDDAYFYFVDYAFDNGVDGEWTYDDATKTFTVTE

SEQ ID NO: 233 - protein, 24 aa, Artificial Sequence
  Synthetic polypeptide
VSFSAAFDVFDFYAFYNDVDYFDW

SEQ ID NO: 234 - protein, 25 aa, Artificial Sequence
  Synthetic polypeptide
VAFDDAFDVFSDYAFSNFDADYADW

SEQ ID NO: 235 - protein, 56 aa, Artificial Sequence
  Synthetic polypeptide
TYKLILNGKTLKGETTTEAVSFSAAFDVFDFYAFYNDVDYFDWTYDDATKTFTVTE

SEQ ID NO: 236 - protein, 57 aa, Artificial Sequence
  Synthetic polypeptide
TYKLILNGKTLKGETTTEAVAFDDAFDVFSDYAFSNFDADYADWTYDDATKTFTVTE

SEQ ID NO: 237 - protein, 18 aa, Artificial Sequence
  Synthetic polypeptide
TYSLFLVYDYFYVDYTDT

SEQ ID NO: 238 - protein, 18 aa, Artificial Sequence
  Synthetic polypeptide
TYVLALYYYFFDYFFTYT

SEQ ID NO: 239 - protein, 6 aa, Artificial Sequence
  Synthetic polypeptide
FSVYFV

SEQ ID NO: 240 - protein, 6 aa, Artificial Sequence
  Synthetic polypeptide
FDVVDV

SEQ ID NO: 241 - protein, 57 aa, Artificial Sequence
  Synthetic polypeptide
TYSLFLVYDYFYVDYTDTEAVDAATAEKVFKQYANDNGVDGEWTYDDATKTFSVYFV

SEQ ID NO: 242 - protein, 57 aa, Artificial Sequence
  Synthetic polypeptide
TYVLALYYYFFDYFFTYTEAVDAATAEKVFKQYANDNGVDGEWTYDDATKTFDVVDV

SEQ ID NO: 243 - protein, 9 aa, Artificial Sequence
  Synthetic polypeptide
SYYDLYLDG

SEQ ID NO: 244 - protein, 17 aa, Artificial Sequence
  Synthetic polypeptide
GFWDYVYYSDYYFAVDE

SEQ ID NO: 245 - protein, 57 aa, Artificial Sequence
  Synthetic polypeptide
SYYDLYLDGKTLKGETTTEAVDAATAEKVFKQYANDNGVDGFWDYVYYSDYYFAVDE

SEQ ID NO: 246 - protein, 10 aa, Artificial Sequence
  Synthetic polypeptide
LYYYAYVYFG

SEQ ID NO: 247 - protein, 10 aa, Artificial Sequence
  Synthetic polypeptide
DVAFSFDADW

SEQ ID NO: 248 - protein, 4 aa, Artificial Sequence
  Synthetic polypeptide
VDVF

SEQ ID NO: 249 - protein, 60 aa, Artificial Sequence
  Synthetic polypeptide
TYKLILYYYAYVYFGETTTEAVDAATAEKVFKQYANDVAFSFDADWTYDDAKKTFTVDVF

SEQ ID NO: 250 - protein, 18 aa, Artificial Sequence
  Synthetic polypeptide
TYFLALFYSYSFFVATAT

SEQ ID NO: 251 - protein, 17 aa, Artificial Sequence
  Synthetic polypeptide
TYYLDLYYFVFSVYTYT

SEQ ID NO: 252 - protein, 6 aa, Artificial Sequence
  Synthetic polypeptide
FFVYFV

SEQ ID NO: 253 - protein, 6 aa, Artificial Sequence
  Synthetic polypeptide
FFVDFD

SEQ ID NO: 254 - protein, 57 aa, Artificial Sequence
  Synthetic polypeptide
TYFLALFYSYSFFVATATEAVDAATAEKVFKQYANDNGVDGEWTYDDATKTFFVYFV

SEQ ID NO: 255 - protein, 56 aa, Artificial Sequence
  Synthetic polypeptide
TYYLDLYYFVFSVYTYTEAVDAATAEKVFKQYANDNGVDGEWTYDDATKTFFVDFD

SEQ ID NO: 256 - protein, 18 aa, Artificial Sequence
  Synthetic polypeptide
TYVLALVADDVYYDFTVT

SEQ ID NO: 257 - protein, 6 aa, Artificial Sequence
  Synthetic polypeptide
FFVVFY

SEQ ID NO: 258 - protein, 57 aa, Artificial Sequence
  Synthetic polypeptide
TYVLALVADDVYYDFTVTEAVDAATAEKVFKQYANDNGVDGEWTYDDATKTFFVVFY

SEQ ID NO: 259 - protein, 18 aa, Artificial Sequence
  Synthetic polypeptide
GQNHHEVVKFMDVYQRSY

SEQ ID NO: 260 - protein, 31 aa, Artificial Sequence
  Synthetic polypeptide
CHPIETLVDIFQEYPDEIEYIFKPSCVPLMR

SEQ ID NO: 261 - protein, 53 aa, Artificial Sequence
  Synthetic polypeptide
CGGCCNDEGLECVPTEESNITMQIMRIKPHQGQHIGEMSFLQHNKCECRPKKD

SEQ ID NO: 262 - DNA, 72 bp, Artificial Sequence
  Synthetic polynucleotide
ggaccatagattatgaaaatcgaagaacaccaccatcatcaccaccatcatcaccacggcggtagcacgtac

SEQ ID NO: 263 - protein, 20 aa, Artificial Sequence
  Synthetic polypeptide
MKIEEHHHHHHHHHHGGSTY

SEQ ID NO: 264 - DNA, 33 bp, Artificial Sequence
  Synthetic polynucleotide
ggcggttctgacaaataagcggccggcccagtg

SEQ ID NO: 265 - protein, 5 aa, Artificial Sequence
  Synthetic polypeptide
GGSDK

SEQ ID NO: 266 - protein, 4 aa, Artificial Sequence
  Synthetic polypeptide
KIEE

SEQ ID NO: 267 - protein, 5 aa, Artificial Sequence
  Synthetic polypeptide
GGSDK

SEQ ID NO: 268 - DNA, 33 bp, Artificial Sequence
  Synthetic polynucleotide
accgaagcagtggatctgtatgatgcatttgat

SEQ ID NO: 269 - protein, 11 aa, Artificial Sequence
  Synthetic polypeptide
TEAVDLYDAFD

SEQ ID NO: 270 - DNA, 33 bp, Artificial Sequence
  Synthetic polynucleotide
gaagcagtggatgtttttgatgcatttgatgtt

SEQ ID NO: 271 - protein, 11 aa, Artificial Sequence
  Synthetic polypeptide
EAVDVFDAFDV

SEQ ID NO: 272 - DNA, 33 bp, Artificial Sequence
  Synthetic polynucleotide
gcagtggatgtttatggtgcatttgatgttttc

SEQ ID NO: 273 - protein, 11 aa, Artificial Sequence
  Synthetic polypeptide
AVDVYGAFDVF

SEQ ID NO: 274 - DNA, 33 bp, Artificial Sequence
  Synthetic polynucleotide
gtttatgatgcatttgaagttttctttgtttac

SEQ ID NO: 275 - protein, 11 aa, Artificial Sequence
  Synthetic polypeptide
VYDAFEVFFVY

SEQ ID NO: 276 - DNA, 33 bp, Artificial Sequence
  Synthetic polynucleotide
tttgatgttttctttgcttacgccgcttctaat

SEQ ID NO: 277 - protein, 11 aa, Artificial Sequence
  Synthetic polypeptide
FDVFFAYAASN

SEQ ID NO: 278 - DNA, 33 bp, Artificial Sequence
  Synthetic polynucleotide
ttctttgtttacgcctcttctaatttttctgat

SEQ ID NO: 279 - protein, 11 aa, Artificial Sequence
  Synthetic polypeptide
FFVYASSNFSD

SEQ ID NO: 280 - DNA, 33 bp, Artificial Sequence
  Synthetic polynucleotide
tttgtttacgccgctcgtaatttttctgatttt

SEQ ID NO: 281 - protein, 11 aa, Artificial Sequence
  Synthetic polypeptide
FVYAARNFSDF

SEQ ID NO: 282 - DNA, 33 bp, Artificial Sequence
  Synthetic polynucleotide
tttgtttacgccgctgaaaatttttctgatttt

SEQ ID NO: 283 - protein, 11 aa, Artificial Sequence
  Synthetic polypeptide
FVYAAENFSDF

SEQ ID NO: 284 - DNA, 33 bp, Artificial Sequence
  Synthetic polynucleotide
tttgtttacgccgctgggaatttttctgatttt

SEQ ID NO: 285 - protein, 11 aa, Artificial Sequence
  Synthetic polypeptide
FVYAAGNFSDF

SEQ ID NO: 286 - DNA, 33 bp, Artificial Sequence
  Synthetic polynucleotide
gccgcttctaattttaaagattttgatgattgg

SEQ ID NO: 287 - protein, 11 aa, Artificial Sequence
  Synthetic polypeptide
AASNFKDFDDW

SEQ ID NO: 288 - DNA, 33 bp, Artificial Sequence
  Synthetic polynucleotide
gccgcttctaattttggggattttgatgattgg

SEQ ID NO: 289 - protein, 11 aa, Artificial Sequence
  Synthetic polypeptide
AASNFGDFDDW

SEQ ID NO: 290 - DNA, 33 bp, Artificial Sequence
  Synthetic polynucleotide
gcttctaatttttctttctttgatgattggacc

SEQ ID NO: 291 - protein, 11 aa, Artificial Sequence
  Synthetic polypeptide
ASNFSFFDDWT

SEQ ID NO: 292 - DNA, 33 bp, Artificial Sequence
  Synthetic polynucleotide
tctaatttttctgatattgatgattggacctac

SEQ ID NO: 293 - protein, 11 aa, Artificial Sequence
  Synthetic polypeptide
SNFSDIDDWTY

SEQ ID NO: 294 - DNA, 33 bp, Artificial Sequence
  Synthetic polynucleotide
aatttttctgattttgttgattggacctacgat

SEQ ID NO: 295 - protein, 11 aa, Artificial Sequence
  Synthetic polypeptide
NFSDFVDWTYD

SEQ ID NO: 296 - protein, 19 aa, Artificial Sequence
  Synthetic polypeptide
VEPNCDIHVMWEWECFERL

SEQ ID NO: 297 - protein, 16 aa, Artificial Sequence
  Synthetic polypeptide
RGWVEICESDVWGRCL

SEQ ID NO: 298 - DNA, 96 bp, Artificial Sequence
  Synthetic polynucleotide
acgaccgaagcagtgnnknnknnknnkgcatttgatgttttctttgtttacgccgcttctaatttttctgattttgatgattggacctacgatgat

SEQ ID NO: 299 - DNA, 96 bp, Artificial Sequence
  Synthetic polynucleotide
acgaccgaagcagtggatgtttatgatgcannknnkgttttcnnknnktacgccgcttctaatttttctgattttgatgattggacctacgatgat

SEQ ID NO: 300 - DNA, 96 bp, Artificial Sequence
  Synthetic polynucleotide
acgaccgaagcagtggatgtttatgatgcatttgatgttttctttgtttacgccnnknnkaatnnknnkgattttgatgattggacctacgatgat

SEQ ID NO: 301 - DNA, 96 bp, Artificial Sequence
  Synthetic polynucleotide
acgaccgaagcagtggatgtttatgatgcatttgatgttttctttgtttacgccgcttctaatttttctnnknnknnknnktggacctacgatgat

SEQ ID NO: 302 - DNA, 75 bp, Artificial Sequence
  Synthetic polynucleotide
ggcggtagcacgtacnnkctgnnkctgnnknnktctgattcttatgctgattataccgttaccgaagcagtggat

SEQ ID NO: 303 - DNA, 45 bp, Artificial Sequence
  Synthetic polynucleotide
gcgacgaaaaccttctttgttgatgttgatggcggttctgacaaa

SEQ ID NO: 304 - DNA, 75 bp, Artificial Sequence
  Synthetic polynucleotide
ggcggtagcacgtactatctgtttctgtttgatnnknnknnknnkgctgattataccgttaccgaagcagtggat

SEQ ID NO: 305 - DNA, 45 bp, Artificial Sequence; identical to SEQ ID NO: 303
  Synthetic polynucleotide

SEQ ID NO: 306 - DNA, 75 bp, Artificial Sequence
  Synthetic polynucleotide
ggcggtagcacgtactatctgtttctgtttgattctgattcttatnnknnknnkaccnnkaccgaagcagtggat

SEQ ID NO: 307 - DNA, 45 bp, Artificial Sequence; identical to SEQ ID NO: 303
  Synthetic polynucleotide

SEQ ID NO: 308 - DNA, 75 bp, Artificial Sequence
  Synthetic polynucleotide
ggcggtagcacgtactatctgtttctgtttgattctgattcttatgctgattataccgttaccgaagcagtggat

SEQ ID NO: 309 - DNA, 45 bp, Artificial Sequence
  Synthetic polynucleotide
gcgacgaaaaccttcnnkgttnnknnknnkggcggttctgacaaa

SEQ ID NO: 310 - DNA, 96 bp, Artificial Sequence
  Synthetic polynucleotide
acgaccgaagcagtgnnknnknnknnkgcattttctgttttcttttcttacgcctcttctaattcttctgattatgtttcttggacctacgatgat

SEQ ID NO: 311 - DNA, 96 bp, Artificial Sequence
  Synthetic polynucleotide
acgaccgaagcagtggatttttattatgcannknnkgttttcnnknnktacgcctcttctaattcttctgattatgtttcttggacctacgatgat

SEQ ID NO: 312 - DNA, 96 bp, Artificial Sequence
  Synthetic polynucleotide
acgaccgaagcagtggatttttattatgcattttctgttttcttttcttacgccnnknnkaatnnknnkgattatgtttcttggacctacgatgat

SEQ ID NO: 313 - DNA, 96 bp, Artificial Sequence
  Synthetic polynucleotide
acgaccgaagcagtggatttttattatgcattttctgttttcttttcttacgcctcttctaattcttctnnknnknnknnktggacctacgatgat

SEQ ID NO: 314 - protein, 55 aa, Artificial Sequence
  Synthetic polypeptide
SYKLVIKGATFSGETATKAVDAAVAEQTFRDYANKNGVDGVWAYDAATKTFTVTE

SEQ ID NO: 315 - protein, 55 aa, Artificial Sequence
  Synthetic polypeptide
TYRLVIKGVTFSGETATKAVDAATAEQTFRQYANDNGITGEWAYDTATKTFTVTE

SEQ ID NO: 316 - protein, 55 aa, Artificial Sequence; identical to SEQ ID NO: 314
  Synthetic polypeptide

SEQ ID NO: 317 - protein, 55 aa, Artificial Sequence
  Synthetic polypeptide
TYRLVIKGVTFSGETATKAVDAATAEQTFRQYANDNGVTGEWAYDAATKTFTVTE

SEQ ID NO: 318 - protein, 55 aa, Artificial Sequence; identical to SEQ ID NO: 314
  Synthetic polypeptide

SEQ ID NO: 319 - protein, 55 aa, Artificial Sequence
  Synthetic polypeptide
TYRLVIKGVTFSGETSTKAVDAATAEQTFRQYANDNGVTGEWAYDAATKTFTVTE

SEQ ID NO: 320 - protein, 21 aa, Artificial Sequence
  Synthetic polypeptide
ANIPAEKAFRQYANDNGVDGV

SEQ ID NO: 321 - protein, 55 aa, Artificial Sequence; identical to SEQ ID NO: 1
  Synthetic polypeptide

SEQ ID NO: 322 - protein, 55 aa, Artificial Sequence
  Synthetic polypeptide
TYKLVINGKTLKGETTTKAVDAETAEKAFKQYANDNGVDGVWTYDDATKTFTVTE

SEQ ID NO: 323 - protein, 55 aa, Artificial Sequence
  Synthetic polypeptide
TYKLVINGKTLKGETTTKTVDAETAEKAFKQYANDNGVDGVWTYDDATKTFTVTE

SEQ ID NO: 324 - protein, 55 aa, Artificial Sequence
  Synthetic polypeptide
TYKLVINGKTLKGETTTKAVDAETAEKAFKQYANENGVDGVWTYDDATKTFTVTE

SEQ ID NO: 325 - protein, 55 aa, Artificial Sequence
  Synthetic polypeptide
TYKLVVKGNTFSGETTTKAIDTATAEKEFKQYATANNVDGEWSYDDATKTFTVTE

SEQ ID NO: 326 - protein, 55 aa, Artificial Sequence
  Synthetic polypeptide
TYKLIVKGNTFSGETTTKAVDAETAEKAFKQYATANNVDGEWSYDDATKTFTVTE

SEQ ID NO: 327 - protein, 55 aa, Artificial Sequence
  Synthetic polypeptide
TYKLIVKGNTFSGETTTKAIDAATAEKEFKQYATANGVDGEWSYDDATKTFTVTE

SEQ ID NO: 328 - protein, 55 aa, Artificial Sequence
  Synthetic polypeptide
TYKLIVKGNTFSGETTTKAVDAETAEKAFKQYANENGVYGEWSYDDATKTFTVTE

SEQ ID NO: 329 - protein, 55 aa, Artificial Sequence; identical to SEQ ID NO: 324
  Synthetic polypeptide

SEQ ID NO: 330 - protein, 45 aa, Artificial Sequence
  Synthetic polypeptide
MKGETTTEAVDAATAEKVFKQYANDNGVDGEWTYDDATKTFTVTE

SEQ ID NO: 331 - protein, 55 aa, Artificial Sequence; identical to SEQ ID NO: 322
  Synthetic polypeptide

SEQ ID NO: 332 - protein, 55 aa, Artificial Sequence
  Synthetic polypeptide
TYKLVINGKTLKGETTTEAVDAATAEKVFKQYANDNGVDGEWTYDDATKTFTVTE

SEQ ID NO: 333 - protein, 53 aa, Artificial Sequence
  Synthetic polypeptide
TYKLILNGKTLKGETTTEAVDAATARSFNFPILENSSSVPGDPLESTCMHVEH

SEQ ID NO: 334 - protein, 56 aa, Artificial Sequence
  Synthetic polypeptide
TYKLILNGKTLKGETTTEAVDAATARSFNFPILENSSSVPGDPLESTCRHASFAQA

SEQ ID NO: 335 - protein, 55 aa, Artificial Sequence; identical to SEQ ID NO: 314
  Synthetic polypeptide

SEQ ID NO: 336 - protein, 55 aa, Artificial Sequence
  Synthetic polypeptide
TYRLVIKGVTFSGETATKAVDAATAEQAFRQYANDNGVTGEWAYDAATKTFTVTE

SEQ ID NO: 337 - protein, 55 aa, Artificial Sequence; identical to SEQ ID NO: 314
  Synthetic polypeptide

SEQ ID NO: 338 - protein, 55 aa, Artificial Sequence; identical to SEQ ID NO: 315
  Synthetic polypeptide

SEQ ID NO: 339 - protein, 55 aa, Artificial Sequence; identical to SEQ ID NO: 325
  Synthetic polypeptide

SEQ ID NO: 340 - protein, 55 aa, Artificial Sequence
  Synthetic polypeptide
TYKLIVKGNTFSGETTTKAIDAATAEKEFKQYATANNVDGEWSYDYATKTFTVTE

SEQ ID NO: 341 - protein, 55 aa, Artificial Sequence
  Synthetic polypeptide
TYKLIVKGNTFSGETTTKAIDAATAEKEFKQYATANNVDGEWSYDDATKTFTVTE

SEQ ID NO: 342 - protein, 55 aa, Artificial Sequence; identical to SEQ ID NO: 326
  Synthetic polypeptide

SEQ ID NO: 343 - protein, 55 aa, Artificial Sequence
  Synthetic polypeptide
TYKLVINGKTLKGETTTKAVDVETAEKAFKQYANENGVDGVWTYDDATKTFTVTE

SEQ ID NO: 344 - protein, 55 aa, Artificial Sequence; identical to SEQ ID NO: 1
  Synthetic polypeptide

SEQ ID NO: 345 - protein, 55 aa, Artificial Sequence; identical to SEQ ID NO: 322
  Synthetic polypeptide

SEQ ID NO: 346 - protein, 55 aa, Artificial Sequence; identical to SEQ ID NO: 1
  Synthetic polypeptide

SEQ ID NO: 347 - protein, 55 aa, Artificial Sequence; identical to SEQ ID NO: 332
  Synthetic polypeptide

SEQ ID NO: 348 - protein, 55 aa, Artificial Sequence; identical to SEQ ID NO: 322
  Synthetic polypeptide

SEQ ID NO: 349 - protein, 56 aa, Artificial Sequence
  Synthetic polypeptide
TYKLILNGKTLKGETTTEAVXXXXAFDVFFVYAASNFSDFDDWTYDDATKTFTVTE

SEQ ID NO: 350 - protein, 56 aa, Artificial Sequence
  Synthetic polypeptide
TYKLILNGKTLKGETTTEAVDVYDAXXVFXXYAASNFSDFDDWTYDDATKTFTVTE

SEQ ID NO: 351 - protein, 56 aa, Artificial Sequence
  Synthetic polypeptide
TYKLILNGKTLKGETTTEAVDVYDAFDVFFVYAXXNXXDFDDWTYDDATKTFTVTE

SEQ ID NO: 352 - protein, 56 aa, Artificial Sequence
  Synthetic polypeptide
TYKLILNGKTLKGETTTEAVDVYDAFDVFFVYAASNFSXXXXWTYDDATKTFTVTE

SEQ ID NO: 353 - protein, 56 aa, Artificial Sequence
  Synthetic polypeptide
TYXLXLXXSDSYADYTVTEAVDAATAEKVFKQYANDNGVDEWTYDDATKTFFVDVD

SEQ ID NO: 354 - protein, 56 aa, Artificial Sequence
  Synthetic polypeptide
TYYLFLFDXXXXADYTVTEAVDAATAEKVFKQYANDNGVDEWTYDDATKTFFVDVD

SEQ ID NO: 355 - protein, 56 aa, Artificial Sequence
  Synthetic polypeptide
TYYLFLFDSDSYXXXTXTEAVDAATAEKVFKQYANDNGVDEWTYDDATKTFFVDVD

SEQ ID NO: 356 - protein, 56 aa, Artificial Sequence
  Synthetic polypeptide
TYYLFLFDSDSYADYTVTEAVDAATAEKVFKQYANDNGVDEWTYDDATKTFXVXXX

SEQ ID NO: 357 - protein, 56 aa, Artificial Sequence
  Synthetic polypeptide
TYKLILNGKTLKGETTTEAVXXXXAFSVFFSYASSNSSDYVSWTYDDATKTFTVTE

SEQ ID NO: 358 - protein, 56 aa, Artificial Sequence
  Synthetic polypeptide
TYKLILNGKTLKGETTTEAVDFYYAXXVFXXYASSNSSDYVSWTYDDATKTFTVTE

SEQ ID NO: 359 - protein, 56 aa, Artificial Sequence
  Synthetic polypeptide
TYKLILNGKTLKGETTTEAVDFYYAFSVFFSYAXXNXXDYVSWTYDDATKTFTVTE

SEQ ID NO: 360 - protein, 56 aa, Artificial Sequence
  Synthetic polypeptide
TYKLILNGKTLKGETTTEAVDFYYAFSVFFSYASSNSSXXXXWTYDDATKTFTVTE

SEQ ID NO: 361 - protein, 55 aa, Artificial Sequence
  Synthetic polypeptide
XYXLXXXGXTXXGETXTXXXDXXXAEXXFXXYAXXNXXXGXWXYDXATKTXTXTE

SEQ ID NO: 362 - protein, 55 aa, Artificial Sequence
  Synthetic polypeptide
XYXLXXXGXTXXGETXTXXXDXXXAEXXFXXYAXXNXXXGXWXYDXATKTFTVTE

SEQ ID NO: 363 - protein, 55 aa, Artificial Sequence
  Synthetic polypeptide
TYKLXXXGXTXXGETXTXAVDXXTAEXXFXQYAXXNXVDGXWXYDXATKTFTVTE

SEQ ID NO: 364 - protein, 55 aa, Artificial Sequence
  Synthetic polypeptide
TXKXXXXXXXXXXXXXXEXVDAXXXEKXXKXXXNXXXXXGXXTYXDXXKTXTXTE

SEQ ID NO: 365 - protein, 55 aa, Artificial Sequence; identical to SEQ ID NO: 364
  Synthetic polypeptide

SEQ ID NO: 366 - protein, 19 aa, Artificial Sequence
  Synthetic polypeptide
TYKMILNGKTLKSECTTEA

SEQ ID NO: 367 - protein, 19 aa, Artificial Sequence
  Synthetic polypeptide
TYKLILNGKTLKGEFTLEA

SEQ ID NO: 368 - protein, 13 aa, Artificial Sequence
  Synthetic polypeptide
XYDDATKTFTVTE

SEQ ID NO: 369 - protein, 24 aa, Artificial Sequence
  Synthetic polypeptide
VDXXXAFXVLXXCAXXXFXXIXXW

SEQ ID NO: 370 - protein, 24 aa, Artificial Sequence
  Synthetic polypeptide
VDXXXAFXVLXXCAXXXFXXIXDW

SEQ ID NO: 371 - protein, 24 aa, Artificial Sequence
  Synthetic polypeptide
VDXFDAFDVLFXCAXXXFSXIDDW

SEQ ID NO: 372 - protein, 56 aa, Artificial Sequence
  Synthetic polypeptide
TYKMILNGKTLKSECTTEAVDLFDAFDVLFVCAASNFSFIDDWTYDDATKTFTVTE

SEQ ID NO: 373 - protein, 24 aa, Artificial Sequence
  Synthetic polypeptide
VDXXXAFXVFXXYAXXXFXXXXDW

SEQ ID NO: 374 - protein, 24 aa, Artificial Sequence
  Synthetic polypeptide
VDXFXAFDVFFXYAXXXFXXFDDW

SEQ ID NO: 375 - protein, 24 aa, Artificial Sequence
  Synthetic polypeptide
VDLFXAFDVFFXYAXXXFSXFDDW

SEQ ID NO: 376 - protein, 56 aa, Artificial Sequence
  Synthetic polypeptide
TYKLILNGKTLKGEFTLEAVDLFDAFDVFFVYAASNFSFFDDWTYDDATKTFTVTE

SEQ ID NO: 377 - protein, 8 aa, Artificial Sequence
  Synthetic polypeptide
LDDYFYYG

SEQ ID NO: 378 - protein, 10 aa, Artificial Sequence
  Synthetic polypeptide
LYYADDFAFG

SEQ ID NO: 379 - protein, 8 aa, Artificial Sequence
  Synthetic polypeptide
LYSVDFFG

SEQ ID NO: 380 - protein, 8 aa, Artificial Sequence
  Synthetic polypeptide
LDAAFFYG

SEQ ID NO: 381 - protein, 9 aa, Artificial Sequence
  Synthetic polypeptide
LSFDSFDYG

SEQ ID NO: 382 - protein, 9 aa, Artificial Sequence
  Synthetic polypeptide
LAFSDFFDG

SEQ ID NO: 383 - protein, 8 aa, Artificial Sequence
  Synthetic polypeptide
LFADDYFG

SEQ ID NO: 384 - protein, 8 aa, Artificial Sequence
  Synthetic polypeptide
LSDSYYFG

SEQ ID NO: 385 - protein, 8 aa, Artificial Sequence
  Synthetic polypeptide
LDFDFSSG

SEQ ID NO: 386 - protein, 8 aa, Artificial Sequence
  Synthetic polypeptide
LDDYFYYG

SEQ ID NO: 387 - protein, 8 aa, Artificial Sequence
  Synthetic polypeptide
LSDYAAFG

SEQ ID NO: 388 - protein, 8 aa, Artificial Sequence
  Synthetic polypeptide
LFDSYSVG

SEQ ID NO: 389 - protein, 9 aa, Artificial Sequence
  Synthetic polypeptide
LDASFAYAG

SEQ ID NO: 390 - protein, 8 aa, Artificial Sequence
  Synthetic polypeptide
LDAAFFYG

SEQ ID NO: 391 - protein, 8 aa, Artificial Sequence
  Synthetic polypeptide
LSDYYVYG

SEQ ID NO: 392 - protein, 9 aa, Artificial Sequence
  Synthetic polypeptide
DFYYYYDFW

SEQ ID NO: 393 - protein, 9 aa, Artificial Sequence
  Synthetic polypeptide
DFYAYYDYW

SEQ ID NO: 394 - protein, 8 aa, Artificial Sequence
  Synthetic polypeptide
DFFSDYYW

SEQ ID NO: 395 - protein, 9 aa, Artificial Sequence
  Synthetic polypeptide
DFYSDFDFW

SEQ ID NO: 396 - protein, 9 aa, Artificial Sequence
  Synthetic polypeptide
DFYAYFDVW

SEQ ID NO: 397 - protein, 9 aa, Artificial Sequence
  Synthetic polypeptide
DFYAYYDYW

SEQ ID NO: 398 - protein, 8 aa, Artificial Sequence
  Synthetic polypeptide
DFYDSVFW

SEQ ID NO: 399 - protein, 9 aa, Artificial Sequence
  Synthetic polypeptide
DFYAYYDFW

SEQ ID NO: 400 - protein, 8 aa, Artificial Sequence
  Synthetic polypeptide
DFYDYFYW

SEQ ID NO: 401 - protein, 9 aa, Artificial Sequence
  Synthetic polypeptide
DFYYYYDFW

SEQ ID NO: 402 - protein, 10 aa, Artificial Sequence
  Synthetic polypeptide
DFYSDFYYDW

SEQ ID NO: 403 - protein, 9 aa, Artificial Sequence
  Synthetic polypeptide
DFYAVFDYW

SEQ ID NO: 404 - protein, 10 aa, Artificial Sequence
  Synthetic polypeptide
DFYDAFYFYW

SEQ ID NO: 405 - protein, 9 aa, Artificial Sequence
  Synthetic polypeptide
DFYSDFDFW

SEQ ID NO: 406 - protein, 10 aa, Artificial Sequence
  Synthetic polypeptide
DFYSDFAVDW

SEQ ID NO: 407 - protein, 4 aa, Artificial Sequence
  Synthetic polypeptide
VSDA

SEQ ID NO: 408 - protein, 4 aa, Artificial Sequence
  Synthetic polypeptide
VDDY

SEQ ID NO: 409 - protein, 4 aa, Artificial Sequence
  Synthetic polypeptide
VDDS

SEQ ID NO: 410 - protein, 4 aa, Artificial Sequence
  Synthetic polypeptide
VDDV

SEQ ID NO: 411 - protein, 4 aa, Artificial Sequence
  Synthetic polypeptide
VDVD

SEQ ID NO: 412 - protein, 4 aa, Artificial Sequence
  Synthetic polypeptide
VDVD

SEQ ID NO: 413 - protein, 4 aa, Artificial Sequence
  Synthetic polypeptide
VDDS

SEQ ID NO: 414 - protein, 3 aa, Artificial Sequence
  Synthetic polypeptide
VTE

SEQ ID NO: 415 - protein, 4 aa, Artificial Sequence
  Synthetic polypeptide
VDDD

SEQ ID NO: 416 - protein, 4 aa, Artificial Sequence
  Synthetic polypeptide
VSDA

SEQ ID NO: 417 - protein, 4 aa, Artificial Sequence
  Synthetic polypeptide
VDVD

SEQ ID NO: 418 - protein, 4 aa, Artificial Sequence
  Synthetic polypeptide
VDSV

SEQ ID NO: 419 - protein, 4 aa, Artificial Sequence
  Synthetic polypeptide
VDDS

SEQ ID NO: 420 - protein, 4 aa, Artificial Sequence
  Synthetic polypeptide
VDDV

SEQ ID NO: 421 - protein, 4 aa, Artificial Sequence
  Synthetic polypeptide
VDDV

SEQ ID NO: 422 - protein, 57 aa, Artificial Sequence
  Synthetic polypeptide
TYKLILDDYFYYGETTTEAVDAATAEKVFKQYANDFYYYYDFWTYDDATKTFTVSDA

SEQ ID NO: 423 - protein, 59 aa, Artificial Sequence
  Synthetic polypeptide
TYKLILYYADDFAFGETTTEAVDAATAEKVFKQYANDFYAYYDYWTYDDATKTFTVDDY

SEQ ID NO: 424 - protein, 56 aa, Artificial Sequence
  Synthetic polypeptide
TYKLILYSVDFFGETTTEAVDAATAEKVFKQYANDFFSDYYWTYDDATKTFTVDDS

SEQ ID NO: 425 - protein, 57 aa, Artificial Sequence
  Synthetic polypeptide
TYKLILDAAFFYGETTTEAVDAATAEKVFKQYANDFYSDFDFWTYDDATKTFTVDDV

SEQ ID NO: 426 - protein, 58 aa, Artificial Sequence
  Synthetic polypeptide
TYKLILSFDSFDYGETTTEAVDAATAEKVFKQYANDFYAYFDVWTYDDATKTFTVDVD

SEQ ID NO: 427 - protein, 58 aa, Artificial Sequence
  Synthetic polypeptide
TYKLILAFSDFFDGETTTEAVDAATAEKVFKQYANDFYAYYDYWTYDDATKTFTVDVD

SEQ ID NO: 428 - protein, 56 aa, Artificial Sequence
  Synthetic polypeptide
TYKLILFADDYFGETTTEAVDAATAEKVFKQYANDFYDSVFWTYDDATKTFTVDDS

SEQ ID NO: 429 - protein, 56 aa, Artificial Sequence
  Synthetic polypeptide
TYKLILSDSYYFGETTTEAVDAATAEKVFKQYANDFYAYYDFWTYDDATKTFTVTE

SEQ ID NO: 430 - protein, 56 aa, Artificial Sequence
  Synthetic polypeptide
TYKLILDFDFSSGETTTEAVDAATAEKVFKQYANDFYDYFYWTYDDATKTFTVDDD

SEQ ID NO: 431 - protein, 57 aa, Artificial Sequence; identical to SEQ ID NO: 422
  Synthetic polypeptide

SEQ ID NO: 432 - protein, 58 aa, Artificial Sequence
  Synthetic polypeptide
TYKLILSDYAAFGETTTEAVDAATAEKVFKQYANDFYSDFYYDWTYDDATKTFTVDVD

SEQ ID NO: 433 - protein, 57 aa, Artificial Sequence
  Synthetic polypeptide
TYKLILFDSYSVGETTTEAVDAATAEKVFKQYANDFYAVFDYWTYDDATKTFTVDSV

SEQ ID NO: 434 - protein, 59 aa, Artificial Sequence
  Synthetic polypeptide
TYKLILDASFAYAGETTTEAVDAATAEKVFKQYANDFYDAFYFYWTYDDATKTFTVDDS

SEQ ID NO: 435 - protein, 57 aa, Artificial Sequence; identical to SEQ ID NO: 425
  Synthetic polypeptide

SEQ ID NO: 436 - protein, 58 aa, Artificial Sequence
  Synthetic polypeptide
TYKLILSDYYVYGETTTEAVDAATAEKVFKQYANDFYSDFAVDWTYDDATKTFTVDDV

SEQ ID NO: 437 - protein, 18 aa, Artificial Sequence
  Synthetic polypeptide
TYYLVLAFFVDYAYVTFT

SEQ ID NO: 438 - protein, 6 aa, Artificial Sequence
  Synthetic polypeptide
FYVSDA

SEQ ID NO: 439 - protein, 57 aa, Artificial Sequence
  Synthetic polypeptide
TYYLVLAFFVDYAYVTFTEAVDAATAEKVFKQYANDNGVDGEWTYDDATKTFYVSDA

SEQ ID NO: 440 - protein, 4 aa, Artificial Sequence
  Synthetic polypeptide
FVSY

SEQ ID NO: 441 - protein, 4 aa, Artificial Sequence
  Synthetic polypeptide
FYDY

SEQ ID NO: 442 - protein, 9 aa, Artificial Sequence
  Synthetic polypeptide
TYVFVVSVA

SEQ ID NO: 443 - protein, 9 aa, Artificial Sequence
  Synthetic polypeptide
TAVYYVSVA

SEQ ID NO: 444 - protein, 7 aa, Artificial Sequence
  Synthetic polypeptide
YDDATKT

SEQ ID NO: 445 - protein, 57 aa, Artificial Sequence
  Synthetic polypeptide
FVSYKLILNGKTLKGETTTYVFVVSVAEKVFKQYANDNGVDGEWTYDDATKTFTVTE

SEQ ID NO: 446 - protein, 57 aa, Artificial Sequence
  Synthetic polypeptide
FYDYKLILNGKTLKGETTTAVYYVSVAEKVFKQYANDNGVDGEWTYDDATKTFTVTE

SEQ ID NO: 447 - protein, 20 aa, Artificial Sequence
  Synthetic polypeptide
TSDFYFYYAYSYFSYFADDN

SEQ ID NO: 448 - protein, 20 aa, Artificial Sequence
  Synthetic polypeptide
TYFSFFYFASSSFDYYAFDN

SEQ ID NO: 449 - protein, 24 aa, Artificial Sequence
  Synthetic polypeptide
VFFSAASVVFFYYAYDNSYFAYSW

SEQ ID NO: 450 - protein, 23 aa, Artificial Sequence
  Synthetic polypeptide
VFYSVAYDVFYSYAYFNSFVFSW

SEQ ID NO: 451 - protein, 24 aa, Artificial Sequence
  Synthetic polypeptide
VFSSFAFSVFYAYAASNASYSYYW

SEQ ID NO: 452 - protein, 25 aa, Artificial Sequence
  Synthetic polypeptide
VDSFSAVFVFDAYAFVNFYYSYSYW

SEQ ID NO: 453 - protein, 23 aa, Artificial Sequence
  Synthetic polypeptide
VFSFYADYVFSDYAYYNVYFSSW

SEQ ID NO: 454 - protein, 4 aa, Artificial Sequence
  Synthetic polypeptide
FSYY

SEQ ID NO: 455 - protein, 4 aa, Artificial Sequence
  Synthetic polypeptide
VFDY

SEQ ID NO: 456 - protein, 4 aa, Artificial Sequence
  Synthetic polypeptide
DFFY

SEQ ID NO: 457 - protein, 4 aa, Artificial Sequence
  Synthetic polypeptide
YDSY

SEQ ID NO: 458 - protein, 10 aa, Artificial Sequence
  Synthetic polypeptide
TFSYFFVYSA

SEQ ID NO: 459 - protein, 9 aa, Artificial Sequence
  Synthetic polypeptide
TYFSFFAYA

SEQ ID NO: 460 - protein, 9 aa, Artificial Sequence
  Synthetic polypeptide
TFSFYYYFA

SEQ ID NO: 461 - protein, 10 aa, Artificial Sequence
  Synthetic polypeptide
TFAFFYYFVA

SEQ ID NO: 462 - protein, 8 aa, Artificial Sequence
  Synthetic polypeptide
YDADDDDT

SEQ ID NO: 463 - protein, 8 aa, Artificial Sequence
  Synthetic polypeptide
YDAVFADT

SEQ ID NO: 464 - protein, 8 aa, Artificial Sequence
  Synthetic polypeptide
YADDADAT

SEQ ID NO: 465 - protein, 8 aa, Artificial Sequence
  Synthetic polypeptide
YDDDADDT

SEQ ID NO: 466 - protein, 55 aa, Artificial Sequence
  Synthetic polypeptide
TYKLILNGKTLKGETTTSDFYFYYAYSYFSYFADDNGVDGEWTYDDATKTFTVTE

SEQ ID NO: 467 - protein, 55 aa, Artificial Sequence
  Synthetic polypeptide
TYKLILNGKTLKGETTTYFSFFYFASSSFDYYAFDNGVDGEWTYDDATKTFTVTE

SEQ ID NO: 468 - protein, 56 aa, Artificial Sequence
  Synthetic polypeptide
TYKLILNGKTLKGETTTEAVFFSAASVVFFYYAYDNSYFAYSWTYDDATKTFTVTE

SEQ ID NO: 469 - protein, 55 aa, Artificial Sequence
  Synthetic polypeptide
TYKLILNGKTLKGETTTEAVFYSVAYDVFYSYAYFNSFVFSWTYDDATKTFTVTE

SEQ ID NO: 470 - protein, 56 aa, Artificial Sequence
  Synthetic polypeptide
TYKLILNGKTLKGETTTEAVFSSFAFSVFYAYAASNASYSYYWTYDDATKTFTVTE

SEQ ID NO: 471 - protein, 57 aa, Artificial Sequence
  Synthetic polypeptide
TYKLILNGKTLKGETTTEAVDSFSAVFVFDAYAFVNFYYSYSYWTYDDATKTFTVTE

SEQ ID NO: 472 - protein, 55 aa, Artificial Sequence
  Synthetic polypeptide
TYKLILNGKTLKGETTTEAVFSFYADYVFSDYAYYNVYFSSWTYDDATKTFTVTE

SEQ ID NO: 473 - protein, 59 aa, Artificial Sequence
  Synthetic polypeptide
FSYYKLILNGKTLKGETTTFSYFFVYSAEKVFKQYANDNGVDGEWTYDADDDDTFTVTE

SEQ ID NO: 474 - protein, 58 aa, Artificial Sequence
  Synthetic polypeptide
VFDYKLILNGKTLKGETTTYFSFFAYAEKVFKQYANDNGVDGEWTYDAVFADTFTVTE

SEQ ID NO: 475 - protein, 58 aa, Artificial Sequence
  Synthetic polypeptide
DFFYKLILNGKTLKGETTTFSFYYYFAEKVFKQYANDNGVDGEWTYADDADATFTVTE

SEQ ID NO: 476 - protein, 59 aa, Artificial Sequence
  Synthetic polypeptide
YDSYKLILNGKTLKGETTTFAFFYYFVAEKVFKQYANDNGVDGEWTYDDDADDTFTVTE

SEQ ID NO: 477 - DNA, 33 bp, Artificial Sequence
  Synthetic polynucleotide
ttctttgtttacgcctcttctaatttttctttc

SEQ ID NO: 478 - protein, 11 aa, Artificial Sequence
  Synthetic polypeptide
FFVYASSNFSF

SEQ ID NO: 479 - DNA, 33 bp, Artificial Sequence
  Synthetic polynucleotide
tttgtttacgccgctcgtaatttttctttcttt

SEQ ID NO: 480 - protein, 11 aa, Artificial Sequence
  Synthetic polypeptide
FVYAARNFSFF

SEQ ID NO: 481 - DNA, 33 bp, Artificial Sequence
  Synthetic polynucleotide
tttgtttacgccgctgaaaatttttctttcttt

SEQ ID NO: 482 - protein, 11 aa, Artificial Sequence
  Synthetic polypeptide
FVYAAENFSFF

SEQ ID NO: 483 - DNA, 33 bp, Artificial Sequence
  Synthetic polynucleotide
tttgtttacgccgctgggaatttttctttcttt

SEQ ID NO: 484 - protein, 11 aa, Artificial Sequence
  Synthetic polypeptide
FVYAAGNFSFF

SEQ ID NO: 485 - DNA, 33 bp, Artificial Sequence
  Synthetic polynucleotide
gccgcttctaattttaaattctttgatgattgg

SEQ ID NO: 486 - protein, 11 aa, Artificial Sequence
  Synthetic polypeptide
AASNFKFFDDW

SEQ ID NO: 487 - DNA, 33 bp, Artificial Sequence
  Synthetic polynucleotide
gccgcttctaattttgggttctttgatgattgg

SEQ ID NO: 488 - protein, 11 aa, Artificial Sequence
  Synthetic polypeptide
AASNFGFFDDW

SEQ ID NO: 489 - DNA, 33 bp, Artificial Sequence
  Synthetic polynucleotide
tctaatttttctttcattgatgattggacctac

SEQ ID NO: 490 - protein, 11 aa, Artificial Sequence
  Synthetic polypeptide
SNFSFIDDWTY

SEQ ID NO: 491 - DNA, 33 bp, Artificial Sequence
  Synthetic polynucleotide
aatttttctttctttgttgattggacctacgat

SEQ ID NO: 492 - protein, 11 aa, Artificial Sequence
  Synthetic polypeptide
NFSFFVDWTYD

SEQ ID NO: 493 - DNA, 33 bp, Artificial Sequence
  Synthetic polynucleotide
aatttttctttctttggtgattggacctacgat

SEQ ID NO: 494 - protein, 11 aa, Artificial Sequence
  Synthetic polypeptide
NFSFFGDWTYD

SEQ ID NO: 495 - protein, 31 aa, Artificial Sequence
  Synthetic polypeptide
TYKMILNGKTLKSECTTEAVDLFDAFDVLFV

SEQ ID NO: 496 - protein, 25 aa, Artificial Sequence
  Synthetic polypeptide
CAASNFSFIDDWTYDDATKTFTVTE

SEQ ID NO: 497 - protein, 56 aa, Artificial Sequence
  Synthetic polypeptide
TYKMILNGKTLKSECTTEAVDWFDAFDVLFVCAASQFSVIDDWTYDDATKTFTVTE

SEQ ID NO: 498 - protein, 56 aa, Artificial Sequence
  Synthetic polypeptide
TYKMILNGKTLKSECTTEAVDWFDAFDVLFACAASNFPLIEDWTYDDATKTFTVTE

SEQ ID NO: 499 - protein, 56 aa, Artificial Sequence
  Synthetic polypeptide
TYKMILNGKTLKSECTTEAVDWFDAFDVLFACAASNFSLIEDWTYDDATKTFTVTE

SEQ ID NO: 500 - protein, 56 aa, Artificial Sequence
  Synthetic polypeptide
TYKMILNGKTLKSECTTEAVDFFAAFEVLFGCADSHFSIIDDWTYDDATKTFTVTE

SEQ ID NO: 501 - protein, 56 aa, Artificial Sequence
  Synthetic polypeptide
TYKLILNGKTLKGETTTEAVDLFDAFDVFFVYAASNFSFFDDWTYDDATKTFTVTE

SEQ ID NO: 502 - DNA, 36 bp, Artificial Sequence
  Synthetic polynucleotide
accgaagcagtggatctgtttgatgcatttgatgtt

SEQ ID NO: 503 - protein, 12 aa, Artificial Sequence
  Synthetic polypeptide
TEAVDLFDAFDV

SEQ ID NO: 504 - DNA, 180 bp, Artificial Sequence
  Synthetic polynucleotide
ggtagcacgtacaaannnattnnnaacggcaaaaccnnnaaannngaannnacgnnngaagcagtggatctgtttgatgcatttgatnnnnnntttgttnnnnnngcttctaatnnntctttcnnngatgattggacctacgatgcgacgaaaaccnnnacgnnnaccgaaggcggttct

SEQ ID NO: 505 - protein, 60 aa, Artificial Sequence
  Synthetic polypeptide
GSTYKLILNGKTLKGETTTEAVDLFDAFDVFFVYAASNFSFFDDWTYDATKTFTVTEGGS

SEQ ID NO: 506 - DNA, 189 bp, Artificial Sequence
  Synthetic polynucleotide
aaaggcggtagcacgnnnaaannnattctgaacggcaaaaccctgaaaggtgaannnacgnnngaannngtggatnnntttgatnnnnnngatnnnnnntttgttnnngccgcttctaatttttctttctttgatgatnnnaccnnngatgatgcgacgaaaaccnnnacgnnnaccgaaggcggttct

SEQ ID NO: 507 - protein, 63 aa, Artificial Sequence
  Synthetic polypeptide
KGGSTYKLILNGKTLKGETTTEAVDLFDAFDVFFVYAASNFSFFDDWTYDDATKTFTVTEGGS

SEQ ID NO: 508 - DNA, 180 bp, Artificial Sequence
  Synthetic polynucleotide
ggtagcacgtacaaakygkygkygaacggcaaaacckygaaagstgaakygacgkyggaagcagtggatctgtttgatgcatttgatkygtwctttgtttwckyggcttctaattwttctttcbttgatgattggacctacgatgcgacgaaaacctwcacgkygaccgaaggcggttct

SEQ ID NO: 509 - protein, 60 aa, Artificial Sequence; identical to SEQ ID NO: 505
  Synthetic polypeptide

SEQ ID NO: 510 - DNA, 189 bp, Artificial Sequence
  Synthetic polynucleotide
aaaggcggtagcacgtwcaaakygattctgaacggcaaaaccctgaaaggtgaakygacgkyggaakyggtggatkygtttgatkygtwtgatkygtwctttgtttwcgccgcttctaatttttctttctttgatgattdkacctwcgatgatgcgacgaaaacctwcacgkygaccgaaggcggttct

SEQ ID NO: 511 - protein, 63 aa, Artificial Sequence; identical to SEQ ID NO: 507
  Synthetic polypeptide

SEQ ID NO: 512 - DNA, 30 bp, Artificial Sequence
  Synthetic polynucleotide
acaaatgcctatgcaacgtacaaactgatt

SEQ ID NO: 513 - protein, 10 aa, Artificial Sequence
  Synthetic polypeptide
TNAYATYKLI

SEQ ID NO: 514 - DNA, 39 bp, Artificial Sequence
  Synthetic polynucleotide
accctgaaaggtgaattcacgctcgaagcagtggatctg

SEQ ID NO: 515 - protein, 13 aa, Artificial Sequence
  Synthetic polypeptide
TLKGEFTLEAVDL

SEQ ID NO: 516 - DNA, 60 bp, Artificial Sequence
  Synthetic polynucleotide
accctgaaaggtgaattcacgctcgaagcagtggattaataataatacgatgatgcgacg

SEQ ID NO: 517 - protein, 20 aa, Artificial Sequence
  Synthetic polypeptide
TLKGEFTLEAVDXXXYDDAT

SEQ ID NO: 518 - DNA, 30 bp, Artificial Sequence
  Synthetic polynucleotide
accgaaggcggttctgattttgattatgaa

SEQ ID NO: 519 - protein, 10 aa, Artificial Sequence
  Synthetic polypeptide
TEGGSDFDYE

SEQ ID NO: 520 - DNA, 30 bp, Artificial Sequence
  Synthetic polynucleotide
acaaatgcctatgcaacgtacaaaatgatt

SEQ ID NO: 521 - protein, 10 aa, Artificial Sequence
  Synthetic polypeptide
TNAYATYKMI

SEQ ID NO: 522 - DNA, 39 bp, Artificial Sequence
  Synthetic polynucleotide
accgaagcagtggattaataataatacgatgatgcgacg

SEQ ID NO: 523 - protein, 13 aa, Artificial Sequence
  Synthetic polypeptide
TEAVDXXXYDDAT

SEQ ID NO: 524 - DNA, 30 bp, Artificial Sequence; identical to SEQ ID NO: 518
  Synthetic polynucleotide

SEQ ID NO: 525 - protein, 10 aa, Artificial Sequence
  Synthetic polypeptide
TEGGSDFDYE

SEQ ID NO: 526 - DNA, 99 bp, Artificial Sequence
  Synthetic polynucleotide
accgaagcagtggatnnnnnnnnngcannnnnngttttannnnnntgcgccnnnnnnnnntttnnnnnnattnnnnnntggnnntacgatgatgcgacg

SEQ ID NO: 527 - DNA, 99 bp, Artificial Sequence
  Synthetic polynucleotide
ctcgaagcagtggatnnnnnnnnngcannnnnngttttcnnnnnntacgccnnnnnnnnntttnnnnnntttnnnnnntggnnntacgatgatgcgacg

SEQ ID NO: 528 - protein, 19 aa, Artificial Sequence
  Synthetic polypeptide
TYKXILNGKTXKXEXTXEA

SEQ ID NO: 529 - protein, 56 aa, Artificial Sequence
  Synthetic polypeptide
TYKLILNGKTLKGETTTEAVDVYDAFDVFFVYAASNFSFIVDWTYDDATKTFTVTE

SEQ ID NO: 530 - protein, 56 aa, Artificial Sequence
  Synthetic polypeptide
TYKLILNGKTLKGETTTEAVDLYDAFDVFFVYAASNFSFFDDWTYDDATKTFTVTE

SEQ ID NO: 531 - protein, 56 aa, Artificial Sequence
  Synthetic polypeptide
TYKLILNGKTLKGETTTEAVDVFDAFDVFFVYAASNFSFFDDWTYDDATKTFTVTE

SEQ ID NO: 532 - protein, 56 aa, Artificial Sequence
  Synthetic polypeptide
TYKLILNGKTLKGETTTEAVDVYGAFDVFFVYAASNFSFFDDWTYDDATKTFTVTE

SEQ ID NO: 533 - protein, 56 aa, Artificial Sequence
  Synthetic polypeptide
TYKLILNGKTLKGETTTEAVDVYDAFEVFFVYAASNFSFFDDWTYDDATKTFTVTE

SEQ ID NO: 534 - protein, 56 aa, Artificial Sequence
  Synthetic polypeptide
TYKLILNGKTLKGETTTEAVDVYDAFDVFFAYAASNFSFFDDWTYDDATKTFTVTE

SEQ ID NO: 535 - protein, 56 aa, Artificial Sequence
  Synthetic polypeptide
TYKLILNGKTLKGETTTEAVDVYDAFDVFFVYASSNFSFFDDWTYDDATKTFTVTE

SEQ ID NO: 536 - protein, 56 aa, Artificial Sequence
  Synthetic polypeptide
TYKLILNGKTLKGETTTEAVDVYDAFDVFFVYAARNFSFFDDWTYDDATKTFTVTE

SEQ ID NO: 537 - protein, 56 aa, Artificial Sequence
  Synthetic polypeptide
TYKLILNGKTLKGETTTEAVDVYDAFDVFFVYAAENFSFFDDWTYDDATKTFTVTE

SEQ ID NO: 538 - protein, 56 aa, Artificial Sequence
  Synthetic polypeptide
TYKLILNGKTLKGETTTEAVDVYDAFDVFFVYAAGNFSFFDDWTYDDATKTFTVTE

SEQ ID NO: 539 - protein, 56 aa, Artificial Sequence
  Synthetic polypeptide
TYKLILNGKTLKGETTTEAVDVYDAFDVFFVYAASNFKFFDDWTYDDATKTFTVTE

SEQ ID NO: 540 - protein, 56 aa, Artificial Sequence
  Synthetic polypeptide
TYKLILNGKTLKGETTTEAVDVYDAFDVFFVYAASNFGFFDDWTYDDATKTFTVTE

SEQ ID NO: 541 - protein, 56 aa, Artificial Sequence
  Synthetic polypeptide
TYKLILNGKTLKGETTTEAVDVYDAFDVFFVYAASNFSFIDDWTYDDATKTFTVTE

SEQ ID NO: 542 - protein, 56 aa, Artificial Sequence
  Synthetic polypeptide
TYKLILNGKTLKGETTTEAVDVYDAFDVFFVYAASNFSFFVDWTYDDATKTFTVTE

SEQ ID NO: 543 - protein, 56 aa, Artificial Sequence; identical to SEQ ID NO: 501
  Synthetic polypeptide

SEQ ID NO: 544 - protein, 56 aa, Artificial Sequence
  Synthetic polypeptide
TYKLILNGKTRKFETTTEAVDLFDAFDVFFVYAASNFSFFDDWTYDDATKTFTVTE

SEQ ID NO: 545 - protein, 56 aa, Artificial Sequence
  Synthetic polypeptide
TYKLILNGKTQKREITTEAVDLFDAFDVFFVYAASNFSFFDDWTYDDATKTFTVTE

SEQ ID NO: 546 - protein, 56 aa, Artificial Sequence
  Synthetic polypeptide
TYKLILNGKTLKDETTIEAVDLFDAFDVFFVYAASNFSFFDDWTYDDATKTFTVTE

SEQ ID NO: 547 - protein, 56 aa, Artificial Sequence
  Synthetic polypeptide
TYKLILNGKTLKGEVTTEAVDLFDAFDVFFVYAASNFSFFDDWTYDDATKTFTVTE

SEQ ID NO: 548 - protein, 56 aa, Artificial Sequence
  Synthetic polypeptide
TYKLILNGKTLKGEITTEAVDLFDAFDVFFVYAASNFSFFDDWTYDDATKTFTVTE

SEQ ID NO: 549 - protein, 56 aa, Artificial Sequence
  Synthetic polypeptide
TYKLILNGKTMKGEITTEAVDLFDAFDVFFVYAASNFSFFDDWTYDDATKTFTVTE

SEQ ID NO: 550 - protein, 56 aa, Artificial Sequence
  Synthetic polypeptide
TYKLILNGKTLKGELTAEAVDLFDAFDVFFVYAASNFSFFDDWTYDDATKTFTVTE

SEQ ID NO: 551 - protein, 56 aa, Artificial Sequence
  Synthetic polypeptide
TYKLILNGKTSKGEVTLEAVDLFDAFDVFFVYAASNFSFFDDWTYDDATKTFTVTE

SEQ ID NO: 552 - protein, 56 aa, Artificial Sequence
  Synthetic polypeptide
TYKLILNGKTLKSEITTEAVDLFDAFDVFFVYAASNFSFFDDWTYDDATKTFTVTE

SEQ ID NO: 553 - protein, 56 aa, Artificial Sequence
  Synthetic polypeptide
TYKLILNGKTCKWETTTEAVDLFDAFDVFFVYAASNFSFFDDWTYDDATKTFTVTE

SEQ ID NO: 554 - protein, 56 aa, Artificial Sequence
  Synthetic polypeptide
TYKLILNGKTSKGELTVEAVDLFDAFDVFFVYAASNFSFFDDWTYDDATKTFTVTE

SEQ ID NO: 555 - protein, 56 aa, Artificial Sequence
  Synthetic polypeptide
TYKLILNGKTQKGEITTEAVDLFDAFDVFFVYAASNFSFFDDWTYDDATKTFTVTE

SEQ ID NO: 556 - protein, 56 aa, Artificial Sequence
  Synthetic polypeptide
TYKLILNGKTLKGEMTTEAVDLFDAFDVFFVYAASNFSFFDDWTYDDATKTFTVTE

SEQ ID NO: 557 - protein, 56 aa, Artificial Sequence
  Synthetic polypeptide
TYKLILNGKTLKGELTTEAVDLFDAFDVFFVYAASNFSFFDDWTYDDATKTFTVTE

SEQ ID NO: 558 - protein, 56 aa, Artificial Sequence
  Synthetic polypeptide
TYKLILNGKTFKDETTTEAVDLFDAFDVFFVYAASNFSFFDDWTYDDATKTFTVTE

SEQ ID NO: 559 - protein, 56 aa, Artificial Sequence
  Synthetic polypeptide
TYKLILNGKTWKGETTTEAVDLFDAFDVFFVYAASNFSFFDDWTYDDATKTFTVTE

SEQ ID NO: 560 - protein, 56 aa, Artificial Sequence
  Synthetic polypeptide
TYKLILNGKTSKGEVTVEAVDLFDAFDVFFVYAASNFSFFDDWTYDDATKTFTVTE

SEQ ID NO: 561 - protein, 56 aa, Artificial Sequence
  Synthetic polypeptide
TYKLILNGKTLKGEATIEAVDLFDAFDVFFVYAASNFSFFDDWTYDDATKTFTVTE

SEQ ID NO: 562 - protein, 56 aa, Artificial Sequence
  Synthetic polypeptide
TYKMILNGKTRKGETTMEAVDLFDAFDVFFVYAASNFSFIDDWTYDDATKTFTVTE

SEQ ID NO: 563 - protein, 56 aa, Artificial Sequence
  Synthetic polypeptide
TYKLILNGKTLKGEVTLEAVDLFDAFDVFFVYAASNFSFFDDWTYDDATKTFTVTE

SEQ ID NO: 564 - protein, 56 aa, Artificial Sequence
  Synthetic polypeptide
TYKLILNGKTVKGETTTEAVDLFDAFDVFFVYAASNFSFFDDWTYDDATKTFTVTE

SEQ ID NO: 565 - protein, 56 aa, Artificial Sequence
  Synthetic polypeptide
TYKLILNGKTRKGEHTLEAVDLFDAFDVFFVYAASNFSFFDDWTYDDATKTFTVTE

SEQ ID NO: 566 - protein, 56 aa, Artificial Sequence
  Synthetic polypeptide
TYKLILNGKTSKAELTTEAVDLFDAFDVFFVYAASNFSFFDDWTYDDATKTFTVTE

SEQ ID NO: 567 - protein, 56 aa, Artificial Sequence
  Synthetic polypeptide
TYKLILNGKTLKGEVTVEAVDLFDAFDVFFVYAASNFSFFDDWTYDDATKTFTVTE

SEQ ID NO: 568 - protein, 56 aa, Artificial Sequence
  Synthetic polypeptide
TYKLILNGKTLKDEYTIEAVDLFDAFDVFFVYAASNFSFFDDWTYDDATKTFTVTE

SEQ ID NO: 569 - protein, 56 aa, Artificial Sequence
  Synthetic polypeptide
TYKLILNGKTVKGELTAEAVDLFDAFDVFFVYAASNFSFFDDWTYDDATKTFTVTE

SEQ ID NO: 570 - protein, 56 aa, Artificial Sequence
  Synthetic polypeptide
TYKLILNGKTLKAELTTEAVDLFDAFDVFFVYAASNFSFFDDWTYDDATKTFTVTE

SEQ ID NO: 571 - protein, 56 aa, Artificial Sequence
  Synthetic polypeptide
TYKLILNGKTLKREITTEAVDLFDAFDVFFVYAASNFSFFDDWTYDDATKTFTVTE

SEQ ID NO: 572 - protein, 56 aa, Artificial Sequence; identical to SEQ ID NO: 372
  Synthetic polypeptide

SEQ ID NO: 573 - protein, 56 aa, Artificial Sequence
  Synthetic polypeptide
TYKLILNGKTMKGETTTEAVDLFDAFDVFFVYAASNFSFFDDWTYDDATKTFTVTE

SEQ ID NO: 574 - protein, 56 aa, Artificial Sequence
  Synthetic polypeptide
TYKLILNGKTVKGEITTEAVDLFDAFDVFFVYAASNFSFFDDWTYDDATKTFTVTE

SEQ ID NO: 575 - protein, 56 aa, Artificial Sequence
  Synthetic polypeptide
TYKLILNGKTSKEEVTAEAVDLFDAFDVFFVYAASNFSFFDDWTYDDATKTFTVTE

SEQ ID NO: 576 - protein, 56 aa, Artificial Sequence
  Synthetic polypeptide
TYKLILNGKTLKREFTTEAVDLFDAFDVFFVYAASNFSFFDDWTYDDATKTFTVTE

SEQ ID NO: 577 - protein, 56 aa, Artificial Sequence
  Synthetic polypeptide
TYKLILNGKTLKDEITTEAVDLFDAFDVFFVYAASNFSFFDDWTYDDATKTFTVTE

SEQ ID NO: 578 - protein, 56 aa, Artificial Sequence
  Synthetic polypeptide
TYKLILNGKTSKAELTLEAVDLFDAFDVFFVYAASNFSFFDDWTYDDATKTFTVTE

SEQ ID NO: 579 - protein, 56 aa, Artificial Sequence
  Synthetic polypeptide
TYKLILNGKTAKGELTVEAVDLFDAFDVFFVYAASNFSFFDDWTYDDATKTFTVTE

SEQ ID NO: 580 - protein, 56 aa, Artificial Sequence
  Synthetic polypeptide
TYKLILNGKTKKREITMEAVDLFDAFDVFFVYAASNFSFIDDWTYDDATKTFTVTE

SEQ ID NO: 581 - protein, 56 aa, Artificial Sequence
  Synthetic polypeptide
TYKLILNGKTMKCEITTEAVDLFDAFDVFFVYAASNFSFFDDWTYDDATKTFTVTE

SEQ ID NO: 582 - protein, 56 aa, Artificial Sequence
  Synthetic polypeptide
TYKLILNGKTLKGETTYEAVDLFDAFDFFFVYAASNFSFFDDWTYDDATKTFTVTE

SEQ ID NO: 583 - protein, 56 aa, Artificial Sequence
  Synthetic polypeptide
TYKLILNGKTMKCETTTEAVDLFDAFDVFFVYAASNFSFFDDWTYDDATKTFTVTE

SEQ ID NO: 584 - protein, 56 aa, Artificial Sequence; identical to SEQ ID NO: 573
  Synthetic polypeptide

SEQ ID NO: 585 - protein, 56 aa, Artificial Sequence
  Synthetic polypeptide
TYKLVLNGKTLKGELTAEAVDLFDAFDVFFVYAASNFSFFDDWTYDDATKTFTVTE

SEQ ID NO: 586 - protein, 56 aa, Artificial Sequence
  Synthetic polypeptide
TYKLLLNGKTSKAEVTLEAVDLFDAFDVFFVYAASNFSFFDDWTYDDATKTFTVTE

SEQ ID NO: 587 - protein, 56 aa, Artificial Sequence; identical to SEQ ID NO: 550
  Synthetic polypeptide

SEQ ID NO: 588 - protein, 56 aa, Artificial Sequence
  Synthetic polypeptide
TYKLILNGKTRKSETTTEAVDLFDAFDVFFVYAASNFSFFDDWTYDDATKTFTVTE

SEQ ID NO: 589 - protein, 56 aa, Artificial Sequence; identical to SEQ ID NO: 548
  Synthetic polypeptide

SEQ ID NO: 590 - protein, 56 aa, Artificial Sequence; identical to SEQ ID NO: 564
  Synthetic polypeptide

SEQ ID NO: 591 - protein, 56 aa, Artificial Sequence
  Synthetic polypeptide
TYKLILNGKTGKGETTTEAVDLFDAFDVFFVYAASNFSFFDDWTYDDATKTFTVTE

SEQ ID NO: 592 - protein, 56 aa, Artificial Sequence; identical to SEQ ID NO: 531
  Synthetic polypeptide

SEQ ID NO: 593 - protein, 56 aa, Artificial Sequence
  Synthetic polypeptide
TYKLILNGKTLKGEATTEAVDLFDAFDVFFVYAASNFSFFDDWTYDDATKTFTVTE

SEQ ID NO: 594 - protein, 56 aa, Artificial Sequence; identical to SEQ ID NO: 564
  Synthetic polypeptide

SEQ ID NO: 595 - protein, 56 aa, Artificial Sequence
  Synthetic polypeptide
TYKLILNGKTHKGETTTEAVDLFDAFDVFFVYAASNFSFFDDWTYDDATKTFTVTE

SEQ ID NO: 596 - protein, 56 aa, Artificial Sequence
  Synthetic polypeptide
TYKLILNGKTRKGEITTEAVDLFDAFDVFFVYAASNFSFFDDWTYDDATKTFTVTE

SEQ ID NO: 597 - protein, 56 aa, Artificial Sequence
  Synthetic polypeptide
TYKLILNGKTCKGEATTEAVDLFDAFDVFFVYAASNFSFIDDWTYDDATKTFTVTE

SEQ ID NO: 598 - protein, 56 aa, Artificial Sequence
  Synthetic polypeptide
TYKLILNGKTSKAEVTLEAVDLFDAFDVFFVYAASNFSFFDDWTYDDATKTFTVTE

SEQ ID NO: 599 - protein, 56 aa, Artificial Sequence
  Synthetic polypeptide
TYKLILNGKTGKVETTTEAVDLFDAFDVFFVYAASNFSFFDDWTYDDATKTFTVTE

SEQ ID NO: 600 - protein, 56 aa, Artificial Sequence
  Synthetic polypeptide
TYKVIMNGKTLKCESTIEAVDLFDAFDIVFVYYASNFSFIDDWTYDDATKTFTVTE

SEQ ID NO: 601 - protein, 56 aa, Artificial Sequence; identical to SEQ ID NO: 548
  Synthetic polypeptide

SEQ ID NO: 602 - protein, 56 aa, Artificial Sequence
  Synthetic polypeptide
TYKLILNGKTQKWETTTEAVDLFDAFDVFFVYAASNFSFFDDWTYDDATKTFTVTE

SEQ ID NO: 603 - protein, 56 aa, Artificial Sequence
  Synthetic polypeptide
TYKLILNGKTLKGEITAEAVDLFDAFDVFFVYAASNFSFFDDWTYDDATKTFTVTE

SEQ ID NO: 604 - protein, 56 aa, Artificial Sequence
  Synthetic polypeptide
TYKLILNGKTLKRETTTEAVDLFDAFDVFFVYAASNFSFFDDWTYDDATKTFTVTE

SEQ ID NO: 605 - protein, 56 aa, Artificial Sequence; identical to SEQ ID NO: 501
  Synthetic polypeptide

SEQ ID NO: 606 - protein, 56 aa, Artificial Sequence
  Synthetic polypeptide
TYKLILNGKTIKGEFTTEAVDLFDAFDVFFVYAASNFSFFDDWTYDDATKTFTVTE

SEQ ID NO: 607 - protein, 56 aa, Artificial Sequence
  Synthetic polypeptide
TYKLILNGKTVKCETTTEAVDLFDAFDVFFVYAASNFSFFDDWTYDDATKTFTVTE

SEQ ID NO: 608 - protein, 56 aa, Artificial Sequence
  Synthetic polypeptide
TYKLILNGKTHKSETTTEAVDLFDAFDVFFVYAASNFSFFDDWTYDDATKTFTVTE

SEQ ID NO: 609 - protein, 56 aa, Artificial Sequence; identical to SEQ ID NO: 376
  Synthetic polypeptide

SEQ ID NO: 610 - protein, 56 aa, Artificial Sequence
  Synthetic polypeptide
TYKMILNGKTLKSECTTEAVDLFEAFDVLYVCAASYFSVIDEWTYDDATKTFTVTE

SEQ ID NO: 611 - protein, 56 aa, Artificial Sequence
  Synthetic polypeptide
TYKMILNGKTLKSECTTEAVDFYDAFDVLFVCATSYFSVIDDWTYDDATKTFTVTE

SEQ ID NO: 612 - protein, 56 aa, Artificial Sequence
  Synthetic polypeptide
TYKMILNGKTLKSECTTEAVDLYDAFDVLYDCAASNFSFIEEWTYDDATKTFTVTE

SEQ ID NO: 613 - protein, 56 aa, Artificial Sequence
  Synthetic polypeptide
TYKMILNGKTLKSECTTEAVDLFDAFDVLWLCATAHFSIIDDWTYDDATKTFTVTE

SEQ ID NO: 614 - protein, 56 aa, Artificial Sequence
  Synthetic polypeptide
TYKMILNGKTLKSECTTEAVDLFQAFDVLYVCATSNFFIIDDWTYDDATKTFTVTE

SEQ ID NO: 615 - protein, 56 aa, Artificial Sequence
  Synthetic polypeptide
TYKMILNGKTLKSECTTEAVDLFTAFDVLSDCATSNFSIIDDWTYDDATKTFTVTE

SEQ ID NO: 616 - protein, 56 aa, Artificial Sequence
  Synthetic polypeptide
TYKMILNGKTLKSECTTEAVDLFDAFDVLFGCAATNFSYIDDWIYDDATKTFTVTE

SEQ ID NO: 617 - protein, 56 aa, Artificial Sequence
  Synthetic polypeptide
TYKMILNGKTLKSECTTEAVDVFEAFDVLFDCAIDNFTLIDDWTYDDATKTFTVTE

SEQ ID NO: 618 - protein, 56 aa, Artificial Sequence
  Synthetic polypeptide
TYKMILNGKTLKSECTTEAVDLFDAFDVLYDCASSNFYLIDDWTYDDATKTFTVTE

SEQ ID NO: 619 - protein, 56 aa, Artificial Sequence
  Synthetic polypeptide
TYKMILNGKTLKSECTTEAVDLFDAFGVLYDCAVDNFFFIDDWTYDDATKTFTVTE

SEQ ID NO: 620 - protein, 56 aa, Artificial Sequence
  Synthetic polypeptide
TYKMILNGKTLKSECTTEAVDLYAAFDVLFDCAAANFFIIDDWTYDDATKTFTVTE

SEQ ID NO: 621 - protein, 56 aa, Artificial Sequence
  Synthetic polypeptide
TYKMILNGKTLKSECTTEAVDFYDAFDVLFGCAASTFSIIDEWTYDDATKTFTVTE

SEQ ID NO: 622 - protein, 56 aa, Artificial Sequence
  Synthetic polypeptide
TYKMILNGKTLKSECTTEAVDFYDAFDVLFDCAASNFSYIDEWTYDDATKTFTVTE

SEQ ID NO: 623 - protein, 56 aa, Artificial Sequence
  Synthetic polypeptide
TYKMILNGKTLKSECTTEAVDLFEAFDVLFDCAASNFLVIDDWTYDDATKTFTVTE

SEQ ID NO: 624 - protein, 56 aa, Artificial Sequence
  Synthetic polypeptide
TYKMILNGKTLKSECTTEAVDWFEAFDVLFICATSKFSLIDDWTYDDATKTFTVTE

SEQ ID NO: 625 - protein, 56 aa, Artificial Sequence
  Synthetic polypeptide
TYKMILNGKTLKSECTTEAVDFFEAFDVLFGCAASNFTFIDDWTYDDATKTFTVTE

SEQ ID NO: 626 - protein, 56 aa, Artificial Sequence
  Synthetic polypeptide
TYKMILNGKTLKSECTTEAVDLFEAFDVLYVCAATNFPFIDDWTYDDATKTFTVTE

SEQ ID NO: 627 - protein, 56 aa, Artificial Sequence
  Synthetic polypeptide
TYKMILNGKTLKSECTTEAVDFFDAFEVLFACAAAHFSFIDDWTYDDATKTFTVTE

SEQ ID NO: 628 - protein, 56 aa, Artificial Sequence; identical to SEQ ID NO: 497
  Synthetic polypeptide

SEQ ID NO: 629 - protein, 56 aa, Artificial Sequence
  Synthetic polypeptide
TYKMILNGKTLKSECTTEAVDFFEAFDVLFVCAASIFTFIDDWTYDDATKTFTVTE

SEQ ID NO: 630 - protein, 56 aa, Artificial Sequence; identical to SEQ ID NO: 497
  Synthetic polypeptide

SEQ ID NO: 631 - protein, 56 aa, Artificial Sequence
  Synthetic polypeptide
TYKMILNGKTLKSECTTEAVDFFQAFDVLSGCAYSNFSFIDDWTYDDATKTFTVTE

SEQ ID NO: 632 - protein, 56 aa, Artificial Sequence
  Synthetic polypeptide
TYKMILNGKTLKSECTTEAVDRFEAFEVLFVCADSNFSFIDDWTYDDATKTFTVTE

SEQ ID NO: 633 - protein, 56 aa, Artificial Sequence
  Synthetic polypeptide
TYKMILNGKTLKSECTTEAVDLFQAFDVLYDCAASHFFLIDDWTYDDATKTFTVTE

SEQ ID NO: 634 - protein, 56 aa, Artificial Sequence
  Synthetic polypeptide
TYKLILNGKTLKGEFTLEAVDLFEAFDVFYAYAASEFFIFDDWRYDDATKTFTVTE

SEQ ID NO: 635 - protein, 56 aa, Artificial Sequence
  Synthetic polypeptide
TYKLILNGKTLKGEFTLEAVDLFDAFDVFFVYAASSFSTFDDWTYDDATKTFTVTE

SEQ ID NO: 636 - protein, 56 aa, Artificial Sequence
  Synthetic polypeptide
TYKLILNGKTLKGEFTLEAVDLFEAFDVFFVYAASTFPFFDDWTYDDATKTFTVTE

SEQ ID NO: 637 - protein, 56 aa, Artificial Sequence
  Synthetic polypeptide
TYKLILNGKTLKGEFTLEAVDLFDAFDVFFGYASATFASFDDWTYDDATKTFTVTE

SEQ ID NO: 638 - protein, 56 aa, Artificial Sequence
  Synthetic polypeptide
TYKLILNGKTLKGEFTLEAVDMFEAFDVFFVYAGSQFGFFEDWTYDDATKTFTVTE

SEQ ID NO: 639 - protein, 56 aa, Artificial Sequence
  Synthetic polypeptide
TYKLILNGKTLKGEFTLEAVDLFEAFDVFFGYAASYFSYFDDWTYDDATKTFTVTE

SEQ ID NO: 640 - protein, 56 aa, Artificial Sequence
  Synthetic polypeptide
TYKLILNGKTLKGEFTLEAVDLFEAFDVFFDYAASHFYYFDDWTYDDATKTFTVTE

SEQ ID NO: 641 - protein, 56 aa, Artificial Sequence
  Synthetic polypeptide
TYKLILNGKTLKGEFTLEAVDLFEAFDVFFVYAASNFSTFDDWTYDDATKTFTVTE

SEQ ID NO: 642 - protein, 56 aa, Artificial Sequence
  Synthetic polypeptide
TYKLILNGKTLKGEFTLEAVDQFHAFDVFYSYAASAFSYFDDWTYDDATKTFTVTE

SEQ ID NO: 643 - protein, 56 aa, Artificial Sequence
  Synthetic polypeptide
TYKLILNGKTLKGEFTLEAVDMFDAFEVFFGYAASNFTQFDDWTYDDATKTFTVTE

SEQ ID NO: 644 - protein, 56 aa, Artificial Sequence
  Synthetic polypeptide
TYKLILNGKTLKGEFTLEAVDLFEAFDVFYVYAASSFPLFDDWTYDDATKTFTVTE

SEQ ID NO: 645 - protein, 56 aa, Artificial Sequence
  Synthetic polypeptide
TYKLILNGKTLKGEFTLEAVDLFEAFDVFFSYASLKFQFFDDWTYDDATKTFTVTE

SEQ ID NO: 646 - protein, 56 aa, Artificial Sequence
  Synthetic polypeptide
TYKLILNGKTLKGEFTLEAVDVFEAFDVFFVYAASNFSFFDDWTYDDATKTFTVTE

SEQ ID NO: 647 - protein, 56 aa, Artificial Sequence; identical to SEQ ID NO: 636
  Synthetic polypeptide

SEQ ID NO: 648 - protein, 56 aa, Artificial Sequence
  Synthetic polypeptide
TYKMILNGKTLKSECTTEAVDLFHAFDVLFVCAATNFSIIDAWTYDDATKTFTVTE

SEQ ID NO: 649 - protein, 56 aa, Artificial Sequence; identical to SEQ ID NO: 500
  Synthetic polypeptide

SEQ ID NO: 650 - protein, 56 aa, Artificial Sequence
  Synthetic polypeptide
TYKMILNGKTLKSECTTEAVDLFDAFEVLYVCAASNFSYIAEWTYDDATKTFTVTE

SEQ ID NO: 651 - protein, 56 aa, Artificial Sequence
  Synthetic polypeptide
TYKMILNGKTLKSECTTEAVDFFDAFDVLFVCAASKFDVIDDWTYDDATKTFTVTE

SEQ ID NO: 652 - protein, 56 aa, Artificial Sequence
  Synthetic polypeptide
TYKMILNGKTLKSECTTEAVDLFDAFEVLYDCASSNFYLIDDWTYDDATKTFTVTE

SEQ ID NO: 653 - protein, 56 aa, Artificial Sequence
  Synthetic polypeptide
TYKMILNGKTLKSECTTEAVDLFDAFDVLFVCAISSFSLIDDWTYDDATKTFTVTE

SEQ ID NO: 654 - protein, 56 aa, Artificial Sequence
  Synthetic polypeptide
TYKMILNGKTLKSECTTEAVDLFDAFGVLYICAASNFSSISSWTYDDATKTFTVTE

SEQ ID NO: 655 - protein, 56 aa, Artificial Sequence
  Synthetic polypeptide
TYKMILNGKTLKSECTTEAVDLFDAFGVLFVCAAANFSSIADWTYDDATKTFTVTE

SEQ ID NO: 656 - protein, 56 aa, Artificial Sequence
  Synthetic polypeptide
TYKMILNGKTLKSECTTEAVDLYEAFDVLFVCAASSFSVIDDWTYDDATKTFTVTE

SEQ ID NO: 657 - protein, 56 aa, Artificial Sequence
  Synthetic polypeptide
TYKMILNGKTLKSECTTEAVDWFDAFDVLFVCATSHFSFIDDWTYDDATKTFTVTE

SEQ ID NO: 658 - protein, 56 aa, Artificial Sequence
  Synthetic polypeptide
TYKMILNGKTLKSECTTEAVDLFEAFDVLFVCAASNFTLIDDWTYDDATKTFTVTE

SEQ ID NO: 659 - protein, 56 aa, Artificial Sequence
  Synthetic polypeptide
TYKMILNGKTLKSECTTEAVDLFDAFDVLFSCAAPNFFDIDDWTYDDATKTFTVTE

SEQ ID NO: 660 - protein, 56 aa, Artificial Sequence
  Synthetic polypeptide
TYKMILNGKTLKSECTTEAVDLFEAFQVLFGCAASSFSIIDEWVYDDATKTFTVTE

SEQ ID NO: 661 - protein, 56 aa, Artificial Sequence
  Synthetic polypeptide
TYKMILNGKTLKSECTTEAVDLFEAFDVLFECAASNFFIIDAWTYDDATKTFTVTE

SEQ ID NO: 662 - protein, 56 aa, Artificial Sequence
  Synthetic polypeptide
TYKMILNGKTLKSECTTEAVDFFDAFHVLFVCAADNFAIIDEWTYDDATKTFTVTE

SEQ ID NO: 663 - protein, 56 aa, Artificial Sequence
  Synthetic polypeptide
TYKMILNGKTLKSECTTEAVDMYEAFDVLFDCAASNFAFIADWTYDDATKTFTVTE

SEQ ID NO: 664 - protein, 56 aa, Artificial Sequence
  Synthetic polypeptide
TYKMILNGKTLKSECTTEAVDMFDAFDVLFVCAASNFSFIDDWTYDDATKTFTVTE

SEQ ID NO: 665 - protein, 56 aa, Artificial Sequence
  Synthetic polypeptide
TYKMILNGKTLKSECTTEAVDLFAAFDVLFACAAASFSFIDDWTYDDATKTFTVTE

SEQ ID NO: 666 - protein, 56 aa, Artificial Sequence
  Synthetic polypeptide
TYKLILNGKTLKGEFTLEAVDLFEAFDVFFGYAASYFQVFDDWTYDDATKTFTVTE

SEQ ID NO: 667 - protein, 56 aa, Artificial Sequence
  Synthetic polypeptide
TYKLILNGKTLKGEFTLEAVDLFEAFDVFFVYAASNFSFFDDWTYDDATKTFTVTE

SEQ ID NO: 668 - protein, 56 aa, Artificial Sequence
  Synthetic polypeptide
TYKLILNGKTLKGEFTLEAVDQFEAFGVFFDYAASRFSLFSDWTYDDATKTFTVTE

SEQ ID NO: 669 - protein, 56 aa, Artificial Sequence
  Synthetic polypeptide
TYKMILNGKTLKSECTTEAVDLFEAFDVLFFCAASNFPIIDDWTYDDATKTFTVTE

SEQ ID NO: 670 - protein, 56 aa, Artificial Sequence
  Synthetic polypeptide
TYKMILNGKTLKSECTTEAVDFFDAFDVLYVCATSHFSFIDGWTYDDATKTFTVTE

SEQ ID NO: 671 - protein, 56 aa, Artificial Sequence
  Synthetic polypeptide
TYKMILNGKTLKSECTTEAVDLYDAFEVLYVCATSNFSFIEDWTYDDATKTFTVTE

SEQ ID NO: 672 - protein, 56 aa, Artificial Sequence
  Synthetic polypeptide
TYKMILNGKTLKSECTTEAVDFFEAFDVLFVCASSNFSLIEDWTYDDATKTFTVTE

SEQ ID NO: 673 - protein, 56 aa, Artificial Sequence; identical to SEQ ID NO: 497
  Synthetic polypeptide

SEQ ID NO: 674 - protein, 56 aa, Artificial Sequence; identical to SEQ ID NO: 498
  Synthetic polypeptide

SEQ ID NO: 675 - protein, 56 aa, Artificial Sequence; identical to SEQ ID NO: 499
  Synthetic polypeptide

SEQ ID NO: 676 - protein, 56 aa, Artificial Sequence
  Synthetic polypeptide
TYKMILNGKTLKSECTTEAVDWFDAFDVLYVCAASNFPIIDDWTYDDATKTFTVTE

SEQ ID NO: 677 - protein, 56 aa, Artificial Sequence
  Synthetic polypeptide
TYKMILNGKTLKSECTTEAVDFFEAFDVLFVCASSNFSFIDDWTYDDATKTFTVTE

SEQ ID NO: 678 - protein, 56 aa, Artificial Sequence; identical to SEQ ID NO: 611
  Synthetic polypeptide

SEQ ID NO: 679 - protein, 56 aa, Artificial Sequence
  Synthetic polypeptide
TYKMILNGKTLKSECTTEAVDMFEAFDVLFLCATSVFPIIDDWTYDDATKTFTVTE

SEQ ID NO: 680 - protein, 56 aa, Artificial Sequence; identical to SEQ ID NO: 657
  Synthetic polypeptide

SEQ ID NO: 681 - protein, 56 aa, Artificial Sequence
  Synthetic polypeptide
TYKMILNGKTLKSECTTEAVDLFEAFDVLYECAAYNFSFIEDWTYDDATKTFTVTE

SEQ ID NO: 682 - protein, 56 aa, Artificial Sequence
  Synthetic polypeptide
TYKMILNGKTLKSECTTEAVDLFDAFDVLFECAERNFSFIDDWTYDDATKTFTVTE

SEQ ID NO: 683 - protein, 56 aa, Artificial Sequence
  Synthetic polypeptide
TYKMILNGKTLKSECTTEAVDVFEAFDVLYGCAASNFTYIVDWTYDDATKTFTVTE

SEQ ID NO: 684 - protein, 56 aa, Artificial Sequence
  Synthetic polypeptide
TYKMILNGKTLKSECTTEAVDMFDAFDVLFDCAASNFSEINDWTYDDATKTFTVTE

SEQ ID NO: 685 - protein, 56 aa, Artificial Sequence
  Synthetic polypeptide
TYKMILNGKTLKSECTTEAVDMYDAFGVLFVCAESKFSLIDDWTYDDATKTFTVTE

SEQ ID NO: 686 - protein, 56 aa, Artificial Sequence
  Synthetic polypeptide
TYKMILNGKTLKSECTTEAVDWYEAFDVLFVCAAANFRFIDDWTYDDATKTFTVTE

SEQ ID NO: 687 - protein, 56 aa, Artificial Sequence
  Synthetic polypeptide
TYKMILNGKTLKSECTTEAVDLFDAFDVLYECAASKFFIIEDWTYDDATKTFTVTE

SEQ ID NO: 688 - protein, 56 aa, Artificial Sequence
  Synthetic polypeptide
TYKMILNGKTLKSECTTEAVDLFDAFDVLYVCATSYFPLIADWTYDDATKTFTVTE

SEQ ID NO: 689 - protein, 56 aa, Artificial Sequence; identical to SEQ ID NO: 611
  Synthetic polypeptide

SEQ ID NO: 690 - protein, 56 aa, Artificial Sequence
  Synthetic polypeptide
TYKMILNGKTLKSECTTEAVDLYEAFDVLFDCAASKFSLIEDWTYDDATKTFTVTE

SEQ ID NO: 691 - protein, 56 aa, Artificial Sequence
  Synthetic polypeptide
TYKMILNGKTLKSECTTEAVDWFEAFDVLFVCAASNFSVIQDWTYDDATKTFTVTE

SEQ ID NO: 692 - protein, 56 aa, Artificial Sequence
  Synthetic polypeptide
TYKLILNGKTLKGEFTLEAVDLHEAFDVFFGYAASNFSDFSDWTYDDATKTFTVTE

SEQ ID NO: 693 - protein, 56 aa, Artificial Sequence
  Synthetic polypeptide
TYKLILNGKTLKGEFTLEAVDLFDAFDVFFEYASSYFYYFEDWTYDDATKTFTVTE

SEQ ID NO: 694 - protein, 56 aa, Artificial Sequence; identical to SEQ ID NO: 376
  Synthetic polypeptide

SEQ ID NO: 695 - protein, 56 aa, Artificial Sequence
  Synthetic polypeptide
TYKLILNGKTLKGEFTLEAVDLFDAFDVFFGYAASIFSDFDDWTYDDATKTFTVTE

SEQ ID NO: 696 - protein, 56 aa, Artificial Sequence
  Synthetic polypeptide
TYKLILNGKTLKGEFTLEAVDLFDAFDVFFVYASAHFSYFDDWTYDDATKTFTVTE

SEQ ID NO: 697 - protein, 56 aa, Artificial Sequence
  Synthetic polypeptide
TYKLILNGKTLKGEFTLEAVDFFEAFDVFFAYAANKFSYFDDWTYDDATKTFTVTE

SEQ ID NO: 698 - protein, 56 aa, Artificial Sequence
  Synthetic polypeptide
TYKLILNGKTLKGEFTLEAVDLYEAFDVFFDYAASHFSVFDDWTYDDATKTFTVTE

SEQ ID NO: 699 - protein, 56 aa, Artificial Sequence
  Synthetic polypeptide
TYKLILNGKTLKGEFTLEAVDVFEAFDVFFVYATSNFAVFDDWTYDDATKTFTVTE

SEQ ID NO: 700 - protein, 56 aa, Artificial Sequence; identical to SEQ ID NO: 376
  Synthetic polypeptide

SEQ ID NO: 701 - protein, 56 aa, Artificial Sequence; identical to SEQ ID NO: 376
  Synthetic polypeptide

SEQ ID NO: 702 - protein, 56 aa, Artificial Sequence; identical to SEQ ID NO: 636
  Synthetic polypeptide

SEQ ID NO: 703 - protein, 56 aa, Artificial Sequence; identical to SEQ ID NO: 646
  Synthetic polypeptide

SEQ ID NO: 704 - protein, 56 aa, Artificial Sequence
  Synthetic polypeptide
TYKLILNGKTLKGEFTLEAVDVFEAFDVFFVYATANFLSIDDWTYDDATKTFTVTE

SEQ ID NO: 705 - protein, 56 aa, Artificial Sequence
  Synthetic polypeptide
TYKLILNGKTLKGEFTLEAVDLFDAFDVFFVYAASNFSYFDDWTYDDATKTFTVTE

SEQ ID NO: 706 - protein, 56 aa, Artificial Sequence; identical to SEQ ID NO: 500
  Synthetic polypeptide

SEQ ID NO: 707 - protein, 56 aa, Artificial Sequence
  Synthetic polypeptide
TYKMILNGKTLKSECTTEAVDIFHAFDVLFDCAANSFPIIEDWTYDDATKTFTVTE

SEQ ID NO: 708 - protein, 56 aa, Artificial Sequence
  Synthetic polypeptide
TYKMILNGKTLKSECTTEAVDFFDAFDVLFVCAVSNFSDIAEWTYDDATKTFTVTE

SEQ ID NO: 709 - protein, 56 aa, Artificial Sequence
  Synthetic polypeptide
TYKMILNGKTLKSECTTEAVDVFAAFHVLFVCAASNFSHIEDWTYDDATKTFTVTE

SEQ ID NO: 710 - protein, 56 aa, Artificial Sequence
  Synthetic polypeptide
TYKMILNGKTLKSECTTEAVDLFDAFDVLFVCAASNFSIIDDWTYDDATKTFTVTE

SEQ ID NO: 711 - protein, 56 aa, Artificial Sequence
  Synthetic polypeptide
TYKMILNGKTLKSECTTEAVDLFEAFDVLFVCAVANFAYIDEWTYDDATKTFTVTE

SEQ ID NO: 712 - protein, 56 aa, Artificial Sequence
  Synthetic polypeptide
TYKMILNGKTLKSECTTEAVDMFDAFEVLFDCAASKFSVIDDWTYDDATKTFTVTE

SEQ ID NO: 713 - protein, 56 aa, Artificial Sequence
  Synthetic polypeptide
TYKMILNGKTLKSECTTEAVDWIHAFDVLFVCATSNFSYIDDWTYDDATKTFTVTE

SEQ ID NO: 714 - protein, 56 aa, Artificial Sequence
  Synthetic polypeptide
TYKMILNGKTLKSECTTEAVDLFEAFDVLFVCAASNFNVIDDWTYDDATKTFTVTE

SEQ ID NO: 715 - protein, 56 aa, Artificial Sequence
  Synthetic polypeptide
TYKMILNGKTLKSECTTEAVDLYEAMDVLFDCATSHFFVIDAWDYDDATKTFTVTE

SEQ ID NO: 716 - protein, 56 aa, Artificial Sequence
  Synthetic polypeptide
TYKMILNGKTLKSECTTEAVDLFHAFDVLFPCAYSNFSIIDDWTYDDATKTFTVTE

SEQ ID NO: 717 - protein, 56 aa, Artificial Sequence
  Synthetic polypeptide
TYKMILNGKTLKSECTTEAVDLFDAFDVLYACAASTFSIIDEWTYDDATKTFTVTE

SEQ ID NO: 718 - protein, 56 aa, Artificial Sequence
  Synthetic polypeptide
TYKMILNGKTLKSECTTEAVDMFEAFDVLYVCASSEFNNIADWTYDDATKTFTVTE

SEQ ID NO: 719 - protein, 56 aa, Artificial Sequence
  Synthetic polypeptide
TYKMILNGKTLKSECTTEAVDFFDAFDVLYDCAASNFSFIDDWTYDDATKTFTVTE

SEQ ID NO: 720 - protein, 56 aa, Artificial Sequence
  Synthetic polypeptide
TYKMILNGKTLKSECTTEAVDMFHAFDVLLLCASSNFSVIDDWTYDDATKTFTVTE

SEQ ID NO: 721 - protein, 56 aa, Artificial Sequence
  Synthetic polypeptide
TYKMILNGKTLKSECTTEAVDLFDAFDVLYDCAISKFEIIDDWTYDDATKTFTVTE

SEQ ID NO: 722 - protein, 56 aa, Artificial Sequence
  Synthetic polypeptide
TYKMILNGKTLKSECTTEAVDVYAAFDVLWVCASSKFQFIDDWTYDDATKTFTVTE

SEQ ID NO: 723 - protein, 56 aa, Artificial Sequence
  Synthetic polypeptide
TYKMILNGKTLKSECTTEAVDLFDAFDVLFVCATSDFSDIEDWTYDDATKTFTVTE

SEQ ID NO: 724 - protein, 56 aa, Artificial Sequence
  Synthetic polypeptide
TYKMILNGKTLKSECTTEAVDLFEAFDVLLVCASSSFSVIEDWTYDDATKTFTVTE

SEQ ID NO: 725 - protein, 56 aa, Artificial Sequence
  Synthetic polypeptide
TYKMILNGKTLKSECTTEAVDLFDAFEVLFICAASYFSVIDDWTYDDATKTFTVTE

SEQ ID NO: 726 - protein, 56 aa, Artificial Sequence
  Synthetic polypeptide
TYKMILNGKTLKSECTTEAVDWFDAFEVLWVCAASNFPFIEDWTYDDATKTFTVTE

SEQ ID NO: 727 - protein, 56 aa, Artificial Sequence; identical to SEQ ID NO: 617
  Synthetic polypeptide

SEQ ID NO: 728 - protein, 56 aa, Artificial Sequence
  Synthetic polypeptide
TYKMILNGKTLKSECTTEAVDLYDAFDVLFVCASSNFPVIDDWTYDDATKTFTVTE

SEQ ID NO: 729 - protein, 56 aa, Artificial Sequence
  Synthetic polypeptide
TYKMILNGKTLKSECTTEAVDLYDAFEVLSVCAYSTFPFIEDWTYDDATKTFTVTE

SEQ ID NO: 730 - protein, 56 aa, Artificial Sequence
  Synthetic polypeptide
TYKMILNGKTLKSECTTEAVDFYEAFDVLFDCASSNFSLIDDWTYDDATKTFTVTE

SEQ ID NO: 731 - protein, 56 aa, Artificial Sequence
  Synthetic polypeptide
TYKMILNGKTLKSECTTEAVDMFNAFDVLYDCATSYFTFIEDWSYDDATKTFTVTE

SEQ ID NO: 732 - protein, 56 aa, Artificial Sequence
  Synthetic polypeptide
TYKMILNGKTLKSECTTEAVDLFEAFDVLFVCAASHFSVIDDWTYDDATKTFTVTE

SEQ ID NO: 733 - protein, 56 aa, Artificial Sequence
  Synthetic polypeptide
TYKMILNGKTLKSECTTEAVDWFDAVDVLFDCAASNFSVIDDWTYDDATKTFTVTE

SEQ ID NO: 734 - protein, 56 aa, Artificial Sequence
  Synthetic polypeptide
TYKMILNGKTLKSECTTEAVDFFAAFDVLFVCAATHFSFIDDWTYDDATKTFTVTE

SEQ ID NO: 735 - protein, 56 aa, Artificial Sequence
  Synthetic polypeptide
TYKMILNGKTLKSECTTEAVDLFDAFDVLFVCASTIFPVIEDWTYDDATKTFTVTE

SEQ ID NO: 736 - protein, 56 aa, Artificial Sequence
  Synthetic polypeptide
TYKMILNGKTLKSECTTEAVDLFEAFDVLFICASSHFSFIDDWTYDDATKTFTVTE

SEQ ID NO: 737 - protein, 56 aa, Artificial Sequence
  Synthetic polypeptide
TYKMILNGKTLKSECTTEAVDFFEAFDVLFGCATTEFSLIDDWTYDDATKTFTVTE

SEQ ID NO: 738 - protein, 56 aa, Artificial Sequence
  Synthetic polypeptide
TYKMILNGKTLKSECTTEAVDLFDAFDVLFVCASYHFTVIDDWTYDDATKTFTVTE

SEQ ID NO: 739 - protein, 56 aa, Artificial Sequence
  Synthetic polypeptide
TYKMILNGKTLKSECTTEAVDLFVAFDVLFDCAESHFSFIDDWTYDDATKTFTVTE

SEQ ID NO: 740 - protein, 56 aa, Artificial Sequence
  Synthetic polypeptide
TYKMILNGKTLKSECTTEAVDLFDAFDVLYLCAASNFSLIDEWTYDDATKTFTVTE

SEQ ID NO: 741 - protein, 56 aa, Artificial Sequence
  Synthetic polypeptide
TYKMILNGKTLKSECTTEAVDWYAAFDVLFICATSHFSFIDDWTYDDATKTFTVTE

SEQ ID NO: 742 - protein, 56 aa, Artificial Sequence
  Synthetic polypeptide
TYKMILNGKTLKSECTTEAVDLFEAFGVLLVCASSNFYSIEDWTYDDATKTFTVTE

SEQ ID NO: 743 - protein, 56 aa, Artificial Sequence
  Synthetic polypeptide
TYKMILNGKTLKSECTTEAVDFFDAFAVLFSCATSNFSLIDDWTYDDATKTFTVTE

SEQ ID NO: 744 - protein, 56 aa, Artificial Sequence
  Synthetic polypeptide
TYKMILNGKTLKSECTTEAVDWNQAFDVLFLCATSRFSFIDDWTYDDATKTFTVTE

SEQ ID NO: 745 - protein, 56 aa, Artificial Sequence
  Synthetic polypeptide
TYKMILNGKTLKSECTTEAVDFFQAFDVLFICATNTFSFIDDWTYDDATKTFTVTE

SEQ ID NO: 746 - protein, 56 aa, Artificial Sequence
  Synthetic polypeptide
TYKMILNGKTLKSECTTEAVDFFGAFDVLSVCAYSKFPFIEDWTYDDATKTFTVTE

SEQ ID NO: 747 - protein, 56 aa, Artificial Sequence
  Synthetic polypeptide
TYKMILNGKTLKSECTTEAVDLFDAFDVLYVCATSHFPVIDDWIYDDATKTFTVTE

SEQ ID NO: 748 - protein, 56 aa, Artificial Sequence
  Synthetic polypeptide
TYKMILNGKTLKSECTTEAVDFFDAFDVLFLCATSKFSVIDDWTYDDATKTFTVTE

SEQ ID NO: 749 - protein, 56 aa, Artificial Sequence
  Synthetic polypeptide
TYKMILNGKTLKSECTTEAVDLFEAFDVLFVCAASTFSIIDDWTYDDATKTFTVTE

SEQ ID NO: 750 - protein, 56 aa, Artificial Sequence
  Synthetic polypeptide
TYKMILNGKTLKSECTTEAVDLFEAFDVLFTCAASHFSVIDDWIYDDATKTFTVTE

SEQ ID NO: 751 - protein, 56 aa, Artificial Sequence
  Synthetic polypeptide
TYKMILNGKTLKSECTTEAVDLFDAFDVLFVCAASQFSIIDDWTYDDATKTFTVTE

SEQ ID NO: 752 - protein, 56 aa, Artificial Sequence
  Synthetic polypeptide
TYKMILNGKTLKSECTTEAVDMFEAFEVLYDCAASHFGFIEDWTYDDATKTFTVTE

SEQ ID NO: 753 - protein, 56 aa, Artificial Sequence
  Synthetic polypeptide
TYKMILNGKTLKSECTTEAVDIFNAFDVLFMCAASHFPIIDDWTYDDATKTFTVTE

SEQ ID NO: 754 - protein, 56 aa, Artificial Sequence
  Synthetic polypeptide
TYKMILNGKTLKSECTTEAVDLFEAFDVLFVCAVSNFSVIDDWTYDDATKTFTVTE

SEQ ID NO: 755 - protein, 56 aa, Artificial Sequence
  Synthetic polypeptide
TYKMILNGKTLKSECTTEAVDLFDAFDVLFVCAASSFSIIDDWTYDDATKTFTVTE

SEQ ID NO: 756 - protein, 56 aa, Artificial Sequence; identical to SEQ ID NO: 718
  Synthetic polypeptide

SEQ ID NO: 757 - protein, 56 aa, Artificial Sequence
  Synthetic polypeptide
TYKMILNGKTLKSECTTEAVDFFDAFDVLFVCAASNFAIIDDWTYDDATKTFTVTE

SEQ ID NO: 758 - protein, 56 aa, Artificial Sequence
  Synthetic polypeptide
TYKMILNGKTLKSECTTEAVDFFDAFDVLFVCAATHFPIIADWTYDDATKTFTVTE

SEQ ID NO: 759 - protein, 56 aa, Artificial Sequence
  Synthetic polypeptide
TYKMILNGKTLKSECTTEAVDLFDAFDVLYDCASSNFALIDDWTYDDATKTFTVTE

SEQ ID NO: 760 - protein, 56 aa, Artificial Sequence; identical to SEQ ID NO: 622
  Synthetic polypeptide

SEQ ID NO: 761 - protein, 56 aa, Artificial Sequence
  Synthetic polypeptide
TYKMILNGKTLKSECTTEAVDWFDAFDVLFHCAASSFEIIDDWTYDDATKTFTVTE

SEQ ID NO: 762 - protein, 56 aa, Artificial Sequence; identical to SEQ ID NO: 710
  Synthetic polypeptide

SEQ ID NO: 763 - protein, 56 aa, Artificial Sequence
  Synthetic polypeptide
TYKMILNGKTLKSECTTEAVDLFDAFDVLFDCAASYFYSIEDWTYDDATKTFTVTE

SEQ ID NO: 764 - protein, 56 aa, Artificial Sequence
  Synthetic polypeptide
TYKMILNGKTLKSECTTEAVDLYEAFDVLFGCASSSFSLIDDWTYDDATKTFTVTE

SEQ ID NO: 765 - protein, 56 aa, Artificial Sequence
  Synthetic polypeptide
TYKMILNGKTLKSECTTEAVDLFEAFDVLFDCAASSFSIIDDWTYDDATKTFTVTE

SEQ ID NO: 766 - protein, 56 aa, Artificial Sequence
  Synthetic polypeptide
TYKMILNGKTLKSECTTEAVDLFHAFDVLFACAASNFSEIDDWTYDDATKTFTVTE

SEQ ID NO: 767 - protein, 56 aa, Artificial Sequence
  Synthetic polypeptide
TYKMILNGKTLKSECTTEAVDLYEAFDVLFVCAASNFSVIDDWTYDDATKTFTVTE

SEQ ID NO: 768 - protein, 56 aa, Artificial Sequence
  Synthetic polypeptide
TYKMILNGKTLKSECTTEAVDFYDAFDVLFDCALSNFYLIDDWTYDDATKTFTVTE

SEQ ID NO: 769 - protein, 56 aa, Artificial Sequence
  Synthetic polypeptide
TYKMILNGKTLKSECTTEAVDLFEAFDVLFVCAASEFSIIDEWTYDDATKTFTVTE

SEQ ID NO: 770 - protein, 56 aa, Artificial Sequence
  Synthetic polypeptide
TYKMILNGKTLKSECTTEAVDFFEAFDVLFVCAAFNFSLIDDWTYDDATKTFTVTE

SEQ ID NO: 771 - protein, 56 aa, Artificial Sequence
  Synthetic polypeptide
TYKMILNGKTLKSECTTEAVDFFVAFDVLSECAYSNFSFIDDWTYDDATKTFTVTE

SEQ ID NO: 772 - protein, 56 aa, Artificial Sequence
  Synthetic polypeptide
TYKMILNGKTLKSECTTEAVDFFEAFDVLFDCAASKFSVIDDWTYDDATKTFTVTE

SEQ ID NO: 773 - protein, 56 aa, Artificial Sequence
  Synthetic polypeptide
TYKMILNGKTLKSECTTEAVDMFEAFDVLFECAASNFSFIEDWTYDDATKTFTVTE

SEQ ID NO: 774 - protein, 56 aa, Artificial Sequence
  Synthetic polypeptide
TYKMILNGKTLKSECTTEAVDLFDAIEVLYDCAATTFYDIDDWTYDDATKTFTVTE

SEQ ID NO: 775 - protein, 56 aa, Artificial Sequence
  Synthetic polypeptide
TYKMILNGKTLKSECTTEAVDLYEAFDVLYVCASSNFPLIDDWTYDDATKTFTVTE

SEQ ID NO: 776 - protein, 56 aa, Artificial Sequence
  Synthetic polypeptide
TYKMILNGKTLKSECTTEAVDLFEAFDVLFICAASTFPLIDNWTYDDATKTFTVTE

SEQ ID NO: 777 - protein, 56 aa, Artificial Sequence
  Synthetic polypeptide
TYKMILNGKTLKSECTTEAVDWFDAFDVLFVCATSNFAFIDDWTYDDATKTFTVTE

SEQ ID NO: 778 - protein, 56 aa, Artificial Sequence
  Synthetic polypeptide
TYKMILNGKTLKSECTTEAVDMYEAFDVLYVCAATNFSVIDEWTYDDATKTFTVTE

SEQ ID NO: 779 - protein, 56 aa, Artificial Sequence
  Synthetic polypeptide
TYKMILNGKTLKSECTTEAVDLFDAFDVLYSCAVSHFSVIDDWTYDDATKTFTVTE

SEQ ID NO: 780 - protein, 56 aa, Artificial Sequence
  Synthetic polypeptide
TYKMILNGKTLKSECTTEAVDMFQAFDVLFGCAATTFPVIDDWTYDDATKTFTVTE

SEQ ID NO: 781 - protein, 56 aa, Artificial Sequence
  Synthetic polypeptide
TYKMILNGKTLKSECTTEAVDFFEAFDVLFVCAASSFTLIDDWTYDDATKTFTVTE

SEQ ID NO: 782 - protein, 56 aa, Artificial Sequence; identical to SEQ ID NO: 95
  Synthetic polypeptide

SEQ ID NO: 783 - protein, 56 aa, Artificial Sequence; identical to SEQ ID NO: 547
  Synthetic polypeptide

SEQ ID NO: 784 - protein, 56 aa, Artificial Sequence; identical to SEQ ID NO: 548
  Synthetic polypeptide

SEQ ID NO: 785 - protein, 56 aa, Artificial Sequence; identical to SEQ ID NO: 557
  Synthetic polypeptide

SEQ ID NO: 786 - protein, 56 aa, Artificial Sequence
  Synthetic polypeptide
TYKLILNGKTLKGEFTTEAVDLFDAFDVFFVYAASNFSFFDDWTYDDATKTFTVTE

SEQ ID NO: 787 - protein, 56 aa, Artificial Sequence
  Synthetic polypeptide
TYKLILNGKTLKGETTVEAVDLFDAFDVFFVYAASNFSFFDDWTYDDATKTFTVTE

SEQ ID NO: 788 - protein, 56 aa, Artificial Sequence
  Synthetic polypeptide
TYKLILNGKTLKGETTIEAVDLFDAFDVFFVYAASNFSFFDDWTYDDATKTFTVTE

SEQ ID NO: 789 - protein, 56 aa, Artificial Sequence
  Synthetic polypeptide
TYKLILNGKTLKGETTLEAVDLFDAFDVFFVYAASNFSFFDDWTYDDATKTFTVTE

SEQ ID NO: 790 - protein, 56 aa, Artificial Sequence
  Synthetic polypeptide
TYKLILNGKTLKGETTFEAVDLFDAFDVFFVYAASNFSFFDDWTYDDATKTFTVTE

SEQ ID NO: 791 - protein, 56 aa, Artificial Sequence; identical to SEQ ID NO: 567
  Synthetic polypeptide

SEQ ID NO: 792 - protein, 56 aa, Artificial Sequence
  Synthetic polypeptide
TYKLILNGKTLKGEVTIEAVDLFDAFDVFFVYAASNFSFFDDWTYDDATKTFTVTE

SEQ ID NO: 793 - protein, 56 aa, Artificial Sequence; identical to SEQ ID NO: 563
  Synthetic polypeptide

SEQ ID NO: 794 - protein, 56 aa, Artificial Sequence
  Synthetic polypeptide
TYKLILNGKTLKGEVTFEAVDLFDAFDVFFVYAASNFSFFDDWTYDDATKTFTVTE

SEQ ID NO: 795 - protein, 56 aa, Artificial Sequence
  Synthetic polypeptide
TYKLILNGKTLKGEITVEAVDLFDAFDVFFVYAASNFSFFDDWTYDDATKTFTVTE

SEQ ID NO: 796 - protein, 56 aa, Artificial Sequence
  Synthetic polypeptide
TYKLILNGKTLKGEITIEAVDLFDAFDVFFVYAASNFSFFDDWTYDDATKTFTVTE

SEQ ID NO: 797 - protein, 56 aa, Artificial Sequence
  Synthetic polypeptide
TYKLILNGKTLKGEITLEAVDLFDAFDVFFVYAASNFSFFDDWTYDDATKTFTVTE

SEQ ID NO: 798 - protein, 56 aa, Artificial Sequence
  Synthetic polypeptide
TYKLILNGKTLKGEITFEAVDLFDAFDVFFVYAASNFSFFDDWTYDDATKTFTVTE

SEQ ID NO: 799 - protein, 56 aa, Artificial Sequence
  Synthetic polypeptide
TYKLILNGKTLKGELTVEAVDLFDAFDVFFVYAASNFSFFDDWTYDDATKTFTVTE

SEQ ID NO: 800 - protein, 56 aa, Artificial Sequence
  Synthetic polypeptide
TYKLILNGKTLKGELTIEAVDLFDAFDVFFVYAASNFSFFDDWTYDDATKTFTVTE

SEQ ID NO: 801 - protein, 56 aa, Artificial Sequence
  Synthetic polypeptide
TYKLILNGKTLKGELTLEAVDLFDAFDVFFVYAASNFSFFDDWTYDDATKTFTVTE

SEQ ID NO: 802 - protein, 56 aa, Artificial Sequence
  Synthetic polypeptide
TYKLILNGKTLKGELTFEAVDLFDAFDVFFVYAASNFSFFDDWTYDDATKTFTVTE

SEQ ID NO: 803 - protein, 56 aa, Artificial Sequence
  Synthetic polypeptide
TYKLILNGKTLKGEFTVEAVDLFDAFDVFFVYAASNFSFFDDWTYDDATKTFTVTE

SEQ ID NO: 804 - protein, 56 aa, Artificial Sequence
  Synthetic polypeptide
TYKLILNGKTLKGEFTIEAVDLFDAFDVFFVYAASNFSFFDDWTYDDATKTFTVTE

SEQ ID NO: 805 - protein, 56 aa, Artificial Sequence
  Synthetic polypeptide
TYKLILNGKTLKGEFTFEAVDLFDAFDVFFVYAASNFSFFDDWTYDDATKTFTVTE

SEQ ID NO: 806 - protein, 56 aa, Artificial Sequence; identical to SEQ ID NO: 497
  Synthetic polypeptide

SEQ ID NO: 807 - protein, 56 aa, Artificial Sequence; identical to SEQ ID NO: 498
  Synthetic polypeptide

SEQ ID NO: 808 - protein, 56 aa, Artificial Sequence; identical to SEQ ID NO: 499
  Synthetic polypeptide

SEQ ID NO: 809 - protein, 56 aa, Artificial Sequence; identical to SEQ ID NO: 500
  Synthetic polypeptide

SEQ ID NO: 810 - protein, 57 aa, Artificial Sequence
  Synthetic polypeptide
TYVLFLFDSDSYADYTVTEAVDAATAEKVFKQYANDNGVDGEWTYDDATKTFFVDVD

SEQ ID NO: 811 - protein, 57 aa, Artificial Sequence
  Synthetic polypeptide
TYVLFLFGSDSYADYTVTEAVDAATAEKVFKQYANDNGVDGEWTYDDATKTFFVDVD

SEQ ID NO: 812 - protein, 57 aa, Artificial Sequence
  Synthetic polypeptide
TYVLFLFDSDSYADYTDTEAVDAATAEKVFKQYANDNGVDGEWTYDDATKTFFVDVD

SEQ ID NO: 813 - protein, 57 aa, Artificial Sequence
  Synthetic polypeptide
TYVLFLFDSDSYADYTVTEAVDAATAEKVFKQYANDNGVDGEWTYDDATKTFFVEVD

SEQ ID NO: 814 - protein, 56 aa, Artificial Sequence
  Synthetic polypeptide
TYKLILNGKTLKGETTTEAVMFLDAFSVFFSYASSNSSDYVSWTYDDATKTFTVTE

SEQ ID NO: 815 - protein, 56 aa, Artificial Sequence
  Synthetic polypeptide
TYKLILNGKTLKGETTTEAVPFLHAFSVFFSYASSNSSDYVSWTYDDATKTFTVTE

SEQ ID NO: 816 - protein, 56 aa, Artificial Sequence
  Synthetic polypeptide
TYKLILNGKTLKGETTTEAVTFLRAFSVFFSYASSNSSDYVSWTYDDATKTFTVTE

SEQ ID NO: 817 - protein, 56 aa, Artificial Sequence
  Synthetic polypeptide
TYKLILNGKTLKGETTTEAVQFLPAFSVFFSYASSNSSDYVSWTYDDATKTFTVTE

SEQ ID NO: 818 - protein, 56 aa, Artificial Sequence
  Synthetic polypeptide
TYKLILNGKTLKGETTTEAVGFLRAFSVFFSYASSNSSDYVSWTYDDATKTFTVTE

SEQ ID NO: 819 - protein, 56 aa, Artificial Sequence
  Synthetic polypeptide
TYKLILNGKTLKGETTTEAVPFIRAFSVFFSYASSNSSDYVSWTYDDATKTFTVTE

SEQ ID NO: 820 - protein, 56 aa, Artificial Sequence
  Synthetic polypeptide
TYKLILNGKTLKDETTTEAVSFIRAFSVFFSYASSNSSDYVSWTYDDATKTFTVTE

SEQ ID NO: 821 - protein, 56 aa, Artificial Sequence
  Synthetic polypeptide
TYKLILNGKTLKGETTTEAVPFLGAFSVFFSYASSNSSDYVSWTYDDATKTFTVTE

SEQ ID NO: 822 - protein, 56 aa, Artificial Sequence
  Synthetic polypeptide
TYKLILNGKTLKGETTTEAVPFISAFSVFFSYASSNSSDYVSWTYDDATKTFTVTE

SEQ ID NO: 823 - protein, 56 aa, Artificial Sequence
  Synthetic polypeptide
TYKLILNGKTLKGETTTEAVPFLNAFSVFFSYASSNSSDYVSWTYDDATKTFTVTE

SEQ ID NO: 824 - protein, 56 aa, Artificial Sequence
  Synthetic polypeptide
TYKLILNGKTLKGETTTEAVGFMRAFSVFFSYASSNSSDYVSWTYNDATKTFTVTE

SEQ ID NO: 825 - protein, 56 aa, Artificial Sequence
  Synthetic polypeptide
TYKLILNGKTLKGETTTEAVGFMSAFSVFFSYASSNSSDYVSWTYDDATKTFTVTE

SEQ ID NO: 826 - protein, 56 aa, Artificial Sequence
  Synthetic polypeptide
TYKLILNGKTLKGETTTEAVGFLNAFSVFFSYASSNSSDYVSWTYDDATKTFTVTE

SEQ ID NO: 827 - protein, 56 aa, Artificial Sequence
  Synthetic polypeptide
TYKLILNGKTLKGETTTEAVDFYYAFSVFFNYASSNSSDYVSWTYDDATKTFTVTE

SEQ ID NO: 828 - protein, 56 aa, Artificial Sequence
  Synthetic polypeptide
TYKLILNGKTLKGETTTEAVDFYYAFSVFFGYASSNSSDYVSWTYDDATKTFTVTE

SEQ ID NO: 829 - protein, 56 aa, Artificial Sequence
  Synthetic polypeptide
TYKLILNGKTLKGETTTEAVDFYYALGVFVAYASSNSSDYVSWTYDDATKTFTVTE

SEQ ID NO: 830 - protein, 56 aa, Artificial Sequence
  Synthetic polypeptide
TYKLILNGKTLKGETTTEAVDFYYALGVFVEYASSNSSDYVSWTYDDATKTFTVTE

SEQ ID NO: 831 - protein, 56 aa, Artificial Sequence
  Synthetic polypeptide
TYKLILNGKTLKGETTTEAVDFYYAFGVFLDYASSNSSDYVSWTYDDATKTFTVTE

SEQ ID NO: 832 - protein, 56 aa, Artificial Sequence
  Synthetic polypeptide
TYKLILNGKTLKGETTTEAVDFYYALGVFVNYASSNSSDYVSWTYDDATKTFTVTE

SEQ ID NO: 833 - protein, 56 aa, Artificial Sequence
  Synthetic polypeptide
TYKLILNGKTLKGETTTEAVDFYYAFGVFMDYASSNSSDYVSWTYDDATKTFTVTE

SEQ ID NO: 834 - protein, 56 aa, Artificial Sequence
  Synthetic polypeptide
TYKLILNGKTLKGETTTEAVDFYYALGVFVDYASSNSSDYVSWTYDDATKTFTVTE

SEQ ID NO: 835 - protein, 56 aa, Artificial Sequence
  Synthetic polypeptide
TYKLILNGKTLKGETTTEAVDFYYAFSVFFSYATTNGSDYVSWTYDDATKTFTVTE

SEQ ID NO: 836 - protein, 56 aa, Artificial Sequence
  Synthetic polypeptide
TYKLILNGKTLKGETTTEAVDFYYAFSVFFSYAVSNGHDYVSWTYDDATKTFTVTE

SEQ ID NO: 837 - protein, 56 aa, Artificial Sequence
  Synthetic polypeptide
TYKLILNGKTLKGETTTEAVDFYYAFSVFFSYAEANRRDYVSWTYDDATKTFTVTE

SEQ ID NO: 838 - protein, 56 aa, Artificial Sequence
  Synthetic polypeptide
TYKLILNGKTLKGETTTEAVDFYYAFSVFFSYASGNGNDYVSWTYDDATKTFTVTE

SEQ ID NO: 839 - protein, 56 aa, Artificial Sequence
  Synthetic polypeptide
TYKLILNGKTLKGETTTEAVDFYYAFSVFFSYATSNRTDYVSWTYDDATKTFTVTE

SEQ ID NO: 840 - protein, 56 aa, Artificial Sequence
  Synthetic polypeptide
TYKLILNGKTLKGETTTEAVDFYYAFSVFFSYALENGRDYVSWTYDDATKTFTVTE

SEQ ID NO: 841 - protein, 56 aa, Artificial Sequence
  Synthetic polypeptide
TYKLILNGKTLKGETTTEAVDFYYAFSVFFSYALDNSVDYVSWTYDDATKTFTVTE

SEQ ID NO: 842 - protein, 56 aa, Artificial Sequence
  Synthetic polypeptide
TYKLILNGKTLKGETTTEAVDFYYAFSVFFSYATDNVRDYVSWTYDDATKTFTVTE

SEQ ID NO: 843 - protein, 56 aa, Artificial Sequence
  Synthetic polypeptide
TYKLILNGKTLKGETTTEAVDFYYAFSVFFSYAVENRFDYVSWTYDDATKTFTVTE

SEQ ID NO: 844 - protein, 56 aa, Artificial Sequence
  Synthetic polypeptide
TYKLILNGKTLKGETTTEAVDFYYAFSVFFSYAVGNTADYVSWTYDDATKTFTVTE

SEQ ID NO: 845 - protein, 56 aa, Artificial Sequence
  Synthetic polypeptide
TYKLILNGKTLKGETTTEAVDFYYAFSVFFSYAEGNRADYVSWTYDDATKTFTVTE

SEQ ID NO: 846 - protein, 56 aa, Artificial Sequence
  Synthetic polypeptide
TYKLILNGKTLKGETTTEAVDFYYAFSVFFSYALGNGTDYVSWTYDDATKTFTVTE

SEQ ID NO: 847 - protein, 56 aa, Artificial Sequence
  Synthetic polypeptide
TYKLILNGKTLKGETTTEAVDFYYAFSVFFSYATSNGPDYVSWTYDDATKTFTVTE

SEQ ID NO: 848 - protein, 56 aa, Artificial Sequence
  Synthetic polypeptide
TYKLILNGKTLKGETTTEAVDFYYAFSVFFSYATGNASDYVSWTYDDATKTFTVTE

SEQ ID NO: 849 - protein, 56 aa, Artificial Sequence
  Synthetic polypeptide
TYKLILNGKTLKGETTTEAVDFYYAFSVFFSYASENGADYVSWTYDDATKTFTVTE

SEQ ID NO: 850 - protein, 56 aa, Artificial Sequence
  Synthetic polypeptide
TYKLILNGKTLKGETTTEAVDFYYAFSVFFSYALGNVRDYVSWTYDDATKTFTVTE

SEQ ID NO: 851 - protein, 56 aa, Artificial Sequence
  Synthetic polypeptide
TYKLILNGKTLKGETTTEAVDFYYAFSVFFSYATTNNSDYVSWTYDDATKTFTVTE

SEQ ID NO: 852 - protein, 56 aa, Artificial Sequence
  Synthetic polypeptide
TYKLILNGKTLKGETTTEAVDFYYAFSVFFSYASSNGQDYVSWTYDDATKTFTVTE

SEQ ID NO: 853 - protein, 56 aa, Artificial Sequence
  Synthetic polypeptide
TYKLILNGKTLKGETTTEAVDFYYAFSVFFSYASMNGADYVSWTYDDATKTFTVTE

SEQ ID NO: 854 - protein, 56 aa, Artificial Sequence
  Synthetic polypeptide
TYKLILNGKTLKGETTTEAVDFYYAFSVFFSYAVWNKIDYVSWTYDDATKTFTVTE

SEQ ID NO: 855 - protein, 56 aa, Artificial Sequence
  Synthetic polypeptide
TYKLILNGKTLKGETTTEAVDFYYAFSVFFSYATGNGTDYVSWTYDDATKTFTVTE

SEQ ID NO: 856 - protein, 56 aa, Artificial Sequence
  Synthetic polypeptide
TYKLILNGKTLKGETTTEAVDFYYAFSVFFSYASLNQADYVSWTYDDATKTFTVTE

SEQ ID NO: 857 - protein, 56 aa, Artificial Sequence
  Synthetic polypeptide
TYKLILNGKTLKGETTTEAVDFYYAFSVFFSYATSNRVDYVSWTYDDATKTFTVTE

SEQ ID NO: 858 - protein, 56 aa, Artificial Sequence
  Synthetic polypeptide
TYKLILNGKTLKGETTTEAVDFYYAFSVFFSYASSNSSKHVEWTYDDATKTFTVTE

SEQ ID NO: 859 - protein, 56 aa, Artificial Sequence
  Synthetic polypeptide
TYKLILNGKTLKGETTTEAVDFYYAFSVFFSYASSNSSSCIGWTYDDATKTFTVTE

SEQ ID NO: 860 - protein, 56 aa, Artificial Sequence
  Synthetic polypeptide
TYKLILNGKTLKGETTTEAVDFYYAFSVFFSYASSNSSECVSWTYDDATKTFTVTE

SEQ ID NO: 861 - protein, 56 aa, Artificial Sequence
  Synthetic polypeptide
TYKLILNGKTLKGETTTEAVDFYYAFSVFFSYASSNSSACVMWTYDDATKTFTVTE

SEQ ID NO: 862 - protein, 56 aa, Artificial Sequence
  Synthetic polypeptide
TYKLILNGKTLKGETTTEAVDFYYAFSVFFSYASSNSSSCVVWTYDDATKTFTVTE

SEQ ID NO: 863 - protein, 56 aa, Artificial Sequence
  Synthetic polypeptide
TYKLILNGKTLKGETTTEAVDFYYAFSVFFSYASSNSSTCISWTYDDATKTFTVTE

SEQ ID NO: 864 - protein, 56 aa, Artificial Sequence
  Synthetic polypeptide
TYKLILNGKTLKGETTTEAVDFYYAFSVFFSYASSNSSSCVYWTYDDATKTFTVTE